(19) United States
(12) Patent Application Publication
(10) Pub. No.: US 2025/0305053 A1
(43) Pub. Date: Oct. 2, 2025

(54) NEW NRG1 FUSIONS, FUSION JUNCTIONS AND METHODS FOR DETECTING THEM

(71) Applicant: MERUS N.V., Utrecht (NL)

(72) Inventors: Ernesto Isaac WASSERMAN, Utrecht (NL); Jeroen Jilles LAMMERTS VAN BUEREN, Utrecht (NL)

(21) Appl. No.: 18/566,982

(22) PCT Filed: Jun. 1, 2022
(86) PCT No.: PCT/NL2022/050302

§ 371 Date: Dec. 4, 2023

(30) Foreign Application Priority Data
Jun. 3, 2021 (NL) 2028384
Dec. 3, 2021 (NL) 2030006

(51) Int. Cl. C12Q 1/6886

(52) U.S. Cl. CPC C12Q 1/6886; C12Q 2600/156

(57) ABSTRACT

The disclosure relates to the field of neuregulin-1 (NRG1) fusions, methods for detecting such, identifying or diagnosing patients with such fusions and methods of treatment of a cancer, a tumor or an aberrant cell comprising an NRG1 fusion. Also, it relates to the field of therapeutic (human) compounds for the treatment of subjects with an ErbB-2/ErbB-3 positive cancer that comprise a NRG1 fusion.

### CROSS REFERENCE TO RELATED APPLICATIONS

This application is a 35 U.S.C. § 371 National Phase Application of PCT/NL2022/050302 filed Jun. 1, 2022, the content of which is hereby incorporated by reference.

### REFERENCE TO A SEQUENCE LISTING

This application includes a Sequence Listing submitted electronically via EFS-Web (Name: 4096_0550002_Seqlisting_ST25.txt, File Size: 1,046,300 bytes, and Creation Date: Nov. 21, 2024), which is hereby incorporated by reference in its entirety.

### FIELD OF THE INVENTION

The disclosure relates to the field of neuregulin-1 (NRG1) fusions, methods for detecting such, identifying or diagnosing patients with such fusions and methods of treatment of a cancer, a tumor or an aberrant cell comprising an NRG1 fusion. Also, it relates to the field of therapeutic (human) compounds for the treatment of subjects with an ErbB-2/ErbB-3 positive cancer that comprise a NRG1 fusion.

### BACKGROUND OF THE INVENTION

Proteolytic processing of the extra cellular domain of transmembrane NRG1 isoforms release soluble factors. HRG1-ß1 is one of the proteins encoded by the NRG1 gene. NRG1 contains an Ig domain and an EGF-like domain that is necessary for direct binding to receptor tyrosine kinases ErbB-3 and ErbB-4. The NRG1 gene and the isoforms are known under a number of different aliases such as: Neuregulin 1; Pro-NRG1; HRGA; SMDF; HGL; GGF; NDF; NRG1 Intronic Transcript 2 (Non-Protein Coding); Heregulin, Alpha (45 kD, ERBB2 P185-Activator); Acetylcholine Receptor-Inducing Activity; Pro-Neuregulin-1, Membrane-Bound Isoform; Sensory And Motor Neuron Derived Factor; Neu Differentiation Factor; Glial Growth Factor 2; NRG1-IT2; MSTP131; MST131; ARIA; GGF2; HRG1; and HRG. External Ids for NRG1 Gene are HGNC: 7997; Entrez Gene: 3084; Ensembl: ENSG00000157168; OMIM: 142445 and UniProtKB: Q02297.

Isoforms of NRG1 are made by alternative splicing, and include forms that are transmembrane, externally membrane bound, shed, secreted or intracellular (Falls in Exp Cell Res 284: 14-30, 2003; Hayes and Gullick in J. Mammary Gland Biol Neoplasia 13: 205-214, 2008). They bind to ErbB-3 or ErbB-4, which is understood to promote heterodimer formation with ErbB-2 (HER2). Although the NRG1-encoded proteins are usually thought of as mitogens, they can also be powerfully proapoptotic: in particular, expressing NRG1 in cells can cause apoptosis of the expressing cell (cf. Weinstein et al. in oncogene 17: 2107-2113, 1998).

Given the diverse array of tumor types that harbor NRG1 fusions, there is a need for quicker and more robust diagnosis, and a need for identification of heretofore unknown NRG1 fusions, based on new fusion partners and new break points between NRG1 and fusion partner.

### SUMMARY OF THE INVENTION

The present disclosure provides, in general terms, fusions involving NRG1 and novel fusion partners, as well as polypeptide fusions encoded therefrom. The present disclosure provides a fusion involving NRG1 and VAPB, NRG1 and PVALB, NRG1 and DAAM1, NRG1 and ASPH, NRG1 and ZFAT or NRG1 and DSCAML1 as novel fusion partners, denoted herein as VAPB-NRG1, PVALB-NRG1, DAAM1-NRG1, ASPH-NRG1, ZFAT-NRG1 and DSCAML1-NRG1, respectively as a general term. More in particular, this aspect of the present disclosure relates
to a polynucleotide comprising a VAPB nucleic acid sequence, or a portion of a VAPB nucleic acid sequence, fused with an NRG1 nucleic acid sequence, or a portion of a NRG1 nucleic acid sequence or
to a polynucleotide comprising a PVALB nucleic acid sequence, or a portion of a PVALB nucleic acid sequence, fused with an NRG1 nucleic acid sequence, or a portion of a NRG1 nucleic acid sequence or
to a polynucleotide comprising a DAAM1 nucleic acid sequence, or a portion of a DAAM1 nucleic acid sequence, fused with an NRG1 nucleic acid sequence, or a portion of a NRG1 nucleic acid sequence or
to a polynucleotide comprising a ASPH nucleic acid sequence, or a portion of a ASPH nucleic acid sequence, fused with an NRG1 nucleic acid sequence, or a portion of a NRG1 nucleic acid sequence or
to a polynucleotide comprising a ZFAT nucleic acid sequence, or a portion of a ZFAT nucleic acid sequence, fused with an NRG1 nucleic acid sequence, or a portion of a NRG1 nucleic acid sequence or
to a polynucleotide comprising a DSCAML1 nucleic acid sequence, or a portion of a DSCAML1 nucleic acid sequence, fused with an NRG1 nucleic acid sequence, or a portion of a NRG1 nucleic acid sequence.

Preferably, the VAPB nucleic acid sequence comprises or consists of (a portion of) any one the sequences SEQ ID NO: 17-23 or an allelic variant of any one of SEQ ID NOs: 17-23. Preferably, the NRG1 nucleic acid sequence comprises or consists of (a portion of) any one of the sequences SEQ ID NO: 125-138 or an allelic variant of any one of sequences SEQ ID NO: 125-138.

Alternatively or additionally, the present disclosure provides a polynucleotide comprising a portion of exon 1 of VAPB, or of an allelic variant of exon 1, fused with a portion of exon 2 of NRG1, or of an allelic variant of exon 2. Preferably, exon 1 of VAPB comprises or consists of SEQ ID NO: 17 and the allelic variant preferably is a variant of SEQ ID NO: 17. Exon 2 of NRG1 preferably comprises or consists of SEQ ID NO: 126 and the allelic variant preferably is a variant of SEQ ID NO: 126.

Preferably, the PVALB nucleic acid sequence comprises or consists of (a portion of) any one the sequences SEQ ID NO: 439-444 or an allelic variant of any one of SEQ ID NOs: 439-444. Preferably, the NRG1 nucleic acid sequence comprises or consists of (a portion of) any one of the sequences SEQ ID NO: 125-138 or an allelic variant of any one of sequences SEQ ID NO: 125-138.

Alternatively or additionally, the present disclosure provides a polynucleotide comprising a portion of exon 4 of PVALB, or of an allelic variant of exon 4, fused with a portion of exon 6 of NRG1, or of an allelic variant of exon 6. Preferably, exon 4 of PVALB comprises or consists of SEQ ID NO: 442 and the allelic variant preferably is a variant of SEQ ID NO: 442. Exon 6 of NRG1 preferably comprises or consists of SEQ ID NO: 130 and the allelic variant preferably is a variant of SEQ ID NO: 130.

Preferably, the DAAM1 nucleic acid sequence comprises or consists of (a portion of) any one the sequences SEQ ID NO: 606-631 or an allelic variant of any one of SEQ ID NOs: 606-631. Preferably, the NRG1 nucleic acid sequence comprises or consists of (a portion of) any one of the sequences SEQ ID NO: 125-138 or an allelic variant of any one of sequences SEQ ID NO: 125-138.

Alternatively or additionally, the present disclosure provides a polynucleotide comprising a portion of exon 1 of DAAM1, or of an allelic variant of exon 1, fused with a portion of exon 1 of NRG1, or of an allelic variant of exon 1. Preferably, exon 1 of DAAM1 comprises or consists of SEQ ID NO: 606 and the allelic variant preferably is a variant of SEQ ID NO: 606. Exon 1 of NRG1 preferably comprises or consists of SEQ ID NO: 125 and the allelic variant preferably is a variant of SEQ ID NO: 125.

Preferably, the ASPH nucleic acid sequence comprises or consists of (a portion of) any one the sequences SEQ ID NO: 637-662 or an allelic variant of any one of SEQ ID NOs: 637-662. Preferably, the NRG1 nucleic acid sequence comprises or consists of (a portion of) any one of the sequences SEQ ID NO: 125-138 or an allelic variant of any one of sequences SEQ ID NO: 125-138.

Alternatively or additionally, the present disclosure provides a polynucleotide comprising a portion of exon 22 of ASPH, or of an allelic variant of exon 22, fused with a portion of exon 2 of NRG1, or of an allelic variant of exon 2. Preferably, exon 22 of ASPH comprises or consists of SEQ ID NO: 658 and the allelic variant preferably is a variant of SEQ ID NO: 658. Exon 2 of NRG1 preferably comprises or consists of SEQ ID NO: 126 and the allelic variant preferably is a variant of SEQ ID NO: 126.

Preferably, the ZFAT nucleic acid sequence comprises or consists of (a portion of) any one the sequences SEQ ID NO: 830-846 or an allelic variant of any one of SEQ ID NOs: 830-846. Preferably, the NRG1 nucleic acid sequence comprises or consists of (a portion of) any one of the sequences SEQ ID NO: 125-138 or an allelic variant of any one of sequences SEQ ID NO: 125-138.

Alternatively or additionally, the present disclosure provides a polynucleotide comprising a portion of exon 12 of ZFAT, or of an allelic variant of exon 12, fused with a portion of exon 6 of NRG1, or of an allelic variant of exon 6. Preferably, exon 12 of ZFAT comprises or consists of SEQ ID NO: 841 and the allelic variant preferably is a variant of SEQ ID NO: 841. Exon 6 of NRG1 preferably comprises or consists of SEQ ID NO: 130 and the allelic variant preferably is a variant of SEQ ID NO: 130.

Preferably, the DSCAML1 nucleic acid sequence comprises or consists of (a portion of) any one the sequences SEQ ID NO: 870-903 or an allelic variant of any one of SEQ ID NOs: 870-903. Preferably, the NRG1 nucleic acid sequence comprises or consists of (a portion of) any one of the sequences SEQ ID NO: 125-138 or an allelic variant of any one of sequences SEQ ID NO: 125-138.

Alternatively or additionally, the present disclosure provides a polynucleotide comprising a portion of exon 3 of DSCAML1, or of an allelic variant of exon 3, fused with a portion of exon 2 of NRG1, or of an allelic variant of exon 2. Preferably, exon 3 of DSCAML1 comprises or consists of SEQ ID NO: 872 and the allelic variant preferably is a variant of SEQ ID NO: 872. Exon 2 of NRG1 preferably comprises or consists of SEQ ID NO: 126 and the allelic variant preferably is a variant of SEQ ID NO: 126.

Additionally, the present disclosure provides fusions involving NRG1 with previously undisclosed fusion junctions, as well as polypeptide fusions encoded therefrom. In particular, the present disclosure additionally provides fusions involving NRG1 and CADM1, denoted herein as CADM1-NRG1 as a general term, the present disclosure also provides a fusion involving NRG1 and CD44, denoted herein as CD44-NRG1 as a general term, the present disclosure provides a fusion involving NRG1 and SLC3A2, denoted herein as SLC3A2-NRG1 as a general term, the present disclosure provides a fusion involving NRG1 and VTCN1, denoted herein as VTCN1-NRG1 as a general term, the present disclosure provides a fusion involving NRG1 and CDH1, denoted herein as CDH1-NRG1 as a general term, the present disclosure provides a fusion involving NRG1 and CXADR, denoted herein as CXADR-NRG1 as a general term, the present disclosure provides a fusion involving NRG1 and GTF2E2, denoted herein as GTF2E2-NRG1 as a general term, the present disclosure provides a fusion involving NRG1 and CSMD1, denoted herein as CSMD1-NRG1 as a general term, the present disclosure provides a fusion involving NRG1 and PTN, denoted herein as PTN-NRG1 as a general term, the present disclosure provides a fusion involving NRG1 and ST14, denoted herein as ST14-NRG1 as a general term, the present disclosure provides a fusion involving NRG1 and THBS1, denoted herein as THBS1-NRG1 as a general term and the present disclosure provides a fusion involving NRG1 and AGRN, denoted herein as AGRN-NRG1 as a general term, the present disclosure also provides a fusion involving NRG1 and APP, denoted herein as APP-NRG1 as a general term, the present disclosure also provides a fusion involving NRG1 and WRN, denoted herein as WRN-NRG1 as a general term, the present disclosure also provides a fusion involving NRG1 and NOTCH2, denoted herein as NOTCH2-NRG1 as a general term, the present disclosure also provides a fusion involving NRG1 and CD74, denoted herein as CD74-NRG1 as a general term, the present disclosure also provides a fusion involving NRG1 and SDC4, denoted herein as SDC4-NRG1 as a general term, the present disclosure also provides a fusion involving NRG1 and SLC4A4, denoted herein as SLC4A4-NRG1 as a general term.

The present disclosure provides the following novel fusion junctions. In particular, the present disclosure provides a polynucleotide comprising a portion of exon 7 of CADM1, or an allelic variant of exon 7, fused with a portion of exon 6 of NRG1, or an allelic variant of exon 6. Preferably, exon 7 of CADM1 comprises or consists of SEQ ID NO: 39 and the allelic variant preferably is a variant of SEQ ID NO: 39. Exon 6 of NRG1 preferably comprises or consists of SEQ ID NO: 130 and the allelic variant preferably is a variant of SEQ ID NO: 130.

In particular, the present disclosure provides a polynucleotide comprising a portion of exon 5 of CD44, or an allelic variant of exon 5, fused with a portion of exon 2 of NRG1, or an allelic variant of exon 2. Preferably, exon 5 of CD44 comprises or consists of SEQ ID NO: 65 and the allelic variant preferably is a variant of SEQ ID NO: 65. Exon 2 of NRG1 preferably comprises or consists of SEQ ID NO: 126 and the allelic variant preferably is a variant of SEQ ID NO: 126.

In particular, the present disclosure provides a polynucleotide comprising a portion of exon 5 of CD44, or an allelic variant of exon 5, fused with a portion of exon 6 of NRG1, or an allelic variant of exon 6. Preferably, exon 5 of CD44 comprises or consists of SEQ ID NO: 65 and the allelic variant preferably is a variant of SEQ ID NO: 65. Exon 6 of NRG1 preferably comprises or consists of SEQ ID NO: 130 and the allelic variant preferably is a variant of SEQ ID NO: 130.

In particular, the present disclosure provides a polynucleotide comprising a portion of exon 1 of transcript version 6 of SLC3A2, or an allelic variant of exon 1 of transcript version 6, fused with a portion of exon 5 of NRG1, or an allelic variant of exon 2. Preferably, said exon of SLC3A2 comprises or consists of SEQ ID NO: 103 and the allelic variant preferably is a variant of SEQ ID NO: 103. Exon 5 of NRG1 preferably comprises or consists of SEQ ID NO: 129 and the allelic variant preferably is a variant of SEQ ID NO: 129.

In particular, the present disclosure provides a polynucleotide comprising a portion of exon 2 of VTCN1, or an allelic variant of exon 2, fused with a portion of exon 2 of NRG1, or an allelic variant of exon 2. Preferably, exon 2 of VTCN1 comprises or consists of SEQ ID NO: 169 and the allelic variant preferably is a variant of SEQ ID NO: 169. Exon 2 of NRG1 preferably comprises or consists of SEQ ID NO: 126 and the allelic variant preferably is a 35 variant of SEQ ID NO: 126.

In particular, the present disclosure provides a polynucleotide comprising a portion of exon 11 of CDH1, or an allelic variant of exon 11, fused with a portion of exon 2 of NRG1, or an allelic variant of exon 2. Preferably, exon 11 of CDH1 comprises or consists of SEQ ID NO: 198 and the allelic variant preferably is a variant of SEQ ID NO: 198. Exon 2 of NRG1 preferably comprises or consists of SEQ ID NO: 126 and the allelic variant preferably is a variant of SEQ ID NO: 126.

In particular, the present disclosure provides a polynucleotide comprising a portion of exon 1 of CXADR, or an allelic variant of exon 1, fused with a portion of exon 2 of NRG1, or an allelic variant of exon 2. Preferably, exon 1 of CXADR comprises or consists of SEQ ID NO: 219 and the allelic variant preferably is a variant of SEQ ID NO: 219. Exon 2 of NRG1 preferably comprises or consists of SEQ ID NO: 126 and the allelic variant preferably is a variant of SEQ ID NO: 126.

In particular, the present disclosure provides a polynucleotide comprising a portion of exon 2 of GTF2E2, or an allelic variant of exon 2, fused with a portion of exon 2 of NRG1, or an allelic variant of exon 2. Preferably, exon 2 of GTF2E2 comprises or consists of SEQ ID NO: 236 and the allelic variant preferably is a variant of SEQ ID NO: 236. Exon 2 of NRG1 preferably comprises or consists of SEQ ID NO: 126 and the allelic variant preferably is a variant of SEQ ID NO: 126.

In particular, the present disclosure provides a polynucleotide comprising a portion of exon 23 of CSMD1, or an allelic variant of exon 23, fused with a portion of exon 6 of NRG1, or an allelic variant of exon 6. Preferably, exon 23 of CSMD1 comprises or consists of SEQ ID NO: 279 and the allelic variant preferably is a variant of SEQ ID NO: 279. Exon 6 of NRG1 preferably comprises or consists of SEQ ID NO: 130 and the allelic variant preferably is a variant of SEQ ID NO: 130.

In particular, the present disclosure provides a polynucleotide comprising a portion of exon 4 of PTN, or an allelic variant of exon 4, fused with a portion of exon 2 of NRG1, or an allelic variant of exon 2. Preferably, exon 4 of PTN comprises or consists of SEQ ID NO: 318 and the allelic variant preferably is a variant of SEQ ID NO: 318. Exon 2 of NRG1 preferably comprises or consists of SEQ ID NO: 126 and the allelic variant preferably is a variant of SEQ ID NO: 126.

In particular, the present disclosure provides a polynucleotide comprising a portion of exon 11 of ST14, or an allelic variant of exon 11, fused with a portion of exon 6 of NRG1, or an allelic variant of exon 6. Preferably, exon 11 of ST14 comprises or consists of SEQ ID NO: 342 and the allelic variant preferably is a variant of SEQ ID NO: 342. Exon 6 of NRG1 preferably comprises or consists of SEQ ID NO: 130 and the allelic variant preferably is a variant of SEQ ID NO: 130.

In particular, the present disclosure provides a polynucleotide comprising a portion of exon 9 of THBS1, or an allelic variant of exon 9, fused with a portion of exon 6 of NRG1, or an allelic variant of exon 6. Preferably, exon 9 of THBS1 comprises or consists of SEQ ID NO: 386 and the allelic variant preferably is a variant of SEQ ID NO: 386. Exon 6 of NRG1 preferably comprises or consists of SEQ ID NO: 130 and the allelic variant preferably is a variant of SEQ ID NO: 130.

In particular, the present disclosure provides a polynucleotide comprising a portion of exon 12 of AGRN, or an allelic variant of exon 12, fused with a portion of exon 6 of NRG1, or an allelic variant of exon 6. Preferably, exon 12 of AGRN comprises or consists of SEQ ID NO: 416 and the allelic variant preferably is a variant of SEQ ID NO: 416. Exon 6 of NRG1 preferably comprises or consists of SEQ ID NO: 130 and the allelic variant preferably is a variant of SEQ ID NO: 130.

In particular, the present disclosure provides a polynucleotide comprising a portion of exon 4 of PVALB, or an allelic variant of exon 4, fused with a portion of exon 6 of NRG1, or an allelic variant of exon 6. Preferably, exon 4 of PVALB comprises or consists of SEQ ID NO: 442 and the allelic variant preferably is a variant of SEQ ID NO: 442. Exon 6 of NRG1 preferably comprises or consists of SEQ ID NO: 130 and the allelic variant preferably is a variant of SEQ ID NO: 130.

In particular, the present disclosure provides a polynucleotide comprising a portion of exon 2 of transcript version 3 of SLC3A2, or an allelic variant of exon 2 of transcript version 3, fused with a portion of exon 6 of NRG1, or an allelic variant of exon 6. Preferably, said exon of SLC3A2 comprises or consists of SEQ ID NO: 457 and the allelic variant preferably is a variant of SEQ ID NO: 457. Exon 6 of NRG1 preferably comprises or consists of SEQ ID NO: 130 and the allelic variant preferably is a variant of SEQ ID NO: 130.

In particular, the present disclosure provides a polynucleotide comprising a portion of exon 14 of APP, or an allelic variant of exon 14, fused with a portion of exon 6 of NRG1, or an allelic variant of exon 6. Preferably, exon 14 of APP comprises or consists of SEQ ID NO: 501 and the allelic variant preferably is a variant of SEQ ID NO: 501. Exon 6 of NRG1 preferably comprises or consists of SEQ ID NO: 130 and the allelic variant preferably is a variant of SEQ ID NO: 130.

In particular, the present disclosure provides a polynucleotide comprising a portion of exon 33 of WRN, or an allelic variant of exon 33, fused with a portion of exon 6 of NRG1, or an allelic variant of exon 6. Preferably, exon 33 of WRN comprises or consists of SEQ ID NO: 562 and the allelic variant preferably is a variant of SEQ ID NO: 562. Exon 6 of NRG1 preferably comprises or consists of SEQ ID NO: 130 and the allelic variant preferably is a variant of SEQ ID NO: 130.

In particular, the present disclosure provides a polynucleotide comprising a portion of exon 6 of NOTCH2, or an allelic variant of exon 6, fused with a portion of exon 6 of NRG1, or an allelic variant of exon 6. Preferably, exon 6 of NOTCH2 comprises or consists of SEQ ID NO: 700 and the allelic variant preferably is a variant of SEQ ID NO: 700. Exon 6 of NRG1 preferably comprises or consists of SEQ ID NO: 130 and the allelic variant preferably is a variant of SEQ ID NO: 130.

In particular, the present disclosure provides a polynucleotide comprising a portion of exon 2 of CD74, or an allelic variant of exon 2, fused with a portion of exon 2 of NRG1, or an allelic variant of exon 2. Preferably, exon 2 of CD74 comprises or consists of SEQ ID NO: 720 and the allelic variant preferably is a variant of SEQ ID NO: 720. Exon 2 of NRG1 preferably comprises or consists of SEQ ID NO: 126 and the allelic variant preferably is a variant of SEQ ID NO: 126.

In particular, the present disclosure provides a polynucleotide comprising a portion of exon 2 of SDC4, or an allelic variant of exon 2, fused with a portion of exon 2 of NRG1, or an allelic variant of exon 2. Preferably, exon 2 of SDC4 comprises or consists of SEQ ID NO: 746 and the allelic variant preferably is a variant of SEQ ID NO: 746. Exon 2 of NRG1 preferably comprises or consists of SEQ ID NO: 126 and the allelic variant preferably is a variant of SEQ ID NO: 126.

In particular, the present disclosure provides a polynucleotide comprising a portion of exon 4 of SDC4, or an allelic variant of exon 4, fused with a portion of exon 2 of NRG1, or an allelic variant of exon 2. Preferably, exon 4 of SDC4 comprises or consists of SEQ ID NO: 748 and the allelic variant preferably is a variant of SEQ ID NO: 748. Exon 2 of NRG1 preferably comprises or consists of SEQ ID NO: 126 and the allelic variant preferably is a variant of SEQ ID NO: 126.

In particular, the present disclosure provides a polynucleotide comprising a portion of exon 14 of SLC4A4, or an allelic variant of exon 14, fused with a portion of exon 6 of NRG1, or an allelic variant of exon 6. Preferably, exon 14 of SLC4A4 comprises or consists of SEQ ID NO: 780 and the allelic variant preferably is a variant of SEQ ID NO: 780. Exon 6 of NRG1 preferably comprises or consists of SEQ ID NO: 130 and the allelic variant preferably is a variant of SEQ ID NO: 130.

The herein provided NRG1 fusions have all been observed in patient derived samples, in particular patients diagnosed with cancer.

The disclosed identification of these NRG1 fusions and genetic rearrangements provide new methods for determining the presence of NRG1 polynucleotide fusions or polypeptides in or from a biological sample, methods for assaying activity of NRG1 polypeptide fusions, methods for diagnosing a cancer, a tumor or an aberrant cell that express an NRG1 polypeptide fusion, and methods for treating a cancer, a tumor or an aberrant cell that express an NRG1 polypeptide fusion and/or inhibiting the progression of oncogenesis characterized by the expression of an NRG1 polynucleotide fusion or polypeptide. These and more aspects are thus also provided by the present disclosure and described in more detail below.

The present disclosure also provides polypeptide fusions encoded by any one of the polynucleotide fusions selected from VAPB-NRG1, CADM1-NRG1 CD44-NRG1, SLC3A2-NRG1, VTCN1-NRG1, CDH1-NRG1, CXADR-NRG1, GTF2E2-NRG1, CSMD1-NRG1, PTN-NRG1, ST14-NRG1, THBS1-NRG1, AGRN-NRG1, PVALB-NRG1, APP-NRG1, WRN-NRG1, ASPH-NRG1, NOTCH2-NRG1, CD74-NRG1, SDC4-NRG1, SLC4A4-NRG1, ZFAT-NRG1 and DSCAML1-NRG1. Any one of the polypeptide fusions of the present disclosure, when expressed by an aberrant cell as mentioned herein, preferably also comprises an EGF-like domain of NRG1. Any polynucleotide fusion of the present disclosure, when contained by an aberrant cell as mentioned herein, preferably comprises the NRG1 exons coding for an EGF-like domain, such as exons 6, 7 or 8 of NRG1, more preferably exons 6 and 7 of the NRG1 polynucleotide sequence.

Also provided is a vector comprising a polynucleotide fusion of the present disclosure, a recombinant host cell comprising said polynucleotide and/or said vector. Also provided is a method of making a polypeptide of the present disclosure, comprising maintaining said recombinant host cell under conditions suitable for expression of the polynucleotide, whereby the polynucleotide fusion is expressed and a polypeptide fusion is produced, followed by isolating or purifying the polypeptide. Also provided is a method for making a recombinant host cell comprising introducing said vector into a host cell.

Also provided is a nucleic acid probe, primer or primer pair for detection of a polynucleotide fusion of the present disclosure and a detection assay comprising a nucleic acid probe, primer or primer pair for detection of the presence of a polynucleotide fusion of the present disclosure. Such a nucleic acid probe, primer or primer pair of the present disclosure has a preferred length of 10-40 nucleotides.

Also provided is a first antibody, or a set of a first and a second antibody, for detection of a polypeptide encoded by a polynucleotide fusion of the present disclosure and a detection assay comprising the first antibody or the set of a first and second antibodies for detection of the presence of a polypeptide encoded by a polynucleotide fusion of the present disclosure. Preferably, the first antibody binds a polypeptide fusion selected from VAPB-NRG1, CADM1-NRG1, CD44-NRG1, SLC3A2-NRG1, VTCN1-NRG1, CDH1-NRG1, CXADR-NRG1, GTF2E2-NRG1, CSMD1-NRG1, PTN-NRG1, ST14-NRG1, THBS1-NRG1, AGRN-NRG1, PVALB-NRG1, APP-NRG1, WRN-NRG1, ASPH-NRG1, NOTCH2-NRG1, CD74-NRG1, SDC4-NRG1, SLC4A4-NRG1, ZFAT-NRG1 and DSCAML1-NRG1 and the set of first and second antibodies binds VAPB and NRG1, or CADM1 and NRG1, or CD44 and NRG1, or SLC3A2 and NRG1, or VTCN1 and NRG1 or CDH1 and NRG1, or CXADR and NRG1, or GTF2E2 and NRG1, or CSMD1 and NRG1, or PTN and NRG1, or ST14 and NRG1, or THBS1 and NRG1, or AGRN and NRG1 or PVALB and NRG1, APP and NRG1, or WRN and NRG1, or ASPH and NRG1, or NOTCH2 and NRG1, or CD74 and NRG1, or SDC4 and NRG1, or SLC4A4 and NRG1, or ZFAT and NRG1, or DSCAML1 and NRG1, respectively.

The present disclosure, including methods to identify or detect NRG1 fusions in human subjects, further encompasses methods of diagnosis, treatment selection, and therapeutic treatment with medicaments and combinations thereof for amelioration of NRG1-fusion related maladies, including solid tumors. Among others, the present disclosure provides straight forward means or assays to quickly assess whether a subject is suffering, or prone to suffering, from a cancer, a tumor, or an aberrant cell. Such NRG1 fusion information can be advantageously used as a biomarker in a diagnostic tool.

Thus, there is also provided a method for identifying in a sample any one of the polynucleotide fusions as mentioned herein, or a polypeptide encoded therefrom, said method comprising testing a sample obtained from a subject to detect the presence of the fusion in the sample.

There is also provided a method for detecting in a sample the presence of any one of the polynucleotide fusions as mentioned herein, or a polypeptide encoded therefrom, said method comprising testing a sample obtained from a subject and detecting the presence of the fusion in the sample.

There is also provided a method for establishing whether an aberrant cell, such as a cancer cell or a tumor cell, from a subject comprises any one of the polynucleotide fusions as mentioned herein, or a polypeptide encoded therefrom, said method comprising testing a cell, or the polynucleotide or polypeptide contents of said cell, obtained from the subject for the presence of the fusion in the sample.

There is also provided a method for identifying a subject as carrying any one of the polynucleotide fusions as mentioned herein, or a polypeptide encoded therefrom, said method comprising testing a sample obtained from a subject and detecting the presence of the fusion in the sample.

The testing of the sample as mentioned herein is preferably part of an in vitro analysis or such method, or an ex vivo analysis or such method.

Preferably, the subject is a mammalian or human subject and the polynucleotides or polypeptides are mammalian or human polynucleotides or polypeptides.

Preferably, any polynucleotide fusion as mentioned herein and/or the polypeptide encoded therefrom as mentioned herein, are isolated and/or purified or substantially isolated or substantially purified.

The present disclosure relates to a subject suffering from, or suspect of suffering from a cancer, in particular a solid cancer or a tumor. In particular, such a cancer is an adenocarcinoma, more in particular mucinous adenocarcinoma, a pancreatic cancer, more in particular a pancreatic adenocarcinoma, or a renal cell carcinoma, cholangiocarcinoma, a brain cancer, glioblastoma, a cholangiocarcinoma, a glioma, a pancreatic ductal adenocarcinoma, a sarcoma, a bladder cancer, a colon cancer, a rectal cancer, a colorectal cancer, a gallbladder cancer, a head and neck cancer, a prostate cancer, a uterus cancer, a breast cancer, an ovarian cancer, a liver cancer, an endometrial cancer, a lung cancer, in particular a non-small cell lung cancer or an invasive mucinous adenocarcinoma. The cancer can be a primary cancer or metastatic cancer.

The present disclosure also provides a method of treating a subject having an ErbB-2 and/or ErbB-3 positive aberrant cell, such as a cancer or tumor, said aberrant cell comprising a polynucleotide fusion as mentioned herein, and/or such a cell expressing a polypeptide fusion encoded therefrom, said method comprising administering to the subject a therapeutic amount of an ErbB-2 and/or ErbB-3 targeting agent. Preferably, the administration of said agent is preceded by detecting a polynucleotide or polypeptide fusion as mentioned herein.

The present disclosure also provides a method for inhibiting the progression in a subject having an ErbB-2 and ErbB-3 positive aberrant cell, said aberrant cell comprising a polynucleotide fusion as mentioned herein, and/or expressing a polypeptide fusion encoded therefrom, said method comprising administering to the subject a therapeutic amount of an ErbB-2 and/or ErbB-3 targeting agent. Preferably, the administration of said agent is preceded by detecting a polynucleotide or polypeptide fusion as mentioned herein.

The present disclosure also provides an ErbB-2 and/or ErbB-3 targeting agent for use in the treatment of a subject that has an ErbB-2 and ErbB-3 positive cancer or tumor comprising a polynucleotide fusion as mentioned herein, and/or expressing a polypeptide fusion encoded therefrom, said treatment comprising administering a therapeutic amount of the ErbB-2 and/or ErbB-3 targeting agent to the subject. Preferably, the administration of said agent is preceded by detecting a polynucleotide or polypeptide fusion as mentioned herein.

The present disclosure also provides a method for diagnosing a subject of having an aberrant cell that comprises a polynucleotide fusion as mentioned herein, or a polypeptide encoded therefrom, the method comprising testing a sample obtained from a subject to detect the presence of the fusion in the sample.

The present disclosure also provides a method of treatment or use of an ERB2 and/or Erb3 targeting agent, wherein a subject is screened for the presence of an NRG1 fusion as mentioned herein, followed by administration of the Erb2 and/or Erb3 agent.

The present disclosure also includes in vivo models, such as xenograft or transgenic animal models expressing within their genome or engrafted aberrant cells comprising a polynucleotide fusion as mentioned herein, and/or expressing a polypeptide fusion encoded therefrom, and treatment of such models with an ERB2 and/or Erb3 targeting agent or other targeting agent for evaluation of therapeutic activity of such agent. Preferably, the animal model is a non-human animal model.

### DETAILED DESCRIPTION OF THE INVENTION

The present disclosure provides fusions involving NRG1 and novel fusion partners, as well as polypeptide fusions encoded therefrom. Also, the present disclosure provides fusions involving NRG1 with previously undisclosed fusion junctions, as well as polypeptide fusions encoded therefrom.

In general terms, the present disclosure provides fusions involving NRG1 and a fusion partner selected from VAPB, CADM1, CD44, SLC3A2, VTCN1, CDH1, CXADR, GTF2E2, CSMD1, PTN, ST14, THBS1, AGRN, PVALB, APP, WRN, DAAM1, ASPH, NOTCH2, CD74, SDC4, SLC4A4, ZFAT or DSCAML1. Said fusions are denoted therein as VAPB-NRG1, CADM1-NRG1, CD44-NRG1, SLC3A2-NRG1, VTCN1-NRG1, CDH1-NRG1, CXADR-NRG1, GTF2E2-NRG1, CSMD1-NRG1, PTN-NRG1, ST14-NRG1, THBS1-NRG1, AGRN-NRG1, PVALB-NRG1, APP-NRG1, WRN-NRG1, DAAM1-NRG1, ASPH-NRG1, NOTCH2-NRG1, CD74-NRG1, SDC4-NRG1, SLC4A4-NRG1, ZFAT-NRG1 or DSCAML1-NRG1. The presence of any one of the NRG1 fusions of the present disclosure, be it a polynucleotide or a polypeptide translated therefrom, is indicative of the presence of an aberrant cell, such as a cancer or a tumor.

### NRG1

The NRG1-gene codes for various isoforms of NRG1. Various isoforms and their expected function are described in Adelaide et al. Genes Chromosomes Cancer, August; 37(4):333-45 (2003). GGF and GGF2 isoforms contain a kringle-like sequence plus Ig and EGF-like domains; and the SMDF isoform shares only the EGF-like domain with other isoforms. The receptors for NRG1 isoforms are the ErbB family of tyrosine kinase transmembrane receptors. The family is also referred to as the human epidermal growth factor (EGF) receptor family (HER). The family has four members: ErbB (Erythroblastoma)-1, ErbB-2, ErbB-3 and ErbB-4. The receptors (reviewed in Yarden and Pines, Nat Rev Cancer. 2012 Jul. 12; 12(8):553-63) are widely expressed on epithelial cells. Upregulation of HER receptors or their ligands, such as heregulin (HRG) or epidermal growth factor (EGF), is a frequent event in human cancer (Wilson, Fridlyand et al., Nature. 2012 Jul. 26; 487(7408): 505-509). Overexpression of ErbB-1 and ErbB-2 in particular occurs in epithelial tumors and is associated with tumor invasion, metastasis, resistance to chemotherapy, and poor prognosis (Zhang, H., Berezov, A., Wang, Q., Zhang, G., Drebin, J., Murali, R., et al. (2007) Erbb receptors: from oncogenes to targeted cancer therapies. J. Clin. Invest., 117, 2051-2058). In the normal breast, ErbB-3 has been shown to be important in the growth and differentiation of luminal epithelium. For instance, loss/inhibition of ErbB-3 results in selective expansion of the basal over the luminal epithelium (Balko, Miller et al., 2012 PNAS Jan. 3, 2012 109 (1) 221-226). Binding of ligand to the extracellular domain of a receptor tyrosine kinase induces receptor dimerization, both between the same (homodimerization) and different (heterodimerization) receptor subtypes. Dimerization can activate the intracellular tyrosine kinase domains, which undergo autophosphorylation and, in turn, can activate a number of downstream pro-proliferative signaling pathways, including those mediated by mitogen-activated protein kinases (MAPK) and the pro-survival pathway Akt (reviewed in Yarden and Pines, 2012). ErbB-3 can be activated by engagement of its ligands. These ligands include but are not limited to neuregulin (NRG) and heregulin (HRG), and NRG1 fusions.

Various NRG1 fusion genes are described in Dhanasekaran et al. Nature Communications 5:5893, 2014, and in Jonna et al., Clin Cancer Res Aug. 15 2019, 25 (16) 4966-4972.

The term “NRG1” as used herein is not meant to be limited to any form, such as a protein, RNA, mRNA, cDNA or DNA sequence, but to include all such forms, unless the context makes clear which form is intended. The NRG1 gene and the isoforms are known under a number of different aliases such as: Neuregulin 1; Pro-NRG1; HRGA; SMDF; HGL; GGF; NDF; NRG1 Intronic Transcript 2 (Non-Protein Coding); Heregulin, Alpha (45 kD, ERBB2 P185-Activator); Acetylcholine Receptor-Inducing Activity; Pro-Neuregulin-1, Membrane-Bound Isoform; Sensory And Motor Neuron Derived Factor; Neu Differentiation Factor; Glial Growth Factor 2; NRG1-IT2; MSTP131; MST131; ARIA; GGF2; HRG1; and HRG. External Ids for NRG1 Gene are HGNC: 7997; Entrez Gene: 3084; Ensembl: ENSG00000157168; OMIM: 142445 and UniProtKB: Q02297. For the avoidance of doubt, with an NRG1 sequence is meant any polynucleotide sequence, including the coding sequence or any exon, but also any polypeptide sequence, or any portion thereof, such as from NCBI Reference Sequence NM_001159999.3 or allelic variants of NM_001159999.3. Preferably, the full polynucleotide sequence of NRG1 of the present disclosure as transcribed is according to SEQ ID NO: 138 and the translated NRG1 polypeptide sequence is according to SEQ ID NO: 152.

Preferably, the part of the polynucleotide fusion that encodes an NRG1 protein sequence, or exon of NRG1, further comprises or encodes an EGF-like domain of NRG1. The NRG1-gene, for example the 3′ end of the NRG1 gene, in the fusion preferably comprises the coding sequence of exons 6, 7 or 8. This domain is encoded by a 3′ located part of the NRG1 gene (e.g. exons 6-8) and is necessary for binding to ErbB-3. An NRG1-fusion gene preferably comprises a nucleic acid sequence coding for an EGF-like domain of NRG1. The NRG1-fusions of the disclosure preferably retain an in-frame coding region for this EGF-like domain at the 3′ end of the fusion molecule. An EGF-like domain is a sequence of typically about thirty to forty amino acid residues long of which the prototype is found in the sequence of epidermal growth factor (EGF) [PMID: 2288911, PMID: 6334307, PMID: 1522591, PMID: 6607417, PMID: 3282918, PMID: 11498013]. It is known to be present, in a more or less conserved form, in a large number of other, mostly animal proteins. A common feature of EGF-like domains is that they are found in the extracellular domain of membrane-bound proteins or in proteins known to be secreted (exception: prostaglandin G/H synthase). The EGF domain typically includes six cysteine residues which have been shown (in EGF) to be involved in disulphide bonds. The main structure is a two-stranded beta-sheet followed by a loop to a C-terminal short two-stranded sheet. Subdomains between the conserved cysteines vary in length. An exemplary EGF-like domain of the present disclosure is preferably comprised by exons 6-8, more preferably 6 and 7 of NCBI Reference Sequence NM_001159999.3 but the present disclosure is relevant for any functional EGF-like domain of NRG1. An EGF-like domain of NRG1 thus preferably comprises exemplary sequence HLVKCAEKEKTFCVNGGECFMVKDLSNPSRYLCKCPNEFTGDRCQNYVMASF (SEQ ID: 163), or an allelic variant thereof having at least 85% identity thereto, or at least 90%, 92%, 94%, 95%, 96% or even at least 98% identity thereto.

Any NRG1 fusion polynucleotide of the present disclosure preferably comprises at least a 5′ portion of VAPB, CADM1, CD44, SLC3A2, VTCN1, CDH1, CXADR, GTF2E2, CSMD1, PTN, ST14, THBS1, AGRN, PVALB, APP, WRN, DAAM1, ASPH, NOTCH2, CD74, SDC4, SLC4A4, ZFAT or DSCAML1 as a fusion partner, fused to a 3′ portion of an NRG1 nucleic acid sequence. The translated polypeptide fusion comprises a free C-terminus of the NRG1 fusion polypeptide and a free N-terminus of the fusion partner. To reflect this orientation at the molecular fusion level, any fusion partner of NRG1 is mentioned first, followed by NRG1 as its fusion partner.

### VAPB

VAPB or Vesicle-associated membrane protein-associated protein B/C is known under a number of different names such as VAMP Associated Protein B and C; VAMP (Vesicle-Associated Membrane Protein)-Associated Protein B And C; VAP-B; VAPB; ALS8; VAMP-Associated 33 KDa Protein; VAMP-Associated Protein B/C; VAMP-B/VAMP-C; VAP-B/VAP-C; VAMP-B and VAP-C. External Ids for VAPB Gene are HGNC: 12649; Entrez Gene: 9217; Ensembl: ENSG00000124164; OMIM: 605704; and UniProtKB: 095292. The term “VAPB” as used herein is not meant to be limited to any form, such as a protein, RNA, mRNA, cDNA or DNA sequence, but to include all such forms, unless the context makes clear which form is intended. For the avoidance of doubt, with a VAPB sequence is meant any polynucleotide sequence, including the coding sequence or any exon, but also any polypeptide sequence, or any portion thereof, such as from NCBI Reference Sequence NM_004738.4, or any allelic variant thereof.

### CADM1

CADM1 or Cell Adhesion Molecule 1 is known under a number of different names such as Tumor Suppressor In Lung Cancer 1; TSLC1; TSLC-1; Spermatogenic Immunoglobulin Superfamily; SgIgSF; SgIGSF; Immunoglobulin Superfamily Member 4; IGSF4; IgSF4; IGSF4A; Synaptic Cell Adhesion Molecule; SynCAM1; SYNCAM1; SynCAM; SYNCAM; Nectin-Like Protein 2; NECL2; NECL-2; Nectin-Like 2; Necl-2; RA175; ST17; BL2; Immunoglobulin Superfamily, Member 4D Variant 1; Immunoglobulin Superfamily, Member 4D Variant 2; Immunoglobulin Superfamily, Member 4; TSLC1/Nectin-Like 2/IGSF4; Truncated CADM1 Protein and STSLC-1. External Ids for CADM1 are HGNC: 5951; Entrez Gene: 23705; Ensembl: ENSG00000182985; OMIM: 605686; and UniProtKB: Q9BY67. The term “CADM1” as used herein is not meant to be limited to any form, such as a protein, RNA, mRNA, cDNA or DNA sequence, but to include all such forms, unless the context specifies the intended form as meant. For the avoidance of doubt, with a CADM1 sequence is meant any polynucleotide sequence, including the coding sequence or any exon, but also any polypeptide sequence, or any portion thereof, such as from NCBI Reference Sequence NM_001301045.1, or any allelic variant thereof.

### CD44

CD44 or is known under a number of different names such as CD44 Molecule (Indian Blood Group); Hematopoietic Cell E- And L-Selectin Ligand; GP90 Lymphocyte Homing/Adhesion Receptor; Chondroitin Sulfate Proteoglycan 8; Extracellular Matrix Receptor III; Heparan Sulfate Proteoglycan; Phagocytic Glycoprotein 1; Hyaluronate Receptor; Hermes Antigen; CD44 Antigen; ECMR-III; HUTCH-I; Epican; MDU2; MDU3; MIC4; LHR; CD44 Antigen (Homing Function And Indian Blood Group System); Homing Function And Indian Blood Group System; Cell Surface Glycoprotein CD44; Indian Blood Group Antigen; Phagocytic Glycoprotein I; Soluble CD44; CDW44; CSPG8; HCELL; CDw44; PGP-1; MC56; Pgp1; and IN. External Ids for CD44 Gene are HGNC: 1681; Entrez Gene: 960; Ensembl: ENSG00000026508; OMIM: 107269; and UniProtKB: P16070. The term “CD44” as used herein is not meant to be limited to any form, such as a protein, RNA, mRNA, cDNA or a DNA sequence, but to include all such forms, unless the context specifies the intended form as meant. For the avoidance of doubt, with a CD44 sequence is meant any polynucleotide sequence, including the coding sequence or any exon, but also any polypeptide sequence, or any portion thereof, such as from NCBI Reference Sequence NM_000610.4, or any allelic variant thereof.

### SLC3A2

SLC3A2 or Solute Carrier Family 3 Member 2 is known under a number of different names such as Lymphocyte Activation Antigen 4F2 Large Subunit; Solute Carrier Family 3 (Activators Of Dibasic And Neutral Amino Acid Transport), Member 2; Antigen Identified By Monoclonal Antibodies 4F2, TRA1.10, TROP4, And T43; Solute Carrier Family 3 (Amino Acid Transporter Heavy Chain), Member 2; 4F2 Cell-Surface Antigen Heavy Chain; CD98 Heavy Chain; 4F2HC; MDU1; Antigen Defined By Monoclonal Antibody 4F2, Heavy Chain; Antigen Defined By Monoclonal Antibody 4F2; 4F2 Heavy Chain Antigen; 4F2 Heavy Chain; CD98 Antigen; CD98HC; 4T2HC; NACAE; CD98 and 4F2. External Ids for SLC3A2 are HGNC: 11026; Entrez Gene: 6520; Ensembl: ENSG00000168003; OMIM: 158070; and UniProtKB: P08195. The term “SLC3A2” as used herein is not meant to be limited to any form, such as a protein, RNA, mRNA, cDNA or a DNA sequence, but to include all such forms, unless the context specifies the intended form as meant. For the avoidance of doubt, with a SCL3A2 transcript version 6 sequence is meant any polynucleotide sequence, including the coding sequence or any exon, but also any polypeptide sequence, or any portion thereof, such as from NCBI Reference Sequence NM_001013251.3, or any allelic variant thereof. And with a SCL3A2 transcript version 3 sequence is meant any polynucleotide sequence, including the coding sequence or any exon, but also any polypeptide sequence, or any portion thereof, such as from NCBI Reference Sequence NM_002394.6, or any allelic variant thereof.

### VTCN1

VTCN1 is known under a number of different names such as V-Set Domain Containing T Cell Activation Inhibitor 1; B7-H4; B7H4; Immune Costimulatory Protein B7-H4; B7 Superfamily Member 1; B7 Family Member, H4; B7 Homolog 4; B7h.5; B7S1; T-Cell Costimulatory Molecule B7x; Protein B7S1; FLJ22418; PRO1291; VCTN1; VTCN1 and B7X. External Ids for VTCN1 are HGNC: 28873; NCBI Entrez Gene: 79679 Ensembl: ENSG00000134258 OMIM®: 608162 and UniProtKB/Swiss-Prot: Q7Z7D3. The term “VTCN1” as used herein is not meant to be limited to any form, such as a protein, RNA, mRNA, cDNA or a DNA sequence, but to include all such forms, unless the context specifies the intended form as meant. For the avoidance of doubt, with a VTCN1 sequence is meant any polynucleotide sequence, including the coding sequence or any exon, but also any polypeptide sequence, or any portion thereof, such as from NCBI Reference Sequence NM_024626.4, or any allelic variant thereof.

### CDH1

CDH1 or Cadherin 1 is known under a number of different names such as Uvomorulin; Cadherin 1, Type 1, E-Cadherin (Epithelial); Epithelial Cadherin; E-Cadherin; Cadherin-1; CAM 120/80; CD324; CDHE; UVO; Calcium-Dependent Adhesion Protein, Epithelial; Epididymis Secretory Sperm Binding Protein; Cadherin 1, E-Cadherin (Epithelial); Cell-CAM 120/80; CD324 Antigen; E-Cadherin; Arc-1; BCDS1; ECAD; and LCAM. External Ids for CDH1 are HGNC: 1748; NCBI Entrez Gene: 999; Ensembl: ENSG00000039068; OMIM®: 192090 and UniProtKB/Swiss-Prot: P12830. The term “CDH1” as used herein is not meant to be limited to any form, such as a protein, RNA, mRNA, cDNA or a DNA sequence, but to include all such forms, unless the context specifies the intended form as meant. For the avoidance of doubt, with a CDH1 sequence is meant any polynucleotide sequence, including the coding sequence or any exon, but also any polypeptide sequence, or any portion thereof, such as from NCBI Reference Sequence NM_001317185.2, or any allelic variant thereof.

### CXADR

CXADR, or coxsackie virus and adenovirus receptor, is a type I membrane receptor for group B coxsackieviruses and subgroup C adenoviruses. Several transcript variants encoding different isoforms have been found for this gene. Diseases associated with CXADR include Myocarditis and Shipyard Eye. Among its related pathways are Adhesion and Allograft rejection. It is a component of the epithelial apical junction complex that may function as a homophilic cell adhesion molecule and is essential for tight junction integrity. Also involved in transepithelial migration of leukocytes through adhesive interactions with JAML a transmembrane protein of the plasma membrane of leukocytes. The interaction between both receptors also mediates the activation of gamma-delta T-cells, a subpopulation of T-cells residing in epithelia and involved in tissue homeostasis and repair. Upon epithelial CXADR-binding, JAML induces downstream cell signaling events in gamma-delta T-cells through PI3-kinase and MAP kinases. It results in proliferation and production of cytokines and growth factors by T-cells that in turn stimulate epithelial tissues repair. External Ids for CXADR include HGNC: 2559 NCBI Entrez Gene: 1525 Ensembl: ENSG00000154639 OMIM®: 602621 UniProtKB/Swiss-Prot: P78310. The term “CXADR” as used herein is not meant to be limited to any form, such as a protein, RNA, mRNA, cDNA or a DNA sequence, but to include all such forms, unless the context specifies the intended form as meant. For the avoidance of doubt, with a CXADR sequence is meant any polynucleotide sequence, including the coding sequence or any exon, but also any polypeptide sequence, or any portion thereof, such as from NCBI Reference Sequence NM_001207063.2, or any allelic variant thereof.

### GTF2E2

GTF2E2, or general transcription factor IIE (TFIIE), is part of the RNA polymerase II transcription initiation complex, recruiting TFIIH and being essential for promoter clearance by RNA polymerase II. TFIIE is a heterodimer (and sometimes heterotetramer) of alpha and beta subunits. The protein encoded by this gene represents the beta subunit of TFIIE. Diseases associated with GTF2E2 include Trichothiodystrophy 6, Nonphotosensitive and Trichothiodystrophy. Among its related pathways are Apoptotic Pathways in Synovial Fibroblasts and CCR5 Pathway in Macrophages. External Ids for GTF2E2 include HGNC: 4651 NCBI Entrez Gene: 2961 Ensembl: ENSG00000197265 OMIM®: 189964 UniProtKB/Swiss-Prot: P29084. The term “GTF2E2” as used herein is not meant to be limited to any form, such as a protein, RNA, mRNA, cDNA or a DNA sequence, but to include all such forms, unless the context specifies the intended form as meant. For the avoidance of doubt, with a GTF2E2 sequence is meant any polynucleotide sequence, including the coding sequence or any exon, but also any polypeptide sequence, or any portion thereof, such as from NCBI Reference Sequence NM_002095.6, or any allelic variant thereof.

### CSMD1

CSMD1 (or CUB And Sushi Multiple Domains 1) is a protein associated with diseases including autism and schizophrenia. External Ids for CSMD1 include HGNC: 14026 NCBI Entrez Gene: 64478 Ensembl: ENSG00000183117 OMIM®: 608397 UniProtKB/Swiss-Prot: Q96PZ7. The term “CSMD1” as used herein is not meant to be limited to any form, such as a protein, RNA, mRNA, cDNA or a DNA sequence, but to include all such forms, unless the context specifies the intended form as meant. For the avoidance of doubt, with a CSMD1 sequence is meant any polynucleotide sequence, including the coding sequence or any exon, but also any polypeptide sequence, or any portion thereof, such as from NCBI Reference Sequence NM_033225.6, or any allelic variant thereof.

### PTN

Pleiotrophin, or PTN, as a protein is a secreted heparin-binding growth factor. The protein has significant roles in cell growth and survival, cell migration, angiogenesis and tumorigenesis. PTN (Pleiotrophin) is a Protein Coding gene. Diseases associated with PTN include Peyronie's Disease and Nasal Cavity Olfactory Neuroblastoma. Among its related pathways are GPCR Pathway and Apoptotic Pathways in Synovial Fibroblasts. External Ids for PTN include HGNC: 9630 NCBI Entrez Gene: 5764 Ensembl: ENSG00000105894 OMIM®: 162095 UniProtKB/Swiss-Prot: P21246. The term “PTN” as used herein is not meant to be limited to any form, such as a protein, RNA, mRNA, cDNA or a DNA sequence, but to include all such forms, unless the context specifies the intended form as meant. For the avoidance of doubt, with a PTN sequence is meant any polynucleotide sequence, including the coding sequence or any exon, but also any polypeptide sequence, or any portion thereof, such as from NCBI Reference Sequence NM_001321386.2, or any allelic variant thereof.

### ST14

ST14 or ST14 Transmembrane Serine Protease Matriptase, is an epithelial-derived, integral membrane serine protease. This protease forms a complex with the Kunitz-type serine protease inhibitor, HAI-1, and is found to be activated by sphingosine 1-phosphate. This protease has been shown to cleave and activate hepatocyte growth factor/scattering factor, and urokinase plasminogen activator, which suggest the function of this protease as an epithelial membrane activator for other proteases and latent growth factors. Diseases associated with ST14 include various types of ichthyosis. Among its related pathways are Developmental Biology and Adhesion. External Ids for ST14 include HGNC: 11344 NCBI Entrez Gene: 6768 Ensembl: ENSG00000149418 OMIM®: 606797 UniProtKB/Swiss-Prot: Q9Y5Y6. The term “ST14” as used herein is not meant to be limited to any form, such as a protein, RNA, mRNA, cDNA or a DNA sequence, but to include all such forms, unless the context specifies the intended form as meant. For the avoidance of doubt, with a ST14 sequence is meant any polynucleotide sequence, including the coding sequence or any exon, but also any polypeptide sequence, or any portion thereof, such as from NCBI Reference Sequence NM_021978.4, or any allelic variant thereof.

### THBS1

THBS1, or Thrombospondin 1, is a subunit of a disulfide-linked homotrimeric protein. This protein is an adhesive glycoprotein that mediates cell-to-cell and cell-to-matrix interactions. This protein can bind to fibrinogen, fibronectin, laminin, type V collagen and integrins alpha-V/beta-1. This protein has been shown to play roles in platelet aggregation, angiogenesis, and tumorigenesis. Diseases associated with THBS1 include Thrombotic Thrombocytopenia Purpura and Peters-Plus Syndrome. Among its related pathways are Proteoglycans in cancer and Degradation of the extracellular matrix. Gene Ontology (GO) annotations related to this gene include calcium ion binding and heparin binding. External Ids for THBS1 include HGNC: 11785 NCBI Entrez Gene: 7057 Ensembl: ENSG00000137801 OMIM®: 188060 UniProtKB/Swiss-Prot: P07996. The term “THBS1” as used herein is not meant to be limited to any form, such as a protein, RNA, mRNA, cDNA or a DNA sequence, but to include all such forms, unless the context specifies the intended form as meant. For the avoidance of doubt, with a THBS1 sequence is meant any polynucleotide sequence, including the coding sequence or any exon, but also any polypeptide sequence, or any portion thereof, such as from NCBI Reference Sequence NM_003246.4, or any allelic variant thereof.

### AGRN

AGRN or Agrin, is one of several proteins that are critical in the development of the neuromuscular junction. The encoded protein contains several laminin G, Kazal type serine protease inhibitor, and epidermal growth factor domains. Additional post-translational modifications occur to add glycosaminoglycans and disulfide bonds. In one family with congenital myasthenic syndrome affecting limb girdle muscles, a mutation in this gene was found. Diseases associated with AGRN include Myasthenic Syndrome, Congenital, 8 and Presynaptic Congenital Myasthenic Syndromes. Among its related pathways are Agrin Interactions at Neuromuscular Junction and Blood-Brain Barrier and Immune Cell Transmigration. External Ids for AGRN include HGNC: 329 NCBI Entrez Gene: 375790 Ensembl: ENSG00000188157 OMIM®: 103320 UniProtKB/Swiss-Prot: 000468. The term “AGRN” as used herein is not meant to be limited to any form, such as a protein, RNA, mRNA, cDNA or a DNA sequence, but to include all such forms, unless the context specifies the intended form as meant. For the avoidance of doubt, with a AGRN sequence is meant any polynucleotide sequence, including the coding sequence or any exon, but also any polypeptide sequence, or any portion thereof, such as from NCBI Reference Sequence NM_001305275.2, or any allelic variant thereof.

### PVALB

PVALP, or Parvalbumin, is a high affinity calcium ion-binding protein that is structurally and functionally similar to calmodulin and troponin C. The encoded protein is thought to be involved in muscle relaxation. Diseases associated with PVALB include Fish allergy and fetal alcohol syndrome. Gene ontology annotations related to this gene include calcium ion binding and protein heterodimerization activity. External Ids for PVALB include HGNC: 9704 NCBI Entrez Gene: 5816 Ensembl: ENSG00000100362 OMIM®: 168890 UniProtKB/Swiss-Prot: P20472. The term “PVALB” as used herein is not meant to be limited to any form, such as a protein, RNA, mRNA, cDNA or a DNA sequence, but to include all such forms, unless the context specifies the intended form as meant. For the avoidance of doubt, with a PVALB sequence is meant any polynucleotide sequence, including the coding sequence or any exon, but also any polypeptide sequence, or any portion thereof, such as from NCBI Reference Sequence NM_002854.3, or any allelic variant thereof.

### APP

APP is known under a number of different names such as Amyloid Beta Precursor Protein, Alpha-SAPP, AD1, Alzheimer Disease Amyloid A4 Protein Homolog, Amyloid Beta (A4) Precursor Protein, Alzheimer Disease Amyloid Protein, Cerebral Vascular Amyloid Peptide, Amyloid-Beta Precursor Protein, Amyloid Precursor Protein, Peptidase Nexin-II, Protease Nexin-II, PN-II, PreA4, ABPP, APPI, CVAP, Beta-Amyloid Precursor Protein, Testicular Tissue Protein Li 2, Beta-Amyloid Peptide (1-40), Beta-Amyloid Peptide (1-42), Amyloid Beta A4 Protein, Beta-Amyloid Peptide, Alzheimer Disease, CTFgamma, ABETA, AAA, PN2, and A4. External Ids for APP Gene are HGNC: 620; Entrez Gene: 351; Ensembl: ENSG00000142192; OMIM®: 104760 and UniProtKB/Swiss-Prot: P05067. The term “APP” as used herein is not meant to be limited to any form, such as a protein, RNA, mRNA, cDNA or a DNA sequence, but to include all such forms, unless the context specifies the intended form as meant. For the avoidance of doubt, with a APP sequence is meant any polynucleotide sequence, including the coding sequence or any exon, but also any polypeptide sequence, or any portion thereof, such as from NCBI Reference Sequence NM_001136130.3, or any allelic variant thereof.

### WRN

WRN or Werner Syndrome ATP-Dependent Helicase is known under a number of different names such as WRN RecQ Like Helicase; RECQL2; RECQ3; Werner Syndrome RecQ Like Helicase; DNA Helicase, RecQ-Like Type 3; RecQ Protein-Like 2; Exonuclease WRN; Werner Syndrome, RecQ Helicase-Like; Werner Syndrome; EC 3.6.4.12; RECQL3; and RecQ3. External Ids for WRN Gene are HGNC: 12791; NCBI Entrez Gene: 7486; Ensembl: ENSG00000165392; OMIM®: 604611; and UniProtKB/Swiss-Prot: Q14191. The term “WRN” as used herein is not meant to be limited to any form, such as a protein, RNA, mRNA, cDNA or a DNA sequence, but to include all such forms, unless the context specifies the intended form as meant. For the avoidance of doubt, with a WRN sequence is meant any polynucleotide sequence, including the coding sequence or any exon, but also any polypeptide sequence, or any portion thereof, such as from NCBI Reference Sequence NM_000553.6, or any allelic variant thereof.

### DAAM1

DAAM1 or Disheveled Associated Activator Of Morphogenesis 1 is known under a number of different names such as KIAA0666; and Disheveled-Associated Activator Of Morphogenesis 1. External Ids for DAAM1 Gene are HGNC: 18142; NCBI Entrez Gene: 23002; Ensembl: ENSG00000100592; OMIM®: 606626; and UniProtKB/Swiss-Prot: Q9Y4D1. The term “DAAM1” as used herein is not meant to be limited to any form, such as a protein, RNA, mRNA, cDNA or a DNA sequence, but to include all such forms, unless the context specifies the intended form as meant. For the avoidance of doubt, with a DAAM1 sequence is meant any polynucleotide sequence, including the coding sequence or any exon, but also any polypeptide sequence, or any portion thereof, such as from NCBI Reference Sequence NM_001270520.2, or any allelic variant thereof.

### ASPH

ASPH or Aspartate Beta-Hydroxylase is known under a number of different names such as BAH, CASQ2BP1, JCTN, HAAH, Aspartyl/Asparaginyl Beta-Hydroxylase, Peptide-Aspartate Beta-Dioxygenase, ASP Beta-Hydroxylase, Junctate, Junctin, Humbug, Cardiac Junctin, EC 1.14.11.16, A Beta H-J-J, FDLAB and AAH. External IDs for ASPH Gene are HGNC: 757; Entrez Gene: 444; Ensembl: ENSG00000198363; OMIM®: 600582 and UniProtKB/Swiss-Prot: Q12797. The term “ASPH” as used herein is not meant to be limited to any form, such as a protein, RNA, mRNA, cDNA or a DNA sequence, but to include all such forms, unless the context specifies the intended form as meant. For the avoidance of doubt, with a ASPH sequence is meant any polynucleotide sequence, including the coding sequence or any exon, but also any polypeptide sequence, or any portion thereof, such as from NCBI Reference Sequence NM_001164750.2, or any allelic variant thereof.

### NOTCH2

NOTCH2 or Notch Receptor 2 is known under a number of different names such as Notch 2; Neurogenic Locus Notch Homolog Protein 2; HN2; Notch (Drosophila) Homolog 2; Notch Homolog 2 (Drosophila); Notch Homolog 2; HJCYS; and AGS2. External Ids for NOTCH2 Gene are HGNC: 7882; NCBI Entrez Gene: 4853; Ensembl: ENSG00000134250; OMIM®: 600275; and UniProtKB/Swiss-Prot: Q04721. The term “NOTCH2” as used herein is not meant to be limited to any form, such as a protein, RNA, mRNA, cDNA or a DNA sequence, but to include all such forms, unless the context specifies the intended form as meant. For the avoidance of doubt, with a NOTCH2 sequence is meant any polynucleotide sequence, including the coding sequence or any exon, but also any polypeptide sequence, or any portion thereof, such as from NCBI Reference Sequence NM_024408.4, or any allelic variant thereof.

### CD74

CD74 is known under a number of different names such as CD74 Molecule; DHLAG; CD74 Molecule, Major Histocompatibility Complex, Class II Invariant Chain; HLA Class II Histocompatibility Antigen Gamma Chain; Class II MHC-Associated Invariant Chain Peptide; HLA-DR Antigens-Associated Invariant Chain; Gamma Chain Of Class II Antigens; Ia-Associated Invariant Chain; MHC HLA-DR Gamma Chain; HLA-DR-Gamma; CLIP; Ia Antigen-Associated Invariant Chain; CD74 Antigen; Ia-GAMMA; HLADG; P33; II; and Ii. External Ids for CD74 Gene are HGNC: 1697; NCBI Entrez Gene: 972; Ensembl: ENSG00000019582; OMIM®: 142790; and UniProtKB/Swiss-Prot: P04233. The term “CD74” as used herein is not meant to be limited to any form, such as a protein, RNA, mRNA, cDNA or a DNA sequence, but to include all such forms, unless the context specifies the intended form as meant. For the avoidance of doubt, with a CD74 sequence is meant any polynucleotide sequence, including the coding sequence or any exon, but also any polypeptide sequence, or any portion thereof, such as from NCBI Reference Sequence NM_001025159.3, or any allelic variant thereof.

### SDC4

SDC4 or Syndecan 4 is known under a number of different names such as Amphiglycan; SYND4; Syndecan 4 (Amphiglycan, Ryudocan); Syndecan Proteoglycan 4; Ryudocan Core Protein; Syndecan-4; Ryudocan; and Ryudocan Amphiglycan. External Ids for SDC4 Gene are HGNC: 10661; NCBI Entrez Gene: 6385; Ensembl: ENSG00000124145; OMIM®: 600017; and UniProtKB/Swiss-Prot: P31431. The term “SDC4” as used herein is not meant to be limited to any form, such as a protein, RNA, mRNA, cDNA or a DNA sequence, but to include all such forms, unless the context specifies the intended form as meant. For the avoidance of doubt, with a SDC4 sequence is meant any polynucleotide sequence, including the coding sequence or any exon, but also any polypeptide sequence, or any portion thereof, such as from NCBI Reference Sequence NM_002999.4, or any allelic variant thereof.

### SLC4A4

SLC4A4 or Solute Carrier Family 4 Member 4 is known under a number of different names such as NBC1; HNBC1; HhNMC; NBC2; PNBC; Solute Carrier Family 4 (Sodium Bicarbonate Cotransporter), Member 4; Electrogenic Sodium Bicarbonate Cotransporter 1; Na(+)/HCO3(−) Cotransporter; SLC4A5; KNBC1; Sodium Bicarbonate Cotransporter 1 (Sodium Bicarbonate Cotransporter, Kidney; Sodium Bicarbonate Cotransporter, Pancreas); Solute Carrier Family 4, Sodium Bicarbonate Cotransporter, Member 4, Brain Type; Solute Carrier Family 4, Sodium Bicarbonate Cotransporter, Member 4; Solute Carrier Family 4, Sodium Bicarbonate Cotransporter, Member 5; Sodium Bicarbonate Cotransporter; NBCe1-A; NBCE1; KNBC; and NBC. External Ids for SLC4A4 Gene are HGNC: 11030; NCBI Entrez Gene: 8671; Ensembl: ENSG00000080493; OMIM®: 603345; and UniProtKB/Swiss-Prot: Q9Y6R1. The term “SLC4A4” as used herein is not meant to be limited to any form, such as a protein, RNA, mRNA, cDNA or a DNA sequence, but to include all such forms, unless the context specifies the intended form as meant. For the avoidance of doubt, with a SLC4A4 sequence is meant any polynucleotide sequence, including the coding sequence or any exon, but also any polypeptide sequence, or any portion thereof, such as from NCBI Reference Sequence NM_001098484.3, or any allelic variant thereof.

### ZFAT

ZFAT or Zing Finger And AT-Hook Domain Containing is known under a number of different names such as Zinc Finger Protein 406; KIAA1485; ZNF406; ZFAT1; Zinc Finger Protein ZFAT; Zinc Finger Gene In Autoimmune Thyroid Disease; Zinc Finger Gene In AITD Susceptibility Region; and AITD3. External Ids for ZFAT Gene are HGNC: 19899; NCBI Entrez Gene: 57623; Ensembl: ENSG00000066827; OMIM®: 610931; and UniProtKB/Swiss-Prot: Q9P243. The term “ZFAT” as used herein is not meant to be limited to any form, such as a protein, RNA, mRNA, cDNA or a DNA sequence, but to include all such forms, unless the context specifies the intended form as meant. For the avoidance of doubt, with a ZFAT sequence is meant any polynucleotide sequence, including the coding sequence or any exon, but also any polypeptide sequence, or any portion thereof, such as from NCBI Reference Sequence NM_020863.4, or any allelic variant thereof.

### DSCAML1

DSCAML1 or DS Cell Adhesion Molecule Like 1 is known under a number of different names such as KIAA1132; Down Syndrome Cell Adhesion Molecule-Like Protein 1; Down Syndrome Cell Adhesion Molecule 2; DSCAM2; Downs Syndrome Cell Adhesion Molecule Like 1; Down Syndrome Cell Adhesion Molecule Like 1; and DSCAM-Like 1. External Ids for DSCAML1 Gene are HGNC: 14656; NCBI Entrez Gene: 57453; Ensembl: ENSG00000177103; OMIM®: 611782; and UniProtKB/Swiss-Prot: Q8TD84. The term “DSCAML1” as used herein is not meant to be limited to any form, such as a protein, RNA, mRNA, cDNA or a DNA sequence, but to include all such forms, unless the context specifies the intended form as meant. For the avoidance of doubt, with a DSCAML1 sequence is meant any polynucleotide sequence, including the coding sequence or any exon, but also any polypeptide sequence, or any portion thereof, such as from NCBI Reference Sequence NM_020693.4, or any allelic variant thereof.

### NRG1 Polynucleotide Fusions

In accordance with the present disclosure, previously unknown genetic rearrangements that result in expression of an NRG1 fusion product, wherein NRG1 is fused with a fusion partner, are now provided. In particular are provided polynucleotide fusions comprising NRG1, including VAPB-NRG1, CADM1-NRG1, CD44-NRG1, SLC3A2-NRG1, VTCN1-NRG1, CDH1-NRG1, CXADR-NRG1, GTF2E2-NRG1, CSMD1-NRG1, PTN-NRG1, ST14-NRG1, THBS1-NRG1, AGRN-NRG1, PVALB-NRG1, APP-NRG1, WRN-NRG1, DAAM1-NRG1, ASPH-NRG1, NOTCH2-NRG1, CD74-NRG1, SDC4-NRG1, SLC4A4-NRG1, ZFAT-NRG1, or DSCAML1-NRG1. In particular, such fusions are present or have been identified in human patients diagnosed with cancer and are mentioned in more details in the following section.

In certain embodiments, the NRG1 fusion may comprise additional downstream fusion partners 3′ of the nucleic acid sequence encoding the EGF-like domain.

### VAPB-NRG1 Polynucleotide Fusions

In accordance with the present disclosure, there is provided a polynucleotide comprising a VAPB nucleic acid sequence (or a portion of a VAPB nucleic acid sequence) fused with an NRG1 nucleic acid sequence (or a portion of an NRG1 nucleic acid sequence). Also included in said fusion are allelic variants of VAPB and NRG1 nucleic acid sequences.

Preferably, the VAPB nucleic acid sequence, or the portion thereof, comprises or consists of any one of SEQ ID NOs: 17-23, or an allelic variant of any one of SEQ ID NOs: 17-23, and the NRG1 nucleic acid sequence, or the portion thereof, comprises or consists of any one of SEQ ID NOs: 125-138, or an allelic variant of any one of SEQ ID NOs: 125-138. More preferably, the VAPB nucleic acid sequence comprises a portion of SEQ ID NOs: 23, or an allelic variant of SEQ ID NOs: 23, and the NRG1 nucleic acid sequence preferably comprises a portion of SEQ ID NOs: 138, or an allelic variant of SEQ ID NOs: 138. SEQ ID NOs: 17-22 correspond to the individual exons 1-6 of VAPB according to NM_004738.4, respectively. SEQ ID NO: 23 corresponds to exons 1-6 of VAPB according to NM_004738.4. SEQ ID NOs: 125-137 correspond to the individual exons 1-13 of the NRG1 sequence according to NM_001159999, respectively. SEQ ID NO: 138 corresponds to exons 1-13 of NRG1 according to NM_001159999.

In a preferred embodiment, the portion of the VAPB nucleic acid sequence comprises 2 to about 10, about 20, about 30, or up to about 40 or even all contiguous nucleic acids from any one of SEQ ID NOs: 17-23, or an allelic variant of any one of SEQ ID NOs: 17-23, and the portion of the NRG1 nucleic acid sequence comprises 2 to about 10, about 20, about 30, or up to about 40 or even all contiguous nucleic acids of any one of SEQ ID NOs: 125-138, or an allelic variant of any one of SEQ ID NOs: 125-138.

Preferably, the VAPB nucleic acid sequence, or the portion thereof, is 5′ to the NRG1 nucleic acid sequence, or the portion thereof.

Preferably, the polynucleotide comprising a VAPB nucleic acid sequence of VAPB, or a portion of said sequence, fused with a NRG1 nucleic acid sequence, or portion of said sequence, comprises or encodes an EGF-like domain of NRG1. An aberrant cell comprising the VAPB-NRG1 polynucleotide fusion or expressing the polypeptide fusion, comprises or encodes an EGF-like domain of NRG1. For detection, diagnosis or identification purposes, it may suffice to show that the fusion junction between VAPB and NRG1 is in-frame and occurs at a position such that the resulting fusion product, nucleic acid or protein, comprises an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

Preferably, the allelic variant of the VAPB nucleic acid sequences has at least 85% identity to any one of SEQ ID NOs: 17-23, preferably at least 90% identity, 92%, 94%, 96% or more preferably at least 98% sequence identity thereto; and the allelic variant of the NRG1 nucleic acid sequences has at least 85% identity to any one of SEQ ID NOs: 125-138, preferably at least 90% identity, 92%, 94%, 96% or more preferably at least 98% sequence identity thereto.

In a preferred embodiment, the polynucleotide comprising the VAPB nucleic acid fused with the NRG1 nucleic acid, comprises 2 to about 10, about 20, about 30, or up to about 40 or even all contiguous nucleic acids from SEQ ID NO: 3, preferably including the nucleic acids at positions 43 and 44. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 3, preferably including at least the nucleic acids at positions 43 and 44.

In a preferred embodiment, there is provided a polynucleotide according to SEQ ID NO: 3, or a polynucleotide which comprises about 20, about 30, about 40 or all contiguous nucleic acids from SEQ ID NO: 3, preferably including the nucleic acids at positions 43 and 44. The number of contiguous nucleic acids may be 18, 19, 20, 21, 22, 23, 24, 25, 26, 27, 28, 29, 30, 31, 32, 33, 34, 35, 36, 37, 38, 39, 40 or all nucleic acids of SEQ ID NO: 3, preferably including at least the nucleic acids at positions 43 and 44.

Preferably, the polynucleotide part of the NRG1 nucleic acid sequence (or the allelic variant thereof), encodes an EGF-like domain of NRG1, preferably the EGF-like domain according to SEQ ID NO: 163.

In an alternative aspect, there is provided a polynucleotide comprising a portion of exon 1 of VAPB, or an allelic variant thereof, fused with a portion of exon 2 of NRG1, or an allelic variant thereof. Preferably, exon 1 of VAPB is that of SEQ ID NO: 17. Preferably, exon 2 of NRG1 is that of SEQ ID NO: 126. Said portion of exon 1 of VAPB preferably comprises or consists of SEQ ID NO: 1, or an allelic variant thereof. Said portion of exon 2 of NRG1 preferably comprises or consists of SEQ ID NO: 2, or an allelic variant thereof. When present in an aberrant cell of a patient or subject, said polynucleotide fusion preferably further includes any sequence 3′ from exon 2 of NRG1, but to allow detection of the fusion junction using a polynucleotide-based detection assay, it may suffice that at least SEQ ID NOs 17 and 126 are present. Any sequence 3′ from exon 2 of NRG1 comprises or consists of one or all of SEQ ID NOs: 127-137 (or any allelic variant of SEQ ID NOs: 127-137).

Preferably, the allelic variant of exon 1 of VAPB has at least 85% identity to SEQ ID NO: 17, preferably at least 90% identity, 92%, 94%, 96% or more preferably at least 98% sequence identity thereto and the allelic variant of exon 2 of NRG1 has at least 85% identity to SEQ ID NO: 126, preferably at least 90% identity, 92%, 94%, 96% or more preferably at least 98% sequence identity thereto.

In a preferred embodiment, the portion of exon 1 of VAPB comprises or consists of SEQ ID NO: 1 and the allelic variant thereof has at least 85% identity to SEQ ID NO: 1, preferably at least 90% identity, 92%, 94%, 96% or more preferably at least 98% sequence identity thereto. The portion of exon 2 of NRG1 in this fusion preferably comprises or consists of SEQ ID NO: 2 and the allelic variant thereof has at least 85% identity to SEQ ID NO: 2, preferably at least 90% identity, 92%, 94%, 96% or more preferably at least 98% sequence identity thereto. Such short polynucleotide sequences are particularly useful for detecting the presence of a larger polynucleotide fusion between VAPB and NRG1 and establishing that such a fusion is an in-frame, oncogenic fusion comprising an EGF-like domain of NRG1. More preferably, the portion of exon 1 of VAPB comprises or consists of 2 to about 10, about 20, about 30, or up to about 40 or even all contiguous nucleic acids from SEQ ID NO: 1, including at least the nucleic acid of position 43. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 1, including at least the nucleic acid of position 43. More preferably, the portion of exon 1 of VAPB comprises or is according to SEQ ID NO: 1, or an allelic variant thereof.

Alternatively, the portion of exon 1 of VAPB comprises or consists of SEQ ID NO: 17 (or an allelic variant of SEQ ID NO: 17), including at least the nucleic acid of position 399. Preferably, the portion of exon 1 of VAPB comprises or consists of 2 to about 10, about 20, about 30, or up to about 40 or even all contiguous nucleic acids from SEQ ID NO: 17, or from the allelic variant thereof, including at least the nucleic acid of position 399. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 17, including at least the nucleic acid of position 399. In this alternative, the portion of exon 1 of VAPB more preferably comprises or is according to SEQ ID NO: 17, or an allelic variant thereof. Preferably, the portion of exon 2 of NRG1 in the fusion with VAPB comprises or consists of 2 to about 10, about 20, about 30, or up to about 40 or even all contiguous nucleic acids from SEQ ID NO: 126, or from the allelic variant thereof, including at least the nucleic acid of position 1. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 126, including at least the nucleic acid of position 1.

In an alternatively preferred embodiment, the polynucleotide comprising a portion of exon 1 of VAPB fused with a portion of exon 2 of NRG1 comprises 2 to about 10, about 20, about 30, or up to about 40 or even all contiguous nucleic acids from SEQ ID NO: 3, including the nucleic acids at positions 43 and 44. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 3, including at least the nucleic acids at positions 43 and 44. SEQ ID NO: 3 includes the junction between VAPB and NRG1, in particular the junction is between the nucleic acid at position 43, which stems from VAPB and the nucleic acid at position 44 which stems from NRG1. Preferably, the polynucleotide comprising a portion of exon 1 of VAPB fused with a portion of exon 2 of NRG1 has the polynucleotide sequence of SEQ ID NO: 3, or an allelic variant thereof.

Preferably, any VAPB-NRG1 polynucleotide fusion provided herein is an in-frame fusion of VAPB with NRG1. More preferably said fusion is an in frame fusion comprising exon 1 of VAPB, or a portion of exon 1, with exon 2 of NRG1, or a portion of exon 2. Said in-frame fusion is preferably the fusion of SEQ ID NO: 3, or an allelic variant thereof.

Preferably, the portion of exon 1 of VAPB, or the allelic variant thereof, is 5′ to the portion of exon 2 of NRG1, or the allelic variant thereof. This orientation at the nucleic acid level results in a fusion polypeptide product that contains an N-terminus of VAPB and a C-terminus of NRG1. Also, the herein provided VAPB-NRG1 polynucleotide fusion produces a protein fusion, wherein the part from the N-terminus to the fusion junction is polypeptide sequence from VAPB and the part from the junction to the C-terminus is NRG1 polypeptide sequence, wherein the NRG1 part also provides its EGF-like domain. The VAPB-NRG1 fusion protein thereby retains the EGF-like domain of NRG1 and the ability to drive proliferation and survival of a subset of human cancers, including lung cancer or adenocarcinoma, in particular lung adenocarcinoma or non-small cell lung cancer.

### CADM1-NRG1 Polynucleotide Fusion

There is also provided a polynucleotide fusion comprising a portion of exon 7 of CADM1 fused with a portion of exon 6 of NRG1. Exon 7 of CADM1 is preferably that of SEQ ID NO: 39, or an allelic variant of SEQ ID NO: 39, and exon 6 of NRG1 is preferably that of SEQ ID NO: 130, or an allelic variant of SEQ ID NO: 130.

Preferably, the allelic variant of exon 7 of CADM1 has at least 85% identity to SEQ ID NO: 39, preferably at least 90% identity, 92%, 94%, 96% or more preferably at least 98% sequence identity thereto and the allelic variant of exon 6 of NRG1 has at least 85% identity to SEQ ID NO: 130, preferably at least 90% identity, 92%, 94%, 96% or more preferably at least 98% sequence identity thereto.

Preferably, the portion of exon 7 of CADM1 comprises or is according to SEQ ID NO: 5 and the allelic variant thereof has at least 85% identity to SEQ ID NO: 5, preferably at least 90% identity, 92%, 94%, 96% or more preferably at least 98% sequence identity thereto. The portion of exon 6 of NRG1 in the fusion with CADM1 preferably is or comprises the sequence according to SEQ ID NO: 6 and the allelic variant thereof has at least 85% identity to SEQ ID NO: 6, preferably at least 90% identity, 92%, 94%, 96% or more preferably at least 98% sequence identity thereto. Such short polynucleotide sequences are particularly useful for detecting the presence of a larger polynucleotide fusion between CADM1 and NRG1 and establishing that such a fusion is an in-frame, oncogenic fusion comprising an EGF-like domain of NRG1. More preferably, the portion of exon 7 of CADM1 comprises or consists of 2 to about 10, about 20, about 30, or up to about 40 or even all contiguous nucleic acids from SEQ ID NO: 5, including at least the nucleic acid of position 53. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO:5, including at least the nucleic acid of position 53. More preferably, the portion of exon 7 of CADM1 comprises or is according to SEQ ID NO: 5, or an allelic variant thereof.

Alternatively, the portion of exon 7 of CADM1 comprises or consists of SEQ ID NO: 39 (or an allelic variant of SEQ ID NO: 39), including at least the nucleic acid of position 173. Preferably, the portion of exon 7 of CADM1 comprises or consists of 2 to about 10, about 20, about 30, or up to about 40 or even all contiguous nucleic acids from SEQ ID NO: 39, or from the allelic variant thereof, including at least the nucleic acid of position 173. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 39, including at least the nucleic acid of position 173. In this alternative, the portion of exon 7 of CADM1 more preferably comprises or is according to SEQ ID NO: 39, or an allelic variant thereof. Preferably, the portion of exon 6 of NRG1 in the fusion with CADM1 comprises or consists of 2 to about 10, about 20, about 30, or up to about 40 or even all contiguous nucleic acids from SEQ ID NO: 130, or from the allelic variant thereof, including at least the nucleic acid of position 1. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 130, including at least the nucleic acid of position 1.

In an alternatively preferred embodiment, the polynucleotide comprising a portion of exon 7 of CADM1 fused with a portion of exon 6 of NRG1 comprises 2 to about 10, about 20, about 30, or up to about 40 or even all contiguous nucleic acids from SEQ ID NO: 7, including the nucleic acids at positions 53 and 54. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 7, including at least the nucleic acids at positions 53 and 54. SEQ ID NO: 7 includes the junction between CADM1 and NRG1, in particular the junction is between the nucleic acid at position 53, which stems from CADM1 and the nucleic acid at position 54 which stems from NRG1. Preferably, the polynucleotide comprising a portion of exon 7 of CADM1 fused with a portion of exon 6 of NRG1 has the polynucleotide sequence of SEQ ID NO: 7, or an allelic variant thereof.

In a preferred embodiment, there is provided a polynucleotide according to SEQ ID NO: 7, or a polynucleotide which comprises about 20, about 30, about 40 or all contiguous nucleic acids from SEQ ID NO: 7, preferably including the nucleic acids at positions 53 and 54. The number of contiguous nucleic acids may be 18, 19, 20, 21, 22, 23, 24, 25, 26, 27, 28, 29, 30, 31, 32, 33, 34, 35, 36, 37, 38, 39, 40 or all nucleic acids of SEQ ID NO: 7, preferably including at least the nucleic acids at positions 53 and 54.

Preferably, any CADM1-NRG1 polynucleotide fusion provided herein is an in-frame fusion of CADM1 with NRG1. More preferably said fusion is an in-frame fusion comprising exon 7 of CADM1, or a portion of exon 7, with exon 6 of NRG1, or a portion of exon 6. Said in frame fusion is preferably the fusion of SEQ ID NO: 7, or an allelic variant thereof having at least 85% identity thereto, preferably at least 90%, 92%, 94%, 96% or even 98% identity thereto.

Preferably, the polynucleotide comprising a portion of exon 7 of CADM1, or an allelic variant thereof, fused with a portion of exon 6 of NRG1, or an allelic variant thereof, is part of a longer polynucleotide that further comprises or encodes an EGF-like domain of NRG1. An aberrant cell comprising the polynucleotide fusion involving CADM1 or expressing the polypeptide fusion, comprises or encodes an EGF-like domain of NRG1. For quick detection, diagnosis or identification purposes, it may suffice to show that the fusion junction between CADM1 and NRG1 is in-frame and occurs at such a position that the resulting fusion product, nucleic acid or protein, comprises an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

Preferably, the portion of exon 7 of CADM1, or the allelic variant thereof, is 5′ to the portion of exon 6 of NRG1, or the allelic variant thereof. This orientation at the nucleic acid level results in a fusion polypeptide product that contains an N-terminus of CADM1 and a C-terminus of NRG1. Also, the herein provided CADM1-NRG1 polynucleotide fusion produces a protein fusion, wherein the part from the N-terminus to the fusion junction is polypeptide sequence from CADM1 and the part from the junction to the C-terminus is NRG1 polypeptide sequence, wherein the NRG1 part also provides its EGF-like domain. The CADM1-NRG1 fusion protein thereby retains the EGF-like domain of NRG1 and the ability to drive proliferation and survival of a subset of human cancers, including lung cancer or adenocarcinoma, in particular lung adenocarcinoma.

When present in an aberrant cell of a patient or subject, said CADM1-NRG1 polynucleotide fusion preferably further includes any sequence 5′ from exon 7 of CADM1 and any sequence 3′ from exon 6 of NRG1, but to allow detection of the fusion junction using a polynucleotide-based detection assay, it may suffice that at least SEQ ID NOs: 39 and 130 are present. Any sequence 5′ from exon 7 of CADM1 comprises or consists of one or all of SEQ ID NOs: 33-38 (or any allelic variant of SEQ ID NOs: 33-38), and any sequence 3′ of exon 6 of NRG1 comprises or consists of any one or all of SEQ ID NOs: 131-137 (or any allelic variant of SEQ ID NOs: 131-137).

### CD44-NRG1 Polynucleotide Fusion

There is also provided a polynucleotide fusion comprising a portion of exon 5 of CD44 fused with a portion of exon 2 of NRG1. Exon 5 of CD44 is preferably that of SEQ ID NO: 65, or an allelic variant of SEQ ID NO: 65, and exon 2 of NRG1 is preferably that of SEQ ID NO: 126, or an allelic variant of SEQ ID NO: 126.

Preferably, the allelic variant of exon 5 of CD44 has at least 85% identity to SEQ ID NO: 65, preferably at least 90% identity, 92%, 94%, 96% or more preferably at least 98% sequence identity thereto and the allelic variant of exon 2 of NRG1 has at least 85% identity to SEQ ID NO: 126, preferably at least 90% identity, 92%, 94%, 96% or more preferably at least 98% sequence identity thereto.

Preferably, the portion of exon 5 of CD44 comprises or is according to SEQ ID NO: 9 and the allelic variant thereof has at least 85% identity to SEQ ID NO: 9, preferably at least 90% identity, 92%, 94%, 96% or more preferably at least 98% sequence identity thereto. The portion of exon 2 of NRG1 in the fusion with CD44 preferably is or comprises the sequence according to SEQ ID NO: 10 and the allelic variant thereof has at least 85% identity to SEQ ID NO: 10, preferably at least 90% identity, 92%, 94%, 96% or more preferably at least 98% sequence identity thereto. More preferably, the portion of exon 5 of CD44 comprises or consists of 2 to about 10, about 20, about 30, or up to about 40 or even all contiguous nucleic acids from SEQ ID NO: 9, including at least the nucleic acid of position 52. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 9, including at least the nucleic acid of position 52. More preferably, the portion of exon 5 of CD44 comprises or is according to SEQ ID NO: 9, or an allelic variant thereof. Such short polynucleotide sequences are particularly useful for detecting the presence of a larger polynucleotide fusion between CD44 and NRG1 and establishing that such a fusion is an in-frame, oncogenic fusion comprising an EGF-like domain of NRG1.

Alternatively, the portion of exon 5 of CD44 comprises or consists of SEQ ID NO: 65 (or an allelic variant of SEQ ID NO: 65), including at least the nucleic acid of position 231. Preferably, the portion of exon 5 of CD44 comprises or consists of 2 to about 10, about 20, about 30, or up to about 40 or even all contiguous nucleic acids from SEQ ID NO: 65, or from the allelic variant thereof, including at least the nucleic acid of position 231. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 65, including at least the nucleic acid of position 231. In this alternative, the portion of exon 5 of CD44 more preferably comprises or is according to SEQ ID NO: 231, or an allelic variant thereof. Preferably, the portion of exon 2 of NRG1 in the fusion with CD44 comprises or consists of 2 to about 10, about 20, about 30, or up to about 40 or even all contiguous nucleic acids from SEQ ID NO: 126, or from the allelic variant thereof, including at least the nucleic acid of position 1. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 126, including at least the nucleic acid of position 1.

In an alternatively preferred embodiment, the polynucleotide comprising a portion of exon 5 of CD44 fused with a portion of exon 2 of NRG1 comprises 2 to about 10, about 20, about 30, or up to about 40 or even all contiguous nucleic acids from SEQ ID NO: 11, including the nucleic acids at positions 52 and 53. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 11, including at least the nucleic acids at positions 52 and 53. SEQ ID NO: 11 includes the junction between CD44 and NRG1, in particular the junction is between the nucleic acid at position 52, which stems from CD44 and the nucleic acid at position 53 which stems from NRG1. Preferably, the polynucleotide comprising a portion of exon 5 of CD44 fused with a portion of exon 2 of NRG1 has the polynucleotide sequence of SEQ ID NO: 15, or an allelic variant thereof.

In a preferred embodiment, there is provided a polynucleotide according to SEQ ID NO: 11, or a polynucleotide which comprises about 20, about 30, about 40 or all contiguous nucleic acids from SEQ ID NO: 11, preferably including the nucleic acids at positions 52 and 53. The number of contiguous nucleic acids may be 18, 19, 20, 21, 22, 23, 24, 25, 26, 27, 28, 29, 30, 31, 32, 33, 34, 35, 36, 37, 38, 39, 40 or all nucleic acids of SEQ ID NO: 11, preferably including at least the nucleic acids at positions 52 and 53.

Preferably, any CD44-NRG1 polynucleotide fusion provided herein is an in-frame fusion of CD44 with NRG1. More preferably said fusion is an in-frame fusion comprising exon 5 of CD44 (or a portion of exon 5) with exon 2 of NRG1 (or a portion of exon 2 of NRG1). Said in-frame fusion is preferably the fusion of SEQ ID NO: 11, or an allelic variant thereof having at least 85% identity thereto, preferably at least 90%, 92%, 94%, 96% or even 98% identity thereto.

Preferably, the polynucleotide comprising a portion of exon 5 of CD44, or an allelic variant thereof, fused with a portion of exon 2 of NRG1, or an allelic variant thereof, is part of a longer polynucleotide that further comprises or encodes an EGF-like domain of NRG1. An aberrant cell comprising the polynucleotide fusion involving CD44 or expressing the polypeptide fusion, comprises or encodes an EGF-like domain of NRG1. For quick detection, diagnosis or identification purposes, it may suffice to show that the fusion junction between CD44 and NRG1 is in-frame and occurs at such a position that the resulting fusion product, nucleic acid or protein, comprises an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

Preferably, the portion of exon 5 of CD44, or the allelic variant thereof, is 5′ to the portion of exon 2 of NRG1, or the allelic variant thereof. This orientation at the nucleic acid level results in a fusion polypeptide product that contains an N-terminus of CD44 and a C-terminus of NRG1. Also, the herein provided CD44-NRG1 polynucleotide fusion produces a protein fusion, wherein the part from the N-terminus to the fusion junction is polypeptide sequence from CD44 and the part from the junction to the C-terminus is NRG1 polypeptide sequence, wherein the NRG1 part also provides its EGF-like domain. The CD44-NRG1 fusion protein thereby retains the EGF-like domain of NRG1 and the ability to drive proliferation and survival of a subset of human cancers, including pancreatic cancer or pancreatic adenocarcinoma, in particular pancreatic ductal adenocarcinoma (or PDAC).

When present in an aberrant cell of a patient or subject, said CD44-NRG1 polynucleotide fusion preferably further includes any sequence 5′ from exon 5 of CD44 and any sequence 3′ from exon 2 of NRG1, but to allow detection of the fusion junction using a polynucleotide-based detection assay, it may suffice that at least SEQ ID NOs: 65 and 126 are present. Any sequence 5′ from exon 5 of CD44 comprises or consists of one or all of SEQ ID NOs: 61-64 (or any allelic variant of SEQ ID NOs: 61-64), and any sequence 3′ of exon 2 of NRG1 comprises or consists of one or all of SEQ ID NOs: 127-137 (or any allelic variant of SEQ ID NOs 127-137).

Further, there is provided a polynucleotide fusion comprising a portion of Exon 5 of CD44 fused with a portion of exon 6 of NRG1. Exon 5 of said CD44 is preferably that of SEQ ID NO: 65, or an allelic variant of SEQ ID NO: 65, and exon 6 of NRG1 is preferably that of SEQ ID NO: 130, or an allelic variant of SEQ ID NO: 130.

Preferably, the allelic variant of Exon 5 of said CD44 has at least 85% identity to SEQ ID NO: 65, preferably at least 90% identity, 92%, 94%, 96% or more preferably at least 98% sequence identity thereto and the allelic variant of exon 6 of NRG1 has at least 85% identity to SEQ ID NO: 130, preferably at least 90% identity, 92%, 94%, 96% or more preferably at least 98% sequence identity thereto.

Preferably, the portion of Exon 5 of said CD44 comprises or is according to SEQ ID NO: 759 and the allelic variant thereof has at least 85% identity to SEQ ID NO: 759, preferably at least 90% identity, 92%, 94%, 96% or more preferably at least 98% sequence identity thereto. The portion of exon 6 of NRG1 in the fusion with CD44 preferably is or comprises the sequence according to SEQ ID NO: 760 and the allelic variant thereof has at least 85% identity to SEQ ID NO: 760, preferably at least 90% identity, 92%, 94%, 96% or more preferably at least 98% sequence identity thereto. More preferably, the portion of Exon 5 of CD44 comprises or consists of 2 to about 10, about 20, about 30, or up to about 40 or even all contiguous nucleic acids from SEQ ID NO: 759, including at least the nucleic acid of position 75. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 759, including at least the nucleic acid of position 75. More preferably, the portion of Exon 5 of said CD44 comprises or is according to SEQ ID NO: 759, or an allelic variant thereof. Such short polynucleotide sequences are particularly useful for detecting the presence of a larger polynucleotide fusion between said CD44 and NRG1 and establishing that such a fusion is an in frame, oncogenic fusion comprising an EGF-like domain of NRG1.

Alternatively, the portion of Exon 5 of said CD44 comprises or consists of SEQ ID NO: 65 (or an allelic variant of SEQ ID NO: 65), including at least the nucleic acid of position 231. Preferably, the portion of Exon 5 of said CD44 comprises or consists of 2 to about 10, about 20, about 30, or up to about 40 or even all contiguous nucleic acids from SEQ ID NO: 65, or from the allelic variant thereof, including at least the nucleic acid of position 231. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 65, including at least the nucleic acid of position 231. In this alternative, the portion of Exon 5 of CD44 more preferably comprises or is according to SEQ ID NO: 65, or an allelic variant thereof. Preferably, the portion of exon 6 of NRG1 in the fusion with of CD44 comprises or consists of 2 to about 10, about 20, about 30, or up to about 40 or even all contiguous nucleic acids from SEQ ID NO: 130, or from the allelic variant thereof, including at least the nucleic acid of position 1. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 130, including at least the nucleic acid of position 1.

Preferably, any CD44-NRG1 polynucleotide fusion provided herein is an in-frame fusion of CD44 with NRG1. More preferably said fusion is an in-frame fusion comprising Exon 5 of CD44, or a portion of Exon 5, with exon 6 of NRG1, or a portion of exon 6 of NRG1. Said in-frame fusion is preferably the fusion of SEQ ID NO: 761, or an allelic variant thereof having at least 85% identity thereto, preferably at least 90%, 92%, 94%, 96% or even 98% identity thereto.

In an alternatively preferred embodiment, the polynucleotide comprising a portion of Exon 5 of said CD44 fused with a portion of exon 6 of NRG1 comprises 2 to about 10, about 20, about 30, or up to about 40 or even all contiguous nucleic acids from SEQ ID NO: 761, including the nucleic acids at positions 75 and 76. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 761, including at least the nucleic acids at positions 75 and 76. SEQ ID NO: 761 includes the junction between said CD44 and NRG1, in particular the junction is between the nucleic acid at position 75, which stems from CD44 and the nucleic acid at position 76 which stems from NRG1. Preferably, the polynucleotide comprising a portion of Exon 5 of CD44 fused with a portion of exon 6 of NRG1 has the polynucleotide sequence of SEQ ID NO: 761, or an allelic variant thereof.

In a preferred embodiment, there is provided a polynucleotide according to SEQ ID NO: 761, or a polynucleotide which comprises about 20, about 30, about 40 or all contiguous nucleic acids from SEQ ID NO: 761, preferably including the nucleic acids at positions 75 and 76. The number of contiguous nucleic acids may be 18, 19, 20, 21, 22, 23, 24, 25, 26, 27, 28, 29, 30, 31, 32, 33, 34, 35, 36, 37, 38, 39, 40 or all nucleic acids of SEQ ID NO: 761, preferably including at least the nucleic acids at positions 75 and 76.

Preferably, the polynucleotide comprising a portion of Exon 5 of CD44, or an allelic variant thereof, fused with a portion of exon 6 of NRG1, or an allelic variant thereof, is part of a longer polynucleotide that further comprises or encodes an EGF-like domain of NRG1. An aberrant cell comprising the polynucleotide fusion involving said CD44 or expressing the polypeptide fusion, comprises or encodes an EGF-like domain of NRG1. For quick detection, diagnosis or identification purposes, it may suffice to show that the fusion junction between said CD44 and NRG1 is in-frame and occurs at such a position that the resulting fusion product, nucleic acid or protein, comprises an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

Preferably, the portion of Exon 5 of CD44, or the allelic variant thereof, is 5′ to the portion of exon 6 of NRG1, or the allelic variant thereof. This orientation at the nucleic acid level results in a fusion polypeptide product that contains an N-terminus of CD44 and a C-terminus of NRG1. Also, the herein provided CD44-NRG1 polynucleotide fusion produces a protein fusion, wherein the part from the N-terminus to the fusion junction is polypeptide sequence from CD44 and the part from the junction to the C-terminus is NRG1 polypeptide sequence, wherein the NRG1 part also provides its EGF-like domain. The CD44-NRG1 fusion protein thereby retains the EGF-like domain of NRG1 and the ability to drive proliferation and survival of a subset of human cancers, in particular a pancreatic cancer.

When present in an aberrant cell of a patient or subject, said polynucleotide fusion preferably further includes any sequence 5′ from Exon 5 of CD44 and any sequence 3′ from exon 6 of NRG1, but to allow detection of the fusion junction using a polynucleotide-based detection assay, it may suffice that at least SEQ ID NOs: 65 and 130 are present. Any sequence 5′ from Exon 5 of CD44 comprises or consists of one or all of SEQ ID NOs: 61-64 (or any allelic variant of SEQ ID NOs: 61-64), and any sequence 3′ of exon 6 of NRG1 comprises or consists of one or all of SEQ ID NOs: 131-137 (or any allelic variant of SEQ ID NOs: 131-137).

### SLC3A2-NRG1 Polynucleotide Fusion

There is also provided a polynucleotide fusion comprising a portion of exon 1 of transcript version 6 SLC3A2 fused with a portion of exon 5 of NRG1. Exon 1 of said SLC3A2 is preferably that of SEQ ID NO: 103, or an allelic variant of SEQ ID NO: 103, and exon 5 of NRG1 is preferably that of SEQ ID NO: 129, or an allelic variant of SEQ ID NO: 129.

Preferably, the allelic variant of exon 1 of said SLC3A2 has at least 85% identity to SEQ ID NO: 103, preferably at least 90% identity, 92%, 94%, 96% or more preferably at least 98% sequence identity thereto and the allelic variant of exon 5 of NRG1 has at least 85% identity to SEQ ID NO: 129, preferably at least 90% identity, 92%, 94%, 96% or more preferably at least 98% sequence identity thereto.

Preferably, the portion of exon 1 of said SLC3A2 comprises or is according to SEQ ID NO: 13 and the allelic variant thereof has at least 85% identity to SEQ ID NO: 13, preferably at least 90% identity, 92%, 94%, 96% or more preferably at least 98% sequence identity thereto. The portion of exon 5 of NRG1 in the fusion with SLC3A2 preferably is or comprises the sequence according to SEQ ID NO: 14 and the allelic variant thereof has at least 85% identity to SEQ ID NO: 14, preferably at least 90% identity, 92%, 94%, 96% or more preferably at least 98% sequence identity thereto. More preferably, the portion of exon 1 of SLC3A2 comprises or consists of 2 to about 10, about 20, about 30, or up to about 40 or even all contiguous nucleic acids from SEQ ID NO: 13, including at least the nucleic acid of position 53. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 13, including at least the nucleic acid of position 53. More preferably, the portion of exon 1 of said SLC3A2 comprises or is according to SEQ ID NO: 13, or an allelic variant thereof. Such short polynucleotide sequences are particularly useful for detecting the presence of a larger polynucleotide fusion between said SLC3A2 and NRG1 and establishing that such a fusion is an in frame, oncogenic fusion comprising an EGF-like domain of NRG1.

Alternatively, the portion of exon 1 of said SLC3A2 comprises or consists of SEQ ID NO: 103 (or an allelic variant of SEQ ID NO: 103), including at least the nucleic acid of position 552. Preferably, the portion of exon 1 of said SLC3A2 comprises or consists of 2 to about 10, about 20, about 30, or up to about 40 or even all contiguous nucleic acids from SEQ ID NO: 103, or from the allelic variant thereof, including at least the nucleic acid of position 552. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 103, including at least the nucleic acid of position 552. In this alternative, the portion of exon 1 of SLC3A2 more preferably comprises or is according to SEQ ID NO: 103, or an allelic variant thereof. Preferably, the portion of exon 5 of NRG1 in the fusion with said SLC3A2 comprises or consists of 2 to about 10, about 20, about 30, or up to about 40 or even all contiguous nucleic acids from SEQ ID NO: 157, or from the allelic variant thereof, including at least the nucleic acid of position 1. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 157, including at least the nucleic acid of position 1.

Preferably, any SLC3A2-NRG1 polynucleotide fusion provided herein is an in-frame fusion of SLC3A2 with NRG1. More preferably said fusion is an in-frame fusion comprising exon 1 of transcript version 6 of SLC3A2, or a portion of exon 1, with exon 5 of NRG1, or a portion of exon 5 of NRG1. Said in frame fusion is preferably the fusion of SEQ ID NO: 15, or an allelic variant thereof having at least 85% identity thereto, preferably at least 90%, 92%, 94%, 96% or even 98% identity thereto.

In an alternatively preferred embodiment, the polynucleotide comprising a portion of exon 1 of said SLC3A2 fused with a portion of exon 5 of NRG1 comprises 2 to about 10, about 20, about 30, or up to about 40 or even all contiguous nucleic acids from SEQ ID NO: 15, including the nucleic acids at positions 53 and 54. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 15, including at least the nucleic acids at positions 53 and 54. SEQ ID NO: 15 includes the junction between said SLC3A2 and NRG1, in particular the junction is between the nucleic acid at position 53, which stems from SLC3A2 and the nucleic acid at position 54 which stems from NRG1. Preferably, the polynucleotide comprising a portion of exon 1 of SLC3A2 fused with a portion of exon 5 of NRG1 has the polynucleotide sequence of SEQ ID NO: 15, or an allelic variant thereof.

In a preferred embodiment, there is provided a polynucleotide according to SEQ ID NO: 15, or a polynucleotide which comprises about 20, about 30, about 40 or all contiguous nucleic acids from SEQ ID NO: 15, preferably including the nucleic acids at positions 53 and 54. The number of contiguous nucleic acids may be 18, 19, 20, 21, 22, 23, 24, 25, 26, 27, 28, 29, 30, 31, 32, 33, 34, 35, 36, 37, 38, 39, 40 or all nucleic acids of SEQ ID NO:15, preferably including at least the nucleic acids at positions 53 and 54.

Preferably, the polynucleotide comprising a portion of exon 1 of transcript version 6 of SLC3A2, or an allelic variant thereof, fused with a portion of exon 5 of NRG1, or an allelic variant thereof, is part of a longer polynucleotide that further comprises or encodes an EGF-like domain of NRG1. An aberrant cell comprising the polynucleotide fusion involving said SLC3A2 or expressing the polypeptide fusion, comprises or encodes an EGF-like domain of NRG1. For quick detection, diagnosis or identification purposes, it may suffice to show that the fusion junction between said SLC3A2 and NRG1 is in-frame and occurs at such a position that the resulting fusion product, nucleic acid or protein, comprises an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

Preferably, the portion of exon 1 of transcript version 6 of SLC3A2, or the allelic variant thereof, is 5′ to the portion of exon 5 of NRG1, or the allelic variant thereof. This orientation at the nucleic acid level results in a fusion polypeptide product that contains an N-terminus of SLC3A2 and a C-terminus of NRG1. Also, the herein provided SLC3A2-NRG1 polynucleotide fusion produces a protein fusion, wherein the part from the N-terminus to the fusion junction is polypeptide sequence from SLC3A2 and the part from the junction to the C-terminus is NRG1 polypeptide sequence, wherein the NRG1 part also provides its EGF-like domain. The SLC3A2-NRG1 fusion protein thereby retains the EGF-like domain of NRG1 and the ability to drive proliferation and survival of a subset of human cancers, including lung cancer or adenocarcinoma, in particular lung adenocarcinoma.

When present in an aberrant cell of a patient or subject, said SCL3A2-NRG1 polynucleotide fusion preferably further includes any sequence 3′ from exon 5 of NRG1, but to allow detection of the fusion junction using a polynucleotide-based detection assay, it may suffice that at least SEQ ID NOs: 103 and 129 are present. Any sequence 3′ of exon 5 of NRG1 comprises or consists of one or all of SEQ ID NOs: 130-137 (or any allelic variant of SEQ ID NOs: 130-137).

Further, there is provided a polynucleotide fusion comprising a portion of exon 2 of transcript version 3 SLC3A2 fused with a portion of exon 6 of NRG1. Exon 2 of said SLC3A2 is preferably that of SEQ ID NO: 457, or an allelic variant of SEQ ID NO: 457, and exon 6 of NRG1 is preferably that of SEQ ID NO: 130, or an allelic variant of SEQ ID NO: 130.

Preferably, the allelic variant of exon 2 of said SLC3A2 has at least 85% identity to SEQ ID NO: 457, preferably at least 90% identity, 92%, 94%, 96% or more preferably at least 98% sequence identity thereto and the allelic variant of exon 6 of NRG1 has at least 85% identity to SEQ ID NO: 130, preferably at least 90% identity, 92%, 94%, 96% or more preferably at least 98% sequence identity thereto.

Preferably, the portion of exon 2 of said SLC3A2 comprises or is according to SEQ ID NO: 452 and the allelic variant thereof has at least 85% identity to SEQ ID NO: 452, preferably at least 90% identity, 92%, 94%, 96% or more preferably at least 98% sequence identity thereto. The portion of exon 6 of NRG1 in the fusion with SLC3A2 preferably is or comprises the sequence according to SEQ ID NO: 453 and the allelic variant thereof has at least 85% identity to SEQ ID NO: 453, preferably at least 90% identity, 92%, 94%, 96% or more preferably at least 98% sequence identity thereto. More preferably, the portion of exon 2 of SLC3A2 comprises or consists of 2 to about 10, about 20, about 30, or up to about 40 or even all contiguous nucleic acids from SEQ ID NO: 452, including at least the nucleic acid of position 93. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 452, including at least the nucleic acid of position 93. More preferably, the portion of exon 2 of said SLC3A2 comprises or is according to SEQ ID NO: 452, or an allelic variant thereof. Such short polynucleotide sequences are particularly useful for detecting the presence of a larger polynucleotide fusion between said SLC3A2 and NRG1 and establishing that such a fusion is an in-frame, oncogenic fusion comprising an EGF-like domain of NRG1.

Alternatively, the portion of exon 2 of said SLC3A2 comprises or consists of SEQ ID NO: 457 (or an allelic variant of SEQ ID NO: 457), including at least the nucleic acid of position 93. Preferably, the portion of exon 2 of said SLC3A2 comprises or consists of 2 to about 10, about 20, about 30, or up to about 40 or even all contiguous nucleic acids from SEQ ID NO: 457, or from the allelic variant thereof, including at least the nucleic acid of position 93. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 457, including at least the nucleic acid of position 93. In this alternative, the portion of exon 2 of SLC3A2 more preferably comprises or is according to SEQ ID NO: 457, or an allelic variant thereof. Preferably, the portion of exon 6 of NRG1 in the fusion with transcript version 3 of SLC3A2 comprises or consists of 2 to about 10, about 20, about 30, or up to about 40 or even all contiguous nucleic acids from SEQ ID NO: 130, or from the allelic variant thereof, including at least the nucleic acid of position 1. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 130, including at least the nucleic acid of position 1.

Preferably, any SLC3A2-NRG1 polynucleotide fusion provided herein is an in-frame fusion of SLC3A2 with NRG1. More preferably said fusion is an in-frame fusion comprising exon 2 of transcript version 3 of SLC3A2, or a portion of exon 2, with exon 6 of NRG1, or a portion of exon 6 of NRG1. Said in-frame fusion is preferably the fusion of SEQ ID NO: 454, or an allelic variant thereof having at least 85% identity thereto, preferably at least 90%, 92%, 94%, 96% or even 98% identity thereto.

In an alternatively preferred embodiment, the polynucleotide comprising a portion of exon 2 of said SLC3A2 fused with a portion of exon 6 of NRG1 comprises 2 to about 10, about 20, about 30, or up to about 40 or even all contiguous nucleic acids from SEQ ID NO: 454, including the nucleic acids at positions 93 and 94. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 454, including at least the nucleic acids at positions 93 and 94. SEQ ID NO: 454 includes the junction between said SLC3A2 and NRG1, in particular the junction is between the nucleic acid at position 93, which stems from SLC3A2 and the nucleic acid at position 94 which stems from NRG1. Preferably, the polynucleotide comprising a portion of exon 2 of SLC3A2 fused with a portion of exon 6 of NRG1 has the polynucleotide sequence of SEQ ID NO: 454, or an allelic variant thereof.

In a preferred embodiment, there is provided a polynucleotide according to SEQ ID NO: 454, or a polynucleotide which comprises about 20, about 30, about 40 or all contiguous nucleic acids from SEQ ID NO: 454, preferably including the nucleic acids at positions 93 and 94. The number of contiguous nucleic acids may be 18, 19, 20, 21, 22, 23, 24, 25, 26, 27, 28, 29, 30, 31, 32, 33, 34, 35, 36, 37, 38, 39, 40 or all nucleic acids of SEQ ID NO: 454, preferably including at least the nucleic acids at positions 93 and 94.

Preferably, the polynucleotide comprising a portion of exon 2 of transcript version 3 of SLC3A2, or an allelic variant thereof, fused with a portion of exon 6 of NRG1, or an allelic variant thereof, is part of a longer polynucleotide that further comprises or encodes an EGF-like domain of NRG1. An aberrant cell comprising the polynucleotide fusion involving said SLC3A2 or expressing the polypeptide fusion, comprises or encodes an EGF-like domain of NRG1. For quick detection, diagnosis or identification purposes, it may suffice to show that the fusion junction between said SLC3A2 and NRG1 is in-frame and occurs at such a position that the resulting fusion product, nucleic acid or protein, comprises an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

Preferably, the portion of exon 2 of transcript version 3 of SLC3A2, or the allelic variant thereof, is 5′ to the portion of exon 6 of NRG1, or the allelic variant thereof. This orientation at the nucleic acid level results in a fusion polypeptide product that contains an N-terminus of SLC3A2 and a C-terminus of NRG1. Also, the herein provided SLC3A2-NRG1 polynucleotide fusion produces a protein fusion, wherein the part from the N-terminus to the fusion junction is polypeptide sequence from SLC3A2 and the part from the junction to the C-terminus is NRG1 polypeptide sequence, wherein the NRG1 part also provides its EGF-like domain. The SLC3A2-NRG1 fusion protein thereby retains the EGF-like domain of NRG1 and the ability to drive proliferation and survival of a subset of human cancers, including lung cancer or adenocarcinoma, in particular lung adenocarcinoma.

When present in an aberrant cell of a patient or subject, said SCL3A2-NRG1 polynucleotide fusion preferably further includes any sequence 3′ from exon 6 of NRG1, but to allow detection of the fusion junction using a polynucleotide-based detection assay, it may suffice that at least SEQ ID NOs: 457 and 130 are present. Any sequence 3′ of exon 6 of NRG1 comprises or consists of one or all of SEQ ID NOs: 131-137 (or any allelic variant of SEQ ID NOs: 131-137).

### VTCN1-NRG1 Polynucleotide Fusion

There is also provided a polynucleotide fusion comprising a portion of exon 2 of VTCN1 fused with a portion of exon 2 of NRG1. Exon 2 of VTCN1 is preferably that of SEQ ID NO: 169, or an allelic variant of SEQ ID NO: 169, and exon 2 of NRG1 is preferably that of SEQ ID NO: 126, or an allelic variant of SEQ ID NO: 126.

Preferably, the allelic variant of exon 2 of VTCN1 has at least 85% identity to SEQ ID NO: 169, preferably at least 90% identity, 92%, 94%, 96% or more preferably at least 98% sequence identity thereto and the allelic variant of exon 2 of NRG1 has at least 85% identity to SEQ ID NO: 126, preferably at least 90% identity, 92%, 94%, 96% or more preferably at least 98% sequence identity thereto.

Preferably, the portion of exon 2 of VTCN1 comprises or is according to SEQ ID NO: 164 and the allelic variant thereof has at least 85% identity to SEQ ID NO: 164, preferably at least 90% identity, 92%, 94%, 96% or more preferably at least 98% sequence identity thereto. The portion of exon 2 of NRG1 in the fusion with VTCN1 preferably is or comprises the sequence according to SEQ ID NO: 165 and the allelic variant thereof has at least 85% identity to SEQ ID NO: 165, preferably at least 90% identity, 92%, 94%, 96% or more preferably at least 98% sequence identity thereto. More preferably, the portion of exon 2 of VTCN1 comprises or consists of 2 to about 10, about 20, about 30, or up to about 40 or even all contiguous nucleic acids from SEQ ID NO: 164, including at least the nucleic acid of position 65. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 164, including at least the nucleic acid of position 65. More preferably, the portion of exon 2 of VTCN1 comprises or is according to SEQ ID NO: 164, or an allelic variant thereof. Such short polynucleotide sequences are particularly useful for detecting the presence of a larger polynucleotide fusion between VTCN1 and NRG1 and establishing that such a fusion is an in-frame, oncogenic fusion comprising an EGF-like domain of NRG1.

Preferably, any VTCN1-NRG1 polynucleotide fusion provided herein is an in-frame fusion of VTCN1 with NRG1. More preferably said fusion is an in-frame fusion comprising exon 2 of VTCN1, or a portion of exon 2, with exon 2 of NRG1, or a portion of exon 2 of NRG1. Said in-frame fusion is preferably the fusion of SEQ ID NO: 166, or an allelic variant thereof having at least 85% identity thereto, preferably at least 90%, 92%, 94%, 96% or even 98% identity thereto.

In an alternatively preferred embodiment, the polynucleotide comprising a portion of exon 2 of VTCN1 fused with a portion of exon 2 of NRG1 comprises 2 to about 10, about 20, about 30, or up to about 40 or even all contiguous nucleic acids from SEQ ID NO: 166, including the nucleic acids at positions 65 and 66. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 166, including at least the nucleic acids at positions 65 and 66. SEQ ID NO: 166 includes the junction between VTCN1 and NRG1, in particular the junction is between the nucleic acid at position 65, which stems from VTCN1 and the nucleic acid at position 66 which stems from NRG1. Preferably, the polynucleotide comprising a portion of exon 2 of VTCN1 fused with a portion of exon 2 of NRG1 has the polynucleotide sequence of SEQ ID NO: 166, or an allelic variant thereof. Preferably, the portion of exon 2 of NRG1 in the fusion with VTCN1 comprises or consists of 2 to about 10, about 20, about 30, or up to about 40 or even all contiguous nucleic acids from SEQ ID NO: 126, or from the allelic variant thereof, including at least the nucleic acid of position 1. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 126, including at least the nucleic acid of position 1.

In a preferred embodiment, there is provided a polynucleotide according to SEQ ID NO: 166, or a polynucleotide which comprises about 20, about 30, about 40 or all contiguous nucleic acids from SEQ ID NO: 166, preferably including the nucleic acids at positions 65 and 66. The number of contiguous nucleic acids may be 18, 19, 20, 21, 22, 23, 24, 25, 26, 27, 28, 29, 30, 31, 32, 33, 34, 35, 36, 37, 38, 39, 40 or all nucleic acids of SEQ ID NO: 166, preferably including at least the nucleic acids at positions 65 and 66.

Preferably, the polynucleotide comprising a portion of exon 2 of VTCN1, or an allelic variant thereof, fused with a portion of exon 2 of NRG1, or an allelic variant thereof, is part of a longer polynucleotide that further comprises or encodes an EGF-like domain of NRG1. An aberrant cell comprising the polynucleotide fusion involving VTCN1 or expressing the polypeptide fusion, comprises or encodes an EGF-like domain of NRG1. For quick detection, diagnosis or identification purposes, it may suffice to show that the fusion junction between VTCN1 and NRG1 is in-frame and occurs at such a position that the resulting fusion product, nucleic acid or protein, comprises an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

Preferably, the portion of exon 2 of VTCN1, or the allelic variant thereof, is 5′ to the portion of exon 2 of NRG1, or the allelic variant thereof. This orientation at the nucleic acid level results in a fusion polypeptide product that contains an N-terminus of VTCN1 and a C-terminus of NRG1. Also, the herein provided VTCN1-NRG1 polynucleotide fusion produces a protein fusion, wherein the part from the N-terminus to the fusion junction is polypeptide sequence from VTCN1 and the part from the junction to the C-terminus is NRG1 polypeptide sequence, wherein the NRG1 part also provides its EGF-like domain. The VTCN1-NRG1 fusion protein thereby retains the EGF-like domain of NRG1 and the ability to drive proliferation and survival of a subset of human cancers, in particular an adenocarcinoma.

When present in an aberrant cell of a patient or subject, said polynucleotide fusion preferably further includes any sequence 5′ from exon 2 of VTCN1 and any sequence 3′ from exon 2 of NRG1, but to allow detection of the fusion junction using a polynucleotide-based detection assay, it may suffice that at least SEQ ID NOs: 169 and 126 are present. Any sequence 5′ from exon 2 of VTCN1 comprises or consists of SEQ ID NOs: 168 (or any allelic variant of SEQ ID NOs: 168), and any sequence 3′ of exon 2 of NRG1 comprises or consists of one or all of SEQ ID NOs: 127-137 (or any allelic variant of SEQ ID NOs: 127-137).

### CDH1-NRG1 Polynucleotide Fusion

There is also provided a polynucleotide fusion comprising a portion of exon 11 of CDH1 fused with a portion of exon 2 of NRG1. Exon 11 of CDH1 is preferably that of SEQ ID NO: 198, or an allelic variant of SEQ ID NO: 198, and exon 2 of NRG1 is preferably that of SEQ ID NO: 126, or an allelic variant of SEQ ID NO:126.

When present in an aberrant cell of a patient or subject, said polynucleotide fusion preferably further includes any sequence 5′ from exon 11 of CDH1 and any sequence 3′ from exon 2 of NRG1, but to allow detection of the fusion junction using a polynucleotide-based detection assay, it may suffice that at least SEQ ID NOs: 198 and 126 are present. Any sequence 5′ from exon 11 of CDH1 comprises or consists of one or all of SEQ ID NOs: 188-197 (or any allelic variant of SEQ ID NOs: 188-197), and any sequence 3′ of exon 2 of NRG1 comprises or consists of one or all of SEQ ID NOs: 127-137 (or any allelic variant of SEQ ID NOs: 127-137).

Preferably, the allelic variant of exon 11 of CDH1 has at least 85% identity to SEQ ID NO: 198, preferably at least 90% identity, 92%, 94%, 96% or more preferably at least 98% sequence identity thereto and the allelic variant of exon 2 of NRG1 has at least 85% identity to SEQ ID NO: 126, preferably at least 90% identity, 92%, 94%, 96% or more preferably at least 98% sequence identity thereto.

Preferably, the portion of exon 11 of CDH1 comprises or is according to SEQ ID NO: 184 and the allelic variant thereof has at least 85% identity to SEQ ID NO: 184, preferably at least 90% identity, 92%, 94%, 96% or more preferably at least 98% sequence identity thereto. The portion of exon 2 of NRG1 in the fusion with CDH1 preferably is or comprises the sequence according to SEQ ID NO: 185 and the allelic variant thereof has at least 85% identity to SEQ ID NO: 185, preferably at least 90% identity, 92%, 94%, 96% or more preferably at least 98% sequence identity thereto. More preferably, the portion of exon 11 of CDH1 comprises or consists of 2 to about 10, about 20, about 30, or up to about 40 or even all contiguous nucleic acids from SEQ ID NO: 184, including at least the nucleic acid of position 119. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 184, including at least the nucleic acid of position 119. More preferably, the portion of exon 11 of CDH1 comprises or is according to SEQ ID NO: 184, or an allelic variant thereof. Such short polynucleotide sequences are particularly useful for detecting the presence of a larger polynucleotide fusion between CDH1 and NRG1 and establishing that such a fusion is an in-frame, oncogenic fusion comprising an EGF-like domain of NRG1.

Alternatively, the portion of exon 11 of CDH1 comprises or consists of SEQ ID NO: 198 (or an allelic variant of SEQ ID NO: 198), including at least the nucleic acid of position 146. Preferably, the portion of exon 11 of CDH1 comprises or consists of 2 to about 10, about 20, about 30, or up to about 40 or even all contiguous nucleic acids from SEQ ID NO: 198, or from the allelic variant thereof, including at least the nucleic acid of position 146. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 198, including at least the nucleic acid of position 146. In this alternative, the portion of exon 11 of CDH1 more preferably comprises or is according to SEQ ID NO: 198, or an allelic variant thereof.

Preferably, any CDH1-NRG1 polynucleotide fusion provided herein is an in-frame fusion of CDH1 with NRG1. More preferably said fusion is an in-frame fusion comprising exon 11 of CDH1, or a portion of exon 2, with exon 2 of NRG1, or a portion of exon 2 of NRG1. Said in-frame fusion is preferably the fusion of SEQ ID NO: 186, or an allelic variant thereof having at least 85% identity thereto, preferably at least 90%, 92%, 94%, 96% or even 98% identity thereto.

In an alternatively preferred embodiment, the polynucleotide comprising a portion of exon 11 of CDH1 fused with a portion of exon 2 of NRG1 comprises 2 to about 10, about 20, about 30, or up to about 40 or even all contiguous nucleic acids from SEQ ID NO: 186, including the nucleic acids at positions 119 and 120. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 186, including at least the nucleic acids at positions 119 and 120. SEQ ID NO: 186 includes the junction between CDH1 and NRG1, in particular the junction is between the nucleic acid at position 119, which stems from CDH1 and the nucleic acid at position 120 which stems from NRG1. Preferably, the polynucleotide comprising a portion of exon 11 of CDH1 fused with a portion of exon 2 of NRG1 has the polynucleotide sequence of SEQ ID NO: 186, or an allelic variant thereof.

In a preferred embodiment, there is provided a polynucleotide according to SEQ ID NO: 186, or a polynucleotide which comprises about 20, about 30, about 40 or all contiguous nucleic acids from SEQ ID NO: 186, preferably including the nucleic acids at positions 119 and 120. The number of contiguous nucleic acids may be 18, 19, 20, 21, 22, 23, 24, 25, 26, 27, 28, 29, 30, 31, 32, 33, 34, 35, 36, 37, 38, 39, 40 or all nucleic acids of SEQ ID NO: 186, preferably including at least the nucleic acids at positions 119 and 120.

Preferably, the polynucleotide comprising a portion of exon 11 of CDH1, or an allelic variant thereof, fused with a portion of exon 2 of NRG1, or an allelic variant thereof, is part of a longer polynucleotide that further comprises or encodes an EGF-like domain of NRG1. An aberrant cell comprising the polynucleotide fusion involving CDH1 or expressing the polypeptide fusion, comprises or encodes an EGF-like domain of NRG1. For quick detection, diagnosis or identification purposes, it may suffice to show that the fusion junction between CDH1 and NRG1 is in-frame and occurs at such a position that the resulting fusion product, nucleic acid or protein, comprises an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

Preferably, the portion of exon 11 of CDH1, or the allelic variant thereof, is 5′ to the portion of exon 2 of NRG1, or the allelic variant thereof. This orientation at the nucleic acid level results in a fusion polypeptide product that contains an N-terminus of CDH1 and a C-terminus of NRG1. Also, the herein provided CDH1-NRG1 polynucleotide fusion produces a protein fusion, wherein the part from the N-terminus to the fusion junction is polypeptide sequence from CDH1 and the part from the junction to the C-terminus is NRG1 polypeptide sequence, wherein the NRG1 part also provides its EGF-like domain. The CDH1-NRG1 fusion protein thereby retains the EGF-like domain of NRG1 and the ability to drive proliferation and survival of a subset of human cancers, in particular an adenocarcinoma, more in particular pancreatic ductal adenocarcinoma.

### CXADR-NRG1 Polynucleotide Fusion

There is also provided a polynucleotide fusion comprising a portion of Exon 1 of CXADR fused with a portion of exon 2 of NRG1. Exon 1 of CXADR is preferably that of SEQ ID NO: 219, or an allelic variant of SEQ ID NO: 219, and exon 2 of NRG1 is preferably that of SEQ ID NO: 126, or an allelic variant of SEQ ID NO:126.

When present in an aberrant cell of a patient or subject, said polynucleotide fusion preferably further includes any sequence 5′ from exon 1 of CXADR and any sequence 3′ from exon 2 of NRG1, but to allow detection of the fusion junction using a polynucleotide-based detection assay, it may suffice that at least SEQ ID NOs: 219 and 126 are present. Any sequence 3′ of exon 2 of NRG1 comprises or consists of one or all of SEQ ID NOs: 127-137 (or any allelic variant of SEQ ID NOs: 127-137).

Preferably, the allelic variant of exon 1 of CXADR has at least 85% identity to SEQ ID NO: 219, preferably at least 90% identity, 92%, 94%, 96% or more preferably at least 98% sequence identity thereto and the allelic variant of exon 2 of NRG1 has at least 85% identity to SEQ ID NO: 126, preferably at least 90% identity, 92%, 94%, 96% or more preferably at least 98% sequence identity thereto.

Preferably, the portion of exon 1 of CXADR comprises or is according to SEQ ID NO: 215 and the allelic variant thereof has at least 85% identity to SEQ ID NO: 215, preferably at least 90% identity, 92%, 94%, 96% or more preferably at least 98% sequence identity thereto. The portion of exon 2 of NRG1 in the fusion with CXADR preferably is or comprises the sequence according to SEQ ID NO: 216 and the allelic variant thereof has at least 85% identity to SEQ ID NO: 216, preferably at least 90% identity, 92%, 94%, 96% or more preferably at least 98% sequence identity thereto. More preferably, the portion of exon 1 of CXADR comprises or consists of 2 to about 10, about 20, about 30, or up to about 40 or even all contiguous nucleic acids from SEQ ID NO: 215, including at least the nucleic acid of position 43. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 215, including at least the nucleic acid of position 43. More preferably, the portion of exon 1 of CXADR comprises or is according to SEQ ID NO: 215, or an allelic variant thereof. Such short polynucleotide sequences are particularly useful for detecting the presence of a larger polynucleotide fusion between CXADR and NRG1 and establishing that such a fusion is an in-frame, oncogenic fusion comprising an EGF-like domain of NRG1.

Alternatively, the portion of exon 1 of CXADR comprises or consists of SEQ ID NO: 219 (or an allelic variant of SEQ ID NO: 219), including at least the nucleic acid of position 130. Preferably, the portion of exon 1 of CXADR comprises or consists of 2 to about 10, about 20, about 30, or up to about 40 or even all contiguous nucleic acids from SEQ ID NO: 219, or from the allelic variant thereof, including at least the nucleic acid of position 130. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 219, including at least the nucleic acid of position 130. In this alternative, the portion of exon 1 of CXADR more preferably comprises or is according to SEQ ID NO: 219, or an allelic variant thereof.

Preferably, any CXADR-NRG1 polynucleotide fusion provided herein is an in-frame fusion of CXADR with NRG1. More preferably said fusion is an in-frame fusion comprising exon 1 of CXADR, or a portion of exon 2, with exon 2 of NRG1, or a portion of exon 2 of NRG1. Said in-frame fusion is preferably the fusion of SEQ ID NO: 217, or an allelic variant thereof having at least 85% identity thereto, preferably at least 90%, 92%, 94%, 96% or even 98% identity thereto.

In an alternatively preferred embodiment, the polynucleotide comprising a portion of exon 1 of CXADR fused with a portion of exon 2 of NRG1 comprises 2 to about 10, about 20, about 30, or up to about 40 or even all contiguous nucleic acids from SEQ ID NO: 217, including the nucleic acids at positions 43 and 44. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 217, including at least the nucleic acids at positions 43 and 44. SEQ ID NO: 217 includes the junction between CXADR and NRG1, in particular the junction is between the nucleic acid at position 43, which stems from CXADR and the nucleic acid at position 44 which stems from NRG1. Preferably, the polynucleotide comprising a portion of exon 1 of CXADR fused with a portion of exon 2 of NRG1 has the polynucleotide sequence of SEQ ID NO: 217, or an allelic variant thereof. Preferably, the portion of exon 2 of NRG1 in the fusion with CXADR comprises or consists of 2 to about 10, about 20, about 30, or up to about 40 or even all contiguous nucleic acids from SEQ ID NO: 126, or from the allelic variant thereof, including at least the nucleic acid of position 1. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 126, including at least the nucleic acid of position 1.

In a preferred embodiment, there is provided a polynucleotide according to SEQ ID NO: 217, or a polynucleotide which comprises about 20, about 30, about 40 or all contiguous nucleic acids from SEQ ID NO: 217, preferably including the nucleic acids at positions 43 and 44. The number of contiguous nucleic acids may be 18, 19, 20, 21, 22, 23, 24, 25, 26, 27, 28, 29, 30, 31, 32, 33, 34, 35, 36, 37, 38, 39, 40 or all nucleic acids of SEQ ID NO: 217, preferably including at least the nucleic acids at positions 43 and 44.

Preferably, the polynucleotide comprising a portion of exon 1 of CXADR, or an allelic variant thereof, fused with a portion of exon 2 of NRG1, or an allelic variant thereof, is part of a longer polynucleotide that further comprises or encodes an EGF-like domain of NRG1. An aberrant cell comprising the polynucleotide fusion involving CXADR or expressing the polypeptide fusion, comprises or encodes an EGF-like domain of NRG1. For quick detection, diagnosis or identification purposes, it may suffice to show that the fusion junction between CXADR and NRG1 is in frame and occurs at such a position that the resulting fusion product, nucleic acid or protein, comprises an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

Preferably, the portion of exon 1 of CXADR, or the allelic variant thereof, is 5′ to the portion of exon 2 of NRG1, or the allelic variant thereof. This orientation at the nucleic acid level results in a fusion polypeptide product that contains an N-terminus of CXADR and a C-terminus of NRG1. Also, the herein provided CXADR-NRG1 polynucleotide fusion produces a protein fusion, wherein the part from the N-terminus to the fusion junction is polypeptide sequence from CXADR and the part from the junction to the C-terminus is NRG1 polypeptide sequence, wherein the NRG1 part also provides its EGF-like domain. The CXADR-NRG1 fusion protein thereby retains the EGF-like domain of NRG1 and the ability to drive proliferation and survival of a subset of human cancers, in particular colon cancer.

### GTF2E2-NRG1 Polynucleotide Fusion

There is also provided a polynucleotide fusion comprising a portion of exon 2 of GTF2E2 fused with a portion of exon 2 of NRG1. Exon 2 of GTF2E2 is preferably that of SEQ ID NO: 236, or an allelic variant of SEQ ID NO: 236, and exon 2 of NRG1 is preferably that of SEQ ID NO: 126, or an allelic variant of SEQ ID NO:126.

When present in an aberrant cell of a patient or subject, said polynucleotide fusion preferably further includes any sequence 5′ from exon 2 of GTF2E2 and any sequence 3′ from exon 2 of NRG1, but to allow detection of the fusion junction using a polynucleotide-based detection assay, it may suffice that at least SEQ ID NOs: 236 and 126 are present. Any sequence 5′ from exon 2 of GTF2E2 comprises or consists of one or all of SEQ ID NO: 235 (or any allelic variant of SEQ ID NO: 235), and any sequence 3′ of exon 2 of NRG1 comprises or consists of one or all of SEQ ID NOs: 127-137 (or any allelic variant of SEQ ID NOs: 127-137).

Preferably, the allelic variant of exon 2 of GTF2E2 has at least 85% identity to SEQ ID NO: 236, preferably at least 90% identity, 92%, 94%, 96% or more preferably at least 98% sequence identity thereto and the allelic variant of exon 2 of NRG1 has at least 85% identity to SEQ ID NO: 126, preferably at least 90% identity, 92%, 94%, 96% or more preferably at least 98% sequence identity thereto.

Preferably, the portion of exon 2 of GTF2E2 comprises or is according to SEQ ID NO: 231 and the allelic variant thereof has at least 85% identity to SEQ ID NO: 231, preferably at least 90% identity, 92%, 94%, 96% or more preferably at least 98% sequence identity thereto. The portion of exon 2 of NRG1 in the fusion with GTF2E2 preferably is or comprises the sequence according to SEQ ID NO: 232 and the allelic variant thereof has at least 85% identity to SEQ ID NO: 232, preferably at least 90% identity, 92%, 94%, 96% or more preferably at least 98% sequence identity thereto. More preferably, the portion of exon 2 of GTF2E2 comprises or consists of 2 to about 10, about 20, about 30, or up to about 40 or even all contiguous nucleic acids from SEQ ID NO: 231, including at least the nucleic acid of position 141. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 231, including at least the nucleic acid of position 141. More preferably, the portion of exon 2 of GTF2E2 comprises or is according to SEQ ID NO: 231, or an allelic variant thereof. Such short polynucleotide sequences are particularly useful for detecting the presence of a larger polynucleotide fusion between GTF2E2 and NRG1 and establishing that such a fusion is an in-frame, oncogenic fusion comprising an EGF-like domain of NRG1.

Alternatively, the portion of exon 2 of GTF2E2 comprises or consists of SEQ ID NO: 236 (or an allelic variant of SEQ ID NO: 236), including at least the nucleic acid of position 170. Preferably, the portion of exon 2 of GTF2E2 comprises or consists of 2 to about 10, about 20, about 30, or up to about 40 or even all contiguous nucleic acids from SEQ ID NO: 236, or from the allelic variant thereof, including at least the nucleic acid of position 170. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 236, including at least the nucleic acid of position 170. In this alternative, the portion of exon 2 of GTF2E2 more preferably comprises or is according to SEQ ID NO: 236, or an allelic variant thereof. Preferably, the portion of exon 2 of NRG1 in the fusion with GTF2E2 comprises or consists of 2 to about 10, about 20, about 30, or up to about 40 or even all contiguous nucleic acids from SEQ ID NO: 126, or from the allelic variant thereof, including at least the nucleic acid of position 1. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 126, including at least the nucleic acid of position 1.

Preferably, any GTF2E2-NRG1 polynucleotide fusion provided herein is an in-frame fusion of GTF2E2 with NRG1. More preferably said fusion is an in-frame fusion comprising exon 2 of GTF2E2, or a portion of exon 2, with exon 2 of NRG1, or a portion of exon 2 of NRG1. Said in frame fusion is preferably the fusion of SEQ ID NO: 233, or an allelic variant thereof having at least 85% identity thereto, preferably at least 90%, 92%, 94%, 96% or even 98% identity thereto.

In an alternatively preferred embodiment, the polynucleotide comprising a portion of exon 2 of GTF2E2 fused with a portion of exon 2 of NRG1 comprises 2 to about 10, about 20, about 30, or up to about 40 or even all contiguous nucleic acids from SEQ ID NO: 233, including the nucleic acids at positions 141 and 142. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 233, including at least the nucleic acids at positions 141 and 142. SEQ ID NO: 233 includes the junction between GTF2E2 and NRG1, in particular the junction is between the nucleic acid at position 141, which stems from GTF2E2 and the nucleic acid at position 142 which stems from NRG1. Preferably, the polynucleotide comprising a portion of exon 2 of GTF2E2 fused with a portion of exon 2 of NRG1 has the polynucleotide sequence of SEQ ID NO: 233, or an allelic variant thereof.

In a preferred embodiment, there is provided a polynucleotide according to SEQ ID NO: 233, or a polynucleotide which comprises about 20, about 30, about 40 or all contiguous nucleic acids from SEQ ID NO: 233, preferably including the nucleic acids at positions 141 and 142. The number of contiguous nucleic acids may be 18, 19, 20, 21, 22, 23, 24, 25, 26, 27, 28, 29, 30, 31, 32, 33, 34, 35, 36, 37, 38, 39, 40 or all nucleic acids of SEQ ID NO: 233, preferably including at least the nucleic acids at positions 141 and 142.

Preferably, the polynucleotide comprising a portion of exon 2 of GTF2E2, or an allelic variant thereof, fused with a portion of exon 2 of NRG1, or an allelic variant thereof, is part of a longer polynucleotide that further comprises or encodes an EGF-like domain of NRG1. An aberrant cell comprising the polynucleotide fusion involving GTF2E2 or expressing the polypeptide fusion, comprises or encodes an EGF-like domain of NRG1. For quick detection, diagnosis or identification purposes, it may suffice to show that the fusion junction between GTF2E2 and NRG1 is in-frame and occurs at such a position that the resulting fusion product, nucleic acid or protein, comprises an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

Preferably, the portion of exon 2 of GTF2E2, or the allelic variant thereof, is 5′ to the portion of exon 2 of NRG1, or the allelic variant thereof. This orientation at the nucleic acid level results in a fusion polypeptide product that contains an N-terminus of GTF2E2 and a C-terminus of NRG1. Also, the herein provided GTF2E2-NRG1 polynucleotide fusion produces a protein fusion, wherein the part from the N-terminus to the fusion junction is polypeptide sequence from GTF2E2 and the part from the junction to the C-terminus is NRG1 polypeptide sequence, wherein the NRG1 part also provides its EGF-like domain. The GTF2E2-NRG1 fusion protein thereby retains the EGF-like domain of NRG1 and the ability to drive proliferation and survival of a subset of human cancers, in particular an adenocarcinoma, more in particular (metastatic) breast adenocarcinoma NOS.

### CSMD1-NRG1 Polynucleotide Fusion

There is also provided a polynucleotide fusion comprising a portion of exon 23 of CSMD1 fused with a portion of exon 6 of NRG1. Exon 23 of CSMD1 is preferably that of SEQ ID NO: 279, or an allelic variant of SEQ ID NO: 279, and exon 6 of NRG1 is preferably that of SEQ ID NO: 130, or an allelic variant of SEQ ID NO:130.

When present in an aberrant cell of a patient or subject, said polynucleotide fusion preferably further includes any sequence 5′ from exon 23 of CSMD1 and any sequence 3′ from exon 6 of NRG1, but to allow detection of the fusion junction using a polynucleotide-based detection assay, it may suffice that at least SEQ ID NOs: 279 and 130 are present. Any sequence 5′ from exon 23 of CSMD1 comprises or consists of one or all of SEQ ID NOs: 257-278 (or any allelic variant of SEQ ID NOs: 257-278), and any sequence 3′ of exon 6 of NRG1 comprises or consists of one or all of SEQ ID NOs: 131-137 (or any allelic variant of SEQ ID NOs: 131-137).

Preferably, the allelic variant of exon 23 of CSMD1 has at least 85% identity to SEQ ID NO: 279, preferably at least 90% identity, 92%, 94%, 96% or more preferably at least 98% sequence identity thereto and the allelic variant of exon 6 of NRG1 has at least 85% identity to SEQ ID NO: 130, preferably at least 90% identity, 92%, 94%, 96% or more preferably at least 98% sequence identity thereto.

Preferably, the portion of exon 23 of CSMD1 comprises or is according to SEQ ID NO: 253 and the allelic variant thereof has at least 85% identity to SEQ ID NO: 253, preferably at least 90% identity, 92%, 94%, 96% or more preferably at least 98% sequence identity thereto. The portion of exon 6 of NRG1 in the fusion with CSMD1 preferably is or comprises the sequence according to SEQ ID NO: 254 and the allelic variant thereof has at least 85% identity to SEQ ID NO: 254, preferably at least 90% identity, 92%, 94%, 96% or more preferably at least 98% sequence identity thereto. More preferably, the portion of exon 23 of CSMD1 comprises or consists of 2 to about 10, about 20, about 30, or up to about 40 or even all contiguous nucleic acids from SEQ ID NO: 253, including at least the nucleic acid of position 88. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 253, including at least the nucleic acid of position 88. More preferably, the portion of exon 23 of CSMD1 comprises or is according to SEQ ID NO: 253, or an allelic variant thereof. Such short polynucleotide sequences are particularly useful for detecting the presence of a larger polynucleotide fusion between CSMD1 and NRG1 and establishing that such a fusion is an in-frame, oncogenic fusion comprising an EGF-like domain of NRG1.

Alternatively, the portion of exon 23 of CSMD1 comprises or consists of SEQ ID NO: 279 (or an allelic variant of SEQ ID NO: 279), including at least the nucleic acid of position 157. Preferably, the portion of exon 23 of CSMD1 comprises or consists of 2 to about 10, about 20, about 30, or up to about 40 or even all contiguous nucleic acids from SEQ ID NO: 279, or from the allelic variant thereof, including at least the nucleic acid of position 157. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 279, including at least the nucleic acid of position 157. In this alternative, the portion of exon 23 of CSMD1 more preferably comprises or is according to SEQ ID NO: 279, or an allelic variant thereof. Preferably, the portion of exon 6 of NRG1 in the fusion with CSMD1 comprises or consists of 2 to about 10, about 20, about 30, or up to about 40 or even all contiguous nucleic acids from SEQ ID NO: 130, or from the allelic variant thereof, including at least the nucleic acid of position 1. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 130, including at least the nucleic acid of position 1.

Preferably, any CSMD1-NRG1 polynucleotide fusion provided herein is an in-frame fusion of CSMD1 with NRG1. More preferably said fusion is an in-frame fusion comprising exon 23 of CSMD1, or a portion of exon 6, with exon 6 of NRG1, or a portion of exon 6 of NRG1. Said in-frame fusion is preferably the fusion of SEQ ID NO: 255, or an allelic variant thereof having at least 85% identity thereto, preferably at least 90%, 92%, 94%, 96% or even 98% identity thereto.

In an alternatively preferred embodiment, the polynucleotide comprising a portion of exon 23 of CSMD1 fused with a portion of exon 6 of NRG1 comprises 2 to about 10, about 20, about 30, or up to about 40 or even all contiguous nucleic acids from SEQ ID NO: 255, including the nucleic acids at positions 88 and 89. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 255, including at least the nucleic acids at positions 88 and 89. SEQ ID NO: 255 includes the junction between CSMD1 and NRG1, in particular the junction is between the nucleic acid at position 88, which stems from CSMD1 and the nucleic acid at position 89 which stems from NRG1. Preferably, the polynucleotide comprising a portion of exon 23 of CSMD1 fused with a portion of exon 6 of NRG1 has the polynucleotide sequence of SEQ ID NO: 255, or an allelic variant thereof.

In a preferred embodiment, there is provided a polynucleotide according to SEQ ID NO: 255, or a polynucleotide which comprises about 20, about 30, about 40 or all contiguous nucleic acids from SEQ ID NO: 255, preferably including the nucleic acids at positions 88 and 89. The number of contiguous nucleic acids may be 18, 19, 20, 21, 22, 23, 24, 25, 26, 27, 28, 29, 30, 31, 32, 33, 34, 35, 36, 37, 38, 39, 40 or all nucleic acids of SEQ ID NO: 255, preferably including at least the nucleic acids at positions 88 and 89.

Preferably, the polynucleotide comprising a portion of exon 23 of CSMD1, or an allelic variant thereof, fused with a portion of exon 6 of NRG1, or an allelic variant thereof, is part of a longer polynucleotide that further comprises or encodes an EGF-like domain of NRG1. An aberrant cell comprising the polynucleotide fusion involving CSMD1 or expressing the polypeptide fusion, comprises or encodes an EGF-like domain of NRG1. For quick detection, diagnosis or identification purposes, it may suffice to show that the fusion junction between CSMD1 and NRG1 is in-frame and occurs at such a position that the resulting fusion product, nucleic acid or protein, comprises an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

Preferably, the portion of exon 23 of CSMD1, or the allelic variant thereof, is 5′ to the portion of exon 6 of NRG1, or the allelic variant thereof. This orientation at the nucleic acid level results in a fusion polypeptide product that contains an N-terminus of CSMD1 and a C-terminus of NRG1. Also, the herein provided CSMD1-NRG1 polynucleotide fusion produces a protein fusion, wherein the part from the N-terminus to the fusion junction is polypeptide sequence from CSMD1 and the part from the junction to the C-terminus is NRG1 polypeptide sequence, wherein the NRG1 part also provides its EGF-like domain. The CSMD1-NRG1 fusion protein thereby retains the EGF-like domain of NRG1 and the ability to drive proliferation and survival of a subset of human cancers, in particular an adenocarcinoma, more in particular pancreatic ductal adenocarcinoma.

### PTN-NRG1 Polynucleotide Fusion

There is also provided a polynucleotide fusion comprising a portion of exon 4 of PTN fused with a portion of exon 2 of NRG1. Exon 4 of PTN is preferably that of SEQ ID NO: 318, or an allelic variant of SEQ ID NO: 318, and exon 2 of NRG1 is preferably that of SEQ ID NO: 126, or an allelic variant of SEQ ID NO:126.

When present in an aberrant cell of a patient or subject, said polynucleotide fusion preferably further includes any sequence 5′ from exon 4 of PTN and any sequence 3′ from exon 2 of NRG1, but to allow detection of the fusion junction using a polynucleotide-based detection assay, it may suffice that at least SEQ ID NOs: 318 and 126 are present. Any sequence 5′ from exon 4 of PTN comprises or consists of one or all of SEQ ID NOs: 315-317 (or any allelic variant of SEQ ID NOs: 315-317), and any sequence 3′ of exon 2 of NRG1 comprises or consists of one or all of SEQ ID NOs: 127-137 (or any allelic variant of SEQ ID NOs: 127-137).

Preferably, the allelic variant of exon 4 of PTN has at least 85% identity to SEQ ID NO: 318, preferably at least 90% identity, 92%, 94%, 96% or more preferably at least 98% sequence identity thereto and the allelic variant of exon 2 of NRG1 has at least 85% identity to SEQ ID NO: 126, preferably at least 90% identity, 92%, 94%, 96% or more preferably at least 98% sequence identity thereto.

Preferably, the portion of exon 4 of PTN comprises or is according to SEQ ID NO: 311 and the allelic variant thereof has at least 85% identity to SEQ ID NO: 311, preferably at least 90% identity, 92%, 94%, 96% or more preferably at least 98% sequence identity thereto. The portion of exon 2 of NRG1 in the fusion with PTN preferably is or comprises the sequence according to SEQ ID NO: 312 and the allelic variant thereof has at least 85% identity to SEQ ID NO: 312, preferably at least 90% identity, 92%, 94%, 96% or more preferably at least 98% sequence identity thereto. More preferably, the portion of exon 4 of PTN comprises or consists of 2 to about 10, about 20, about 30, or up to about 40 or even all contiguous nucleic acids from SEQ ID NO: 311, including at least the nucleic acid of position 102. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 311, including at least the nucleic acid of position 102. More preferably, the portion of exon 4 of PTN comprises or is according to SEQ ID NO: 311, or an allelic variant thereof. Such short polynucleotide sequences are particularly useful for detecting the presence of a larger polynucleotide fusion between PTN and NRG1 and establishing that such a fusion is an in-frame, oncogenic fusion comprising an EGF-like domain of NRG1.

Alternatively, the portion of exon 4 of PTN comprises or consists of SEQ ID NO: 318 (or an allelic variant of SEQ ID NO: 318), including at least the nucleic acid of position 162. Preferably, the portion of exon 4 of PTN comprises or consists of 2 to about 10, about 20, about 30, or up to about 40 or even all contiguous nucleic acids from SEQ ID NO: 318, or from the allelic variant thereof, including at least the nucleic acid of position 162. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 318, including at least the nucleic acid of position 162. In this alternative, the portion of exon 4 of PTN more preferably comprises or is according to SEQ ID NO: 318, or an allelic variant thereof. Preferably, the portion of exon 2 of NRG1 in the fusion with PTN comprises or consists of 2 to about 10, about 20, about 30, or up to about 40 or even all contiguous nucleic acids from SEQ ID NO: 126, or from the allelic variant thereof, including at least the nucleic acid of position 1. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 126, including at least the nucleic acid of position 1.

Preferably, any PTN-NRG1 polynucleotide fusion provided herein is an in-frame fusion of PTN with NRG1. More preferably said fusion is an in frame fusion comprising exon 4 of PTN, or a portion of exon 2, with exon 2 of NRG1, or a portion of exon 2 of NRG1. Said in-frame fusion is preferably the fusion of SEQ ID NO: 313, or an allelic variant thereof having at least 85% identity thereto, preferably at least 90%, 92%, 94%, 96% or even 98% identity thereto.

In an alternatively preferred embodiment, the polynucleotide comprising a portion of exon 4 of PTN fused with a portion of exon 2 of NRG1 comprises 2 to about 10, about 20, about 30, or up to about 40 or even all contiguous nucleic acids from SEQ ID NO: 313, including the nucleic acids at positions 102 and 103. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 313, including at least the nucleic acids at positions 102 and 103. SEQ ID NO: 313 includes the junction between PTN and NRG1, in particular the junction is between the nucleic acid at position 102, which stems from PTN and the nucleic acid at position 103 which stems from NRG1. Preferably, the polynucleotide comprising a portion of exon 4 of PTN fused with a portion of exon 2 of NRG1 has the polynucleotide sequence of SEQ ID NO: 313, or an allelic variant thereof.

In a preferred embodiment, there is provided a polynucleotide according to SEQ ID NO: 313, or a polynucleotide which comprises about 20, about 30, about 40 or all contiguous nucleic acids from SEQ ID NO: 313, preferably including the nucleic acids at positions 102 and 103. The number of contiguous nucleic acids may be 18, 19, 20, 21, 22, 23, 24, 25, 26, 27, 28, 29, 30, 31, 32, 33, 34, 35, 36, 37, 38, 39, 40 or all nucleic acids of SEQ ID NO: 313, preferably including at least the nucleic acids at positions 102 and 103.

Preferably, the polynucleotide comprising a portion of exon 4 of PTN, or an allelic variant thereof, fused with a portion of exon 2 of NRG1, or an allelic variant thereof, is part of a longer polynucleotide that further comprises or encodes an EGF-like domain of NRG1. An aberrant cell comprising the polynucleotide fusion involving PTN or expressing the polypeptide fusion, comprises or encodes an EGF-like domain of NRG1. For quick detection, diagnosis or identification purposes, it may suffice to show that the fusion junction between PTN and NRG1 is in-frame and occurs at such a position that the resulting fusion product, nucleic acid or protein, comprises an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

Preferably, the portion of exon 4 of PTN, or the allelic variant thereof, is 5′ to the portion of exon 2 of NRG1, or the allelic variant thereof. This orientation at the nucleic acid level results in a fusion polypeptide product that contains an N-terminus of PTN and a C-terminus of NRG1. Also, the herein provided PTN-NRG1 polynucleotide fusion produces a protein fusion, wherein the part from the N-terminus to the fusion junction is polypeptide sequence from PTN and the part from the junction to the C-terminus is NRG1 polypeptide sequence, wherein the NRG1 part also provides its EGF-like domain. The PTN-NRG1 fusion protein thereby retains the EGF-like domain of NRG1 and the ability to drive proliferation and survival of a subset of human cancers, in particular an adenocarcinoma, more in particular pancreatic ductal adenocarcinoma.

### ST14-NRG1 Polynucleotide Fusion

There is also provided a polynucleotide fusion comprising a portion of exon 11 of ST14 fused with a portion of exon 6 of NRG1. Exon 11 of ST14 is preferably that of SEQ ID NO: 342, or an allelic variant of SEQ ID NO: 342, and exon 6 of NRG1 is preferably that of SEQ ID NO: 130, or an allelic variant of SEQ ID NO:130.

When present in an aberrant cell of a patient or subject, said polynucleotide fusion preferably further includes any sequence 5′ from exon 11 of ST14 and any sequence 3′ from exon 6 of NRG1, but to allow detection of the fusion junction using a polynucleotide-based detection assay, it may suffice that at least SEQ ID NOs: 342 and 130 are present. Any sequence 5′ from exon 11 of ST14 comprises or consists of one or all of SEQ ID NOs: 332-341 (or any allelic variant of SEQ ID NOs: 332-341), and any sequence 3′ of exon 6 of NRG1 comprises or consists of one or all of SEQ ID NOs: 131-137 (or any allelic variant of SEQ ID NOs: 131-137).

Preferably, the allelic variant of exon 11 of ST14 has at least 85% identity to SEQ ID NO: 342, preferably at least 90% identity, 92%, 94%, 96% or more preferably at least 98% sequence identity thereto and the allelic variant of exon 6 of NRG1 has at least 85% identity to SEQ ID NO: 130, preferably at least 90% identity, 92%, 94%, 96% or more preferably at least 98% sequence identity thereto.

Preferably, the portion of exon 11 of ST14 comprises or is according to SEQ ID NO: 328 and the allelic variant thereof has at least 85% identity to SEQ ID NO: 328, preferably at least 90% identity, 92%, 94%, 96% or more preferably at least 98% sequence identity thereto. The portion of exon 6 of NRG1 in the fusion with ST14 preferably is or comprises the sequence according to SEQ ID NO: 329 and the allelic variant thereof has at least 85% identity to SEQ ID NO: 329, preferably at least 90% identity, 92%, 94%, 96% or more preferably at least 98% sequence identity thereto. More preferably, the portion of exon 11 of ST14 comprises or consists of 2 to about 10, about 20, about 30, or up to about 40 or even all contiguous nucleic acids from SEQ ID NO: 328, including at least the nucleic acid of position 95. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 328, including at least the nucleic acid of position 95. More preferably, the portion of exon 11 of ST14 comprises or is according to SEQ ID NO: 328, or an allelic variant thereof. Such short polynucleotide sequences are particularly useful for detecting the presence of a larger polynucleotide fusion between ST14 and NRG1 and establishing that such a fusion is an in-frame, oncogenic fusion comprising an EGF-like domain of NRG1.

Alternatively, the portion of exon 11 of ST14 comprises or consists of SEQ ID NO: 342 (or an allelic variant of SEQ ID NO: 342), including at least the nucleic acid of position 131. Preferably, the portion of exon 11 of ST14 comprises or consists of 2 to about 10, about 20, about 30, or up to about 40 or even all contiguous nucleic acids from SEQ ID NO: 342, or from the allelic variant thereof, including at least the nucleic acid of position 131. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 342, including at least the nucleic acid of position 131. In this alternative, the portion of exon 11 of ST14 more preferably comprises or is according to SEQ ID NO: 342, or an allelic variant thereof. Preferably, the portion of exon 6 of NRG1 in the fusion with ST14 comprises or consists of 2 to about 10, about 20, about 30, or up to about 40 or even all contiguous nucleic acids from SEQ ID NO: 130, or from the allelic variant thereof, including at least the nucleic acid of position 1. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 130, including at least the nucleic acid of position 1.

Preferably, any ST14-NRG1 polynucleotide fusion provided herein is an in-frame fusion of ST14 with NRG1. More preferably said fusion is an in-frame fusion comprising exon 11 of ST14, or a portion of exon 6, with exon 6 of NRG1, or a portion of exon 6 of NRG1. Said in-frame fusion is preferably the fusion of SEQ ID NO: 330, or an allelic variant thereof having at least 85% identity thereto, preferably at least 90%, 92%, 94%, 96% or even 98% identity thereto.

In an alternatively preferred embodiment, the polynucleotide comprising a portion of exon 11 of ST14 fused with a portion of exon 6 of NRG1 comprises 2 to about 10, about 20, about 30, or up to about 40 or even all contiguous nucleic acids from SEQ ID NO: 330, including the nucleic acids at positions 95 and 96. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 330, including at least the nucleic acids at positions 95 and 96. SEQ ID NO: 330 includes the junction between ST14 and NRG1, in particular the junction is between the nucleic acid at position 95, which stems from ST14 and the nucleic acid at position 96 which stems from NRG1. Preferably, the polynucleotide comprising a portion of exon 11 of ST14 fused with a portion of exon 6 of NRG1 has the polynucleotide sequence of SEQ ID NO: 330, or an allelic variant thereof.

In a preferred embodiment, there is provided a polynucleotide according to SEQ ID NO: 330, or a polynucleotide which comprises about 20, about 30, about 40 or all contiguous nucleic acids from SEQ ID NO: 330, preferably including the nucleic acids at positions 95 and 96. The number of contiguous nucleic acids may be 18, 19, 20, 21, 22, 23, 24, 25, 26, 27, 28, 29, 30, 31, 32, 33, 34, 35, 36, 37, 38, 39, 40 or all nucleic acids of SEQ ID NO: 330, preferably including at least the nucleic acids at positions 95 and 96.

Preferably, the polynucleotide comprising a portion of exon 11 of ST14, or an allelic variant thereof, fused with a portion of exon 6 of NRG1, or an allelic variant thereof, is part of a longer polynucleotide that further comprises or encodes an EGF-like domain of NRG1. An aberrant cell comprising the polynucleotide fusion involving ST14 or expressing the polypeptide fusion, comprises or encodes an EGF-like domain of NRG1. For quick detection, diagnosis or identification purposes, it may suffice to show that the fusion junction between ST14 and NRG1 is in-frame and occurs at such a position that the resulting fusion product, nucleic acid or protein, comprises an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

Preferably, the portion of exon 11 of ST14, or the allelic variant thereof, is 5′ to the portion of exon 6 of NRG1, or the allelic variant thereof. This orientation at the nucleic acid level results in a fusion polypeptide product that contains an N-terminus of ST14 and a C-terminus of NRG1. Also, the herein provided ST14-NRG1 polynucleotide fusion produces a protein fusion, wherein the part from the N-terminus to the fusion junction is polypeptide sequence from ST14 and the part from the junction to the C-terminus is NRG1 polypeptide sequence, wherein the NRG1 part also provides its EGF-like domain. The ST14-NRG1 fusion protein thereby retains the EGF-like domain of NRG1 and the ability to drive proliferation and survival of a subset of human cancers, in particular an adenocarcinoma, more in particular pancreatic ductal adenocarcinoma.

### THBS1-NRG1 Polynucleotide Fusion

There is also provided a polynucleotide fusion comprising a portion of exon 9 of THBS1 fused with a portion of exon 6 of NRG1. Exon 9 of THBS1 is preferably that of SEQ ID NO: 386, or an allelic variant of SEQ ID NO: 386, and exon 6 of NRG1 is preferably that of SEQ ID NO: 130, or an allelic variant of SEQ ID NO:130.

When present in an aberrant cell of a patient or subject, said polynucleotide fusion preferably further includes any sequence 5′ from exon 9 of THBS1 and any sequence 3′ from exon 6 of NRG1, but to allow detection of the fusion junction using a polynucleotide-based detection assay, it may suffice that at least SEQ ID NOs: 386 and 130 are present. Any sequence 5′ from exon 9 of THBS1 comprises or consists of one or all of SEQ ID NOs: 378-385 (or any allelic variant of SEQ ID NOs: 378-385), and any sequence 3′ of exon 6 of NRG1 comprises or consists of one or all of SEQ ID NOs: 131-137 (or any allelic variant of SEQ ID NOs: 131-137).

Preferably, the allelic variant of exon 9 of THBS1 has at least 85% identity to SEQ ID NO: 386, preferably at least 90% identity, 92%, 94%, 96% or more preferably at least 98% sequence identity thereto and the allelic variant of exon 6 of NRG1 has at least 85% identity to SEQ ID NO: 130, preferably at least 90% identity, 92%, 94%, 96% or more preferably at least 98% sequence identity thereto.

Preferably, the portion of exon 9 of THBS1 comprises or is according to SEQ ID NO: 374 and the allelic variant thereof has at least 85% identity to SEQ ID NO: 374, preferably at least 90% identity, 92%, 94%, 96% or more preferably at least 98% sequence identity thereto. The portion of exon 6 of NRG1 in the fusion with THBS1 preferably is or comprises the sequence according to SEQ ID NO: 375 and the allelic variant thereof has at least 85% identity to SEQ ID NO: 375, preferably at least 90% identity, 92%, 94%, 96% or more preferably at least 98% sequence identity thereto. More preferably, the portion of exon 9 of THBS1 comprises or consists of 2 to about 10, about 20, about 30, or up to about 40 or even all contiguous nucleic acids from SEQ ID NO: 374, including at least the nucleic acid of position 56. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 374, including at least the nucleic acid of position 56. More preferably, the portion of exon 9 of THBS1 comprises or is according to SEQ ID NO: 374, or an allelic variant thereof. Such short polynucleotide sequences are particularly useful for detecting the presence of a larger polynucleotide fusion between THBS1 and NRG1 and establishing that such a fusion is an in-frame, oncogenic fusion comprising an EGF-like domain of NRG1.

Alternatively, the portion of exon 9 of THBS1 comprises or consists of SEQ ID NO: 386 (or an allelic variant of SEQ ID NO: 386), including at least the nucleic acid of position 177. Preferably, the portion of exon 9 of THBS1 comprises or consists of 2 to about 10, about 20, about 30, or up to about 40 or even all contiguous nucleic acids from SEQ ID NO: 386, or from the allelic variant thereof, including at least the nucleic acid of position 177. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 386, including at least the nucleic acid of position 177. In this alternative, the portion of exon 9 of THBS1 more preferably comprises or is according to SEQ ID NO: 386, or an allelic variant thereof. Preferably, the portion of exon 6 of NRG1 in the fusion with THBS1 comprises or consists of 2 to about 10, about 20, about 30, or up to about 40 or even all contiguous nucleic acids from SEQ ID NO: 130, or from the allelic variant thereof, including at least the nucleic acid of position 1. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 130, including at least the nucleic acid of position 1.

Preferably, any THBS1-NRG1 polynucleotide fusion provided herein is an in-frame fusion of THBS1 with NRG1. More preferably said fusion is an in-frame fusion comprising exon 9 of THBS1, or a portion of exon 6, with exon 6 of NRG1, or a portion of exon 6 of NRG1. Said in-frame fusion is preferably the fusion of SEQ ID NO: 376, or an allelic variant thereof having at least 85% identity thereto, preferably at least 90%, 92%, 94%, 96% or even 98% identity thereto.

In an alternatively preferred embodiment, the polynucleotide comprising a portion of exon 9 of THBS1 fused with a portion of exon 6 of NRG1 comprises 2 to about 10, about 20, about 30, or up to about 40 or even all contiguous nucleic acids from SEQ ID NO: 376, including the nucleic acids at positions 56 and 57. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 376, including at least the nucleic acids at positions 56 and 57. SEQ ID NO: 376 includes the junction between THBS1 and NRG1, in particular the junction is between the nucleic acid at position 56, which stems from THBS1 and the nucleic acid at position 57 which stems from NRG1. Preferably, the polynucleotide comprising a portion of exon 9 of THBS1 fused with a portion of exon 6 of NRG1 has the polynucleotide sequence of SEQ ID NO: 376, or an allelic variant thereof.

In a preferred embodiment, there is provided a polynucleotide according to SEQ ID NO: 376, or a polynucleotide which comprises about 20, about 30, about 40 or all contiguous nucleic acids from SEQ ID NO: 376, preferably including the nucleic acids at positions 56 and 57. The number of contiguous nucleic acids may be 18, 19, 20, 21, 22, 23, 24, 25, 26, 27, 28, 29, 30, 31, 32, 33, 34, 35, 36, 37, 38, 39, 40 or all nucleic acids of SEQ ID NO: 376, preferably including at least the nucleic acids at positions 56 and 57.

Preferably, the polynucleotide comprising a portion of exon 9 of THBS1, or an allelic variant thereof, fused with a portion of exon 6 of NRG1, or an allelic variant thereof, is part of a longer polynucleotide that further comprises or encodes an EGF-like domain of NRG1. An aberrant cell comprising the polynucleotide fusion involving THBS1 or expressing the polypeptide fusion, comprises or encodes an EGF-like domain of NRG1. For quick detection, diagnosis or identification purposes, it may suffice to show that the fusion junction between THBS1 and NRG1 is in-frame and occurs at such a position that the resulting fusion product, nucleic acid or protein, comprises an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

Preferably, the portion of exon 9 of THBS1, or the allelic variant thereof, is 5′ to the portion of exon 6 of NRG1, or the allelic variant thereof. This orientation at the nucleic acid level results in a fusion polypeptide product that contains an N-terminus of THBS1 and a C-terminus of NRG1. Also, the herein provided THBS1-NRG1 polynucleotide fusion produces a protein fusion, wherein the part from the N-terminus to the fusion junction is polypeptide sequence from THBS1 and the part from the junction to the C-terminus is NRG1 polypeptide sequence, wherein the NRG1 part also provides its EGF-like domain. The THBS1-NRG1 fusion protein thereby retains the EGF-like domain of NRG1 and the ability to drive proliferation and survival of a subset of human cancers, in particular an adenocarcinoma, more in particular pancreatic ductal adenocarcinoma.

### AGRN-NRG1 Polynucleotide Fusion

There is also provided a polynucleotide fusion comprising a portion of exon 12 of AGRN fused with a portion of exon 6 of NRG1. Exon 12 of AGRN is preferably that of SEQ ID NO: 416, or an allelic variant of SEQ ID NO: 416, and exon 6 of NRG1 is preferably that of SEQ ID NO: 130, or an allelic variant of SEQ ID NO:130.

When present in an aberrant cell of a patient or subject, said polynucleotide fusion preferably further includes any sequence 5′ from exon 12 of AGRN and any sequence 3′ from exon 6 of NRG1, but to allow detection of the fusion junction using a polynucleotide-based detection assay, it may suffice that at least SEQ ID NOs: 416 and 130 are present. Any sequence 5′ from exon 12 of AGRN comprises or consists of one or all of SEQ ID NOs: 405-415 (or any allelic variant of SEQ ID NOs: 405-415), and any sequence 3′ of exon 6 of NRG1 comprises or consists of one or all of SEQ ID NOs: 131-137 (or any allelic variant of SEQ ID NOs: 131-137).

Preferably, the allelic variant of exon 12 of AGRN has at least 85% identity to SEQ ID NO: 416, preferably at least 90% identity, 92%, 94%, 96% or more preferably at least 98% sequence identity thereto and the allelic variant of exon 6 of NRG1 has at least 85% identity to SEQ ID NO: 130, preferably at least 90% identity, 92%, 94%, 96% or more preferably at least 98% sequence identity thereto.

Preferably, the portion of exon 12 of AGRN comprises or is according to SEQ ID NO: 401 and the allelic variant thereof has at least 85% identity to SEQ ID NO: 401, preferably at least 90% identity, 92%, 94%, 96% or more preferably at least 98% sequence identity thereto. The portion of exon 6 of NRG1 in the fusion with AGRN preferably is or comprises the sequence according to SEQ ID NO: 402 and the allelic variant thereof has at least 85% identity to SEQ ID NO: 402, preferably at least 90% identity, 92%, 94%, 96% or more preferably at least 98% sequence identity thereto. More preferably, the portion of exon 12 of AGRN comprises or consists of 2 to about 10, about 20, about 30, or up to about 40 or even all contiguous nucleic acids from SEQ ID NO: 401, including at least the nucleic acid of position 106. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 401, including at least the nucleic acid of position 106. More preferably, the portion of exon 12 of AGRN comprises or is according to SEQ ID NO: 401, or an allelic variant thereof. Such short polynucleotide sequences are particularly useful for detecting the presence of a larger polynucleotide fusion between AGRN and NRG1 and establishing that such a fusion is an in-frame, oncogenic fusion comprising an EGF-like domain of NRG1.

Alternatively, the portion of exon 12 of AGRN comprises or consists of SEQ ID NO: 416 (or an allelic variant of SEQ ID NO: 416), including at least the nucleic acid of position 106. Preferably, the portion of exon 12 of AGRN comprises or consists of 2 to about 10, about 20, about 30, or up to about 40 or even all contiguous nucleic acids from SEQ ID NO: 416, or from the allelic variant thereof, including at least the nucleic acid of position 106. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 416, including at least the nucleic acid of position 106. In this alternative, the portion of exon 12 of AGRN more preferably comprises or is according to SEQ ID NO: 416, or an allelic variant thereof. Preferably, the portion of exon 6 of NRG1 in the fusion with AGRN comprises or consists of 2 to about 10, about 20, about 30, or up to about 40 or even all contiguous nucleic acids from SEQ ID NO: 130, or from the allelic variant thereof, including at least the nucleic acid of position 1. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 130, including at least the nucleic acid of position 1.

Preferably, any AGRN-NRG1 polynucleotide fusion provided herein is an in-frame fusion of AGRN with NRG1. More preferably said fusion is an in frame fusion comprising exon 12 of AGRN, or a portion of exon 12, with exon 6 of NRG1, or a portion of exon 6 of NRG1. Said in-frame fusion is preferably the fusion of SEQ ID NO: 403, or an allelic variant thereof having at least 85% identity thereto, preferably at least 90%, 92%, 94%, 96% or even 98% identity thereto.

In an alternatively preferred embodiment, the polynucleotide comprising a portion of exon 12 of AGRN fused with a portion of exon 6 of NRG1 comprises 2 to about 10, about 20, about 30, or up to about 40 or even all contiguous nucleic acids from SEQ ID NO: 403, including the nucleic acids at positions 106 and 107. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 403, including at least the nucleic acids at positions 106 and 107. SEQ ID NO: 403 includes the junction between AGRN and NRG1, in particular the junction is between the nucleic acid at position 106, which stems from AGRN and the nucleic acid at position 107 which stems from NRG1. Preferably, the polynucleotide comprising a portion of exon 12 of AGRN fused with a portion of exon 6 of NRG1 has the polynucleotide sequence of SEQ ID NO: 403, or an allelic variant thereof.

In a preferred embodiment, there is provided a polynucleotide according to SEQ ID NO: 403, or a polynucleotide which comprises about 20, about 30, about 40 or all contiguous nucleic acids from SEQ ID NO: 403, preferably including the nucleic acids at positions 106 and 107. The number of contiguous nucleic acids may be 18, 19, 20, 21, 22, 23, 24, 25, 26, 27, 28, 29, 30, 31, 32, 33, 34, 35, 36, 37, 38, 39, 40 or all nucleic acids of SEQ ID NO: 403, preferably including at least the nucleic acids at positions 106 and 107.

Preferably, the polynucleotide comprising a portion of exon 12 of AGRN, or an allelic variant thereof, fused with a portion of exon 6 of NRG1, or an allelic variant thereof, is part of a longer polynucleotide that further comprises or encodes an EGF-like domain of NRG1. An aberrant cell comprising the polynucleotide fusion involving AGRN or expressing the polypeptide fusion, comprises or encodes an EGF-like domain of NRG1. For quick detection, diagnosis or identification purposes, it may suffice to show that the fusion junction between AGRN and NRG1 is in-frame and occurs at such a position that the resulting fusion product, nucleic acid or protein, comprises an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

Preferably, the portion of exon 12 of AGRN, or the allelic variant thereof, is 5′ to the portion of exon 6 of NRG1, or the allelic variant thereof. This orientation at the nucleic acid level results in a fusion polypeptide product that contains an N-terminus of AGRN and a C-terminus of NRG1. Also, the herein provided AGRN-NRG1 polynucleotide fusion produces a protein fusion, wherein the part from the N-terminus to the fusion junction is polypeptide sequence from AGRN and the part from the junction to the C-terminus is NRG1 polypeptide sequence, wherein the NRG1 part also provides its EGF-like domain. The AGRN-NRG1 fusion protein thereby retains the EGF-like domain of NRG1 and the ability to drive proliferation and survival of a subset of human cancers, in particular an adenocarcinoma, more in particular pancreatic ductal adenocarcinoma.

### PVALB-NRG1 Polynucleotide Fusion

In accordance with the present disclosure, there is also provided a polynucleotide comprising a PVALB nucleic acid sequence (or a portion of a PVALB nucleic acid sequence) fused with an NRG1 nucleic acid sequence (or a portion of an NRG1 nucleic acid sequence). Also included in said fusion are allelic variants of PVALB and NRG1 nucleic acid sequences.

Preferably, the PVALB nucleic acid sequence, or the portion thereof, comprises or consists of any one of SEQ ID NOs: 439-444, or an allelic variant of any one of SEQ ID NOs: 439-444, and the NRG1 nucleic acid sequence, or the portion thereof, comprises or consists of any one of SEQ ID NOs: 125-138, or an allelic variant of any one of SEQ ID NOs: 125-138. More preferably, the PVALB nucleic acid sequence comprises a portion of SEQ ID NOs: 444, or an allelic variant of SEQ ID NOs: 444, and the NRG1 nucleic acid sequence preferably comprises a portion of SEQ ID NOs: 138, or an allelic variant of SEQ ID NOs: 138. SEQ ID NOs: 439-443 correspond to the individual exons 1-5 of PVALB according to NM_002854.3, respectively. SEQ ID NO: 444 corresponds to exons 1-5 of PVALB according to NM_002854.3. SEQ ID NOs: 125-137 correspond to the individual exons 1-13 of the NRG1 sequence according to NM_001159999, respectively. SEQ ID NO: 138 corresponds to exons 1-13 of NRG1 according to NM_001159999.

In a preferred embodiment, the portion of the PVALB nucleic acid sequence comprises 2 to about 10, about 20, about 30, or up to about 40 or even all contiguous nucleic acids from any one of SEQ ID NOs: 439-444, or an allelic variant of any one of SEQ ID NOs: 439-444, and the portion of the NRG1 nucleic acid sequence comprises 2 to about 10, about 20, about 30, or up to about 40 or even all contiguous nucleic acids of any one of SEQ ID NOs: 125-138, or an allelic variant of any one of SEQ ID NOs: 125-138.

There is also provided a polynucleotide fusion comprising a portion of exon 4 of PVALB fused with a portion of exon 6 of NRG1. Exon 4 of PVALB is preferably that of SEQ ID NO: 442, or an allelic variant of SEQ ID NO: 442, and exon 6 of NRG1 is preferably that of SEQ ID NO: 130, or an allelic variant of SEQ ID NO:130.

When present in an aberrant cell of a patient or subject, said polynucleotide fusion preferably further includes any sequence 5′ from exon 4 of PVALB and any sequence 3′ from exon 6 of NRG1, but to allow detection of the fusion junction using a polynucleotide-based detection assay, it may suffice that at least SEQ ID NOs: 442 and 130 are present. Any sequence 5′ from exon 4 of PVALB comprises or consists of one or all of SEQ ID NOs: 439-441 (or any allelic variant of SEQ ID NOs: 439-441), and any sequence 3′ of exon 6 of NRG1 comprises or consists of one or all of SEQ ID NOs: 131-137 (or any allelic variant of SEQ ID NOs: 131-137).

Preferably, the allelic variant of exon 4 of PVALB has at least 85% identity to SEQ ID NO: 442, preferably at least 90% identity, 92%, 94%, 96% or more preferably at least 98% sequence identity thereto and the allelic variant of exon 6 of NRG1 has at least 85% identity to SEQ ID NO: 130, preferably at least 90% identity, 92%, 94%, 96% or more preferably at least 98% sequence identity thereto.

Preferably, the portion of exon 4 of PVALB comprises or is according to SEQ ID NO: 435 and the allelic variant thereof has at least 85% identity to SEQ ID NO: 435, preferably at least 90% identity, 92%, 94%, 96% or more preferably at least 98% sequence identity thereto. The portion of exon 6 of NRG1 in the fusion with PVALB preferably is or comprises the sequence according to SEQ ID NO: 436 and the allelic variant thereof has at least 85% identity to SEQ ID NO: 436, preferably at least 90% identity, 92%, 94%, 96% or more preferably at least 98% sequence identity thereto. More preferably, the portion of exon 4 of PVALB comprises or consists of 2 to about 10, about 20, about 30, or up to about 40 or even all contiguous nucleic acids from SEQ ID NO: 435, including at least the nucleic acid of position 102. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 435, including at least the nucleic acid of position 102. More preferably, the portion of exon 4 of PVALB comprises or is according to SEQ ID NO: 435, or an allelic variant thereof. Such short polynucleotide sequences are particularly useful for detecting the presence of a larger polynucleotide fusion between PVALB and NRG1 and establishing that such a fusion is an in-frame, oncogenic fusion comprising an EGF-like domain of NRG1.

Alternatively, the portion of exon 4 of PVALB comprises or consists of SEQ ID NO: 442 (or an allelic variant of SEQ ID NO: 442), including at least the nucleic acid of position 110. Preferably, the portion of exon 4 of PVALB comprises or consists of 2 to about 10, about 20, about 30, or up to about 40 or even all contiguous nucleic acids from SEQ ID NO: 442, or from the allelic variant thereof, including at least the nucleic acid of position 110. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 442, including at least the nucleic acid of position 110. In this alternative, the portion of exon 4 of PVALB more preferably comprises or is according to SEQ ID NO: 442, or an allelic variant thereof. Preferably, the portion of exon 6 of NRG1 in the fusion with PVALB comprises or consists of 2 to about 10, about 20, about 30, or up to about 40 or even all contiguous nucleic acids from SEQ ID NO: 130, or from the allelic variant thereof, including at least the nucleic acid of position 1. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 130, including at least the nucleic acid of position 1.

Preferably, any PVALB-NRG1 polynucleotide fusion provided herein is an in-frame fusion of PVALB with NRG1. More preferably said fusion is an in-frame fusion comprising exon 4 of PVALB, or a portion of exon 4, with exon 6 of NRG1, or a portion of exon 6 of NRG1. Said in frame fusion is preferably the fusion of SEQ ID NO: 437, or an allelic variant thereof having at least 85% identity thereto, preferably at least 90%, 92%, 94%, 96% or even 98% identity thereto.

In an alternatively preferred embodiment, the polynucleotide comprising a portion of exon 4 of PVALB fused with a portion of exon 6 of NRG1 comprises 2 to about 10, about 20, about 30, or up to about 40 or even all contiguous nucleic acids from SEQ ID NO: 437, including the nucleic acids at positions 102 and 103. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 437, including at least the nucleic acids at positions 102 and 103. SEQ ID NO: 437 includes the junction between PVALB and NRG1, in particular the junction is between the nucleic acid at position 102, which stems from PVALB and the nucleic acid at position 103 which stems from NRG1. Preferably, the polynucleotide comprising a portion of exon 4 of PVALB fused with a portion of exon 6 of NRG1 has the polynucleotide sequence of SEQ ID NO: 437, or an allelic variant thereof.

Preferably, the polynucleotide comprising a portion of exon 4 of PVALB, or an allelic variant thereof, fused with a portion of exon 6 of NRG1, or an allelic variant thereof, is part of a longer polynucleotide that further comprises or encodes an EGF-like domain of NRG1. An aberrant cell comprising the polynucleotide fusion involving PVALB or expressing the polypeptide fusion, comprises or encodes an EGF-like domain of NRG1. For quick detection, diagnosis or identification purposes, it may suffice to show that the fusion junction between PVALB and NRG1 is in-frame and occurs at such a position that the resulting fusion product, nucleic acid or protein, comprises an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

Preferably, the portion of exon 4 of PVALB, or the allelic variant thereof, is 5′ to the portion of exon 6 of NRG1, or the allelic variant thereof. This orientation at the nucleic acid level results in a fusion polypeptide product that contains an N-terminus of PVALB and a C-terminus of NRG1. Also, the herein provided PVALB-NRG1 polynucleotide fusion produces a protein fusion, wherein the part from the N-terminus to the fusion junction is polypeptide sequence from PVALB and the part from the junction to the C-terminus is NRG1 polypeptide sequence, wherein the NRG1 part also provides its EGF-like domain. The PVALB-NRG1 fusion protein thereby retains the EGF-like domain of NRG1 and the ability to drive proliferation and survival of a subset of human cancers, in particular an adenocarcinoma, more in particular pancreatic ductal adenocarcinoma.

### APP-NRG1 Polynucleotide Fusion

There is also provided a polynucleotide fusion comprising a portion of Exon 14 of APP fused with a portion of exon 6 of NRG1. Exon 14 of APP is preferably that of SEQ ID NO: 501, or an allelic variant of SEQ ID NO: 501, and exon 6 of NRG1 is preferably that of SEQ ID NO: 130, or an allelic variant of SEQ ID NO:130.

When present in an aberrant cell of a patient or subject, said polynucleotide fusion preferably further includes any sequence 5′ from Exon 14 of APP and any sequence 3′ from exon 6 of NRG1, but to allow detection of the fusion junction using a polynucleotide-based detection assay, it may suffice that at least SEQ ID NOs: 501 and 130 are present. Any sequence 5′ from Exon 14 of APP comprises or consists of one or all of SEQ ID NOs: 488-500 (or any allelic variant of SEQ ID NOs: 488-500), and any sequence 3′ of exon 6 of NRG1 comprises or consists of one or all of SEQ ID NOs: 131-137 (or any allelic variant of SEQ ID NOs: 131-137).

Preferably, the allelic variant of Exon 14 of APP has at least 85% identity to SEQ ID NO: 501, preferably at least 90% identity, 92%, 94%, 96% or more preferably at least 98% sequence identity thereto and the allelic variant of exon 6 of NRG1 has at least 85% identity to SEQ ID NO: 130, preferably at least 90% identity, 92%, 94%, 96% or more preferably at least 98% sequence identity thereto.

Preferably, the portion of Exon 14 of APP comprises or is according to SEQ ID NO: 484 and the allelic variant thereof has at least 85% identity to SEQ ID NO: 484, preferably at least 90% identity, 92%, 94%, 96% or more preferably at least 98% sequence identity thereto. The portion of exon 6 of NRG1 in the fusion with APP preferably is or comprises the sequence according to SEQ ID NO: 485 and the allelic variant thereof has at least 85% identity to SEQ ID NO: 485, preferably at least 90% identity, 92%, 94%, 96% or more preferably at least 98% sequence identity thereto. More preferably, the portion of Exon 14 of APP comprises or consists of 2 to about 10, about 20, about 30, or up to about 40 or even all contiguous nucleic acids from SEQ ID NO: 484, including at least the nucleic acid of position 54. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 484, including at least the nucleic acid of position 54. More preferably, the portion of Exon 14 of APP comprises or is according to SEQ ID NO: 484, or an allelic variant thereof. Such short polynucleotide sequences are particularly useful for detecting the presence of a larger polynucleotide fusion between APP and NRG1 and establishing that such a fusion is an in-frame, oncogenic fusion comprising an EGF-like domain of NRG1.

Alternatively, the portion of Exon 14 of APP comprises or consists of SEQ ID NO: 501 (or an allelic variant of SEQ ID NO: 501), including at least the nucleic acid of position 54. Preferably, the portion of Exon 14 of APP comprises or consists of 2 to about 10, about 20, about 30, or up to about 40 or even all contiguous nucleic acids from SEQ ID NO: 501, or from the allelic variant thereof, including at least the nucleic acid of position 54. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 501, including at least the nucleic acid of position 54. In this alternative, the portion of Exon 14 of APP more preferably comprises or is according to SEQ ID NO: 501, or an allelic variant thereof. Preferably, the portion of exon 6 of NRG1 in the fusion with APP comprises or consists of 2 to about 10, about 20, about 30, or up to about 40 or even all contiguous nucleic acids from SEQ ID NO: 130, or from the allelic variant thereof, including at least the nucleic acid of position 1. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 130, including at least the nucleic acid of position 1.

Preferably, any APP-NRG1 polynucleotide fusion provided herein is an in-frame fusion of APP with NRG1. More preferably said fusion is an in-frame fusion comprising Exon 14 of APP, or a portion of Exon 14, with exon 6 of NRG1, or a portion of exon 6 of NRG1. Said in-frame fusion is preferably the fusion of SEQ ID NO: 486, or an allelic variant thereof having at least 85% identity thereto, preferably at least 90%, 92%, 94%, 96% or even 98% identity thereto.

In an alternatively preferred embodiment, the polynucleotide comprising a portion of Exon 14 of APP fused with a portion of exon 6 of NRG1 comprises 2 to about 10, about 20, about 30, or up to about 40 or even all contiguous nucleic acids from SEQ ID NO: 486, including the nucleic acids at positions 54 and 55. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 486, including at least the nucleic acids at positions 54 and 55. SEQ ID NO: 486 includes the junction between APP and NRG1, in particular the junction is between the nucleic acid at position 54, which stems from APP and the nucleic acid at position 55 which stems from NRG1. Preferably, the polynucleotide comprising a portion of Exon 14 of APP fused with a portion of exon 6 of NRG1 has the polynucleotide sequence of SEQ ID NO: 486, or an allelic variant thereof.

In a preferred embodiment, there is provided a polynucleotide according to SEQ ID NO: 486, or a polynucleotide which comprises about 20, about 30, about 40 or all contiguous nucleic acids from SEQ ID NO: 486, preferably including the nucleic acids at positions 54 and 55. The number of contiguous nucleic acids may be 18, 19, 20, 21, 22, 23, 24, 25, 26, 27, 28, 29, 30, 31, 32, 33, 34, 35, 36, 37, 38, 39, 40 or all nucleic acids of SEQ ID NO: 486, preferably including at least the nucleic acids at positions 54 and 55.

Preferably, the polynucleotide comprising a portion of Exon 14 of APP, or an allelic variant thereof, fused with a portion of exon 6 of NRG1, or an allelic variant thereof, is part of a longer polynucleotide that further comprises or encodes an EGF-like domain of NRG1. An aberrant cell comprising the polynucleotide fusion involving APP or expressing the polypeptide fusion, comprises or encodes an EGF-like domain of NRG1. For quick detection, diagnosis or identification purposes, it may suffice to show that the fusion junction between APP and NRG1 is in-frame and occurs at such a position that the resulting fusion product, nucleic acid or protein, comprises an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

Preferably, the portion of Exon 14 of APP, or the allelic variant thereof, is 5′ to the portion of exon 6 of NRG1, or the allelic variant thereof. This orientation at the nucleic acid level results in a fusion polypeptide product that contains an N-terminus of APP and a C-terminus of NRG1. Also, the herein provided APP-NRG1 polynucleotide fusion produces a protein fusion, wherein the part from the N-terminus to the fusion junction is polypeptide sequence from APP and the part from the junction to the C-terminus is NRG1 polypeptide sequence, wherein the NRG1 part also provides its EGF-like domain. The APP-NRG1 fusion protein thereby retains the EGF-like domain of NRG1 and the ability to drive proliferation and survival of a subset of human cancers, in particular an adenocarcinoma, more in particular pancreatic ductal adenocarcinoma.

### WRN-NRG1 Polynucleotide Fusion

There is also provided a polynucleotide fusion comprising a portion of Exon 33 of WRN fused with a portion of exon 6 of NRG1. Exon 33 of WRN is preferably that of SEQ ID NO: 562, or an allelic variant of SEQ ID NO: 562, and exon 6 of NRG1 is preferably that of SEQ ID NO: 130, or an allelic variant of SEQ ID NO:130.

When present in an aberrant cell of a patient or subject, said polynucleotide fusion preferably further includes any sequence 5′ from Exon 33 of WRN and any sequence 3′ from exon 6 of NRG1, but to allow detection of the fusion junction using a polynucleotide-based detection assay, it may suffice that at least SEQ ID NOs: 562 and 130 are present. Any sequence 5′ from Exon 33 of WRN comprises or consists of one or all of SEQ ID NOs: 530-561 (or any allelic variant of SEQ ID NOs: 530-561), and any sequence 3′ of exon 6 of NRG1 comprises or consists of one or all of SEQ ID NOs: 131-137 (or any allelic variant of SEQ ID NOs: 131-137).

Preferably, the allelic variant of Exon 33 of WRN has at least 85% identity to SEQ ID NO: 562, preferably at least 90% identity, 92%, 94%, 96% or more preferably at least 98% sequence identity thereto and the allelic variant of exon 6 of NRG1 has at least 85% identity to SEQ ID NO: 130, preferably at least 90% identity, 92%, 94%, 96% or more preferably at least 98% sequence identity thereto.

Preferably, the portion of Exon 33 of WRN comprises or is according to SEQ ID NO: 526 and the allelic variant thereof has at least 85% identity to SEQ ID NO: 526, preferably at least 90% identity, 92%, 94%, 96% or more preferably at least 98% sequence identity thereto. The portion of exon 6 of NRG1 in the fusion with WRN preferably is or comprises the sequence according to SEQ ID NO: 527 and the allelic variant thereof has at least 85% identity to SEQ ID NO: 527, preferably at least 90% identity, 92%, 94%, 96% or more preferably at least 98% sequence identity thereto. More preferably, the portion of Exon 33 of WRN comprises or consists of 2 to about 10, about 20, about 30, or up to about 40 or even all contiguous nucleic acids from SEQ ID NO: 526, including at least the nucleic acid of position 96. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 526, including at least the nucleic acid of position 96. More preferably, the portion of Exon 33 of WRN comprises or is according to SEQ ID NO: 526, or an allelic variant thereof. Such short polynucleotide sequences are particularly useful for detecting the presence of a larger polynucleotide fusion between WRN and NRG1 and establishing that such a fusion is an in-frame, oncogenic fusion comprising an EGF-like domain of NRG1.

Alternatively, the portion of Exon 33 of WRN comprises or consists of SEQ ID NO: 562 (or an allelic variant of SEQ ID NO: 562), including at least the nucleic acid of position 163. Preferably, the portion of Exon 33 of WRN comprises or consists of 2 to about 10, about 20, about 30, or up to about 40 or even all contiguous nucleic acids from SEQ ID NO: 562, or from the allelic variant thereof, including at least the nucleic acid of position 163. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 562, including at least the nucleic acid of position 163. In this alternative, the portion of Exon 33 of WRN more preferably comprises or is according to SEQ ID NO: 562, or an allelic variant thereof. Preferably, the portion of exon 6 of NRG1 in the fusion with WRN comprises or consists of 2 to about 10, about 20, about 30, or up to about 40 or even all contiguous nucleic acids from SEQ ID NO: 130, or from the allelic variant thereof, including at least the nucleic acid of position 1. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 130, including at least the nucleic acid of position 1.

Preferably, any WRN-NRG1 polynucleotide fusion provided herein is an in-frame fusion of WRN with NRG1. More preferably said fusion is an in frame fusion comprising Exon 33 of WRN, or a portion of Exon 33, with exon 6 of NRG1, or a portion of exon 6 of NRG1. Said in frame fusion is preferably the fusion of SEQ ID NO: 528, or an allelic variant thereof having at least 85% identity thereto, preferably at least 90%, 92%, 94%, 96% or even 98% identity thereto.

In an alternatively preferred embodiment, the polynucleotide comprising a portion of Exon 33 of WRN fused with a portion of exon 6 of NRG1 comprises 2 to about 10, about 20, about 30, or up to about 40 or even all contiguous nucleic acids from SEQ ID NO: 528, including the nucleic acids at positions 96 and 97. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 528, including at least the nucleic acids at positions 96 and 97. SEQ ID NO: 528 includes the junction between WRN and NRG1, in particular the junction is between the nucleic acid at position 96, which stems from WRN and the nucleic acid at position 97 which stems from NRG1. Preferably, the polynucleotide comprising a portion of Exon 33 of WRN fused with a portion of exon 6 of NRG1 has the polynucleotide sequence of SEQ ID NO: 528, or an allelic variant thereof.

In a preferred embodiment, there is provided a polynucleotide according to SEQ ID NO: 528, or a polynucleotide which comprises about 20, about 30, about 40 or all contiguous nucleic acids from SEQ ID NO: 528, preferably including the nucleic acids at positions 96 and 97. The number of contiguous nucleic acids may be 18, 19, 20, 21, 22, 23, 24, 25, 26, 27, 28, 29, 30, 31, 32, 33, 34, 35, 36, 37, 38, 39, 40 or all nucleic acids of SEQ ID NO: 528, preferably including at least the nucleic acids at positions 96 and 97.

Preferably, the polynucleotide comprising a portion of Exon 33 of WRN, or an allelic variant thereof, fused with a portion of exon 6 of NRG1, or an allelic variant thereof, is part of a longer polynucleotide that further comprises or encodes an EGF-like domain of NRG1. An aberrant cell comprising the polynucleotide fusion involving WRN or expressing the polypeptide fusion, comprises or encodes an EGF-like domain of NRG1. For quick detection, diagnosis or identification purposes, it may suffice to show that the fusion junction between

WRN and NRG1 is in-frame and occurs at such a position that the resulting fusion product, nucleic acid or protein, comprises an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

Preferably, the portion of Exon 33 of WRN, or the allelic variant thereof, is 5′ to the portion of exon 6 of NRG1, or the allelic variant thereof. This orientation at the nucleic acid level results in a fusion polypeptide product that contains an N-terminus of WRN and a C-terminus of NRG1. Also, the herein provided WRN-NRG1 polynucleotide fusion produces a protein fusion, wherein the part from the N-terminus to the fusion junction is polypeptide sequence from WRN and the part from the junction to the C-terminus is NRG1 polypeptide sequence, wherein the NRG1 part also provides its EGF-like domain. The WRN-NRG1 fusion protein thereby retains the EGF-like domain of NRG1 and the ability to drive proliferation and survival of a subset of human cancers, in particular a breast cancer.

### DAAM1-NRG1 Polynucleotide Fusion

In accordance with the present disclosure, there is also provided a polynucleotide comprising a DAAM1 nucleic acid sequence (or a portion of a DAAM1 nucleic acid sequence) fused with an NRG1 nucleic acid sequence (or a portion of an NRG1 nucleic acid sequence). Also included in said fusion are allelic variants of DAAM1 and NRG1 nucleic acid sequences.

Preferably, the DAAM1 nucleic acid sequence, or the portion thereof, comprises or consists of any one of SEQ ID NOs: 606-631, or an allelic variant of any one of SEQ ID NOs: 606-631, and the NRG1 nucleic acid sequence, or the portion thereof, comprises or consists of any one of SEQ ID NOs: 125-138, or an allelic variant of any one of SEQ ID NOs: 125-138. More preferably, the DAAM1 nucleic acid sequence comprises a portion of SEQ ID NO: 631, or an allelic variant of SEQ ID NO: 631, and the NRG1 nucleic acid sequence preferably comprises a portion of SEQ ID NO: 138, or an allelic variant of SEQ ID NOs: 138. SEQ ID NOs: 606-630 correspond to the individual exons 1-25 of DAAM1 according to NM_001270520.2, respectively. SEQ ID NO: 631 corresponds to exons 1-25 of DAAM1 according to NM_001270520.2. SEQ ID NOs: 125-137 correspond to the individual exons 1-13 of the NRG1 sequence according to NM_001159999.3, respectively. SEQ ID NO: 138 corresponds to exons 1-13 of NRG1 according to NM_001159999.3.

In a preferred embodiment, the portion of the DAAM1 nucleic acid sequence comprises 2 to about 10, about 20, about 30, or up to about 40 or even all contiguous nucleic acids from any one of SEQ ID NOs: 606-631, or an allelic variant of any one of SEQ ID NOs: 606-631, and the portion of the NRG1 nucleic acid sequence comprises 2 to about 10, about 20, about 30, or up to about 40 or even all contiguous nucleic acids of any one of SEQ ID NOs: 125-138, or an allelic variant of any one of SEQ ID NOs: 125-138.

Preferably, the allelic variant of the DAAM1 nucleic acid sequences has at least 85% identity to any one of SEQ ID NOs: 606-631, preferably at least 90% identity, 92%, 94%, 96% or more preferably at least 98% sequence identity thereto; and the allelic variant of the NRG1 nucleic acid sequences has at least 85% identity to any one of SEQ ID NOs: 125-138, preferably at least 90% identity, 92%, 94%, 96% or more preferably at least 98% sequence identity thereto.

Preferably, the DAAM1 nucleic acid sequence, or the portion thereof, is 5′ to the NRG1 nucleic acid sequence, or the portion thereof.

Preferably, the polynucleotide comprising a DAAM1 nucleic acid sequence of DAAM1, or a portion of said sequence, fused with a NRG1 nucleic acid sequence, or portion of said sequence, comprises or encodes an EGF-like domain of NRG1. An aberrant cell comprising the DAAM1-NRG1 polynucleotide fusion, comprises or encodes an EGF-like domain of NRG1. For detection, diagnosis or identification purposes, it may suffice to show that the fusion junction between DAAM1 and NRG1 is in-frame and occurs at a position such that the resulting fusion nucleic acid codes for an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

There is also provided a polynucleotide fusion comprising a portion of Exon 1 of DAAM1 fused with a portion of exon 1 of NRG1. Exon 1 of DAAM1 is preferably that of SEQ ID NO: 606, or an allelic variant of SEQ ID NO: 606, and exon 1 of NRG1 is preferably that of SEQ ID NO: 125, or an allelic variant of SEQ ID NO: 125.

When present in an aberrant cell of a patient or subject, said polynucleotide fusion preferably further includes any sequence 5′ from Exon 1 of DAAM1 and any sequence 3′ from exon 1 of NRG1, but to allow detection of the fusion junction using a polynucleotide-based detection assay, it may suffice that at least SEQ ID NOs: 606 and 125 are present. Any sequence 5′ from Exon 1 of DAAM1 comprises or consists of SEQ ID NOs: 606 (or any allelic variant of SEQ ID NOs: 606), and any sequence 3′ of exon 1 of NRG1 comprises or consists of one or all of SEQ ID NOs: 126-137 (or any allelic variant of SEQ ID NOs: 126-137).

Preferably, the allelic variant of Exon 1 of DAAM1 has at least 85% identity to SEQ ID NO: 606, preferably at least 90% identity, 92%, 94%, 96% or more preferably at least 98% sequence identity thereto and the allelic variant of exon 1 of NRG1 has at least 85% identity to SEQ ID NO: 125, preferably at least 90% identity, 92%, 94%, 96% or more preferably at least 98% sequence identity thereto.

Preferably, the portion of Exon 1 of DAAM1 comprises or is according to SEQ ID NO: 603 and the allelic variant thereof has at least 85% identity to SEQ ID NO: 603, preferably at least 90% identity, 92%, 94%, 96% or more preferably at least 98% sequence identity thereto. The portion of exon 1 of NRG1 in the fusion with DAAM1 preferably is or comprises the sequence according to SEQ ID NO: 604 and the allelic variant thereof has at least 85% identity to SEQ ID NO: 604, preferably at least 90% identity, 92%, 94%, 96% or more preferably at least 98% sequence identity thereto. More preferably, the portion of Exon 1 of DAAM1 comprises or consists of 2 to about 10, about 20, about 30, or up to about 40 or even all contiguous nucleic acids from SEQ ID NO: 603, including at least the nucleic acid of position 75. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 603, including at least the nucleic acid of position 75. More preferably, the portion of Exon 1 of DAAM1 comprises or is according to SEQ ID NO: 603, or an allelic variant thereof. Such short polynucleotide sequences are particularly useful for detecting the presence of a larger polynucleotide fusion between DAAM1 and NRG1 and establishing that such a fusion is an in-frame, oncogenic fusion comprising an EGF-like domain of NRG1.

Alternatively, the portion of Exon 1 of DAAM1 comprises or consists of SEQ ID NO: 606 (or an allelic variant of SEQ ID NO: 606), including at least the nucleic acid of position 102. Preferably, the portion of Exon 1 of DAAM1 comprises or consists of 2 to about 10, about 20, about 30, or up to about 40 or even all contiguous nucleic acids from SEQ ID NO: 606, or from the allelic variant thereof, including at least the nucleic acid of position 102. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 606, including at least the nucleic acid of position 102. In this alternative, the portion of Exon 1 of DAAM1 more preferably comprises or is according to SEQ ID NO: 606, or an allelic variant thereof. Preferably, the portion of exon 1 of NRG1 in the fusion with DAAM1 comprises or consists of 2 to about 10, about 20, about 30, or up to about 40 or even all contiguous nucleic acids from SEQ ID NO: 125, or from the allelic variant thereof, including at least the nucleic acid of position 1. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 125, including at least the nucleic acid of position 1.

In an alternatively preferred embodiment, the polynucleotide comprising a portion of Exon 1 of DAAM1 fused with a portion of exon 1 of NRG1 comprises 2 to about 10, about 20, about 30, or up to about 40 or even all contiguous nucleic acids from SEQ ID NO: 605, including the nucleic acids at positions 75 and 76. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 605, including at least the nucleic acids at positions 75 and 76. SEQ ID NO: 605 includes the junction between DAAM1 and NRG1, in particular the junction is between the nucleic acid at position 75, which stems from DAAM1 and the nucleic acid at position 76 which stems from NRG1. Preferably, the polynucleotide comprising a portion of Exon 1 of DAAM1 fused with a portion of exon 1 of NRG1 has the polynucleotide sequence of SEQ ID NO: 605, or an allelic variant thereof.

Preferably, any DAAM1-NRG1 polynucleotide fusion provided herein is a fusion of a portion of the untranslated region of DAAM1 with a portion of the untranslated region of NRG1. More preferably said fusion is a fusion comprising exon 1 of DAAM1, or a portion of exon 1, with exon 1 of NRG1, or a portion of exon 1 of NRG1. Said fusion is preferably the fusion of SEQ ID NO: 605, or an allelic variant thereof having at least 85% identity thereto, preferably at least 90%, 92%, 94%, 96% or even 98% identity thereto.

Preferably, the portion of exon 1 of DAAM1, or the allelic variant thereof, is 5′ to the portion of exon 1 of NRG1, or the allelic variant thereof. The NRG1 protein thereby has become positioned downstream of the DAAM1 promoter and is expected to have become transcriptionally controlled by said promoter. The resulting fusion thus results in expression of a non-protein fused NRG1 protein and thus contains the EGF-like domain. Therewith, the ability to drive proliferation and survival of a subset of human cancers, in particular breast cancer.

### ASPH-NRG1 Polynucleotide Fusion

In accordance with the present disclosure, there is also provided a polynucleotide comprising a ASPH nucleic acid sequence (or a portion of a ASPH nucleic acid sequence) fused with an NRG1 nucleic acid sequence (or a portion of an NRG1 nucleic acid sequence). Also included in said fusion are allelic variants of ASPH and NRG1 nucleic acid sequences.

Preferably, the ASPH nucleic acid sequence, or the portion thereof, comprises or consists of any one of SEQ ID NOs: 637-662, or an allelic variant of any one of SEQ ID NOs: 637-662, and the NRG1 nucleic acid sequence, or the portion thereof, comprises or consists of any one of SEQ ID NOs: 125-138, or an allelic variant of any one of SEQ ID NOs: 125-138. More preferably, the ASPH nucleic acid sequence comprises a portion of SEQ ID NOs: 662, or an allelic variant of SEQ ID NOs: 662, and the NRG1 nucleic acid sequence preferably comprises a portion of SEQ ID NOs: 138, or an allelic variant of SEQ ID NOs: 138. SEQ ID NOs: 637-661 correspond to the individual exons 1-25 of ASPH according to NM_001164750.2, respectively. SEQ ID NO: 662 corresponds to exons 1-25 of ASPH according to NM_001164750.2. SEQ ID NOs: 125-137 correspond to the individual exons 1-13 of the NRG1 sequence according to NM_001159999.3, respectively. SEQ ID NO: 138 corresponds to exons 1-13 of NRG1 according to NM_001159999.3.

In a preferred embodiment, the portion of the ASPH nucleic acid sequence comprises 2 to about 10, about 20, about 30, or up to about 40 or even all contiguous nucleic acids from any one of SEQ ID NOs: 637-662, or an allelic variant of any one of SEQ ID NOs: 637-662, and the portion of the NRG1 nucleic acid sequence comprises 2 to about 10, about 20, about 30, or up to about 40 or even all contiguous nucleic acids of any one of SEQ ID NOs: 125-138, or an allelic variant of any one of SEQ ID NOs: 125-138.

Preferably, the allelic variant of the ASPH nucleic acid sequences has at least 85% identity to any one of SEQ ID NOs: 637-662, preferably at least 90% identity, 92%, 94%, 96% or more preferably at least 98% sequence identity thereto; and the allelic variant of the NRG1 nucleic acid sequences has at least 85% identity to any one of SEQ ID NOs: 125-138, preferably at least 90% identity, 92%, 94%, 96% or more preferably at least 98% sequence identity thereto.

Preferably, the ASPH nucleic acid sequence, or the portion thereof, is 5′ to the NRG1 nucleic acid sequence, or the portion thereof.

Preferably, the polynucleotide comprising a ASPH nucleic acid sequence of ASPH, or a portion of said sequence, fused with a NRG1 nucleic acid sequence, or portion of said sequence, comprises or encodes an EGF-like domain of NRG1. An aberrant cell comprising the ASPH-NRG1 polynucleotide fusion or expressing the polypeptide fusion, comprises or encodes an EGF-like domain of NRG1. For detection, diagnosis or identification purposes, it may suffice to show that the fusion junction between ASPH and NRG1 is in-frame and occurs at a position such that the resulting fusion product, nucleic acid or protein, comprises an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

There is also provided a polynucleotide fusion comprising a portion of Exon 22 of ASPH fused with a portion of exon 2 of NRG1. Exon 22 of ASPH is preferably that of SEQ ID NO: 658, or an allelic variant of SEQ ID NO: 658, and exon 2 of NRG1 is preferably that of SEQ ID NO: 126, or an allelic variant of SEQ ID NO: 126.

When present in an aberrant cell of a patient or subject, said polynucleotide fusion preferably further includes any sequence 5′ from Exon 22 of ASPH and any sequence 3′ from exon 2 of NRG1, but to allow detection of the fusion junction using a polynucleotide-based detection assay, it may suffice that at least SEQ ID NOs: 658 and 126 are present. Any sequence 5′ from Exon 22 of ASPH comprises or consists of one or all of SEQ ID NOs: 637-657 (or any allelic variant of SEQ ID NOs: 637-657), and any sequence 3′ of exon 2 of NRG1 comprises or consists of one or all of SEQ ID NOs: 127-137 (or any allelic variant of SEQ ID NOs: 127-137).

Preferably, the allelic variant of Exon 22 of ASPH has at least 85% identity to SEQ ID NO: 658, preferably at least 90% identity, 92%, 94%, 96% or more preferably at least 98% sequence identity thereto and the allelic variant of exon 2 of NRG1 has at least 85% identity to SEQ ID NO: 126, preferably at least 90% identity, 92%, 94%, 96% or more preferably at least 98% sequence identity thereto.

Preferably, the portion of Exon 22 of ASPH comprises or is according to SEQ ID NO: 633 and the allelic variant thereof has at least 85% identity to SEQ ID NO: 633, preferably at least 90% identity, 92%, 94%, 96% or more preferably at least 98% sequence identity thereto. The portion of exon 2 of NRG1 in the fusion with ASPH preferably is or comprises the sequence according to SEQ ID NO: 634 and the allelic variant thereof has at least 85% identity to SEQ ID NO: 634, preferably at least 90% identity, 92%, 94%, 96% or more preferably at least 98% sequence identity thereto. More preferably, the portion of Exon 22 of ASPH comprises or consists of 2 to about 10, about 20, about 30, or up to about 40 or even all contiguous nucleic acids from SEQ ID NO: 633, including at least the nucleic acid of position 75. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 633, including at least the nucleic acid of position 75. More preferably, the portion of Exon 22 of ASPH comprises or is according to SEQ ID NO: 633, or an allelic variant thereof. Such short polynucleotide sequences are particularly useful for detecting the presence of a larger polynucleotide fusion between ASPH and NRG1 and establishing that such a fusion is an in-frame, oncogenic fusion comprising an EGF-like domain of NRG1.

Alternatively, the portion of Exon 22 of ASPH comprises or consists of SEQ ID NO: 658 (or an allelic variant of SEQ ID NO: 658), including at least the nucleic acid of position 136. Preferably, the portion of Exon 22 of ASPH comprises or consists of 2 to about 10, about 20, about 30, or up to about 40 or even all contiguous nucleic acids from SEQ ID NO: 658, or from the allelic variant thereof, including at least the nucleic acid of position 136. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 658, including at least the nucleic acid of position 136. In this alternative, the portion of Exon 22 of ASPH more preferably comprises or is according to SEQ ID NO: 658, or an allelic variant thereof. Preferably, the portion of exon 2 of NRG1 in the fusion with ASPH comprises or consists of 2 to about 10, about 20, about 30, or up to about 40 or even all contiguous nucleic acids from SEQ ID NO: 126, or from the allelic variant thereof, including at least the nucleic acid of position 1. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 126, including at least the nucleic acid of position 1.

In an alternatively preferred embodiment, the polynucleotide comprising a portion of Exon 22 of ASPH fused with a portion of exon 2 of NRG1 comprises 2 to about 10, about 20, about 30, or up to about 40 or even all contiguous nucleic acids from SEQ ID NO: 635, including the nucleic acids at positions 75 and 76. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 635, including at least the nucleic acids at positions 75 and 76. SEQ ID NO: 635 includes the junction between ASPH and NRG1, in particular the junction is between the nucleic acid at position 75, which stems from ASPH and the nucleic acid at position 76 which stems from NRG1. Preferably, the polynucleotide comprising a portion of Exon 22 of ASPH fused with a portion of exon 2 of NRG1 has the polynucleotide sequence of SEQ ID NO: 635, or an allelic variant thereof.

Preferably, any ASPH-NRG1 polynucleotide fusion provided herein is an in-frame fusion of ASPH with NRG1. More preferably said fusion is an in-frame fusion comprising Exon 22 of ASPH, or a portion of Exon 22, with exon 2 of NRG1, or a portion of exon 2 of NRG1. Said in-frame fusion is preferably the fusion of SEQ ID NO: 635, or an allelic variant thereof having at least 85% identity thereto, preferably at least 90%, 92%, 94%, 96% or even 98% identity thereto.

Preferably, the portion of Exon 22 of ASPH, or the allelic variant thereof, is 5′ to the portion of exon 2 of NRG1, or the allelic variant thereof. This orientation at the nucleic acid level results in a fusion polypeptide product that contains an N-terminus of ASPH and a C-terminus of NRG1. Also, the herein provided ASPH-NRG1 polynucleotide fusion produces a protein fusion, wherein the part from the N-terminus to the fusion junction is polypeptide sequence from ASPH and the part from the junction to the C-terminus is NRG1 polypeptide sequence, wherein the NRG1 part also provides its EGF-like domain. The ASPH-NRG1 fusion protein thereby retains the EGF-like domain of NRG1 and the ability to drive proliferation and survival of a subset of human cancers, in particular an adenocarcinoma, more in particular colorectal adenocarcinoma.

### NOTCH2-NRG1 Polynucleotide Fusion

There is also provided a polynucleotide fusion comprising a portion of Exon 6 of NOTCH2 fused with a portion of exon 6 of NRG1. Exon 6 of NOTCH2 is preferably that of SEQ ID NO: 700, or an allelic variant of SEQ ID NO: 700, and exon 6 of NRG1 is preferably that of SEQ ID NO: 130, or an allelic variant of SEQ ID NO:130.

When present in an aberrant cell of a patient or subject, said polynucleotide fusion preferably further includes any sequence 5′ from Exon 6 of NOTCH2 and any sequence 3′ from exon 6 of NRG1, but to allow detection of the fusion junction using a polynucleotide-based detection assay, it may suffice that at least SEQ ID NOs: 700 and 130 are present. Any sequence 5′ from Exon 6 of NOTCH2 comprises or consists of one or all of SEQ ID NOs: 695-699 (or any allelic variant of SEQ ID NOs: 695-699), and any sequence 3′ of exon 6 of NRG1 comprises or consists of one or all of SEQ ID NOs: 131-137 (or any allelic variant of SEQ ID NOs: 131-137).

Preferably, the allelic variant of Exon 6 of NOTCH2 has at least 85% identity to SEQ ID NO: 700, preferably at least 90% identity, 92%, 94%, 96% or more preferably at least 98% sequence identity thereto and the allelic variant of exon 6 of NRG1 has at least 85% identity to SEQ ID NO: 130, preferably at least 90% identity, 92%, 94%, 96% or more preferably at least 98% sequence identity thereto.

Preferably, the portion of Exon 6 of NOTCH2 comprises or is according to SEQ ID NO: 691 and the allelic variant thereof has at least 85% identity to SEQ ID NO: 691, preferably at least 90% identity, 92%, 94%, 96% or more preferably at least 98% sequence identity thereto. The portion of exon 6 of NRG1 in the fusion with NOTCH2 preferably is or comprises the sequence according to SEQ ID NO: 692 and the allelic variant thereof has at least 85% identity to SEQ ID NO: 692, preferably at least 90% identity, 92%, 94%, 96% or more preferably at least 98% sequence identity thereto. More preferably, the portion of Exon 6 of NOTCH2 comprises or consists of 2 to about 10, about 20, about 30, or up to about 40 or even all contiguous nucleic acids from SEQ ID NO: 691, including at least the nucleic acid of position 75. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 691, including at least the nucleic acid of position 75. More preferably, the portion of Exon 6 of NOTCH2 comprises or is according to SEQ ID NO: 691, or an allelic variant thereof. Such short polynucleotide sequences are particularly useful for detecting the presence of a larger polynucleotide fusion between NOTCH2 and NRG1 and establishing that such a fusion is an in frame, oncogenic fusion comprising an EGF-like domain of NRG1.

Alternatively, the portion of Exon 6 of NOTCH2 comprises or consists of SEQ ID NO: 700 (or an allelic variant of SEQ ID NO: 700), including at least the nucleic acid of position 234. Preferably, the portion of Exon 6 of NOTCH2 comprises or consists of 2 to about 10, about 20, about 30, or up to about 40 or even all contiguous nucleic acids from SEQ ID NO: 700, or from the allelic variant thereof, including at least the nucleic acid of position 234. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 700, including at least the nucleic acid of position 234. In this alternative, the portion of Exon 6 of NOTCH2 more preferably comprises or is according to SEQ ID NO: 700, or an allelic variant thereof. Preferably, the portion of exon 6 of NRG1 in the fusion with NOTCH2 comprises or consists of 2 to about 10, about 20, about 30, or up to about 40 or even all contiguous nucleic acids from SEQ ID NO: 130, or from the allelic variant thereof, including at least the nucleic acid of position 1. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 130, including at least the nucleic acid of position 1.

Preferably, any NOTCH2-NRG1 polynucleotide fusion provided herein is an in-frame fusion of NOTCH2 with NRG1. More preferably said fusion is an in-frame fusion comprising Exon 6 of NOTCH2, or a portion of Exon 6, with exon 6 of NRG1, or a portion of exon 6 of NRG1. Said in frame fusion is preferably the fusion of SEQ ID NO: 693, or an allelic variant thereof having at least 85% identity thereto, preferably at least 90%, 92%, 94%, 96% or even 98% identity thereto.

In an alternatively preferred embodiment, the polynucleotide comprising a portion of Exon 6 of NOTCH2 fused with a portion of exon 6 of NRG1 comprises 2 to about 10, about 20, about 30, or up to about 40 or even all contiguous nucleic acids from SEQ ID NO: 693, including the nucleic acids at positions 75 and 76. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 693, including at least the nucleic acids at positions 75 and 76. SEQ ID NO: 693 includes the junction between NOTCH2 and NRG1, in particular the junction is between the nucleic acid at position 75, which stems from NOTCH2 and the nucleic acid at position 76 which stems from NRG1. Preferably, the polynucleotide comprising a portion of Exon 6 of NOTCH2 fused with a portion of exon 6 of NRG1 has the polynucleotide sequence of SEQ ID NO: 693, or an allelic variant thereof.

In a preferred embodiment, there is provided a polynucleotide according to SEQ ID NO: 693, or a polynucleotide which comprises about 20, about 30, about 40 or all contiguous nucleic acids from SEQ ID NO: 693, preferably including the nucleic acids at positions 75 and 76. The number of contiguous nucleic acids may be 18, 19, 20, 21, 22, 23, 24, 25, 26, 27, 28, 29, 30, 31, 32, 33, 34, 35, 36, 37, 38, 39, 40 or all nucleic acids of SEQ ID NO: 693, preferably including at least the nucleic acids at positions 75 and 76.

Preferably, the polynucleotide comprising a portion of Exon 6 of NOTCH2, or an allelic variant thereof, fused with a portion of exon 6 of NRG1, or an allelic variant thereof, is part of a longer polynucleotide that further comprises or encodes an EGF-like domain of NRG1. An aberrant cell comprising the polynucleotide fusion involving NOTCH2 or expressing the polypeptide fusion, comprises or encodes an EGF-like domain of NRG1. For quick detection, diagnosis or identification purposes, it may suffice to show that the fusion junction between NOTCH2 and NRG1 is in-frame and occurs at such a position that the resulting fusion product, nucleic acid or protein, comprises an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

Preferably, the portion of Exon 6 of NOTCH2, or the allelic variant thereof, is 5′ to the portion of exon 6 of NRG1, or the allelic variant thereof. This orientation at the nucleic acid level results in a fusion polypeptide product that contains an N-terminus of NOTCH2 and a C-terminus of NRG1. Also, the herein provided NOTCH2-NRG1 polynucleotide fusion produces a protein fusion, wherein the part from the N-terminus to the fusion junction is polypeptide sequence from NOTCH2 and the part from the junction to the C-terminus is NRG1 polypeptide sequence, wherein the NRG1 part also provides its EGF-like domain. The NOTCH2-NRG1 fusion protein thereby retains the EGF-like domain of NRG1 and the ability to drive proliferation and survival of a subset of human cancers, in particular an adenocarcinoma, more in particular pancreatic ductal adenocarcinoma.

### CD74-NRG1 Polynucleotide Fusion

There is also provided a polynucleotide fusion comprising a portion of Exon 2 of CD74 fused with a portion of exon 2 of NRG1. Exon 2 of CD74 is preferably that of SEQ ID NO: 720, or an allelic variant of SEQ ID NO: 720, and exon 2 of NRG1 is preferably that of SEQ ID NO: 126, or an allelic variant of SEQ ID NO:126.

When present in an aberrant cell of a patient or subject, said polynucleotide fusion preferably further includes any sequence 5′ from Exon 2 of CD74 and any sequence 3′ from exon 2 of NRG1, but to allow detection of the fusion junction using a polynucleotide-based detection assay, it may suffice that at least SEQ ID NOs: 720 and 126 are present. Any sequence 5′ from Exon 2 of CD74 comprises or consists of SEQ ID NO: 719 (or any allelic variant of SEQ ID NO: 719), and any sequence 3′ of exon 2 of NRG1 comprises or consists of one or all of SEQ ID NOs: 127-137 (or any allelic variant of SEQ ID NOs: 127-137).

Preferably, the allelic variant of Exon 2 of CD74 has at least 85% identity to SEQ ID NO: 720, preferably at least 90% identity, 92%, 94%, 96% or more preferably at least 98% sequence identity thereto and the allelic variant of exon 2 of NRG1 has at least 85% identity to SEQ ID NO: 126, preferably at least 90% identity, 92%, 94%, 96% or more preferably at least 98% sequence identity thereto.

Preferably, the portion of Exon 2 of CD74 comprises or is according to SEQ ID NO: 715 and the allelic variant thereof has at least 85% identity to SEQ ID NO: 715, preferably at least 90% identity, 92%, 94%, 96% or more preferably at least 98% sequence identity thereto. The portion of exon 2 of NRG1 in the fusion with CD74 preferably is or comprises the sequence according to SEQ ID NO: 716 and the allelic variant thereof has at least 85% identity to SEQ ID NO: 716, preferably at least 90% identity, 92%, 94%, 96% or more preferably at least 98% sequence identity thereto. More preferably, the portion of Exon 2 of CD74 comprises or consists of 2 to about 10, about 20, about 30, or up to about 40 or even all contiguous nucleic acids from SEQ ID NO: 715, including at least the nucleic acid of position 75. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 715, including at least the nucleic acid of position 75. More preferably, the portion of Exon 2 of CD74 comprises or is according to SEQ ID NO: 715, or an allelic variant thereof. Such short polynucleotide sequences are particularly useful for detecting the presence of a larger polynucleotide fusion between CD74 and NRG1 and establishing that such a fusion is an in-frame, oncogenic fusion comprising an EGF-like domain of NRG1.

Alternatively, the portion of Exon 2 of CD74 comprises or consists of SEQ ID NO: 720 (or an allelic variant of SEQ ID NO: 720), including at least the nucleic acid of position 173. Preferably, the portion of Exon 2 of CD74 comprises or consists of 2 to about 10, about 20, about 30, or up to about 40 or even all contiguous nucleic acids from SEQ ID NO: 720, or from the allelic variant thereof, including at least the nucleic acid of position 173. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 720, including at least the nucleic acid of position 173. In this alternative, the portion of Exon 2 of CD74 more preferably comprises or is according to SEQ ID NO: 720, or an allelic variant thereof. Preferably, the portion of exon 2 of NRG1 in the fusion with CD74 comprises or consists of 2 to about 10, about 20, about 30, or up to about 40 or even all contiguous nucleic acids from SEQ ID NO: 126, or from the allelic variant thereof, including at least the nucleic acid of position 1. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 126, including at least the nucleic acid of position 1.

Preferably, any CD74-NRG1 polynucleotide fusion provided herein is an in-frame fusion of CD74 with NRG1. More preferably said fusion is an in-frame fusion comprising Exon 2 of CD74, or a portion of Exon 2, with exon 2 of NRG1, or a portion of exon 2 of NRG1. Said in-frame fusion is preferably the fusion of SEQ ID NO: 717, or an allelic variant thereof having at least 85% identity thereto, preferably at least 90%, 92%, 94%, 96% or even 98% identity thereto.

In an alternatively preferred embodiment, the polynucleotide comprising a portion of Exon 2 of CD74 fused with a portion of exon 2 of NRG1 comprises 2 to about 10, about 20, about 30, or up to about 40 or even all contiguous nucleic acids from SEQ ID NO: 717, including the nucleic acids at positions 75 and 76. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 717, including at least the nucleic acids at positions 75 and 76. SEQ ID NO: 717 includes the junction between CD74 and NRG1, in particular the junction is between the nucleic acid at position 75, which stems from CD74 and the nucleic acid at position 76 which stems from NRG1. Preferably, the polynucleotide comprising a portion of Exon 2 of CD74 fused with a portion of exon 2 of NRG1 has the polynucleotide sequence of SEQ ID NO: 717, or an allelic variant thereof.

In a preferred embodiment, there is provided a polynucleotide according to SEQ ID NO: 717, or a polynucleotide which comprises about 20, about 30, about 40 or all contiguous nucleic acids from SEQ ID NO: 717, preferably including the nucleic acids at positions 75 and 76. The number of contiguous nucleic acids may be 18, 19, 20, 21, 22, 23, 24, 25, 26, 27, 28, 29, 30, 31, 32, 33, 34, 35, 36, 37, 38, 39, 40 or all nucleic acids of SEQ ID NO: 717, preferably including at least the nucleic acids at positions 75 and 76.

Preferably, the polynucleotide comprising a portion of Exon 2 of CD74, or an allelic variant thereof, fused with a portion of exon 2 of NRG1, or an allelic variant thereof, is part of a longer polynucleotide that further comprises or encodes an EGF-like domain of NRG1. An aberrant cell comprising the polynucleotide fusion involving CD74 or expressing the polypeptide fusion, comprises or encodes an EGF-like domain of NRG1. For quick detection, diagnosis or identification purposes, it may suffice to show that the fusion junction between CD74 and NRG1 is in-frame and occurs at such a position that the resulting fusion product, nucleic acid or protein, comprises an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

Preferably, the portion of Exon 2 of CD74, or the allelic variant thereof, is 5′ to the portion of exon 2 of NRG1, or the allelic variant thereof. This orientation at the nucleic acid level results in a fusion polypeptide product that contains an N-terminus of CD74 and a C-terminus of NRG1. Also, the herein provided CD74-NRG1 polynucleotide fusion produces a protein fusion, wherein the part from the N-terminus to the fusion junction is polypeptide sequence from CD74 and the part from the junction to the C-terminus is NRG1 polypeptide sequence, wherein the NRG1 part also provides its EGF-like domain. The CD74-NRG1 fusion protein thereby retains the EGF-like domain of NRG1 and the ability to drive proliferation and survival of a subset of human cancers, in particular lung cancer.

### SDC4-NRG1 Polynucleotide Fusion

There is also provided a polynucleotide fusion comprising a portion of Exon 2 of SDC4 fused with a portion of exon 2 of NRG1. Exon 2 of said SDC4 is preferably that of SEQ ID NO: 746, or an allelic variant of SEQ ID NO: 746, and exon 2 of NRG1 is preferably that of SEQ ID NO: 126, or an allelic variant of SEQ ID NO: 126.

Preferably, the allelic variant of Exon 2 of said SDC4 has at least 85% identity to SEQ ID NO: 746, preferably at least 90% identity, 92%, 94%, 96% or more preferably at least 98% sequence identity thereto and the allelic variant of exon 2 of NRG1 has at least 85% identity to SEQ ID NO: 126, preferably at least 90% identity, 92%, 94%, 96% or more preferably at least 98% sequence identity thereto.

Preferably, the portion of Exon 2 of said SDC4 comprises or is according to SEQ ID NO: 741 and the allelic variant thereof has at least 85% identity to SEQ ID NO: 741, preferably at least 90% identity, 92%, 94%, 96% or more preferably at least 98% sequence identity thereto. The portion of exon 2 of NRG1 in the fusion with SDC4 preferably is or comprises the sequence according to SEQ ID NO: 742 and the allelic variant thereof has at least 85% identity to SEQ ID NO: 742, preferably at least 90% identity, 92%, 94%, 96% or more preferably at least 98% sequence identity thereto. More preferably, the portion of Exon 2 of SDC4 comprises or consists of 2 to about 10, about 20, about 30, or up to about 40 or even all contiguous nucleic acids from SEQ ID NO: 741, including at least the nucleic acid of position 75. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 741, including at least the nucleic acid of position 75. More preferably, the portion of Exon 2 of said SDC4 comprises or is according to SEQ ID NO: 741, or an allelic variant thereof. Such short polynucleotide sequences are particularly useful for detecting the presence of a larger polynucleotide fusion between said SDC4 and NRG1 and establishing that such a fusion is an in-frame, oncogenic fusion comprising an EGF-like domain of NRG1.

Alternatively, the portion of Exon 2 of said SDC4 comprises or consists of SEQ ID NO: 746 (or an allelic variant of SEQ ID NO: 746), including at least the nucleic acid of position 139. Preferably, the portion of Exon 2 of said SDC4 comprises or consists of 2 to about 10, about 20, about 30, or up to about 40 or even all contiguous nucleic acids from SEQ ID NO: 746, or from the allelic variant thereof, including at least the nucleic acid of position 139. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 746, including at least the nucleic acid of position 139. In this alternative, the portion of Exon 2 of SDC4 more preferably comprises or is according to SEQ ID NO: 746, or an allelic variant thereof. Preferably, the portion of exon 2 of NRG1 in the fusion with said SDC4 comprises or consists of 2 to about 10, about 20, about 30, or up to about 40 or even all contiguous nucleic acids from SEQ ID NO: 126, or from the allelic variant thereof, including at least the nucleic acid of position 1. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 126, including at least the nucleic acid of position 1.

Preferably, any SDC4-NRG1 polynucleotide fusion provided herein is an in-frame fusion of SDC4 with NRG1. More preferably said fusion is an in-frame fusion comprising Exon 2 of SDC4, or a portion of Exon 2, with exon 2 of NRG1, or a portion of exon 2 of NRG1. Said in-frame fusion is preferably the fusion of SEQ ID NO: 743, or an allelic variant thereof having at least 85% identity thereto, preferably at least 90%, 92%, 94%, 96% or even 98% identity thereto.

In an alternatively preferred embodiment, the polynucleotide comprising a portion of Exon 2 of said SDC4 fused with a portion of exon 2 of NRG1 comprises 2 to about 10, about 20, about 30, or up to about 40 or even all contiguous nucleic acids from SEQ ID NO: 743, including the nucleic acids at positions 75 and 76. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 743, including at least the nucleic acids at positions 75 and 76. SEQ ID NO: 743 includes the junction between said SDC4 and NRG1, in particular the junction is between the nucleic acid at position 75, which stems from SDC4 and the nucleic acid at position 76 which stems from NRG1. Preferably, the polynucleotide comprising a portion of Exon 2 of SDC4 fused with a portion of exon 2 of NRG1 has the polynucleotide sequence of SEQ ID NO: 743, or an allelic variant thereof.

In a preferred embodiment, there is provided a polynucleotide according to SEQ ID NO: 743, or a polynucleotide which comprises about 20, about 30, about 40 or all contiguous nucleic acids from SEQ ID NO: 743, preferably including the nucleic acids at positions 75 and 76. The number of contiguous nucleic acids may be 18, 19, 20, 21, 22, 23, 24, 25, 26, 27, 28, 29, 30, 31, 32, 33, 34, 35, 36, 37, 38, 39, 40 or all nucleic acids of SEQ ID NO: 743, preferably including at least the nucleic acids at positions 75 and 76.

Preferably, the polynucleotide comprising a portion of Exon 2 of SDC4, or an allelic variant thereof, fused with a portion of exon 2 of NRG1, or an allelic variant thereof, is part of a longer polynucleotide that further comprises or encodes an EGF-like domain of NRG1. An aberrant cell comprising the polynucleotide fusion involving said SDC4 or expressing the polypeptide fusion, comprises or encodes an EGF-like domain of NRG1. For quick detection, diagnosis or identification purposes, it may suffice to show that the fusion junction between said SDC4 and NRG1 is in-frame and occurs at such a position that the resulting fusion product, nucleic acid or protein, comprises an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

Preferably, the portion of Exon 2 of SDC4, or the allelic variant thereof, is 5′ to the portion of exon 2 of NRG1, or the allelic variant thereof. This orientation at the nucleic acid level results in a fusion polypeptide product that contains an N-terminus of SDC4 and a C-terminus of NRG1. Also, the herein provided SDC4-NRG1 polynucleotide fusion produces a protein fusion, wherein the part from the N-terminus to the fusion junction is polypeptide sequence from SDC4 and the part from the junction to the C-terminus is NRG1 polypeptide sequence, wherein the NRG1 part also provides its EGF-like domain. The SDC4-NRG1 fusion protein thereby retains the EGF-like domain of NRG1 and the ability to drive proliferation and survival of a subset of human cancers, in particular lung cancer.

When present in an aberrant cell of a patient or subject, said polynucleotide fusion preferably further includes any sequence 5′ from Exon 2 of SDC4 and any sequence 3′ from exon 2 of NRG1, but to allow detection of the fusion junction using a polynucleotide-based detection assay, it may suffice that at least SEQ ID NOs: 746 and 126 are present. Any sequence 5′ from Exon 2 of SDC4 comprises or consists of SEQ ID NO: 745 (or any allelic variant of SEQ ID NO: 745), and any sequence 3′ of exon 2 of NRG1 comprises or consists of one or all of SEQ ID NOs: 127-137 (or any allelic variant of SEQ ID NOs: 127-137).

Further, there is provided a polynucleotide fusion comprising a portion of Exon 4 of SDC4 fused with a portion of exon 2 of NRG1. Exon 4 of said SDC4 is preferably that of SEQ ID NO: 748, or an allelic variant of SEQ ID NO: 748, and exon 2 of NRG1 is preferably that of SEQ ID NO: 126, or an allelic variant of SEQ ID NO: 126.

Preferably, the allelic variant of exon 4 of said SDC4 has at least 85% identity to SEQ ID NO: 748, preferably at least 90% identity, 92%, 94%, 96% or more preferably at least 98% sequence identity thereto and the allelic variant of exon 2 of NRG1 has at least 85% identity to SEQ ID NO: 126, preferably at least 90% identity, 92%, 94%, 96% or more preferably at least 98% sequence identity thereto.

Preferably, the portion of exon 4 of said SDC4 comprises or is according to SEQ ID NO: 822 and the allelic variant thereof has at least 85% identity to SEQ ID NO: 822, preferably at least 90% identity, 92%, 94%, 96% or more preferably at least 98% sequence identity thereto. The portion of exon 2 of NRG1 in the fusion with SDC4 preferably is or comprises the sequence according to SEQ ID NO: 823 and the allelic variant thereof has at least 85% identity to SEQ ID NO: 823, preferably at least 90% identity, 92%, 94%, 96% or more preferably at least 98% sequence identity thereto. More preferably, the portion of exon 4 of SDC4 comprises or consists of 2 to about 10, about 20, about 30, or up to about 40 or even all contiguous nucleic acids from SEQ ID NO: 822, including at least the nucleic acid of position 75. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 822, including at least the nucleic acid of position 75. More preferably, the portion of exon 4 of said SDC4 comprises or is according to SEQ ID NO: 822, or an allelic variant thereof. Such short polynucleotide sequences are particularly useful for detecting the presence of a larger polynucleotide fusion between said SDC4 and NRG1 and establishing that such a fusion is an in-frame, oncogenic fusion comprising an EGF-like domain of NRG1.

Alternatively, the portion of exon 4 of said SDC4 comprises or consists of SEQ ID NO: 748 (or an allelic variant of SEQ ID NO: 748), including at least the nucleic acid of position 199. Preferably, the portion of exon 4 of said SDC4 comprises or consists of 2 to about 10, about 20, about 30, or up to about 40 or even all contiguous nucleic acids from SEQ ID NO: 748, or from the allelic variant thereof, including at least the nucleic acid of position 199. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 748, including at least the nucleic acid of position 199. In this alternative, the portion of exon 4 of SDC4 more preferably comprises or is according to SEQ ID NO: 748, or an allelic variant thereof. Preferably, the portion of exon 2 of NRG1 in the fusion with of SDC4 comprises or consists of 2 to about 10, about 20, about 30, or up to about 40 or even all contiguous nucleic acids from SEQ ID NO: 126, or from the allelic variant thereof, including at least the nucleic acid of position 1. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 126, including at least the nucleic acid of position 1.

Preferably, any SDC4-NRG1 polynucleotide fusion provided herein is an in-frame fusion of SDC4 with NRG1. More preferably said fusion is an in-frame fusion comprising exon 4 of SDC4, or a portion of exon 4, with exon 2 of NRG1, or a portion of exon 2 of NRG1. Said in-frame fusion is preferably the fusion of SEQ ID NO: 824, or an allelic variant thereof having at least 85% identity thereto, preferably at least 90%, 92%, 94%, 96% or even 98% identity thereto.

In an alternatively preferred embodiment, the polynucleotide comprising a portion of exon 4 of said SDC4 fused with a portion of exon 2 of NRG1 comprises 2 to about 10, about 20, about 30, or up to about 40 or even all contiguous nucleic acids from SEQ ID NO: 824, including the nucleic acids at positions 75 and 76. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 824, including at least the nucleic acids at positions 75 and 76. SEQ ID NO: 824 includes the junction between said SDC4 and NRG1, in particular the junction is between the nucleic acid at position 75, which stems from SDC4 and the nucleic acid at position 76 which stems from NRG1. Preferably, the polynucleotide comprising a portion of exon 4 of SDC4 fused with a portion of exon 2 of NRG1 has the polynucleotide sequence of SEQ ID NO: 824, or an allelic variant thereof.

In a preferred embodiment, there is provided a polynucleotide according to SEQ ID NO: 824, or a polynucleotide which comprises about 20, about 30, about 40 or all contiguous nucleic acids from SEQ ID NO: 824, preferably including the nucleic acids at positions 75 and 76. The number of contiguous nucleic acids may be 18, 19, 20, 21, 22, 23, 24, 25, 26, 27, 28, 29, 30, 31, 32, 33, 34, 35, 36, 37, 38, 39, 40 or all nucleic acids of SEQ ID NO: 824, preferably including at least the nucleic acids at positions 75 and 76.

Preferably, the polynucleotide comprising a portion of exon 4 of SDC4, or an allelic variant thereof, fused with a portion of exon 2 of NRG1, or an allelic variant thereof, is part of a longer polynucleotide that further comprises or encodes an EGF-like domain of NRG1. An aberrant cell comprising the polynucleotide fusion involving said SDC4 or expressing the polypeptide fusion, comprises or encodes an EGF-like domain of NRG1. For quick detection, diagnosis or identification purposes, it may suffice to show that the fusion junction between said SDC4 and NRG1 is in-frame and occurs at such a position that the resulting fusion product, nucleic acid or protein, comprises an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

Preferably, the portion of exon 4 of SDC4, or the allelic variant thereof, is 5′ to the portion of exon 2 of NRG1, or the allelic variant thereof. This orientation at the nucleic acid level results in a fusion polypeptide product that contains an N-terminus of SDC4 and a C-terminus of NRG1. Also, the herein provided SDC4-NRG1 polynucleotide fusion produces a protein fusion, wherein the part from the N-terminus to the fusion junction is polypeptide sequence from SDC4 and the part from the junction to the C-terminus is NRG1 polypeptide sequence, wherein the NRG1 part also provides its EGF-like domain. The SDC4-NRG1 fusion protein thereby retains the EGF-like domain of NRG1 and the ability to drive proliferation and survival of a subset of human cancers, including lung cancer, in particular non-small cell lung cancer.

When present in an aberrant cell of a patient or subject, said polynucleotide fusion preferably further includes any sequence 5′ from Exon 4 of SDC4 and any sequence 3′ from exon 2 of NRG1, but to allow detection of the fusion junction using a polynucleotide-based detection assay, it may suffice that at least SEQ ID NOs: 748 and 126 are present. Any sequence 5′ from Exon 4 of SDC4 comprises or consists of one or all of SEQ ID NOs: 745-747 (or any allelic variant of SEQ ID NOs: 745-747), and any sequence 3′ of exon 2 of NRG1 comprises or consists of one or all of SEQ ID NOs: 127-137 (or any allelic variant of SEQ ID NOs: 127-137).

### SLC4A4-NRG1 Polynucleotide Fusion

There is also provided a polynucleotide fusion comprising a portion of Exon 14 of SLC4A4 fused with a portion of exon 6 of NRG1. Exon 14 of SLC4A4 is preferably that of SEQ ID NO: 780, or an allelic variant of SEQ ID NO: 780, and exon 6 of NRG1 is preferably that of SEQ ID NO: 130, or an allelic variant of SEQ ID NO:130.

When present in an aberrant cell of a patient or subject, said polynucleotide fusion preferably further includes any sequence 5′ from Exon 14 of SLC4A4 and any sequence 3′ from exon 6 of NRG1, but to allow detection of the fusion junction using a polynucleotide-based detection assay, it may suffice that at least SEQ ID NOs: 780 and 130 are present. Any sequence 5′ from Exon 14 of SLC4A4 comprises or consists of one or all of SEQ ID NOs: 767-779 (or any allelic variant of SEQ ID NOs: 767-779), and any sequence 3′ of exon 6 of NRG1 comprises or consists of one or all of SEQ ID NOs: 131-137 (or any allelic variant of SEQ ID NOs: 131-137).

Preferably, the allelic variant of Exon 14 of SLC4A4 has at least 85% identity to SEQ ID NO: 780, preferably at least 90% identity, 92%, 94%, 96% or more preferably at least 98% sequence identity thereto and the allelic variant of exon 6 of NRG1 has at least 85% identity to SEQ ID NO: 130, preferably at least 90% identity, 92%, 94%, 96% or more preferably at least 98% sequence identity thereto.

Preferably, the portion of Exon 14 of SLC4A4 comprises or is according to SEQ ID NO: 763 and the allelic variant thereof has at least 85% identity to SEQ ID NO: 763, preferably at least 90% identity, 92%, 94%, 96% or more preferably at least 98% sequence identity thereto. The portion of exon 6 of NRG1 in the fusion with SLC4A4 preferably is or comprises the sequence according to SEQ ID NO: 764 and the allelic variant thereof has at least 85% identity to SEQ ID NO: 764, preferably at least 90% identity, 92%, 94%, 96% or more preferably at least 98% sequence identity thereto. More preferably, the portion of Exon 14 of SLC4A4 comprises or consists of 2 to about 10, about 20, about 30, or up to about 40 or even all contiguous nucleic acids from SEQ ID NO: 763, including at least the nucleic acid of position 75. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 763, including at least the nucleic acid of position 75. More preferably, the portion of Exon 14 of SLC4A4 comprises or is according to SEQ ID NO: 763, or an allelic variant thereof. Such short polynucleotide sequences are particularly useful for detecting the presence of a larger polynucleotide fusion between SLC4A4 and NRG1 and establishing that such a fusion is an in-frame, oncogenic fusion comprising an EGF-like domain of NRG1.

Alternatively, the portion of Exon 14 of SLC4A4 comprises or consists of SEQ ID NO: 780 (or an allelic variant of SEQ ID NO: 780), including at least the nucleic acid of position 272. Preferably, the portion of Exon 14 of SLC4A4 comprises or consists of 2 to about 10, about 20, about 30, or up to about 40 or even all contiguous nucleic acids from SEQ ID NO: 780, or from the allelic variant thereof, including at least the nucleic acid of position 272. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 780, including at least the nucleic acid of position 272. In this alternative, the portion of Exon 14 of SLC4A4 more preferably comprises or is according to SEQ ID NO: 780, or an allelic variant thereof. Preferably, the portion of exon 6 of NRG1 in the fusion with SLC4A4 comprises or consists of 2 to about 10, about 20, about 30, or up to about 40 or even all contiguous nucleic acids from SEQ ID NO: 130, or from the allelic variant thereof, including at least the nucleic acid of position 1. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 130, including at least the nucleic acid of position 1.

Preferably, any SLC4A4-NRG1 polynucleotide fusion provided herein is an in-frame fusion of SLC4A4 with NRG1. More preferably said fusion is an in frame fusion comprising Exon 14 of SLC4A4, or a portion of Exon 14, with exon 6 of NRG1, or a portion of exon 6 of NRG1. Said in-frame fusion is preferably the fusion of SEQ ID NO: 765, or an allelic variant thereof having at least 85% identity thereto, preferably at least 90%, 92%, 94%, 96% or even 98% identity thereto.

In an alternatively preferred embodiment, the polynucleotide comprising a portion of Exon 14 of SLC4A4 fused with a portion of exon 6 of NRG1 comprises 2 to about 10, about 20, about 30, or up to about 40 or even all contiguous nucleic acids from SEQ ID NO: 765, including the nucleic acids at positions 75 and 76. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 765, including at least the nucleic acids at positions 75 and 76. SEQ ID NO: 765 includes the junction between SLC4A4 and NRG1, in particular the junction is between the nucleic acid at position 75, which stems from SLC4A4 and the nucleic acid at position 76 which stems from NRG1. Preferably, the polynucleotide comprising a portion of Exon 14 of SLC4A4 fused with a portion of exon 6 of NRG1 has the polynucleotide sequence of SEQ ID NO: 765, or an allelic variant thereof.

In a preferred embodiment, there is provided a polynucleotide according to SEQ ID NO: 765, or a polynucleotide which comprises about 20, about 30, about 40 or all contiguous nucleic acids from SEQ ID NO: 765, preferably including the nucleic acids at positions 75 and 76. The number of contiguous nucleic acids may be 18, 19, 20, 21, 22, 23, 24, 25, 26, 27, 28, 29, 30, 31, 32, 33, 34, 35, 36, 37, 38, 39, 40 or all nucleic acids of SEQ ID NO: 765, preferably including at least the nucleic acids at positions 75 and 76.

Preferably, the polynucleotide comprising a portion of Exon 14 of SLC4A4, or an allelic variant thereof, fused with a portion of exon 6 of NRG1, or an allelic variant thereof, is part of a longer polynucleotide that further comprises or encodes an EGF-like domain of NRG1. An aberrant cell comprising the polynucleotide fusion involving SLC4A4 or expressing the polypeptide fusion, comprises or encodes an EGF-like domain of NRG1. For quick detection, diagnosis or identification purposes, it may suffice to show that the fusion junction between SLC4A4 and NRG1 is in-frame and occurs at such a position that the resulting fusion product, nucleic acid or protein, comprises an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

Preferably, the portion of Exon 14 of SLC4A4, or the allelic variant thereof, is 5′ to the portion of exon 6 of NRG1, or the allelic variant thereof. This orientation at the nucleic acid level results in a fusion polypeptide product that contains an N-terminus of SLC4A4 and a C-terminus of NRG1. Also, the herein provided SLC4A4-NRG1 polynucleotide fusion produces a protein fusion, wherein the part from the N-terminus to the fusion junction is polypeptide sequence from SLC4A4 and the part from the junction to the C-terminus is NRG1 polypeptide sequence, wherein the NRG1 part also provides its EGF-like domain. The SLC4A4-NRG1 fusion protein thereby retains the EGF-like domain of NRG1 and the ability to drive proliferation and survival of a subset of human cancers, in particular a pancreatic cancer.

### ZFAT-NRG1 Polynucleotide Fusion

In accordance with the present disclosure, there is also provided a polynucleotide comprising a ZFAT nucleic acid sequence (or a portion of a ZFAT nucleic acid sequence) fused with an NRG1 nucleic acid sequence (or a portion of an NRG1 nucleic acid sequence). Also included in said fusion are allelic variants of ZFAT and NRG1 nucleic acid sequences.

Preferably, the ZFAT nucleic acid sequence, or the portion thereof, comprises or consists of any one of SEQ ID NOs: 830-846, or an allelic variant of any one of SEQ ID NOs: 830-846, and the NRG1 nucleic acid sequence, or the portion thereof, comprises or consists of any one of SEQ ID NOs: 125-138, or an allelic variant of any one of SEQ ID NOs: 125-138. More preferably, the ZFAT nucleic acid sequence comprises a portion of SEQ ID NOs: 846, or an allelic variant of SEQ ID NOs: 846, and the NRG1 nucleic acid sequence preferably comprises a portion of SEQ ID NOs: 138, or an allelic variant of SEQ ID NOs: 138. SEQ ID NOs: 830-845 correspond to the individual exons 1-16 of ZFAT according to NM_020863.4, respectively. SEQ ID NO: 846 corresponds to exons 1-16 of ZFAT according to NM_020863.4. SEQ ID NOs: 125-137 correspond to the individual exons 1-13 of the NRG1 sequence according to NM_001159999.3, respectively. SEQ ID NO: 138 corresponds to exons 1-13 of NRG1 according to NM_001159999.3.

In a preferred embodiment, the portion of the ZFAT nucleic acid sequence comprises 2 to about 10, about 20, about 30, or up to about 40 or even all contiguous nucleic acids from any one of SEQ ID NOs: 830-846, or an allelic variant of any one of SEQ ID NOs: 830-846, and the portion of the NRG1 nucleic acid sequence comprises 2 to about 10, about 20, about 30, or up to about 40 or even all contiguous nucleic acids of any one of SEQ ID NOs: 125-138, or an allelic variant of any one of SEQ ID NOs: 125-138.

Preferably, the allelic variant of the ZFAT nucleic acid sequences has at least 85% identity to any one of SEQ ID NOs: 830-846, preferably at least 90% identity, 92%, 94%, 96% or more preferably at least 98% sequence identity thereto; and the allelic variant of the NRG1 nucleic acid sequences has at least 85% identity to any one of SEQ ID NOs: 125-138, preferably at least 90% identity, 92%, 94%, 96% or more preferably at least 98% sequence identity thereto.

Preferably, the ZFAT nucleic acid sequence, or the portion thereof, is 5′ to the NRG1 nucleic acid sequence, or the portion thereof.

Preferably, the polynucleotide comprising a ZFAT nucleic acid sequence of ZFAT, or a portion of said sequence, fused with a NRG1 nucleic acid sequence, or portion of said sequence, comprises or encodes an EGF-like domain of NRG1.

An aberrant cell comprising the ZFAT-NRG1 polynucleotide fusion or expressing the polypeptide fusion, comprises or encodes an EGF-like domain of NRG1. For detection, diagnosis or identification purposes, it may suffice to show that the fusion junction between ZFAT and NRG1 is in-frame and occurs at a position such that the resulting fusion product, nucleic acid or protein, comprises an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

There is also provided a polynucleotide fusion comprising a portion of Exon 12 of ZFAT fused with a portion of exon 6 of NRG1. Exon 12 of ZFAT is preferably that of SEQ ID NO: 841, or an allelic variant of SEQ ID NO: 841, and exon 6 of NRG1 is preferably that of SEQ ID NO: 130, or an allelic variant of SEQ ID NO:130.

When present in an aberrant cell of a patient or subject, said polynucleotide fusion preferably further includes any sequence 5′ from Exon 12 of ZFAT and any sequence 3′ from exon 6 of NRG1, but to allow detection of the fusion junction using a polynucleotide-based detection assay, it may suffice that at least SEQ ID NOs: 841 and 130 are present. Any sequence 5′ from Exon 12 of ZFAT comprises or consists of one or all of SEQ ID NOs: 830-840 (or any allelic variant of SEQ ID NOs: 830-840), and any sequence 3′ of exon 6 of NRG1 comprises or consists of one or all of SEQ ID NOs: 131-137 (or any allelic variant of SEQ ID NOs: 131-137).

Preferably, the allelic variant of Exon 12 of ZFAT has at least 85% identity to SEQ ID NO: 841, preferably at least 90% identity, 92%, 94%, 96% or more preferably at least 98% sequence identity thereto and the allelic variant of exon 6 of NRG1 has at least 85% identity to SEQ ID NO: 130, preferably at least 90% identity, 92%, 94%, 96% or more preferably at least 98% sequence identity thereto.

Preferably, the portion of Exon 12 of ZFAT comprises or is according to SEQ ID NO: 826 and the allelic variant thereof has at least 85% identity to SEQ ID NO: 826, preferably at least 90% identity, 92%, 94%, 96% or more preferably at least 98% sequence identity thereto. The portion of exon 6 of NRG1 in the fusion with ZFAT preferably is or comprises the sequence according to SEQ ID NO: 827 and the allelic variant thereof has at least 85% identity to SEQ ID NO: 827, preferably at least 90% identity, 92%, 94%, 96% or more preferably at least 98% sequence identity thereto. More preferably, the portion of Exon 12 of ZFAT comprises or consists of 2 to about 10, about 20, about 30, or up to about 40 or even all contiguous nucleic acids from SEQ ID NO: 826, including at least the nucleic acid of position 75. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 826, including at least the nucleic acid of position 75. More preferably, the portion of Exon 12 of ZFAT comprises or is according to SEQ ID NO: 826, or an allelic variant thereof. Such short polynucleotide sequences are particularly useful for detecting the presence of a larger polynucleotide fusion between ZFAT and NRG1 and establishing that such a fusion is an in-frame, oncogenic fusion comprising an EGF-like domain of NRG1.

Alternatively, the portion of Exon 12 of ZFAT comprises or consists of SEQ ID NO: 841 (or an allelic variant of SEQ ID NO: 841), including at least the nucleic acid of position 139. Preferably, the portion of Exon 12 of ZFAT comprises or consists of 2 to about 10, about 20, about 30, or up to about 40 or even all contiguous nucleic acids from SEQ ID NO: 841, or from the allelic variant thereof, including at least the nucleic acid of position 139. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 841, including at least the nucleic acid of position 139. In this alternative, the portion of Exon 12 of ZFAT more preferably comprises or is according to SEQ ID NO: 841, or an allelic variant thereof. Preferably, the portion of exon 6 of NRG1 in the fusion with ZFAT comprises or consists of 2 to about 10, about 20, about 30, or up to about 40 or even all contiguous nucleic acids from SEQ ID NO: 130, or from the allelic variant thereof, including at least the nucleic acid of position 1. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 130, including at least the nucleic acid of position 1.

In an alternatively preferred embodiment, the polynucleotide comprising a portion of Exon 12 of ZFAT fused with a portion of exon 6 of NRG1 comprises 2 to about 10, about 20, about 30, or up to about 40 or even all contiguous nucleic acids from SEQ ID NO: 828, including the nucleic acids at positions 75 and 76. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 828, including at least the nucleic acids at positions 75 and 76. SEQ ID NO: 828 includes the junction between ZFAT and NRG1, in particular the junction is between the nucleic acid at position 75, which stems from ZFAT and the nucleic acid at position 76 which stems from NRG1. Preferably, the polynucleotide comprising a portion of Exon 12 of ZFAT fused with a portion of exon 6 of NRG1 has the polynucleotide sequence of SEQ ID NO: 828, or an allelic variant thereof.

Preferably, any ZFAT-NRG1 polynucleotide fusion provided herein is an in-frame fusion of ZFAT with NRG1. More preferably said fusion is an in frame fusion comprising Exon 12 of ZFAT, or a portion of Exon 12, with exon 6 of NRG1, or a portion of exon 6 of NRG1. Said in-frame fusion is preferably the fusion of SEQ ID NO: 828, or an allelic variant thereof having at least 85% identity thereto, preferably at least 90%, 92%, 94%, 96% or even 98% identity thereto.

Preferably, the portion of Exon 12 of ZFAT, or the allelic variant thereof, is 5′ to the portion of exon 6 of NRG1, or the allelic variant thereof. This orientation at the nucleic acid level results in a fusion polypeptide product that contains an N-terminus of ZFAT and a C-terminus of NRG1. Also, the herein provided ZFAT-NRG1 polynucleotide fusion produces a protein fusion, wherein the part from the N-terminus to the fusion junction is polypeptide sequence from ZFAT and the part from the junction to the C-terminus is NRG1 polypeptide sequence, wherein the NRG1 part also provides its EGF-like domain. The ZFAT-NRG1 fusion protein thereby retains the EGF-like domain of NRG1 and the ability to drive proliferation and survival of a subset of human cancers, in particular non-small cell lung cancer.

### DSCAML1-NRG1 Polynucleotide Fusion

In accordance with the present disclosure, there is also provided a polynucleotide comprising a DSCAML1 nucleic acid sequence (or a portion of a DSCAML1 nucleic acid sequence) fused with an NRG1 nucleic acid sequence (or a portion of an NRG1 nucleic acid sequence). Also included in said fusion are allelic variants of DSCAML1 and NRG1 nucleic acid sequences.

Preferably, the DSCAML1 nucleic acid sequence, or the portion thereof, comprises or consists of any one of SEQ ID NOs: 870-903, or an allelic variant of any one of SEQ ID NOs: 870-903, and the NRG1 nucleic acid sequence, or the portion thereof, comprises or consists of any one of SEQ ID NOs: 125-138, or an allelic variant of any one of SEQ ID NOs: 125-138. More preferably, the DSCAML1 nucleic acid sequence comprises a portion of SEQ ID NOs: 903, or an allelic variant of SEQ ID NOs: 903, and the NRG1 nucleic acid sequence preferably comprises a portion of SEQ ID NOs: 138, or an allelic variant of SEQ ID NOs: 138. SEQ ID NOs: 870-902 correspond to the individual exons 1-33 of DSCAML1 according to NM_020693.4, respectively. SEQ ID NO: 903 corresponds to exons 1-33 of DSCAML1 according to NM_020693.4. SEQ ID NOs: 125-137 correspond to the individual exons 1-13 of the NRG1 sequence according to NM_001159999.3, respectively. SEQ ID NO: 138 corresponds to exons 1-13 of NRG1 according to NM_001159999.3.

In a preferred embodiment, the portion of the DSCAML1 nucleic acid sequence comprises 2 to about 10, about 20, about 30, or up to about 40 or even all contiguous nucleic acids from any one of SEQ ID NOs: 870-903, or an allelic variant of any one of SEQ ID NOs: 870-903, and the portion of the NRG1 nucleic acid sequence comprises 2 to about 10, about 20, about 30, or up to about 40 or even all contiguous nucleic acids of any one of SEQ ID NOs: 125-138, or an allelic variant of any one of SEQ ID NOs: 125-138.

Preferably, the allelic variant of the DSCAML1 nucleic acid sequences has at least 85% identity to any one of SEQ ID NOs: 870-903, preferably at least 90% identity, 92%, 94%, 96% or more preferably at least 98% sequence identity thereto; and the allelic variant of the NRG1 nucleic acid sequences has at least 85% identity to any one of SEQ ID NOs: 125-138, preferably at least 90% identity, 92%, 94%, 96% or more preferably at least 98% sequence identity thereto.

Preferably, the DSCAML1 nucleic acid sequence, or the portion thereof, is 5′ to the NRG1 nucleic acid sequence, or the portion thereof.

Preferably, the polynucleotide comprising a DSCAML1 nucleic acid sequence of DSCAML1, or a portion of said sequence, fused with a NRG1 nucleic acid sequence, or portion of said sequence, comprises or encodes an EGF-like domain of NRG1.

An aberrant cell comprising the DSCAML1-NRG1 polynucleotide fusion or expressing the polypeptide fusion, comprises or encodes an EGF-like domain of NRG1. For detection, diagnosis or identification purposes, it may suffice to show that the fusion junction between DSCAML1 and NRG1 is in-frame and occurs at a position such that the resulting fusion product, nucleic acid or protein, comprises an EGF-like domain of NRG1. Said EGF-like domain is preferably the EGF-like domain according to SEQ ID NO: 163, or an allelic variant thereof having at least 85% identity to SEQ ID NO: 163, preferably at least 90% identity, 92%, 94%, 96% or more preferably at least 98% sequence identity thereto.

There is also provided a polynucleotide fusion comprising a portion of Exon 3 of DSCAML1 fused with a portion of exon 2 of NRG1. Exon 3 of DSCAML1 is preferably that of SEQ ID NO: 872, or an allelic variant of SEQ ID NO: 872, and exon 2 of NRG1 is preferably that of SEQ ID NO: 126, or an allelic variant of SEQ ID NO:126.

When present in an aberrant cell of a patient or subject, said polynucleotide fusion preferably further includes any sequence 5′ from Exon 3 of DSCAML1 and any sequence 3′ from exon 2 of NRG1, but to allow detection of the fusion junction using a polynucleotide-based detection assay, it may suffice that at least SEQ ID NOs: 872 and 126 are present. Any sequence 5′ from Exon 3 of DSCAML1 comprises or consists of one or all of SEQ ID NOs: 870-871 (or any allelic variant of SEQ ID NOs: 870-871), and any sequence 3′ of exon 2 of NRG1 comprises or consists of one or all of SEQ ID NOs: 127-137 (or any allelic variant of SEQ ID NOs: 127-137).

Preferably, the allelic variant of Exon 3 of DSCAML1 has at least 85% identity to SEQ ID NO: 872, preferably at least 90% identity, 92%, 94%, 96% or more preferably at least 98% sequence identity thereto and the allelic variant of exon 2 of NRG1 has at least 85% identity to SEQ ID NO: 126, preferably at least 90% identity, 92%, 94%, 96% or more preferably at least 98% sequence identity thereto.

Preferably, the portion of Exon 3 of DSCAML1 comprises or is according to SEQ ID NO: 866 and the allelic variant thereof has at least 85% identity to SEQ ID NO: 866, preferably at least 90% identity, 92%, 94%, 96% or more preferably at least 98% sequence identity thereto. The portion of exon 2 of NRG1 in the fusion with DSCAML1 preferably is or comprises the sequence according to SEQ ID NO: 867 and the allelic variant thereof has at least 85% identity to SEQ ID NO: 867, preferably at least 90% identity, 92%, 94%, 96% or more preferably at least 98% sequence identity thereto. More preferably, the portion of Exon 3 of DSCAML1 comprises or consists of 2 to about 10, about 20, about 30, or up to about 40 or even all contiguous nucleic acids from SEQ ID NO: 866, including at least the nucleic acid of position 75. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 866, including at least the nucleic acid of position 75. More preferably, the portion of Exon 3 of DSCAML1 comprises or is according to SEQ ID NO: 866, or an allelic variant thereof. Such short polynucleotide sequences are particularly useful for detecting the presence of a larger polynucleotide fusion between DSCAML1 and NRG1 and establishing that such a fusion is an in-frame, oncogenic fusion comprising an EGF-like domain of NRG1.

Alternatively, the portion of Exon 3 of DSCAML1 comprises or consists of SEQ ID NO: 872 (or an allelic variant of SEQ ID NO: 872), including at least the nucleic acid of position 147. Preferably, the portion of Exon 3 of DSCAML1 comprises or consists of 2 to about 10, about 20, about 30, or up to about 40 or even all contiguous nucleic acids from SEQ ID NO: 872, or from the allelic variant thereof, including at least the nucleic acid of position 147. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 872, including at least the nucleic acid of position 147. In this alternative, the portion of Exon 3 of DSCAML1 more preferably comprises or is according to SEQ ID NO: 872, or an allelic variant thereof. Preferably, the portion of exon 2 of NRG1 in the fusion with DSCAML1 comprises or consists of 2 to about 10, about 20, about 30, or up to about 40 or even all contiguous nucleic acids from SEQ ID NO: 126, or from the allelic variant thereof, including at least the nucleic acid of position 1. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 126, including at least the nucleic acid of position 1.

In an alternatively preferred embodiment, the polynucleotide comprising a portion of Exon 3 of DSCAML1 fused with a portion of exon 2 of NRG1 comprises 2 to about 10, about 20, about 30, or up to about 40 or even all contiguous nucleic acids from SEQ ID NO: 868, including the nucleic acids at positions 75 and 76. The number of contiguous nucleic acids may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or all nucleic acids of SEQ ID NO: 868, including at least the nucleic acids at positions 75 and 76. SEQ ID NO: 868 includes the junction between DSCAML1 and NRG1, in particular the junction is between the nucleic acid at position 75, which stems from DSCAML1 and the nucleic acid at position 76 which stems from NRG1. Preferably, the polynucleotide comprising a portion of Exon 3 of DSCAML1 fused with a portion of exon 2 of NRG1 has the polynucleotide sequence of SEQ ID NO: 868, or an allelic variant thereof.

Preferably, any DSCAML1-NRG1 polynucleotide fusion provided herein is an in-frame fusion of DSCAML1 with NRG1. More preferably said fusion is an in-frame fusion comprising Exon 3 of DSCAML1, or a portion of Exon 3, with exon 2 of NRG1, or a portion of exon 2 of NRG1. Said in frame fusion is preferably the fusion of SEQ ID NO: 868, or an allelic variant thereof having at least 85% identity thereto, preferably at least 90%, 92%, 94%, 96% or even 98% identity thereto.

Preferably, the portion of Exon 3 of DSCAML1, or the allelic variant thereof, is 5′ to the portion of exon 2 of NRG1, or the allelic variant thereof. This orientation at the nucleic acid level results in a fusion polypeptide product that contains an N-terminus of DSCAML1 and a C-terminus of NRG1. Also, the herein provided DSCAML1-NRG1 polynucleotide fusion produces a protein fusion, wherein the part from the N-terminus to the fusion junction is polypeptide sequence from DSCAML1 and the part from the junction to the C-terminus is NRG1 polypeptide sequence, wherein the NRG1 part also provides its EGF-like domain. The DSCAML1-NRG1 fusion protein thereby retains the EGF-like domain of NRG1 and the ability to drive proliferation and survival of a subset of human cancers, in particular an adenocarcinoma, more in particular pancreatic ductal adenocarcinoma.

Each of the herein mentioned polynucleotide fusions comprising NRG1, including VAPB-NRG1, CADM1-NRG1, CD44-NRG1, SLC3A2-NRG1, VTCN1-NRG1, CDH1-NRG1, CXADR-NRG1, GTF2E2-NRG1, CSMD1-NRG1, PTN-NRG1, ST14-NRG1, THBS1-NRG1, AGRN-NRG1, PVALB-NRG1, APP-NRG1, WRN-NRG1, DAAM1-NRG1, ASPH-NRG1, NOTCH2-NRG1, CD74-NRG1, SDC4-NRG1, SLC4A4-NRG1, ZFAT-NRG1 or DSCAML1-NRG1, is preferably isolated. Any method of the invention preferably comprises isolating one or more polynucleotide-containing components from a sample. The one or more polynucleotide-containing components are typically isolated from any cells or cellular material in the sample.

### NRG1 Polypeptide Fusions

In accordance with the present disclosure are now provided, polypeptide fusions comprising NRG1, including VAPB-NRG1, CADM1-NRG1, CD44-NRG1, SLC3A2-NRG1, VTCN1-NRG1, CDH1-NRG1, CXADR-NRG1, GTF2E2-NRG1, CSMD1-NRG1, PTN-NRG1, ST14-NRG1, THBS1-NRG1, AGRN-NRG1, PVALB-NRG1, APP-NRG1, WRN-NRG1, ASPH-NRG1, NOTCH2-NRG1, CD74-NRG1, SDC4-NRG1, SLC4A4-NRG1, ZFAT-NRG1 and DSCAML1-NRG1. In particular, such fusions are present or have been identified in human patients diagnosed with cancer and are mentioned in more details in the following section.

### VAPB-NRG1 Polypeptide Fusions

In accordance with the present disclosure, there is provided a polypeptide fusion encoded by a polynucleotide comprising a VAPB nucleic acid sequence (or a portion of a VAPB nucleic acid sequence) fused with an NRG1 nucleic acid sequence (or a portion of a NRG1 nucleic acid sequence). The VAPB nucleic acid sequence, or portion thereof, preferably encodes a sequence comprising or consisting of any one of SEQ ID NOs 24-30, or an allelic variant of any one of these SEQ ID NOs. The NRG1 nucleic acid sequence, or portion thereof, preferably encodes a sequence comprising or consisting of any one of SEQ ID NOs 139-152, or an allelic variant of any one of these SEQ ID NOs.

The VAPB allelic variant of any one of SEQ ID NOs: 24-30, preferably has at least 85% sequence identity therewith, more preferably 90%, 92%, 94%, 96% or even more preferably at least 98% sequence identity therewith. The NRG1 allelic variant of any one of SEQ ID NOs: 139-152, preferably has at least 85% sequence identity therewith, more preferably 90%, 92%, 94%, 96% or even more preferably at least 98% sequence identity therewith.

Preferably, the portion of the VAPB nucleic acid sequence of said fusion encodes a polypeptide part of VAPB which comprises or consists of 8, 9, 10, 11, 12, 13 or 14 contiguous amino acids from any one of SEQ ID NOs 24-30, or an allelic variant of any one of SEQ ID NOs 24-30. Preferably, the portion of the NRG1 nucleic acid sequence of said fusion encodes a polypeptide part of NRG1 which comprises or consists 8, 9, 10, 11, 12, 13 or 14 contiguous amino acids from of any one of SEQ ID NOs 139-152, or an allelic variant of any one of SEQ ID NOs 139-152.

Preferably, any VAPB-NRG1 polypeptide fusion of the present disclosure includes a polypeptide sequence of any one of SEQ ID NOs: 24-30 having one or more (i.e. 1, 2, 3, 4, 5, 6, 7, 8, 9 or 10) point mutations that add, delete or substitute any of the amino acids of the polypeptides of SEQ ID NOs: 24-30. Preferably, said polypeptide fusion includes a polypeptide sequence of any one of SEQ ID NOs: 24-34 having 1, 2, 3, 4, or 5 point mutations that add, delete or substitute any of the amino acids of polypeptides of SEQ ID NO: 24-30. More preferably, said polynucleotide fusion includes a polypeptide sequence of any one of SEQ ID NOs: 24-30 having 1, 2, or 3 point mutations that add, delete or substitute any of the amino acids of the polypeptides of SEQ ID NO: 24-30.

In a preferred embodiment, there is provided a polypeptide fusion encoded by a polynucleotide which comprises a portion of exon 1 of VAPB, or an allelic variant thereof, and a portion of exon 2 of NRG1, or an allelic variant thereof. The polypeptide encoded by exon 1 of VAPB preferably comprises or consists of SEQ ID NO: 24, or an allelic variant of SEQ ID NO: 24. The polypeptide encoded by exon 2 of NRG1 preferably comprises or consists of SEQ ID NO: 140, or an allelic variant of SEQ ID NO: 140. Preferably, said polypeptide fusion further includes any one or all of SEQ ID NOs: 141-151, or any allelic variant of SEQ ID NOs: 141-151. SEQ ID NOs: 141-151 correspond to the individual polypeptide sequences encoded by exons 3-13 of NRG1, respectively. Said portion of exon 2 of NRG1 may also be according to SEQ ID NO: 154, or an allelic variant of SEQ ID NO: 154, which sequence corresponds to the polypeptide sequence encoded by all of exons 2-13.

Preferably, the polypeptide encoded by the portion of exon 1 of VAPB comprises or consists of 8, 9, 10, 11, 12, 13 or 14 contiguous amino acids from SEQ ID NO: 24, or from an allelic variant SEQ ID NO: 24. The allelic variant has at least 85% identity to SEQ ID NO: 24, preferably at least 90% identity, 92%, 94%, 96% or more preferably at least 98% sequence identity thereto. Also, the polypeptide encoded by the portion of exon 2 of NRG1 comprises or consists of 8, 9, 10, 11, 12, 13 or 14 contiguous amino acids from SEQ ID NO: 140, or from an allelic variant SEQ ID NO: 140. The allelic variant has at least 85% identity to SEQ ID NO: 140, preferably at least 90% identity, 92%, 94%, 96% or more preferably at least 98% sequence identity thereto.

Preferably, any VAPB-NRG1 polypeptide fusion of the present disclosure includes the polypeptide sequence according to SEQ ID NO: 4, or an allelic variant thereof. SEQ ID NO: 4 contains a fusion junction between VAPB and NRG1, which fusion is located between amino acid at position 14 (which, together with amino acids at positions 1-13, stems from VAPB) and the amino acid at position 16 (which, together with amino acids at positions 17-31 stems from NRG1). At position 15, an alanine (A, Ala) residue is present due to an unpredicted in frame fusion of NRG1 with VAPB. Preferably, the VAPB-NRG1 polypeptide fusion includes 8, 9, 10, 11, 12, 13 or 14 contiguous amino acids from SEQ ID NO: 4, or of an allelic variant thereof, including at least the amino acids at position 14, 15 and 16.

In a preferred embodiment, there is provided a polypeptide sequence according to SEQ ID NO: 4, or a polypeptide which comprises 8, 9, 10, 11, 12, 13, 14 or all contiguous amino acids from SEQ ID NO: 4, or of an allelic variant thereof, including at least the amino acids at position 14, 15 and 16. Said polypeptide sequence has at least 85% identity to SEQ ID NO: 4, preferably at least 90% identity, 92%, 94%, 96% or more preferably at least 98% sequence identity thereto.

Preferably, any VAPB-NRG1 polypeptide fusion of the present disclosure includes the polypeptide sequence of SEQ ID NO: 4 having one or more (i.e. 1, 2, 3, 4, 5, 6, 7, 8, 9 or 10) point mutations that add, delete or substitute any of the amino acids of the polypeptide comprising SEQ ID NO: 4. Preferably, said polypeptide fusion includes the polypeptide sequence of SEQ ID NO: 4 having 1, 2, 3, 4, or 5 point mutations that add, delete or substitute any of the amino acids of the polypeptide comprising SEQ ID NO: 4. More preferably, said polynucleotide fusion includes the polypeptide sequence of SEQ ID NO: 4 polypeptide having 1, 2 or 3 point mutations that add, delete or substitute any of the amino acids of the polypeptide comprising SEQ ID NO: 4.

Preferably, the herein provided polypeptide fusion between NRG1 and VAPB is oriented such that the part spanning the N-terminus to the fusion junction is polypeptide sequence from VAPB and the part spanning the fusion junction to the C-terminus is NRG1 polypeptide sequence. Preferably, the NRG1 polypeptide sequence comprises or encodes an EGF-like domain. Said EGF-like domain containing VAPB-NRG1 polypeptide fusion is preferably comprised by an aberrant cell as mentioned herein.

### CADM1-NRG1 Polypeptide Fusion

Also provided is a polypeptide fusion encoded by a polynucleotide which comprises a portion of exon 7 of CADM1, or an allelic variant thereof, and a portion of exon 6 of NRG1, or an allelic variant thereof. The polypeptide encoded by exon 7 of CADM1 preferably comprises or consists of SEQ ID NO: 51, or an allelic variant of SEQ ID NO: 51. The polypeptide encoded by exon 6 of NRG1 preferably comprises or consists of SEQ ID NO: 144, or an allelic variant of SEQ ID NO: 144. Preferably, said polypeptide fusion further includes any one or all of SEQ ID NOs: 45-50, or any allelic variant of any one of SEQ ID NOs: 45-50, and preferably further includes any one of SEQ ID NOs: 145-151, or any allelic variant of any one of SEQ ID NOs: 145-151. SEQ ID NOs: 45-50 correspond to the individual polypeptide sequences encoded by exons 1-7 of CADM1, respectively. SEQ ID NOs: 145-151 correspond to the individual polypeptide sequences encoded by exons 7-13 of NRG1, respectively. Said portion of exon 6 of NRG1 may also be comprised by SEQ ID NO: 156, or an allelic variant of SEQ ID NO: 156, which sequence corresponds to the polypeptide sequence encoded by all of exons 6-13.

Preferably, the polypeptide encoded by the portion of exon 7 of CADM1 comprises or consists of 8, 9, 10, 11, 12, 13 or 14 contiguous amino acids from SEQ ID NO: 51, or from an allelic variant SEQ ID NO: 51. The allelic variant has at least 85% identity to SEQ ID NO: 51, preferably at least 90% identity, 92%, 94%, 96% or more preferably at least 98% sequence identity thereto. Also, the polypeptide encoded by the portion of exon 6 of NRG1 comprises or consists of 8, 9, 10, 11, 12, 13 or 14 contiguous amino acids from SEQ ID NO: 144, or from an allelic variant SEQ ID NO: 144. The allelic variant has at least 85% identity to SEQ ID NO: 144, preferably at least 90% identity, 92%, 94%, 96% or more preferably at least 98% sequence identity thereto.

Preferably, any CADM1-NRG1 polypeptide fusion of the present disclosure includes the polypeptide sequence according to SEQ ID NO: 8, or an allelic variant thereof. SEQ ID NO: 8 contains a fusion junction between CADM1 and NRG1, which fusion is located between amino acid at position 17 (which, together with amino acids at positions 1-16, stems from CADM1) and the amino acid at position 19 (which, together with amino acids at positions 20-28 stems from NRG1). At position 18, an alanine (A, Ala) residue is present due to an unpredicted in-frame fusion of NRG1 with CADM1. Preferably, the CADM1. NRG1 polypeptide fusion includes 8, 9, 10, 11, 12, 13 or 14 contiguous amino acids from SEQ ID NO: 8, or of an allelic variant thereof, including at least the amino acids at position 17, 18 and 19 thereof.

In a preferred embodiment, there is provided a polypeptide sequence according to SEQ ID NO: 8, or a polypeptide which comprises 8, 9, 10, 11, 12, 13, 14 or all contiguous amino acids from SEQ ID NO: 8, or of an allelic variant thereof, including at least the amino acids at position 17, 18 and 19. Said polypeptide sequence has at least 85% identity to SEQ ID NO: 8, preferably at least 90% identity, 92%, 94%, 96% or more preferably at least 98% sequence identity thereto.

Preferably, any CADM1-NRG1 polypeptide fusion of the present disclosure includes the polypeptide sequence of SEQ ID NO: 8 having one or more (i.e. 1, 2, 3, 4, 5, 6, 7, 8, 9 or 10) point mutations that add, delete or substitute any of the amino acids of the polypeptide comprising SEQ ID NO: 8. Preferably, said polypeptide fusion includes the polypeptide sequence of SEQ ID NO: 8 having 1, 2, 3, 4, or 5 point mutations that add, delete or substitute any of the amino acids of the polypeptide comprising SEQ ID NO: 8. More preferably, said polynucleotide fusion includes the polypeptide sequence of SEQ ID NO: 8 polypeptide having 1, 2 or 3 point mutations that add, delete or substitute any of the amino acids of the polypeptide comprising SEQ ID NO: 8.

Preferably, the herein provided polypeptide fusion between NRG1 and CADM1 is oriented such that the part spanning the N-terminus to the fusion junction is polypeptide sequence from CADM1 and the part spanning the fusion junction to the C-terminus is NRG1 polypeptide sequence. Preferably, the NRG1 polypeptide sequence comprises or encodes an EGF-like domain. Said EGF-like domain containing CADM1-NRG1 polypeptide fusion is preferably comprised by an aberrant cell as mentioned herein.

### CD44-NRG1 Polypeptide Fusion

Also provided is a polypeptide fusion encoded by a polynucleotide which comprises a portion of exon 5 of CD44, or an allelic variant thereof, and a portion of exon 2 of NRG1, or an allelic variant thereof. The polypeptide encoded by exon 5 of CD44 preferably comprises or consists of SEQ ID NO: 84, or an allelic variant of SEQ ID NO: 84. The polypeptide encoded by exon 2 of NRG1 preferably comprises or consists of SEQ ID NO: 140, or an allelic variant of SEQ ID NO: 140. Preferably, said polypeptide fusion further includes any one or all of SEQ ID NOs: 80-83, or any allelic variant of any one of SEQ ID NOs: 80-83, and preferably further includes any one of SEQ ID NOs: 141-151, or any allelic variant of any one of SEQ ID NOs: 141-151. SEQ ID NOs: 80-83 correspond to the individual polypeptide sequences encoded by exons 1-4 of CD44, respectively. SEQ ID NOs: 141-151 correspond to the individual polypeptide sequences encoded by exons 3-13 of NRG1, respectively. Said portion of exon 2 of NRG1 may also be comprised by SEQ ID NO: 154, or an allelic variant of SEQ ID NO: 154, which sequence corresponds to the polypeptide sequence encoded by all of exons 2-13.

Preferably, the polypeptide encoded by the portion of exon 5 of CD44 comprises or consists of 8, 9, 10, 11, 12, 13 or 14 contiguous amino acids from SEQ ID NO: 84, or from an allelic variant SEQ ID NO: 84. The allelic variant has at least 85% identity to SEQ ID NO: 84, preferably at least 90% identity, 92%, 94%, 96% or more preferably at least 98% sequence identity thereto. Also, the polypeptide encoded by the portion of exon 2 of NRG1 comprises or consists of 8, 9, 10, 11, 12, 13 or 14 contiguous amino acids from SEQ ID NO: 140, or from an allelic variant SEQ ID NO: 140. The allelic variant has at least 85% identity to SEQ ID NO: 140, preferably at least 90% identity, 92%, 94%, 96% or more preferably at least 98% sequence identity thereto.

Preferably, any CD44-NRG1 polypeptide fusion of the present disclosure includes the polypeptide sequence according to SEQ ID NO: 12, or an allelic variant thereof. SEQ ID NO: 12 contains a fusion junction between CD44 and NRG1, which fusion is located between amino acid at position 17 (which, together with amino acids at positions 1-16, stems from CD44) and the amino acid at position 19 (which, together with amino acids at positions 20-36 stems from NRG1). At position 18, a threonine (T, Thr) residue is present due to an unpredicted in-frame fusion of NRG1 with CD44. Preferably, the CD44-NRG1 polypeptide fusion includes 8, 9, 10, 11, 12, 13 or 14 contiguous amino acids from SEQ ID NO: 12, or of an allelic variant thereof, including at least the amino acids at position 17, 18 and 19 thereof.

In a preferred embodiment, there is provided a polypeptide sequence according to SEQ ID NO: 12, or a polypeptide which comprises 8, 9, 10, 11, 12, 13, 14 or all contiguous amino acids from SEQ ID NO: 12, or of an allelic variant thereof, including at least the amino acids at position 17, 18 and 19. Said polypeptide sequence has at least 85% identity to SEQ ID NO: 12, preferably at least 90% identity, 92%, 94%, 96% or more preferably at least 98% sequence identity thereto.

Preferably, any CD44-NRG1 polypeptide fusion of the present disclosure includes the polypeptide sequence of SEQ ID NO: 12 having one or more (i.e. 1, 2, 3, 4, 5, 6, 7, 8, 9 or 10) point mutations that add, delete or substitute any of the amino acids of the polypeptide comprising SEQ ID NO: 12. Preferably, said polypeptide fusion includes the polypeptide sequence of SEQ ID NO: 12 having 1, 2, 3, 4, or 5 point mutations that add, delete or substitute any of the amino acids of the polypeptide comprising SEQ ID NO: 12. More preferably, said polynucleotide fusion includes the polypeptide sequence of SEQ ID NO: 12 polypeptide having 1, 2 or 3 point mutations that add, delete or substitute any of the amino acids of the polypeptide comprising SEQ ID NO: 12.

Preferably, the herein provided polypeptide fusion between NRG1 and CD44 is oriented such that the part spanning the N-terminus to the fusion junction is polypeptide sequence from CD44 and the part spanning the fusion junction to the C-terminus is NRG1 polypeptide sequence. Preferably, the NRG1 polypeptide sequence comprises or encodes an EGF-like domain. Said EGF-like domain containing CD44-NRG1 polypeptide fusion is preferably comprised by an aberrant cell as mentioned herein.

Also provided is a polypeptide fusion encoded by a polynucleotide which comprises a portion of exon 5 of CD44, or an allelic variant thereof, and a portion of exon 6 of NRG1, or an allelic variant thereof. The polypeptide encoded by exon 5 of CD44 preferably comprises or consists of SEQ ID NO: 84, or an allelic variant of SEQ ID NO: 84. The polypeptide encoded by exon 6 of NRG1 preferably comprises or consists of SEQ ID NO: 144, or an allelic variant of SEQ ID NO: 144. Preferably, said polypeptide fusion further includes any one of SEQ ID NOs: 80-83, or any allelic variant of SEQ ID NO: 80-83, and preferably further includes SEQ ID NOs: 145-151, or any allelic variant of SEQ ID NOs: 145-151. SEQ ID NOs: 80-83 correspond to the individual polypeptide sequences encoded by exons 1-4 of CD44. SEQ ID NOs: 145-151 correspond to the individual polypeptide sequences encoded by exons 7-13 of NRG1, respectively. Said portion of exon 6 of NRG1 may also be comprised by SEQ ID NO: 156, or an allelic variant of SEQ ID NO: 156, which sequence corresponds to the polypeptide sequence encoded by all of exons 6-13.

Preferably, the polypeptide encoded by the portion of exon 5 of CD44 comprises or consists of 8, 9, 10, 11, 12, 13 or 14 contiguous amino acids from SEQ ID NO: 84, or from an allelic variant SEQ ID NO: 84. The allelic variant has at least 85% identity to SEQ ID NO: 84, preferably at least 90% identity, 92%, 94%, 96% or more preferably at least 98% sequence identity thereto. Also, the polypeptide encoded by the portion of exon 6 of NRG1 comprises or consists of 8, 9, 10, 11, 12, 13 or 14 contiguous amino acids from SEQ ID NO: 144, or from an allelic variant SEQ ID NO: 144. The allelic variant has at least 85% identity to SEQ ID NO: 144, preferably at least 90% identity, 92%, 94%, 96% or more preferably at least 98% sequence identity thereto.

Preferably, any CD44-NRG1 polypeptide fusion of the present disclosure includes the polypeptide sequence according to SEQ ID NO: 762, or an allelic variant thereof. SEQ ID NO: 762 contains a fusion junction between CD44 and NRG1, which fusion is located between amino acid at position 24 (which, together with amino acids at positions 1-23, stems from CD44) and the amino acid at position 26 (which, together with amino acids at positions 27-49 stems from NRG1). At position 25, an threonine (T, Thr) residue is present due to an unpredicted in-frame fusion of NRG1 with CD44. Preferably, the CD44-NRG1 polypeptide fusion includes 8, 9, 10, 11, 12, 13 or 14 contiguous amino acids from SEQ ID NO: 762, or of an allelic variant thereof, including at least the amino acids at position 24, 25 and 26 thereof.

In a preferred embodiment, there is provided a polypeptide sequence according to SEQ ID NO: 762, or a polypeptide which comprises 8, 9, 10, 11, 12, 13, 14 or all contiguous amino acids from SEQ ID NO: 762, or of an allelic variant thereof, including at least the amino acids at position 24, 25 and 26. Said polypeptide sequence has at least 85% identity to SEQ ID NO: 762, preferably at least 90% identity, 92%, 94%, 96% or more preferably at least 98% sequence identity thereto.

Preferably, any CD44-NRG1 polypeptide fusion of the present disclosure includes the polypeptide sequence of SEQ ID NO: 762 having one or more (i.e. 1, 2, 3, 4, 5, 6, 7, 8, 9 or 10) point mutations that add, delete or substitute any of the amino acids of the polypeptide comprising SEQ ID NO: 762. Preferably, said polypeptide fusion includes the polypeptide sequence of SEQ ID NO: 762 having 1, 2, 3, 4, or 5 point mutations that add, delete or substitute any of the amino acids of the polypeptide comprising SEQ ID NO: 762. More preferably, said polynucleotide fusion includes the polypeptide sequence of SEQ ID NO: 762 having 1, 2 or 3 point mutations that add, delete or substitute any of the amino acids of the polypeptide comprising SEQ ID NO: 762.

Preferably, the herein provided polypeptide fusion between CD44 and NRG1 is oriented such that the part spanning the N-terminus to the fusion junction is polypeptide sequence from CD44 and the part spanning the fusion junction to the C-terminus is NRG1 polypeptide sequence. Preferably, the NRG1 polypeptide sequence comprises or encodes an EGF-like domain. Said EGF-like domain containing CD44-NRG1 polypeptide fusion is preferably comprised by an aberrant cell as mentioned herein.

### SLC3A2-NRG1 Polypeptide Fusion

Also provided is a polypeptide fusion encoded by a polynucleotide which comprises a portion of exon 1 of transcript version 6 of SLC3A2, or an allelic variant thereof, and a portion of exon 5 of NRG1, or an allelic variant thereof. The polypeptide encoded by exon 1 of said SLC3A2 preferably comprises or consists of SEQ ID NO: 113, or an allelic variant of SEQ ID NO: 113. The polypeptide encoded by exon 5 of NRG1 preferably comprises or consists of SEQ ID NO: 143, or an allelic variant of SEQ ID NO: 143. Preferably, said polypeptide fusion further includes any one of SEQ ID NOs: 144-151, or any allelic variant of any one of SEQ ID NOs: 144-151. SEQ ID NOs: 144-151 correspond to the individual polypeptide sequences encoded by exons 6-13 of NRG1, respectively. Said portion of exon 5 of NRG1 may also be comprised by SEQ ID NO: 158, or an allelic variant of SEQ ID NO: 158, which sequence corresponds to the polypeptide sequence encoded by all of exons 6-13 and includes an additional serine residue encoded by the most 3′ in frame triplet of exon 5.

Preferably, the polypeptide encoded by the portion of exon 1 of transcript version 6 of SLC3A2 comprises or consists of 8, 9, 10, 11, 12, 13 or 14 contiguous amino acids from SEQ ID NO: 113, or from an allelic variant SEQ ID NO: 113. The allelic variant has at least 85% identity to SEQ ID NO: 113, preferably at least 90% identity, 92%, 94%, 96% or more preferably at least 98% sequence identity thereto. In a preferred embodiment, the portion of exon 5 of NRG1 in the SLC3A2-NRG1 fusion comprises at least the amino acid at position 16 of SEQ ID NO: 143, or the corresponding amino acid of an allelic variant of SEQ ID NO: 143. The allelic variant has at least 85% identity to SEQ ID NO: 143, preferably at least 90% identity, 92%, 94%, 96% or more preferably at least 98% sequence identity thereto.

Preferably, any transcript version 6 of SLC3A2-NRG1 polypeptide fusion of the present disclosure includes the polypeptide sequence according to SEQ ID NO: 16, or an allelic variant thereof. SEQ ID NO: 16 contains a fusion junction between SLC3A2 and NRG1, which fusion is located between amino acid at position 17 (which, together with amino acids at positions 1-16, stems from SLC3A2) and the amino acid at position 19 (which, together with amino acids at positions 20-29 stems from NRG1). At position 18, an alanine (A, Ale) residue is present due to an unpredicted in-frame fusion of NRG1 with said SLC3A2 version. Preferably, the SLC3A2-NRG1 polypeptide fusion includes 8, 9, 10, 11, 12, 13 or 14 contiguous amino acids from SEQ ID NO: 16, or of an allelic variant thereof, including at least the amino acids at position 17, 18 and 19 of SEQ ID NO: 16.

In a preferred embodiment, there is provided a polypeptide sequence according to SEQ ID NO: 16, or a polypeptide which comprises 8, 9, 10, 11, 12, 13, 14 or all contiguous amino acids from SEQ ID NO: 16, or of an allelic variant thereof, including at least the amino acids at position 17, 18 and 19. Said polypeptide sequence has at least 85% identity to SEQ ID NO: 16, preferably at least 90% identity, 92%, 94%, 96% or more preferably at least 98% sequence identity thereto.

Preferably, any transcript version 6 of SLC3A2-NRG1 polypeptide fusion of the present disclosure includes the polypeptide sequence of SEQ ID NO: 16 having one or more (i.e. 1, 2, 3, 4, 5, 6, 7, 8, 9 or 10) point mutations that add, delete or substitute any of the amino acids of the polypeptide comprising SEQ ID NO: 16. Preferably, said polypeptide fusion includes the polypeptide sequence of SEQ ID NO: 16 having 1, 2, 3, 4, or 5 point mutations that add, delete or substitute any of the amino acids of the polypeptide comprising SEQ ID NO: 16. More preferably, said polynucleotide fusion includes the polypeptide sequence of SEQ ID NO: 16 polypeptide having 1, 2 or 3 point mutations that add, delete or substitute any of the amino acids of the polypeptide comprising SEQ ID NO: 16.

Preferably, the herein provided polypeptide fusion between NRG1 and transcript version 6 of SLC3A2 is oriented such that the part spanning the N-terminus to the fusion junction is polypeptide sequence from SLC3A2 and the part spanning the fusion junction to the C-terminus is NRG1 polypeptide sequence. Preferably, the NRG1 polypeptide sequence comprises or encodes an EGF-like domain. Said EGF-like domain containing SLC3A2-NRG1 polypeptide fusion is preferably comprised by an aberrant cell as mentioned herein.

Also provided is a polypeptide fusion encoded by a polynucleotide which comprises a portion of exon 2 of transcript version 3 of SLC3A2, or an allelic variant thereof, and a portion of exon 6 of NRG1, or an allelic variant thereof. The polypeptide encoded by exon 2 of said SLC3A2 preferably comprises or consists of SEQ ID NO: 470, or an allelic variant of SEQ ID NO: 470. The polypeptide encoded by exon 6 of NRG1 preferably comprises or consists of SEQ ID NO: 144, or an allelic variant of SEQ ID NO: 144. Preferably, said polypeptide fusion further includes any one of SEQ ID NOs: 469 or 145-151, or any allelic variant of SEQ ID NOs: 469 or 145-151. SEQ ID NOs: 145-151 correspond to the individual polypeptide sequences encoded by exons 7-13 of NRG1, respectively. Said portion of exon 6 of NRG1 may also be comprised by SEQ ID NO: 156, or an allelic variant of SEQ ID NO: 156, which sequence corresponds to the polypeptide sequence encoded by all of exons 6-13.

Preferably, the polypeptide encoded by the portion of exon 2 of transcript version 3 of SLC3A2 comprises or consists of 8, 9, 10, 11, 12, 13 or 14 contiguous amino acids from SEQ ID NO: 470, or from an allelic variant SEQ ID NO: 470. The allelic variant has at least 85% identity to SEQ ID NO: 470, preferably at least 90% identity, 92%, 94%, 96% or more preferably at least 98% sequence identity thereto. In a preferred embodiment, the portion of exon 6 of NRG1 in the SLC3A2-NRG1 fusion comprises or is the sequence according to SEQ ID NO: 144, or allelic variant of SEQ ID NO: 144. The allelic variant has at least 85% identity to SEQ ID NO: 144, preferably at least 90% identity, 92%, 94%, 96% or more preferably at least 98% sequence identity thereto.

Preferably, any transcript version 3 of SLC3A2-NRG1 polypeptide fusion of the present disclosure includes the polypeptide sequence according to SEQ ID NO: 455, or an allelic variant thereof. SEQ ID NO: 455 contains a fusion junction between SLC3A2 and NRG1, which fusion is located between amino acid at position 30 (which, together with amino acids at positions 1-29, stems from said SLC3A2 version) and the amino acid at position 32 (which, together with amino acids at positions 33-39 stems from NRG1). At position 31, an alanine (A, ala) residue is present due to an unpredicted in frame fusion of NRG1 with said SLC3A2 version. Preferably, the SLC3A2-NRG1 polypeptide fusion includes 8, 9, 10, 11, 12, 13 or 14 contiguous amino acids from SEQ ID NO: 455, or of an allelic variant thereof, including at least the amino acids at position 30, 31 and 32 of SEQ ID NO: 455.

In a preferred embodiment, there is provided a polypeptide sequence according to SEQ ID NO: 455, or a polypeptide which comprises 8, 9, 10, 11, 12, 13, 14 or all contiguous amino acids from SEQ ID NO: 455, or of an allelic variant thereof, including at least the amino acids at position 30, 31 and 32. Said polypeptide sequence has at least 85% identity to SEQ ID NO: 455, preferably at least 90% identity, 92%, 94%, 96% or more preferably at least 98% sequence identity thereto.

Preferably, any transcript version 3 of SLC3A2-NRG1 polypeptide fusion of the present disclosure includes the polypeptide sequence of SEQ ID NO: 455 having one or more (i.e. 1, 2, 3, 4, 5, 6, 7, 8, 9 or 10) point mutations that add, delete or substitute any of the amino acids of the polypeptide comprising SEQ ID NO: 455. Preferably, said polypeptide fusion includes the polypeptide sequence of SEQ ID NO: 455 having 1, 2, 3, 4, or 5 point mutations that add, delete or substitute any of the amino acids of the polypeptide comprising SEQ ID NO: 455. More preferably, said polynucleotide fusion includes the polypeptide sequence of SEQ ID NO: 455 polypeptide having 1, 2 or 3 point mutations that add, delete or substitute any of the amino acids of the polypeptide comprising SEQ ID NO: 455.

Preferably, the herein provided polypeptide fusion between NRG1 and transcript version 6 of SLC3A2 is oriented such that the part spanning the N-terminus to the fusion junction is polypeptide sequence from SLC3A2 and the part spanning the fusion junction to the C-terminus is NRG1 polypeptide sequence. Preferably, the NRG1 polypeptide sequence comprises or encodes an EGF-like domain. Said EGF-like domain containing SLC3A2-NRG1 polypeptide fusion is preferably comprised by an aberrant cell as mentioned herein.

### VTCN1-NRG1 Polypeptide Fusion

Also provided is a polypeptide fusion encoded by a polynucleotide which comprises a portion of exon 2 of VTCN1, or an allelic variant thereof, and a portion of exon 2 of NRG1, or an allelic variant thereof. The polypeptide encoded by exon 2 of VTCN1 preferably comprises or consists of SEQ ID NO: 176, or an allelic variant of SEQ ID NO: 176. The polypeptide encoded by exon 2 of NRG1 preferably comprises or consists of SEQ ID NO: 140, or an allelic variant of SEQ ID NO: 140. Preferably, said polypeptide fusion further includes SEQ ID NOs: 175, or any allelic variant of SEQ ID NO: 175, and preferably further includes any one of SEQ ID NOs: 141-151, or any allelic variant of any one of SEQ ID NOs: 141-151. SEQ ID NO: 175 correspond to the polypeptide sequence encoded by exon 1 of VTCN1. SEQ ID NOs: 141-151 correspond to the individual polypeptide sequences encoded by exons 3-13 of NRG1, respectively. Said portion of exon 2 of NRG1 may also be comprised by SEQ ID NO: 154, or an allelic variant of SEQ ID NO: 154, which sequence corresponds to the polypeptide sequence encoded by all of exons 2-13

Preferably, the polypeptide encoded by the portion of exon 2 of VTCN1 comprises or consists of 8, 9, 10, 11, 12, 13 or 14 contiguous amino acids from SEQ ID NO: 176, or from an allelic variant SEQ ID NO: 176. The allelic variant has at least 85% identity to SEQ ID NO: 176, preferably at least 90% identity, 92%, 94%, 96% or more preferably at least 98% sequence identity thereto. Also, the polypeptide encoded by the portion of exon 2 of NRG1 comprises or consists of 8, 9, 10, 11, 12, 13 or 14 contiguous amino acids from SEQ ID NO: 140, or from an allelic variant SEQ ID NO: 140. The allelic variant has at least 85% identity to SEQ ID NO: 140, preferably at least 90% identity, 92%, 94%, 96% or more preferably at least 98% sequence identity thereto.

Preferably, any VTCN1-NRG1 polypeptide fusion of the present disclosure includes the polypeptide sequence according to SEQ ID NO: 167, or an allelic variant thereof. SEQ ID NO: 167 contains a fusion junction between VTCN1 and NRG1, which fusion is located between amino acid at position 21 (which, together with amino acids at positions 1-20, stems from VTCN1) and the amino acid at position 23 (which, together with amino acids at positions 24-30 stems from NRG1). At position 22, an alanine (A, ala) residue is present due to an unpredicted in-frame fusion of NRG1 with VTCN1. Preferably, the VTCN1-NRG1 polypeptide fusion includes 8, 9, 10, 11, 12, 13 or 14 contiguous amino acids from SEQ ID NO: 167, or of an allelic variant thereof, including at least the amino acids at position 21, 22 and 23 thereof.

In a preferred embodiment, there is provided a polypeptide sequence according to SEQ ID NO: 167, or a polypeptide which comprises 8, 9, 10, 11, 12, 13, 14 or all contiguous amino acids from SEQ ID NO: 167, or of an allelic variant thereof, including at least the amino acids at position 21, 22 and 23. Said polypeptide sequence has at least 85% identity to SEQ ID NO: 167, preferably at least 90% identity, 92%, 94%, 96% or more preferably at least 98% sequence identity thereto.

Preferably, any VTCN1-NRG1 polypeptide fusion of the present disclosure includes the polypeptide sequence of SEQ ID NO: 167 having one or more (i.e. 1, 2, 3, 4, 5, 6, 7, 8, 9 or 10) point mutations that add, delete or substitute any of the amino acids of the polypeptide comprising SEQ ID NO: 167. Preferably, said polypeptide fusion includes the polypeptide sequence of SEQ ID NO: 167 having 1, 2, 3, 4, or 5 point mutations that add, delete or substitute any of the amino acids of the polypeptide comprising SEQ ID NO: 167. More preferably, said polynucleotide fusion includes the polypeptide sequence of SEQ ID NO: 167 having 1, 2 or 3 point mutations that add, delete or substitute any of the amino acids of the polypeptide comprising SEQ ID NO: 167.

Preferably, the herein provided polypeptide fusion between VTCN1 and NRG1 is oriented such that the part spanning the N-terminus to the fusion junction is polypeptide sequence from VTCN1 and the part spanning the fusion junction to the C-terminus is NRG1 polypeptide sequence. Preferably, the NRG1 polypeptide sequence comprises or encodes an EGF-like domain. Said EGF-like domain containing VTCN1-NRG1 polypeptide fusion is preferably comprised by an aberrant cell as mentioned herein.

### CDH1-NRG1 Polypeptide Fusion

Also provided is a polypeptide fusion encoded by a polynucleotide which comprises a portion of exon 11 of CDH1, or an allelic variant thereof, and a portion of exon 2 of NRG1, or an allelic variant thereof. The polypeptide encoded by exon 11 of CDH1 preferably comprises or consists of SEQ ID NO: 206, or an allelic variant of SEQ ID NO: 206. The polypeptide encoded by exon 2 of NRG1 preferably comprises or consists of SEQ ID NO: 140, or an allelic variant of SEQ ID NO: 140. Preferably, said polypeptide fusion further includes SEQ ID NO: 205, or any allelic variant of SEQ ID NO: 205, and preferably further includes any one of SEQ ID NOs: 141-151, or any allelic variant of any one of SEQ ID NOs: 141-151. SEQ ID NO: 205 correspond to the polypeptide sequence encoded by exon 10 of CDH1. SEQ ID NOs: 141-151 correspond to the individual polypeptide sequences encoded by exons 3-13 of NRG1, respectively. Said portion of exon 2 of NRG1 may also be comprised by SEQ ID NO: 154, or an allelic variant of SEQ ID NO: 154, which sequence corresponds to the polypeptide sequence encoded by all of exons 2-13

Preferably, the polypeptide encoded by the portion of exon 11 of CDH1 comprises or consists of 8, 9, 10, 11, 12, 13 or 14 contiguous amino acids from SEQ ID NO: 206, or from an allelic variant SEQ ID NO: 206. The allelic variant has at least 85% identity to SEQ ID NO: 206, preferably at least 90% identity, 92%, 94%, 96% or more preferably at least 98% sequence identity thereto. Also, the polypeptide encoded by the portion of exon 2 of NRG1 comprises or consists of 8, 9, 10, 11, 12, 13 or 14 contiguous amino acids from SEQ ID NO: 140, or from an allelic variant SEQ ID NO: 140. The allelic variant has at least 85% identity to SEQ ID NO: 140, preferably at least 90% identity, 92%, 94%, 96% or more preferably at least 98% sequence identity thereto.

Preferably, any CDH1-NRG1 polypeptide fusion of the present disclosure includes the polypeptide sequence according to SEQ ID NO: 187, or an allelic variant thereof. SEQ ID NO: 187 contains a fusion junction between CDH1 and NRG1, which fusion is located between amino acid at position 39 (which, together with amino acids at positions 1-38, stems from CDH1) and the amino acid at position 41 (which, together with amino acids at positions 42-49 stems from NRG1). At position 40, an alanine (A, ala) residue is present due to an unpredicted in-frame fusion of NRG1 with CDH1. Preferably, the CDH1-NRG1 polypeptide fusion includes 8, 9, 10, 11, 12, 13 or 14 contiguous amino acids from SEQ ID NO: 187, or of an allelic variant thereof, including at least the amino acids at position 39, 40 and 41 thereof.

In a preferred embodiment, there is provided a polypeptide sequence according to SEQ ID NO: 187, or a polypeptide which comprises 8, 9, 10, 11, 12, 13, 14 or all contiguous amino acids from SEQ ID NO: 187, or of an allelic variant thereof, including at least the amino acids at position 39, 40 and 41. Said polypeptide sequence has at least 85% identity to SEQ ID NO: 187, preferably at least 90% identity, 92%, 94%, 96% or more preferably at least 98% sequence identity thereto.

Preferably, any CDH1-NRG1 polypeptide fusion of the present disclosure includes the polypeptide sequence of SEQ ID NO: 187 having one or more (i.e. 1, 2, 3, 4, 5, 6, 7, 8, 9 or 10) point mutations that add, delete or substitute any of the amino acids of the polypeptide comprising SEQ ID NO: 187. Preferably, said polypeptide fusion includes the polypeptide sequence of SEQ ID NO: 187 having 1, 2, 3, 4, or 5 point mutations that add, delete or substitute any of the amino acids of the polypeptide comprising SEQ ID NO: 187. More preferably, said polynucleotide fusion includes the polypeptide sequence of SEQ ID NO: 187 having 1, 2 or 3 point mutations that add, delete or substitute any of the amino acids of the polypeptide comprising SEQ ID NO: 187.

Preferably, the herein provided polypeptide fusion between CDH1 and NRG1 is oriented such that the part spanning the N-terminus to the fusion junction is polypeptide sequence from CDH1 and the part spanning the fusion junction to the C-terminus is NRG1 polypeptide sequence. Preferably, the NRG1 polypeptide sequence comprises or encodes an EGF-like domain. Said EGF-like domain containing CDH1-NRG1 polypeptide fusion is preferably comprised by an aberrant cell as mentioned herein.

### CXADR-NRG1 Polypeptide Fusion

Also provided is a polypeptide fusion encoded by a polynucleotide which comprises a portion of exon 1 of CXADR, or an allelic variant thereof, and a portion of exon 2 of NRG1, or an allelic variant thereof. The polypeptide encoded by exon 1 of CXADR preferably comprises or consists of SEQ ID NO: 225, or an allelic variant of SEQ ID NO: 225. The polypeptide encoded by exon 2 of NRG1 preferably comprises or consists of SEQ ID NO: 140, or an allelic variant of SEQ ID NO: 140. Preferably, said polypeptide fusion further includes SEQ ID NOs: 141-151, or any allelic variant of SEQ ID NOs: 141-151. SEQ ID NO: 225 correspond to the polypeptide sequence encoded by exon 1 of CXADR. SEQ ID NOs: 141-151 correspond to the individual polypeptide sequences encoded by exons 3-13 of NRG1, respectively. Said portion of exon 2 of NRG1 may also be comprised by SEQ ID NO: 154, or an allelic variant of SEQ ID NO: 154, which sequence corresponds to the polypeptide sequence encoded by all of exons 2-13.

Preferably, the polypeptide encoded by the portion of exon 1 of CXADR comprises or consists of 8, 9, 10, 11, 12, 13 or 14 contiguous amino acids from SEQ ID NO: 225, or from an allelic variant SEQ ID NO: 225. The allelic variant has at least 85% identity to SEQ ID NO: 225, preferably at least 90% identity, 92%, 94%, 96% or more preferably at least 98% sequence identity thereto. Also, the polypeptide encoded by the portion of exon 2 of NRG1 comprises or consists of 8, 9, 10, 11, 12, 13 or 14 contiguous amino acids from SEQ ID NO: 140, or from an allelic variant SEQ ID NO: 140. The allelic variant has at least 85% identity to SEQ ID NO: 140, preferably at least 90% identity, 92%, 94%, 96% or more preferably at least 98% sequence identity thereto.

Preferably, any CXADR-NRG1 polypeptide fusion of the present disclosure includes the polypeptide sequence according to SEQ ID NO: 218, or an allelic variant thereof. SEQ ID NO: 218 contains a fusion junction between CXADR and NRG1, which fusion is located between amino acid at position 14 (which, together with amino acids at positions 1-13, stems from CXADR) and the amino acid at position 16 (which, together with amino acids at positions 17-33 stems from NRG1). At position 15, an alanine (A, ala) residue is present due to an unpredicted in-frame fusion of NRG1 with CXADR. Preferably, the CXADR-NRG1 polypeptide fusion includes 8, 9, 10, 11, 12, 13 or 14 contiguous amino acids from SEQ ID NO: 218, or of an allelic variant thereof, including at least the amino acids at position 14, 15 and 16 thereof.

In a preferred embodiment, there is provided a polypeptide sequence according to SEQ ID NO: 218, or a polypeptide which comprises 8, 9, 10, 11, 12, 13, 14 or all contiguous amino acids from SEQ ID NO: 218, or of an allelic variant thereof, including at least the amino acids at position 14, 15 and 16. Said polypeptide sequence has at least 85% identity to SEQ ID NO: 218, preferably at least 90% identity, 92%, 94%, 96% or more preferably at least 98% sequence identity thereto.

Preferably, any CXADR-NRG1 polypeptide fusion of the present disclosure includes the polypeptide sequence of SEQ ID NO: 218 having one or more (i.e. 1, 2, 3, 4, 5, 6, 7, 8, 9 or 10) point mutations that add, delete or substitute any of the amino acids of the polypeptide comprising SEQ ID NO: 218. Preferably, said polypeptide fusion includes the polypeptide sequence of SEQ ID NO: 218 having 1, 2, 3, 4, or 5 point mutations that add, delete or substitute any of the amino acids of the polypeptide comprising SEQ ID NO: 218. More preferably, said polynucleotide fusion includes the polypeptide sequence of SEQ ID NO: 218 having 1, 2 or 3 point mutations that add, delete or substitute any of the amino acids of the polypeptide comprising SEQ ID NO: 218.

Preferably, the herein provided polypeptide fusion between CXADR and NRG1 is oriented such that the part spanning the N-terminus to the fusion junction is polypeptide sequence from CXADR and the part spanning the fusion junction to the C-terminus is NRG1 polypeptide sequence. Preferably, the NRG1 polypeptide sequence comprises or encodes an EGF-like domain. Said EGF-like domain containing CXADR-NRG1 polypeptide fusion is preferably comprised by an aberrant cell as mentioned herein.

### GTF2E2-NRG1 Polypeptide Fusion

Also provided is a polypeptide fusion encoded by a polynucleotide which comprises a portion of exon 2 of GTF2E2, or an allelic variant thereof, and a portion of exon 2 of NRG1, or an allelic variant thereof. The polypeptide encoded by exon 2 of GTF2E2 preferably comprises or consists of SEQ ID NO: 244, or an allelic variant of SEQ ID NO: 244. The polypeptide encoded by exon 2 of NRG1 preferably comprises or consists of SEQ ID NO: 140, or an allelic variant of SEQ ID NO: 140. Preferably, said polypeptide fusion further includes SEQ ID NOs: 141-151, or any allelic variant of SEQ ID NOs: 141-151. SEQ ID NOs: 141-151 correspond to the individual polypeptide sequences encoded by exons 3-13 of NRG1, respectively. Said portion of exon 2 of NRG1 may also be comprised by SEQ ID NO: 154, or an allelic variant of SEQ ID NO: 154, which sequence corresponds to the polypeptide sequence encoded by all of exons 2-13

Preferably, the polypeptide encoded by the portion of exon 2 of GTF2E2 comprises or consists of 8, 9, 10, 11, 12, 13 or 14 contiguous amino acids from SEQ ID NO: 244, or from an allelic variant SEQ ID NO: 244. The allelic variant has at least 85% identity to SEQ ID NO: 244, preferably at least 90% identity, 92%, 94%, 96% or more preferably at least 98% sequence identity thereto. Also, the polypeptide encoded by the portion of exon 2 of NRG1 comprises or consists of 8, 9, 10, 11, 12, 13 or 14 contiguous amino acids from SEQ ID NO: 140, or from an allelic variant SEQ ID NO: 140. The allelic variant has at least 85% identity to SEQ ID NO: 140, preferably at least 90% identity, 92%, 94%, 96% or more preferably at least 98% sequence identity thereto.

Preferably, any GTF2E2-NRG1 polypeptide fusion of the present disclosure includes the polypeptide sequence according to SEQ ID NO: 234, or an allelic variant thereof. SEQ ID NO: 234 contains a fusion junction between GTF2E2 and NRG1, which fusion is located between amino acid at position 46 (which, together with amino acids at positions 1-45, stems from GTF2E2) and the amino acid at position 48 (which, together with amino acids at positions 49-88 stems from NRG1). At position 47, an alanine (A, ala) residue is present due to an unpredicted in-frame fusion of NRG1 with GTF2E2. Preferably, the GTF2E2-NRG1 polypeptide fusion includes 8, 9, 10, 11, 12, 13 or 14 contiguous amino acids from SEQ ID NO: 234, or of an allelic variant thereof, including at least the amino acids at position 46, 47 and 48 thereof.

In a preferred embodiment, there is provided a polypeptide sequence according to SEQ ID NO: 234, or a polypeptide which comprises 8, 9, 10, 11, 12, 13, 14 or all contiguous amino acids from SEQ ID NO: 234, or of an allelic variant thereof, including at least the amino acids at position 46, 47 and 48. Said polypeptide sequence has at least 85% identity to SEQ ID NO: 234, preferably at least 90% identity, 92%, 94%, 96% or more preferably at least 98% sequence identity thereto.

Preferably, any GTF2E2-NRG1 polypeptide fusion of the present disclosure includes the polypeptide sequence of SEQ ID NO: 234 having one or more (i.e. 1, 2, 3, 4, 5, 6, 7, 8, 9 or 10) point mutations that add, delete or substitute any of the amino acids of the polypeptide comprising SEQ ID NO: 234. Preferably, said polypeptide fusion includes the polypeptide sequence of SEQ ID NO: 234 having 1, 2, 3, 4, or 5 point mutations that add, delete or substitute any of the amino acids of the polypeptide comprising SEQ ID NO: 234. More preferably, said polynucleotide fusion includes the polypeptide sequence of SEQ ID NO: 234 having 1, 2 or 3 point mutations that add, delete or substitute any of the amino acids of the polypeptide comprising SEQ ID NO: 234.

Preferably, the herein provided polypeptide fusion between GTF2E2 and NRG1 is oriented such that the part spanning the N-terminus to the fusion junction is polypeptide sequence from GTF2E2 and the part spanning the fusion junction to the C-terminus is NRG1 polypeptide sequence. Preferably, the NRG1 polypeptide sequence comprises or encodes an EGF-like domain. Said EGF-like domain containing GTF2E2-NRG1 polypeptide fusion is preferably comprised by an aberrant cell as mentioned herein.

### CSMD1-NRG1 Polypeptide Fusion

Also provided is a polypeptide fusion encoded by a polynucleotide which comprises a portion of exon 23 of CSMD1, or an allelic variant thereof, and a portion of exon 6 of NRG1, or an allelic variant thereof. The polypeptide encoded by exon 23 of CSMD1 preferably comprises or consists of SEQ ID NO: 305, or an allelic variant of SEQ ID NO: 305. The polypeptide encoded by exon 6 of NRG1 preferably comprises or consists of SEQ ID NO: 144, or an allelic variant of SEQ ID NO: 144. Preferably, said polypeptide fusion further includes any one of SEQ ID NOs: 283-304, or any allelic variant of SEQ ID NOs: 283-304, and preferably further includes SEQ ID NOs: 145-151, or any allelic variant of SEQ ID NOs: 145-151. SEQ ID NOs: 283-304 correspond to the individual polypeptide sequences encoded by exons 1-22 of CSMD1, respectively. SEQ ID NOs: 145-151 correspond to the individual polypeptide sequences encoded by exons 7-13 of NRG1, respectively. Said portion of exon 6 of NRG1 may also be comprised by SEQ ID NO: 156, or an allelic variant of SEQ ID NO: 156, which sequence corresponds to the polypeptide sequence encoded by all of exons 6-13.

Preferably, the polypeptide encoded by the portion of exon 23 of CSMD1 comprises or consists of 8, 9, 10, 11, 12, 13 or 14 contiguous amino acids from SEQ ID NO: 305, or from an allelic variant SEQ ID NO: 305. The allelic variant has at least 85% identity to SEQ ID NO: 305, preferably at least 90% identity, 92%, 94%, 96% or more preferably at least 98% sequence identity thereto. Also, the polypeptide encoded by the portion of exon 6 of NRG1 comprises or consists of 8, 9, 10, 11, 12, 13 or 14 contiguous amino acids from SEQ ID NO: 144, or from an allelic variant SEQ ID NO: 144. The allelic variant has at least 85% identity to SEQ ID NO: 144, preferably at least 90% identity, 92%, 94%, 96% or more preferably at least 98% sequence identity thereto.

Preferably, any CSMD1-NRG1 polypeptide fusion of the present disclosure includes the polypeptide sequence according to SEQ ID NO: 256, or an allelic variant thereof. SEQ ID NO: 256 contains a fusion junction between CSMD1 and NRG1, which fusion is located between amino acid at position 29 (which, together with amino acids at positions 1-28, stems from CSMD1) and the amino acid at position 31 (which, together with amino acids at positions 32-50 stems from NRG1). At position 30, a Threonine (T, thr) residue is present due to an unpredicted in-frame fusion of NRG1 with CSMD1. Preferably, the CSMD1-NRG1 polypeptide fusion includes 8, 9, 10, 11, 12, 13 or 14 contiguous amino acids from SEQ ID NO: 256, or of an allelic variant thereof, including at least the amino acids at position 29, 30 and 31 thereof.

In a preferred embodiment, there is provided a polypeptide sequence according to SEQ ID NO: 256, or a polypeptide which comprises 8, 9, 10, 11, 12, 13, 14 or all contiguous amino acids from SEQ ID NO: 256, or of an allelic variant thereof, including at least the amino acids at position 29, 30 and 31. Said polypeptide sequence has at least 85% identity to SEQ ID NO: 256, preferably at least 90% identity, 92%, 94%, 96% or more preferably at least 98% sequence identity thereto.

Preferably, any CSMD1-NRG1 polypeptide fusion of the present disclosure includes the polypeptide sequence of SEQ ID NO: 256 having one or more (i.e. 1, 2, 3, 4, 5, 6, 7, 8, 9 or 10) point mutations that add, delete or substitute any of the amino acids of the polypeptide comprising SEQ ID NO: 256. Preferably, said polypeptide fusion includes the polypeptide sequence of SEQ ID NO: 256 having 1, 2, 3, 4, or 5 point mutations that add, delete or substitute any of the amino acids of the polypeptide comprising SEQ ID NO: 256. More preferably, said polynucleotide fusion includes the polypeptide sequence of SEQ ID NO: 256 having 1, 2 or 3 point mutations that add, delete or substitute any of the amino acids of the polypeptide comprising SEQ ID NO: 256.

Preferably, the herein provided polypeptide fusion between CSMD1 and NRG1 is oriented such that the part spanning the N-terminus to the fusion junction is polypeptide sequence from CSMD1 and the part spanning the fusion junction to the C-terminus is NRG1 polypeptide sequence. Preferably, the NRG1 polypeptide sequence comprises or encodes an EGF-like domain. Said EGF-like domain containing CSMD1-NRG1 polypeptide fusion is preferably comprised by an aberrant cell as mentioned herein.

### PTN-NRG1 Polypeptide Fusion

Also provided is a polypeptide fusion encoded by a polynucleotide which comprises a portion of exon 4 of PTN, or an allelic variant thereof, and a portion of exon 2 of NRG1, or an allelic variant thereof. The polypeptide encoded by exon 4 of PTN preferably comprises or consists of SEQ ID NO: 323, or an allelic variant of SEQ ID NO: 323. The polypeptide encoded by exon 2 of NRG1 preferably comprises or consists of SEQ ID NO: 140, or an allelic variant of SEQ ID NO: 140. Preferably, said polypeptide fusion further includes SEQ ID NO: 321 and/or 322, or any allelic variant of SEQ ID NO: 321 and/or 322, and preferably further includes SEQ ID NOs: 141-151, or any allelic variant of SEQ ID NOs: 141-151. SEQ ID NO: 321, 322 and 323 correspond to the individual polypeptide sequences encoded by exons 2,3,4 of PTN, respectively. SEQ ID NOs: 141-151 correspond to the individual polypeptide sequences encoded by exons 3-13 of NRG1, respectively. Said portion of exon 2 of NRG1 may also be comprised by SEQ ID NO: 154, or an allelic variant of SEQ ID NO: 154, which sequence corresponds to the polypeptide sequence encoded by all of exons 2-13.

Preferably, the polypeptide encoded by the portion of exon 4 of PTN comprises or consists of 8, 9, 10, 11, 12, 13 or 14 contiguous amino acids from SEQ ID NO: 323, or from an allelic variant SEQ ID NO: 323. The allelic variant has at least 85% identity to SEQ ID NO: 323, preferably at least 90% identity, 92%, 94%, 96% or more preferably at least 98% sequence identity thereto. Also, the polypeptide encoded by the portion of exon 2 of NRG1 comprises or consists of 8, 9, 10, 11, 12, 13 or 14 contiguous amino acids from SEQ ID NO: 140, or from an allelic variant SEQ ID NO: 140. The allelic variant has at least 85% identity to SEQ ID NO: 140, preferably at least 90% identity, 92%, 94%, 96% or more preferably at least 98% sequence identity thereto.

Preferably, any PTN-NRG1 polypeptide fusion of the present disclosure includes the polypeptide sequence according to SEQ ID NO: 314, or an allelic variant thereof. SEQ ID NO: 314 contains a fusion junction between PTN and NRG1, which fusion is located between amino acid at position 33 (which, together with amino acids at positions 1-32, stems from PTN) and the amino acid at position 35 (which, together with amino acids at positions 36-67 stems from NRG1). At position 34, an alanine (A, ala) residue is present due to an unpredicted in-frame fusion of NRG1 with PTN. Preferably, the PTN-NRG1 polypeptide fusion includes 8, 9, 10, 11, 12, 13 or 14 contiguous amino acids from SEQ ID NO: 314, or of an allelic variant thereof, including at least the amino acids at position 33, 34 and 35 thereof.

In a preferred embodiment, there is provided a polypeptide sequence according to SEQ ID NO: 314, or a polypeptide which comprises 8, 9, 10, 11, 12, 13, 14 or all contiguous amino acids from SEQ ID NO: 314, or of an allelic variant thereof, including at least the amino acids at position 33, 34 and 35. Said polypeptide sequence has at least 85% identity to SEQ ID NO: 314, preferably at least 90% identity, 92%, 94%, 96% or more preferably at least 98% sequence identity thereto.

Preferably, any PTN-NRG1 polypeptide fusion of the present disclosure includes the polypeptide sequence of SEQ ID NO: 314 having one or more (i.e. 1, 2, 3, 4, 5, 6, 7, 8, 9 or 10) point mutations that add, delete or substitute any of the amino acids of the polypeptide comprising SEQ ID NO: 314. Preferably, said polypeptide fusion includes the polypeptide sequence of SEQ ID NO: 314 having 1, 2, 3, 4, or 5 point mutations that add, delete or substitute any of the amino acids of the polypeptide comprising SEQ ID NO: 314. More preferably, said polynucleotide fusion includes the polypeptide sequence of SEQ ID NO: 314 having 1, 2 or 3 point mutations that add, delete or substitute any of the amino acids of the polypeptide comprising SEQ ID NO: 314.

Preferably, the herein provided polypeptide fusion between PTN and NRG1 is oriented such that the part spanning the N-terminus to the fusion junction is polypeptide sequence from PTN and the part spanning the fusion junction to the C-terminus is NRG1 polypeptide sequence. Preferably, the NRG1 polypeptide sequence comprises or encodes an EGF-like domain. Said EGF-like domain containing PTN-NRG1 polypeptide fusion is preferably comprised by an aberrant cell as mentioned herein.

### ST14-NRG1 Polypeptide Fusion

Also provided is a polypeptide fusion encoded by a polynucleotide which comprises a portion of exon 11 of ST14, or an allelic variant thereof, and a portion of exon 6 of NRG1, or an allelic variant thereof. The polypeptide encoded by exon 11 of ST14 preferably comprises or consists of SEQ ID NO: 362, or an allelic variant of SEQ ID NO: 362. The polypeptide encoded by exon 6 of NRG1 preferably comprises or consists of SEQ ID NO: 144, or an allelic variant of SEQ ID NO: 144. Preferably, said polypeptide fusion further includes any one of SEQ ID NOs: 352-361, or any allelic variant of SEQ ID NO: 352-361, and preferably further includes SEQ ID NOs: 145-151, or any allelic variant of SEQ ID NOs: 145-151. SEQ ID NOs: 352-361 correspond to the individual polypeptide sequences encoded by exons 1-10 of ST14, respectively. SEQ ID NOs: 145-151 correspond to the individual polypeptide sequences encoded by exons 7-13 of NRG1, respectively. Said portion of exon 6 of NRG1 may also be comprised by SEQ ID NO: 156, or an allelic variant of SEQ ID NO: 156, which sequence corresponds to the polypeptide sequence encoded by all of exons 6-13.

Preferably, the polypeptide encoded by the portion of exon 11 of ST14 comprises or consists of 8, 9, 10, 11, 12, 13 or 14 contiguous amino acids from SEQ ID NO: 362, or from an allelic variant SEQ ID NO: 362. The allelic variant has at least 85% identity to SEQ ID NO: 362, preferably at least 90% identity, 92%, 94%, 96% or more preferably at least 98% sequence identity thereto. Also, the polypeptide encoded by the portion of exon 6 of NRG1 comprises or consists of 8, 9, 10, 11, 12, 13 or 14 contiguous amino acids from SEQ ID NO: 144, or from an allelic variant SEQ ID NO: 144. The allelic variant has at least 85% identity to SEQ ID NO: 144, preferably at least 90% identity, 92%, 94%, 96% or more preferably at least 98% sequence identity thereto.

Preferably, any ST14-NRG1 polypeptide fusion of the present disclosure includes the polypeptide sequence according to SEQ ID NO: 331, or an allelic variant thereof. SEQ ID NO: 331 contains a fusion junction between ST14 and NRG1, which fusion is located between amino acid at position 31 (which, together with amino acids at positions 1-30, stems from ST14) and the amino acid at position 33 (which, together with amino acids at positions 34-60 stems from NRG1). At position 32, a proline (P, pro) residue is present due to an unpredicted in-frame fusion of NRG1 with ST14. Preferably, the ST14-NRG1 polypeptide fusion includes 8, 9, 10, 11, 12, 13 or 14 contiguous amino acids from SEQ ID NO: 331, or of an allelic variant thereof, including at least the amino acids at position 31, 32 and 33 thereof.

In a preferred embodiment, there is provided a polypeptide sequence according to SEQ ID NO: 331, or a polypeptide which comprises 8, 9, 10, 11, 12, 13, 14 or all contiguous amino acids from SEQ ID NO: 331, or of an allelic variant thereof, including at least the amino acids at position 31, 32 and 33. Said polypeptide sequence has at least 85% identity to SEQ ID NO: 455, preferably at least 90% identity, 92%, 94%, 96% or more preferably at least 98% sequence identity thereto.

Preferably, any ST14-NRG1 polypeptide fusion of the present disclosure includes the polypeptide sequence of SEQ ID NO: 331 having one or more (i.e. 1, 2, 3, 4, 5, 6, 7, 8, 9 or 10) point mutations that add, delete or substitute any of the amino acids of the polypeptide comprising SEQ ID NO: 331. Preferably, said polypeptide fusion includes the polypeptide sequence of SEQ ID NO: 331 having 1, 2, 3, 4, or 5 point mutations that add, delete or substitute any of the amino acids of the polypeptide comprising SEQ ID NO: 331. More preferably, said polynucleotide fusion includes the polypeptide sequence of SEQ ID NO: 331 having 1, 2 or 3 point mutations that add, delete or substitute any of the amino acids of the polypeptide comprising SEQ ID NO: 331.

Preferably, the herein provided polypeptide fusion between ST14 and NRG1 is oriented such that the part spanning the N-terminus to the fusion junction is polypeptide sequence from ST14 and the part spanning the fusion junction to the C-terminus is NRG1 polypeptide sequence. Preferably, the NRG1 polypeptide sequence comprises or encodes an EGF-like domain. Said EGF-like domain containing ST14-NRG1 polypeptide fusion is preferably comprised by an aberrant cell as mentioned herein.

### THBS1-NRG1 Polypeptide Fusion

Also provided is a polypeptide fusion encoded by a polynucleotide which comprises a portion of exon 9 of THBS1, or an allelic variant thereof, and a portion of exon 6 of NRG1, or an allelic variant thereof. The polypeptide encoded by exon 9 of THBS1 preferably comprises or consists of SEQ ID NO: 396, or an allelic variant of SEQ ID NO: 396. The polypeptide encoded by exon 6 of NRG1 preferably comprises or consists of SEQ ID NO: 144, or an allelic variant of SEQ ID NO: 144. Preferably, said polypeptide fusion further includes any one of SEQ ID NOs: 389-395, or any allelic variant of SEQ ID NO: 389-395, and preferably further includes SEQ ID NOs: 145-151, or any allelic variant of SEQ ID NOs: 145-151. SEQ ID NO: 389-395 correspond to the individual polypeptide sequences encoded by exons 2-8 of THBS1, respectively. SEQ ID NOs: 145-151 correspond to the individual polypeptide sequences encoded by exons 7-13 of NRG1, respectively. Said portion of exon 6 of NRG1 may also be comprised by SEQ ID NO: 156, or an allelic variant of SEQ ID NO: 156, which sequence corresponds to the polypeptide sequence encoded by all of exons 6-13.

Preferably, the polypeptide encoded by the portion of exon 9 of THBS1 comprises or consists of 8, 9, 10, 11, 12, 13 or 14 contiguous amino acids from SEQ ID NO: 396, or from an allelic variant SEQ ID NO: 396. The allelic variant has at least 85% identity to SEQ ID NO: 396, preferably at least 90% identity, 92%, 94%, 96% or more preferably at least 98% sequence identity thereto. Also, the polypeptide encoded by the portion of exon 6 of NRG1 comprises or consists of 8, 9, 10, 11, 12, 13 or 14 contiguous amino acids from SEQ ID NO: 144, or from an allelic variant SEQ ID NO: 144. The allelic variant has at least 85% identity to SEQ ID NO: 144, preferably at least 90% identity, 92%, 94%, 96% or more preferably at least 98% sequence identity thereto.

Preferably, any THBS1-NRG1 polypeptide fusion of the present disclosure includes the polypeptide sequence according to SEQ ID NO: 377, or an allelic variant thereof. SEQ ID NO: 377 contains a fusion junction between THBS1 and NRG1, which fusion is located between amino acid at position 18 (which, together with amino acids at positions 1-16, stems from THBS1) and the amino acid at position 20 (which, together with amino acids at positions 20-48 stems from NRG1). At position 19, a threonine (T, the) residue is present due to an unpredicted in-frame fusion of NRG1 with THBS1. Preferably, the THBS1-NRG1 polypeptide fusion includes 8, 9, 10, 11, 12, 13 or 14 contiguous amino acids from SEQ ID NO: 377, or of an allelic variant thereof, including at least the amino acids at position 18, 19 and 20 thereof.

In a preferred embodiment, there is provided a polypeptide sequence according to SEQ ID NO: 377, or a polypeptide which comprises 8, 9, 10, 11, 12, 13, 14 or all contiguous amino acids from SEQ ID NO: 377, or of an allelic variant thereof, including at least the amino acids at position 18, 19 and 20. Said polypeptide sequence has at least 85% identity to SEQ ID NO: 377, preferably at least 90% identity, 92%, 94%, 96% or more preferably at least 98% sequence identity thereto.

Preferably, any THBS1-NRG1 polypeptide fusion of the present disclosure includes the polypeptide sequence of SEQ ID NO: 377 having one or more (i.e. 1, 2, 3, 4, 5, 6, 7, 8, 9 or 10) point mutations that add, delete or substitute any of the amino acids of the polypeptide comprising SEQ ID NO: 377. Preferably, said polypeptide fusion includes the polypeptide sequence of SEQ ID NO: 377 having 1, 2, 3, 4, or 5 point mutations that add, delete or substitute any of the amino acids of the polypeptide comprising SEQ ID NO: 377. More preferably, said polynucleotide fusion includes the polypeptide sequence of SEQ ID NO: 377 having 1, 2 or 3 point mutations that add, delete or substitute any of the amino acids of the polypeptide comprising SEQ ID NO: 377.

Preferably, the herein provided polypeptide fusion between THBS1 and NRG1 is oriented such that the part spanning the N-terminus to the fusion junction is polypeptide sequence from THBS1 and the part spanning the fusion junction to the C-terminus is NRG1 polypeptide sequence. Preferably, the NRG1 polypeptide sequence comprises or encodes an EGF-like domain. Said EGF-like domain containing THBS1-NRG1 polypeptide fusion is preferably comprised by an aberrant cell as mentioned herein.

### AGRN-NRG1 Polypeptide Fusion

Also provided is a polypeptide fusion encoded by a polynucleotide which comprises a portion of exon 12 of AGRN, or an allelic variant thereof, and a portion of exon 6 of NRG1, or an allelic variant thereof. The polypeptide encoded by exon 12 of AGRN preferably comprises or consists of SEQ ID NO: 430, or an allelic variant of SEQ ID NO: 430. The polypeptide encoded by exon 6 of NRG1 preferably comprises or consists of SEQ ID NO: 144, or an allelic variant of SEQ ID NO: 144. Preferably, said polypeptide fusion further includes any one of SEQ ID NOs: 419-429, or any allelic variant of SEQ ID NO: 419-429, and preferably further includes SEQ ID NOs: 145-151, or any allelic variant of SEQ ID NOs: 145-151. SEQ ID NOs: 419-429 correspond to the individual polypeptide sequences encoded by exons 1-11 of AGRN. SEQ ID NOs: 145-151 correspond to the individual polypeptide sequences encoded by exons 7-13 of NRG1, respectively. Said portion of exon 6 of NRG1 may also be comprised by SEQ ID NO: 156, or an allelic variant of SEQ ID NO: 156, which sequence corresponds to the polypeptide sequence encoded by all of exons 6-13.

Preferably, the polypeptide encoded by the portion of exon 12 of AGRN comprises or consists of 8, 9, 10, 11, 12, 13 or 14 contiguous amino acids from SEQ ID NO: 430, or from an allelic variant SEQ ID NO: 430. The allelic variant has at least 85% identity to SEQ ID NO: 430, preferably at least 90% identity, 92%, 94%, 96% or more preferably at least 98% sequence identity thereto. Also, the polypeptide encoded by the portion of exon 6 of NRG1 comprises or consists of 8, 9, 10, 11, 12, 13 or 14 contiguous amino acids from SEQ ID NO: 144, or from an allelic variant SEQ ID NO: 144. The allelic variant has at least 85% identity to SEQ ID NO: 1440, preferably at least 90% identity, 92%, 94%, 96% or more preferably at least 98% sequence identity thereto.

Preferably, any AGRN-NRG1 polypeptide fusion of the present disclosure includes the polypeptide sequence according to SEQ ID NO: 404, or an allelic variant thereof. SEQ ID NO: 404 contains a fusion junction between AGRN and NRG1, which fusion is located between amino acid at position 35 (which, together with amino acids at positions 1-34, stems from AGRN) and the amino acid at position 37 (which, together with amino acids at positions 38-69 stems from NRG1). At position 36, an alanine (A, ala) residue is present due to an unpredicted in frame fusion of NRG1 with AGRN. Preferably, the AGRN-NRG1 polypeptide fusion includes 8, 9, 10, 11, 12, 13 or 14 contiguous amino acids from SEQ ID NO: 404, or of an allelic variant thereof, including at least the amino acids at position 35, 36 and 37 thereof.

In a preferred embodiment, there is provided a polypeptide sequence according to SEQ ID NO: 404, or a polypeptide which comprises 8, 9, 10, 11, 12, 13, 14 or all contiguous amino acids from SEQ ID NO: 404, or of an allelic variant thereof, including at least the amino acids at position 35, 36 and 37. Said polypeptide sequence has at least 85% identity to SEQ ID NO: 404, preferably at least 90% identity, 92%, 94%, 96% or more preferably at least 98% sequence identity thereto.

Preferably, any AGRN-NRG1 polypeptide fusion of the present disclosure includes the polypeptide sequence of SEQ ID NO: 404 having one or more (i.e. 1, 2, 3, 4, 5, 6, 7, 8, 9 or 10) point mutations that add, delete or substitute any of the amino acids of the polypeptide comprising SEQ ID NO: 404. Preferably, said polypeptide fusion includes the polypeptide sequence of SEQ ID NO: 404 having 1, 2, 3, 4, or 5 point mutations that add, delete or substitute any of the amino acids of the polypeptide comprising SEQ ID NO: 404. More preferably, said polynucleotide fusion includes the polypeptide sequence of SEQ ID NO: 404 having 1, 2 or 3 point mutations that add, delete or substitute any of the amino acids of the polypeptide comprising SEQ ID NO: 404.

Preferably, the herein provided polypeptide fusion between AGRN and NRG1 is oriented such that the part spanning the N-terminus to the fusion junction is polypeptide sequence from AGRN and the part spanning the fusion junction to the C-terminus is NRG1 polypeptide sequence. Preferably, the NRG1 polypeptide sequence comprises or encodes an EGF-like domain. Said EGF-like domain containing AGRN-NRG1 polypeptide fusion is preferably comprised by an aberrant cell as mentioned herein.

### PVALB-NRG1 Polypeptide Fusion

In accordance with the present disclosure, there is provided a polypeptide fusion encoded by a polynucleotide comprising a PVALB nucleic acid sequence (or a portion of a PVALB nucleic acid sequence) fused with an NRG1 nucleic acid sequence (or a portion of a NRG1 nucleic acid sequence). The PVALB nucleic acid sequence, or portion thereof, preferably encodes a sequence comprising or consisting of any one of SEQ ID NOs 445-449, or an allelic variant of any one of these SEQ ID NOs. The NRG1 nucleic acid sequence, or portion thereof, preferably encodes a sequence comprising or consisting of any one of SEQ ID NOs 139-152, or an allelic variant of any one of these SEQ ID NOs.

The PVALB allelic variant of any one of SEQ ID NOs: 445-449, preferably has at least 85% sequence identity therewith, more preferably 90%, 92%, 94%, 96% or even more preferably at least 98% sequence identity therewith. The NRG1 allelic variant of any one of SEQ ID NOs: 139-152, preferably has at least 85% sequence identity therewith, more preferably 90%, 92%, 94%, 96% or even more preferably at least 98% sequence identity therewith.

Preferably, the portion of the PVALB nucleic acid sequence of said fusion encodes a polypeptide part of PVALB which comprises or consists of 8, 9, 10, 11, 12, 13 or 14 contiguous amino acids from any one of SEQ ID NOs 445-449, or an allelic variant of any one of SEQ ID NOs 445-449. Preferably, the portion of the NRG1 nucleic acid sequence of said fusion encodes a polypeptide part of NRG1 which comprises or consists 8, 9, 10, 11, 12, 13 or 14 contiguous amino acids from of any one of SEQ ID NOs 139-152, or an allelic variant of any one of SEQ ID NOs 139-152.

Preferably, any PVALB-NRG1 polypeptide fusion of the present disclosure includes a polypeptide sequence of any one of SEQ ID NOs: 445-449 having one or more (i.e. 1, 2, 3, 4, 5, 6, 7, 8, 9 or 10) point mutations that add, delete or substitute any of the amino acids of the polypeptides of SEQ ID NOs: 445-449. Preferably, said polypeptide fusion includes a polypeptide sequence of any one of SEQ ID NOs: 445-449 having 1, 2, 3, 4, or 5 point mutations that add, delete or substitute any of the amino acids of polypeptides of SEQ ID NO: 445-449. More preferably, said polynucleotide fusion includes a polypeptide sequence of any one of SEQ ID NOs: 445-449 having 1, 2, or 3 point mutations that add, delete or substitute any of the amino acids of the polypeptides of SEQ ID NO: 445-449.

In a preferred embodiment, there is provided a polypeptide fusion encoded by a polynucleotide which comprises a portion of exon 4 of PVALB, or an allelic variant thereof, and a portion of exon 6 of NRG1, or an allelic variant thereof. The polypeptide encoded by exon 4 of PVALB preferably comprises or consists of SEQ ID NO: 447, or an allelic variant of SEQ ID NO: 447. The polypeptide encoded by exon 6 of NRG1 preferably comprises or consists of SEQ ID NO: 144, or an allelic variant of SEQ ID NO: 144. Preferably, said polypeptide fusion further includes any one of SEQ ID NOs 445 and 446, and any one of SEQ ID NOs: 145-151, or any allelic variant of SEQ ID NOs: 145-151. SEQ ID NOs: 145-151 correspond to the individual polypeptide sequences encoded by exons 7-13 of NRG1, respectively. Said portion of exon 6 of NRG1 may also be according to SEQ ID NO: 156, or an allelic variant of SEQ ID NO: 156, which sequence corresponds to the polypeptide sequence encoded by all of exons 6-13.

Preferably, the polypeptide encoded by the portion of exon 4 of PVALB comprises or consists of 8, 9, 10, 11, 12, 13 or 14 contiguous amino acids from SEQ ID NO: 447, or from an allelic variant SEQ ID NO: 447. The allelic variant has at least 85% identity to SEQ ID NO: 447, preferably at least 90% identity, 92%, 94%, 96% or more preferably at least 98% sequence identity thereto. Also, the polypeptide encoded by the portion of exon 6 of NRG1 comprises or consists of 8, 9, 10, 11, 12, 13 or 14 contiguous amino acids from SEQ ID NO: 144, or from an allelic variant SEQ ID NO: 144. The allelic variant has at least 85% identity to SEQ ID NO: 144, preferably at least 90% identity, 92%, 94%, 96% or more preferably at least 98% sequence identity thereto.

Preferably, any PVALB-NRG1 polypeptide fusion of the present disclosure includes the polypeptide sequence according to SEQ ID NO: 438, or an allelic variant thereof. SEQ ID NO: 438 contains a fusion junction between PVALB and NRG1, which fusion is located between amino acid at position 33 (which, together with amino acids at positions 1-32, stems from PVALB) and the amino acid at position 35 (which, together with amino acids at positions 36-75 stems from NRG1). At position 34, an alanine (A, Ala) residue is present due to an unpredicted in-frame fusion of NRG1 with PVALB. Preferably, the PVALB-NRG1 polypeptide fusion includes 8, 9, 10, 11, 12, 13 or 14 contiguous amino acids from SEQ ID NO: 438, or of an allelic variant thereof, including at least the amino acids at position 33, 34 and 35.

In a preferred embodiment, there is provided a polypeptide sequence according to SEQ ID NO: 438, or a polypeptide which comprises 8, 9, 10, 11, 12, 13, 14 or all contiguous amino acids from SEQ ID NO: 438, or of an allelic variant thereof, including at least the amino acids at position 33, 34 and 35. Said polypeptide sequence has at least 85% identity to SEQ ID NO: 438, preferably at least 90% identity, 92%, 94%, 96% or more preferably at least 98% sequence identity thereto.

Preferably, any PVALB-NRG1 polypeptide fusion of the present disclosure includes the polypeptide sequence of SEQ ID NO: 438 having one or more (i.e. 1, 2, 3, 4, 5, 6, 7, 8, 9 or 10) point mutations that add, delete or substitute any of the amino acids of the polypeptide comprising SEQ ID NO: 438. Preferably, said polypeptide fusion includes the polypeptide sequence of SEQ ID NO: 438 having 1, 2, 3, 4, or 5 point mutations that add, delete or substitute any of the amino acids of the polypeptide comprising SEQ ID NO: 438. More preferably, said polynucleotide fusion includes the polypeptide sequence of SEQ ID NO: 438 polypeptide having 1, 2 or 3 point mutations that add, delete or substitute any of the amino acids of the polypeptide comprising SEQ ID NO: 438.

Preferably, the herein provided polypeptide fusion between NRG1 and PVALB is oriented such that the part spanning the N-terminus to the fusion junction is polypeptide sequence from PVALB and the part spanning the fusion junction to the C-terminus is NRG1 polypeptide sequence. Preferably, the NRG1 polypeptide sequence comprises or encodes an EGF-like domain. Said EGF-like domain containing PVALB-NRG1 polypeptide fusion is preferably comprised by an aberrant cell as mentioned herein.

### APP-NRG1 Polypeptide Fusion

Also provided is a polypeptide fusion encoded by a polynucleotide which comprises a portion of exon 14 of APP, or an allelic variant thereof, and a portion of exon 6 of NRG1, or an allelic variant thereof. The polypeptide encoded by exon 14 of APP preferably comprises or consists of SEQ ID NO: 519, or an allelic variant of SEQ ID NO: 519. The polypeptide encoded by exon 6 of NRG1 preferably comprises or consists of SEQ ID NO: 144, or an allelic variant of SEQ ID NO: 144. Preferably, said polypeptide fusion further includes any one of SEQ ID NOs: 506-518, or any allelic variant of SEQ ID NO: 506-518, and preferably further includes SEQ ID NOs: 145-151, or any allelic variant of SEQ ID NOs: 145-151. SEQ ID NOs: 506-518 correspond to the individual polypeptide sequences encoded by exons 1-13 of APP. SEQ ID NOs: 145-151 correspond to the individual polypeptide sequences encoded by exons 7-13 of NRG1, respectively. Said portion of exon 6 of NRG1 may also be comprised by SEQ ID NO: 156, or an allelic variant of SEQ ID NO: 156, which sequence corresponds to the polypeptide sequence encoded by all of exons 6-13.

Preferably, the polypeptide encoded by the portion of exon 14 of APP comprises or consists of 8, 9, 10, 11, 12, 13 or 14 contiguous amino acids from SEQ ID NO: 519, or from an allelic variant SEQ ID NO: 519. The allelic variant has at least 85% identity to SEQ ID NO: 519, preferably at least 90% identity, 92%, 94%, 96% or more preferably at least 98% sequence identity thereto. Also, the polypeptide encoded by the portion of exon 6 of NRG1 comprises or consists of 8, 9, 10, 11, 12, 13 or 14 contiguous amino acids from SEQ ID NO: 144, or from an allelic variant SEQ ID NO: 144. The allelic variant has at least 85% identity to SEQ ID NO: 144, preferably at least 90% identity, 92%, 94%, 96% or more preferably at least 98% sequence identity thereto.

Preferably, any APP-NRG1 polypeptide fusion of the present disclosure includes the polypeptide sequence according to SEQ ID NO: 487, or an allelic variant thereof. SEQ ID NO: 487 contains a fusion junction between APP and NRG1, which fusion is located between amino acid at position 17 (which, together with amino acids at positions 1-16, stems from APP) and the amino acid at position 19 (which, together with amino acids at positions 20-46 stems from NRG1). At position 18, an alanine (A, ala) residue is present due to an unpredicted in-frame fusion of NRG1 with APP. Preferably, the APP-NRG1 polypeptide fusion includes 8, 9, 10, 11, 12, 13 or 14 contiguous amino acids from SEQ ID NO: 487, or of an allelic variant thereof, including at least the amino acids at position 17, 18 and 19 thereof.

In a preferred embodiment, there is provided a polypeptide sequence according to SEQ ID NO: 487, or a polypeptide which comprises 8, 9, 10, 11, 12, 13, 14 or all contiguous amino acids from SEQ ID NO: 487, or of an allelic variant thereof, including at least the amino acids at position 17, 18 and 19. Said polypeptide sequence has at least 85% identity to SEQ ID NO: 487, preferably at least 90% identity, 92%, 94%, 96% or more preferably at least 98% sequence identity thereto.

Preferably, any APP-NRG1 polypeptide fusion of the present disclosure includes the polypeptide sequence of SEQ ID NO: 487 having one or more (i.e. 1, 2, 3, 4, 5, 6, 7, 8, 9 or 10) point mutations that add, delete or substitute any of the amino acids of the polypeptide comprising SEQ ID NO: 487. Preferably, said polypeptide fusion includes the polypeptide sequence of SEQ ID NO: 487 having 1, 2, 3, 4, or 5 point mutations that add, delete or substitute any of the amino acids of the polypeptide comprising SEQ ID NO: 487. More preferably, said polynucleotide fusion includes the polypeptide sequence of SEQ ID NO: 487 having 1, 2 or 3 point mutations that add, delete or substitute any of the amino acids of the polypeptide comprising SEQ ID NO: 487.

Preferably, the herein provided polypeptide fusion between APP and NRG1 is oriented such that the part spanning the N-terminus to the fusion junction is polypeptide sequence from APP and the part spanning the fusion junction to the C-terminus is NRG1 polypeptide sequence. Preferably, the NRG1 polypeptide sequence comprises or encodes an EGF-like domain. Said EGF-like domain containing APP-NRG1 polypeptide fusion is preferably comprised by an aberrant cell as mentioned herein.

### WRN-NRG1 Polypeptide Fusion

Also provided is a polypeptide fusion encoded by a polynucleotide which comprises a portion of exon 33 of WRN, or an allelic variant thereof, and a portion of exon 6 of NRG1, or an allelic variant thereof. The polypeptide encoded by exon 33 of WRN preferably comprises or consists of SEQ ID NO: 597, or an allelic variant of SEQ ID NO: 597. The polypeptide encoded by exon 6 of NRG1 preferably comprises or consists of SEQ ID NO: 144, or an allelic variant of SEQ ID NO: 144. Preferably, said polypeptide fusion further includes any one of SEQ ID NOs: 566-596, or any allelic variant of SEQ ID NO: 566-596, and preferably further includes SEQ ID NOs: 145-151, or any allelic variant of SEQ ID NOs: 145-151. SEQ ID NOs: 566-596 correspond to the individual polypeptide sequences encoded by exons 2-32 of WRN. SEQ ID NOs: 145-151 correspond to the individual polypeptide sequences encoded by exons 7-13 of NRG1, respectively. Said portion of exon 6 of NRG1 may also be comprised by SEQ ID NO: 156, or an allelic variant of SEQ ID NO: 156, which sequence corresponds to the polypeptide sequence encoded by all of exons 6-13.

Preferably, the polypeptide encoded by the portion of exon 33 of WRN comprises or consists of 8, 9, 10, 11, 12, 13 or 14 contiguous amino acids from SEQ ID NO: 597, or from an allelic variant SEQ ID NO: 597. The allelic variant has at least 85% identity to SEQ ID NO: 597, preferably at least 90% identity, 92%, 94%, 96% or more preferably at least 98% sequence identity thereto. Also, the polypeptide encoded by the portion of exon 6 of NRG1 comprises or consists of 8, 9, 10, 11, 12, 13 or 14 contiguous amino acids from SEQ ID NO: 144, or from an allelic variant SEQ ID NO: 144. The allelic variant has at least 85% identity to SEQ ID NO: 144, preferably at least 90% identity, 92%, 94%, 96% or more preferably at least 98% sequence identity thereto.

Preferably, any WRN-NRG1 polypeptide fusion of the present disclosure includes the polypeptide sequence according to SEQ ID NO: 528, or an allelic variant thereof. SEQ ID NO: 528 contains a fusion junction between WRN and NRG1, which fusion is located between amino acid at position 31 (which, together with amino acids at positions 1-30, stems from WRN) and the amino acid at position 33 (which, together with amino acids at positions 34-60 stems from NRG1). At position 32, an alanine (A, ala) residue is present due to an unpredicted in-frame fusion of NRG1 with WRN. Preferably, the WRN-NRG1 polypeptide fusion includes 8, 9, 10, 11, 12, 13 or 14 contiguous amino acids from SEQ ID NO: 528, or of an allelic variant thereof, including at least the amino acids at position 31, 32 and 33 thereof.

In a preferred embodiment, there is provided a polypeptide sequence according to SEQ ID NO: 528, or a polypeptide which comprises 8, 9, 10, 11, 12, 13, 14 or all contiguous amino acids from SEQ ID NO: 528, or of an allelic variant thereof, including at least the amino acids at position 31, 32 and 33. Said polypeptide sequence has at least 85% identity to SEQ ID NO: 528, preferably at least 90% identity, 92%, 94%, 96% or more preferably at least 98% sequence identity thereto.

Preferably, any WRN-NRG1 polypeptide fusion of the present disclosure includes the polypeptide sequence of SEQ ID NO: 528 having one or more (i.e. 1, 2, 3, 4, 5, 6, 7, 8, 9 or 10) point mutations that add, delete or substitute any of the amino acids of the polypeptide comprising SEQ ID NO: 528. Preferably, said polypeptide fusion includes the polypeptide sequence of SEQ ID NO: 528 having 1, 2, 3, 4, or 5 point mutations that add, delete or substitute any of the amino acids of the polypeptide comprising SEQ ID NO: 528. More preferably, said polynucleotide fusion includes the polypeptide sequence of SEQ ID NO: 528 having 1, 2 or 3 point mutations that add, delete or substitute any of the amino acids of the polypeptide comprising SEQ ID NO: 528.

Preferably, the herein provided polypeptide fusion between WRN and NRG1 is oriented such that the part spanning the N-terminus to the fusion junction is polypeptide sequence from WRN and the part spanning the fusion junction to the C-terminus is NRG1 polypeptide sequence. Preferably, the NRG1 polypeptide sequence comprises or encodes an EGF-like domain. Said EGF-like domain containing WRN-NRG1 polypeptide fusion is preferably comprised by an aberrant cell as mentioned herein.

### ASPH-NRG1 Polypeptide Fusion

In accordance with the present disclosure, there is provided a polypeptide fusion encoded by a polynucleotide comprising a ASPH nucleic acid sequence (or a portion of a ASPH nucleic acid sequence) fused with an NRG1 nucleic acid sequence (or a portion of a NRG1 nucleic acid sequence). The ASPH nucleic acid sequence, or portion thereof, preferably encodes a sequence comprising or consisting of any one of SEQ ID NOs: 663-688, or an allelic variant of any one of these SEQ ID NOs. The NRG1 nucleic acid sequence, or portion thereof, preferably encodes a sequence comprising or consisting of any one of SEQ ID NOs 139-152, or an allelic variant of any one of these SEQ ID NOs.

The ASPH allelic variant of any one of SEQ ID NOs: 663-688, preferably has at least 85% sequence identity therewith, more preferably 90%, 92%, 94%, 96% or even more preferably at least 98% sequence identity therewith. The NRG1 allelic variant of any one of SEQ ID NOs: 139-152, preferably has at least 85% sequence identity therewith, more preferably 90%, 92%, 94%, 96% or even more preferably at least 98% sequence identity therewith.

Preferably, the portion of the ASPH nucleic acid sequence of said fusion encodes a polypeptide part of ASPH which comprises or consists of 8, 9, 10, 11, 12, 13 or 14 contiguous amino acids from any one of SEQ ID NOs: 663-688, or an allelic variant of any one of SEQ ID NOs: 663-688. Preferably, the portion of the NRG1 nucleic acid sequence of said fusion encodes a polypeptide part of NRG1 which comprises or consists 8, 9, 10, 11, 12, 13 or 14 contiguous amino acids from of any one of SEQ ID NOs: 139-152, or an allelic variant of any one of SEQ ID NOs: 139-152.

Preferably, any ASPH-NRG1 polypeptide fusion of the present disclosure includes a polypeptide sequence of any one of SEQ ID NOs: 663-688 having one or more (i.e. 1, 2, 3, 4, 5, 6, 7, 8, 9 or 10) point mutations that add, delete or substitute any of the amino acids of the polypeptides of SEQ ID NOs: 663-688. Preferably, said polypeptide fusion includes a polypeptide sequence of any one of SEQ ID NOs: 663-688 having 1, 2, 3, 4, or 5 point mutations that add, delete or substitute any of the amino acids of polypeptides of SEQ ID NOs: 663-688. More preferably, said polynucleotide fusion includes a polypeptide sequence of any one of SEQ ID NOs: 663-688 having 1, 2, or 3 point mutations that add, delete or substitute any of the amino acids of the polypeptides of SEQ ID NOs: 663-688.

In a preferred embodiment, there is provided a polypeptide fusion encoded by a polynucleotide which comprises a portion of exon 22 of ASPH, or an allelic variant thereof, and a portion of exon 2 of NRG1, or an allelic variant thereof. The polypeptide encoded by exon 22 of ASPH preferably comprises or consists of SEQ ID NO: 684, or an allelic variant of SEQ ID NO: 684. The polypeptide encoded by exon 2 of NRG1 preferably comprises or consists of SEQ ID NO: 140, or an allelic variant of SEQ ID NO: 140. Preferably, said polypeptide fusion further includes any one of SEQ ID NOs: 663-683, and any one of SEQ ID NOs: 141-151, or any allelic variant of SEQ ID NOs: 663-683 or SEQ ID NOs: 141-151. SEQ ID NOs: 663-683 correspond to the individual polypeptide sequences encoded by exons 1-21 of ASPH and SEQ ID NOs: 141-151 correspond to the individual polypeptide sequences encoded by exons 3-13 of NRG1, respectively. Said portion of exon 2 of NRG1 may also be according to SEQ ID NO: 154, or an allelic variant of SEQ ID NO: 154, which sequence corresponds to the polypeptide sequence encoded by all of exons 3-13.

Preferably, the polypeptide encoded by the portion of exon 22 of ASPH comprises or consists of 8, 9, 10, 11, 12, 13 or 14 contiguous amino acids from SEQ ID NO: 684, or from an allelic variant SEQ ID NO: 684. The allelic variant has at least 85% identity to SEQ ID NO: 684, preferably at least 90% identity, 92%, 94%, 96% or more preferably at least 98% sequence identity thereto. Also, the polypeptide encoded by the portion of exon 2 of NRG1 comprises or consists of 8, 9, 10, 11, 12, 13 or 14 contiguous amino acids from SEQ ID NO: 140, or from an allelic variant SEQ ID NO: 140. The allelic variant has at least 85% identity to SEQ ID NO: 140, preferably at least 90% identity, 92%, 94%, 96% or more preferably at least 98% sequence identity thereto.

Preferably, any ASPH-NRG1 polypeptide fusion of the present disclosure includes the polypeptide sequence according to SEQ ID NO: 636, or an allelic variant thereof. SEQ ID NO: 636 contains a fusion junction between ASPH and NRG1, which fusion is located between amino acid at position 24 (which, together with amino acids at positions 1-23, stems from ASPH) and the amino acid at position 26 (which, together with amino acids at positions 27-49 stems from NRG1). At position 25, an alanine (A, Ala) residue is present due to an unpredicted in frame fusion of NRG1 with ASPH. Preferably, the ASPH-NRG1 polypeptide fusion includes 8, 9, 10, 11, 12, 13 or 14 contiguous amino acids from SEQ ID NO: 636, or of an allelic variant thereof, including at least the amino acids at position 24, 25 and 26.

In a preferred embodiment, there is provided a polypeptide sequence according to SEQ ID NO: 636, or a polypeptide which comprises 8, 9, 10, 11, 12, 13, 14 or all contiguous amino acids from SEQ ID NO: 636, or of an allelic variant thereof, including at least the amino acids at position 24, 25 and 26. Said polypeptide sequence has at least 85% identity to SEQ ID NO: 636, preferably at least 90% identity, 92%, 94%, 96% or more preferably at least 98% sequence identity thereto.

Preferably, any ASPH-NRG1 polypeptide fusion of the present disclosure includes the polypeptide sequence of SEQ ID NO: 636 having one or more (i.e. 1, 2, 3, 4, 5, 6, 7, 8, 9 or 10) point mutations that add, delete or substitute any of the amino acids of the polypeptide comprising SEQ ID NO: 636. Preferably, said polypeptide fusion includes the polypeptide sequence of SEQ ID NO: 636 having 1, 2, 3, 4, or 5 point mutations that add, delete or substitute any of the amino acids of the polypeptide comprising SEQ ID NO: 636. More preferably, said polynucleotide fusion includes the polypeptide sequence of SEQ ID NO: 636 polypeptide having 1, 2 or 3 point mutations that add, delete or substitute any of the amino acids of the polypeptide comprising SEQ ID NO: 636.

Preferably, the herein provided polypeptide fusion between NRG1 and ASPH is oriented such that the part spanning the N-terminus to the fusion junction is polypeptide sequence from ASPH and the part spanning the fusion junction to the C-terminus is NRG1 polypeptide sequence. Preferably, the NRG1 polypeptide sequence comprises or encodes an EGF-like domain. Said EGF-like domain containing ASPH-NRG1 polypeptide fusion is preferably comprised by an aberrant cell as mentioned herein.

### NOTCH2-NRG1 Polypeptide Fusion

Also provided is a polypeptide fusion encoded by a polynucleotide which comprises a portion of exon 6 of NOTCH2, or an allelic variant thereof, and a portion of exon 6 of NRG1, or an allelic variant thereof. The polypeptide encoded by exon 6 of NOTCH2 preferably comprises or consists of SEQ ID NO: 709, or an allelic variant of SEQ ID NO: 709. The polypeptide encoded by exon 6 of NRG1 preferably comprises or consists of SEQ ID NO: 144, or an allelic variant of SEQ ID NO: 144. Preferably, said polypeptide fusion further includes any one of SEQ ID NOs: 704-708, or any allelic variant of SEQ ID NO: 704-708, and preferably further includes SEQ ID NOs: 145-151, or any allelic variant of SEQ ID NOs: 145-151. SEQ ID NOs: 566-596 correspond to the individual polypeptide sequences encoded by exons 1-5 of NOTCH2. SEQ ID NOs: 145-151 correspond to the individual polypeptide sequences encoded by exons 7-13 of NRG1, respectively. Said portion of exon 6 of NRG1 may also be comprised by SEQ ID NO: 156, or an allelic variant of SEQ ID NO: 156, which sequence corresponds to the polypeptide sequence encoded by all of exons 6-13.

Preferably, the polypeptide encoded by the portion of exon 6 of NOTCH2 comprises or consists of 8, 9, 10, 11, 12, 13 or 14 contiguous amino acids from SEQ ID NO: 709, or from an allelic variant SEQ ID NO: 709. The allelic variant has at least 85% identity to SEQ ID NO: 709, preferably at least 90% identity, 92%, 94%, 96% or more preferably at least 98% sequence identity thereto. Also, the polypeptide encoded by the portion of exon 6 of NRG1 comprises or consists of 8, 9, 10, 11, 12, 13 or 14 contiguous amino acids from SEQ ID NO: 144, or from an allelic variant SEQ ID NO: 144. The allelic variant has at least 85% identity to SEQ ID NO: 144, preferably at least 90% identity, 92%, 94%, 96% or more preferably at least 98% sequence identity thereto.

Preferably, any NOTCH2-NRG1 polypeptide fusion of the present disclosure includes the polypeptide sequence according to SEQ ID NO: 694, or an allelic variant thereof. SEQ ID NO: 694 contains a fusion junction between NOTCH2 and NRG1, which fusion is located between amino acid at position 24 (which, together with amino acids at positions 1-23, stems from NOTCH2) and the amino acid at position 26 (which, together with amino acids at positions 27-49 stems from NRG1). At position 25, an alanine (A, ala) residue is present due to an unpredicted in-frame fusion of NRG1 with NOTCH2. Preferably, the NOTCH2-NRG1 polypeptide fusion includes 8, 9, 10, 11, 12, 13 or 14 contiguous amino acids from SEQ ID NO: 694, or of an allelic variant thereof, including at least the amino acids at position 24, 25 and 26 thereof.

In a preferred embodiment, there is provided a polypeptide sequence according to SEQ ID NO: 694, or a polypeptide which comprises 8, 9, 10, 11, 12, 13, 14 or all contiguous amino acids from SEQ ID NO: 694, or of an allelic variant thereof, including at least the amino acids at position 24, 25 and 26. Said polypeptide sequence has at least 85% identity to SEQ ID NO: 694, preferably at least 90% identity, 92%, 94%, 96% or more preferably at least 98% sequence identity thereto.

Preferably, any NOTCH2-NRG1 polypeptide fusion of the present disclosure includes the polypeptide sequence of SEQ ID NO: 694 having one or more (i.e. 1, 2, 3, 4, 5, 6, 7, 8, 9 or 10) point mutations that add, delete or substitute any of the amino acids of the polypeptide comprising SEQ ID NO: 694. Preferably, said polypeptide fusion includes the polypeptide sequence of SEQ ID NO: 694 having 1, 2, 3, 4, or 5 point mutations that add, delete or substitute any of the amino acids of the polypeptide comprising SEQ ID NO: 694. More preferably, said polynucleotide fusion includes the polypeptide sequence of SEQ ID NO: 694 having 1, 2 or 3 point mutations that add, delete or substitute any of the amino acids of the polypeptide comprising SEQ ID NO: 694.

Preferably, the herein provided polypeptide fusion between NOTCH2 and NRG1 is oriented such that the part spanning the N-terminus to the fusion junction is polypeptide sequence from NOTCH2 and the part spanning the fusion junction to the C-terminus is NRG1 polypeptide sequence. Preferably, the NRG1 polypeptide sequence comprises or encodes an EGF-like domain. Said EGF-like domain containing NOTCH2-NRG1 polypeptide fusion is preferably comprised by an aberrant cell as mentioned herein.

### CD74-NRG1 Polypeptide Fusion

Also provided is a polypeptide fusion encoded by a polynucleotide which comprises a portion of exon 2 of CD74, or an allelic variant thereof, and a portion of exon 2 of NRG1, or an allelic variant thereof. The polypeptide encoded by exon 2 of CD74 preferably comprises or consists of SEQ ID NO: 730, or an allelic variant of SEQ ID NO: 730. The polypeptide encoded by exon 2 of NRG1 preferably comprises or consists of SEQ ID NO: 140, or an allelic variant of SEQ ID NO: 140. Preferably, said polypeptide fusion further includes any one of SEQ ID NO: 729 or SEQ ID NOs: 141-151, or any allelic variant of SEQ ID NO: 729 or SEQ ID NOs: 141-151. SEQ ID NO: 729 corresponds to the polypeptide sequence encoded by exon 1 of CD74. SEQ ID NOs: 141-151 correspond to the individual polypeptide sequences encoded by exons 3-13 of NRG1, respectively. Said portion of exon 2 of NRG1 may also be comprised by SEQ ID NO: 154, or an allelic variant of SEQ ID NO: 154, which sequence corresponds to the polypeptide sequence encoded by all of exons 2-13.

Preferably, the polypeptide encoded by the portion of exon 2 of CD74 comprises or consists of 8, 9, 10, 11, 12, 13 or 14 contiguous amino acids from SEQ ID NO: 730, or from an allelic variant SEQ ID NO: 730. The allelic variant has at least 85% identity to SEQ ID NO: 730, preferably at least 90% identity, 92%, 94%, 96% or more preferably at least 98% sequence identity thereto. Also, the polypeptide encoded by the portion of exon 2 of NRG1 comprises or consists of 8, 9, 10, 11, 12, 13 or 14 contiguous amino acids from SEQ ID NO: 140, or from an allelic variant SEQ ID NO: 140. The allelic variant has at least 85% identity to SEQ ID NO: 140, preferably at least 90% identity, 92%, 94%, 96% or more preferably at least 98% sequence identity thereto.

Preferably, any CD74-NRG1 polypeptide fusion of the present disclosure includes the polypeptide sequence according to SEQ ID NO: 718, or an allelic variant thereof. SEQ ID NO: 718 contains a fusion junction between CD74 and NRG1, which fusion is located between amino acid at position 24 (which, together with amino acids at positions 1-23, stems from CD74) and the amino acid at position 26 (which, together with amino acids at positions 27-49 stems from NRG1). At position 25, a proline (P, pro) residue is present due to an unpredicted in frame fusion of NRG1 with CD74. Preferably, the CD74-NRG1 polypeptide fusion includes 8, 9, 10, 11, 12, 13 or 14 contiguous amino acids from SEQ ID NO: 718, or of an allelic variant thereof, including at least the amino acids at position 24, 25 and 26 thereof.

In a preferred embodiment, there is provided a polypeptide sequence according to SEQ ID NO: 718, or a polypeptide which comprises 8, 9, 10, 11, 12, 13, 14 or all contiguous amino acids from SEQ ID NO: 718, or of an allelic variant thereof, including at least the amino acids at position 24, 25 and 26. Said polypeptide sequence has at least 85% identity to SEQ ID NO: 718, preferably at least 90% identity, 92%, 94%, 96% or more preferably at least 98% sequence identity thereto.

Preferably, any CD74-NRG1 polypeptide fusion of the present disclosure includes the polypeptide sequence of SEQ ID NO: 718 having one or more (i.e. 1, 2, 3, 4, 5, 6, 7, 8, 9 or 10) point mutations that add, delete or substitute any of the amino acids of the polypeptide comprising SEQ ID NO: 718. Preferably, said polypeptide fusion includes the polypeptide sequence of SEQ ID NO: 718 having 1, 2, 3, 4, or 5 point mutations that add, delete or substitute any of the amino acids of the polypeptide comprising SEQ ID NO: 718. More preferably, said polynucleotide fusion includes the polypeptide sequence of SEQ ID NO: 718 having 1, 2 or 3 point mutations that add, delete or substitute any of the amino acids of the polypeptide comprising SEQ ID NO: 718.

Preferably, the herein provided polypeptide fusion between CD74 and NRG1 is oriented such that the part spanning the N-terminus to the fusion junction is polypeptide sequence from CD74 and the part spanning the fusion junction to the C-terminus is NRG1 polypeptide sequence. Preferably, the NRG1 polypeptide sequence comprises or encodes an EGF-like domain. Said EGF-like domain containing CD74-NRG1 polypeptide fusion is preferably comprised by an aberrant cell as mentioned herein.

### SDC4-NRG1 Polypeptide Fusion

Also provided is a polypeptide fusion encoded by a polynucleotide which comprises a portion of exon 2 of SDC4, or an allelic variant thereof, and a portion of exon 2 of NRG1, or an allelic variant thereof. The polypeptide encoded by exon 2 of SDC4 preferably comprises or consists of SEQ ID NO: 752, or an allelic variant of SEQ ID NO: 752. The polypeptide encoded by exon 2 of NRG1 preferably comprises or consists of SEQ ID NO: 140, or an allelic variant of SEQ ID NO: 140. Preferably, said polypeptide fusion further includes any one of SEQ ID NO: 751 or SEQ ID NOs: 141-151, or any allelic variant of SEQ ID NO: 751 or SEQ ID NOs: 141-151. SEQ ID NO: 751 corresponds to the polypeptide sequence encoded by exon 1 of SDC4. SEQ ID NOs: 141-151 correspond to the individual polypeptide sequences encoded by exons 3-13 of NRG1, respectively. Said portion of exon 2 of NRG1 may also be comprised by SEQ ID NO: 154, or an allelic variant of SEQ ID NO: 154, which sequence corresponds to the polypeptide sequence encoded by all of exons 2-13.

Preferably, the polypeptide encoded by the portion of exon 2 of SDC4 comprises or consists of 8, 9, 10, 11, 12, 13 or 14 contiguous amino acids from SEQ ID NO: 752, or from an allelic variant SEQ ID NO: 752. The allelic variant has at least 85% identity to SEQ ID NO: 752, preferably at least 90% identity, 92%, 94%, 96% or more preferably at least 98% sequence identity thereto. Also, the polypeptide encoded by the portion of exon 2 of NRG1 comprises or consists of 8, 9, 10, 11, 12, 13 or 14 contiguous amino acids from SEQ ID NO: 140, or from an allelic variant SEQ ID NO: 140. The allelic variant has at least 85% identity to SEQ ID NO: 140, preferably at least 90% identity, 92%, 94%, 96% or more preferably at least 98% sequence identity thereto.

Preferably, any SDC4-NRG1 polypeptide fusion of the present disclosure includes the polypeptide sequence according to SEQ ID NO: 744, or an allelic variant thereof. SEQ ID NO: 744 contains a fusion junction between SDC4 and NRG1, which fusion is located between amino acid at position 24 (which, together with amino acids at positions 1-23, stems from SDC4) and the amino acid at position 26 (which, together with amino acids at positions 27-49 stems from NRG1). At position 25, an alanine (A, ala) residue is present due to an unpredicted in-frame fusion of NRG1 with SDC4. Preferably, the SDC4-NRG1 polypeptide fusion includes 8, 9, 10, 11, 12, 13 or 14 contiguous amino acids from SEQ ID NO: 744, or of an allelic variant thereof, including at least the amino acids at position 24, 25 and 26 thereof.

In a preferred embodiment, there is provided a polypeptide sequence according to SEQ ID NO: 744, or a polypeptide which comprises 8, 9, 10, 11, 12, 13, 14 or all contiguous amino acids from SEQ ID NO: 744, or of an allelic variant thereof, including at least the amino acids at position 24, 25 and 26. Said polypeptide sequence has at least 85% identity to SEQ ID NO: 744, preferably at least 90% identity, 92%, 94%, 96% or more preferably at least 98% sequence identity thereto.

Preferably, any SDC4-NRG1 polypeptide fusion of the present disclosure includes the polypeptide sequence of SEQ ID NO: 744 having one or more (i.e. 1, 2, 3, 4, 5, 6, 7, 8, 9 or 10) point mutations that add, delete or substitute any of the amino acids of the polypeptide comprising SEQ ID NO: 744. Preferably, said polypeptide fusion includes the polypeptide sequence of SEQ ID NO: 744 having 1, 2, 3, 4, or 5 point mutations that add, delete or substitute any of the amino acids of the polypeptide comprising SEQ ID NO: 744. More preferably, said polynucleotide fusion includes the polypeptide sequence of SEQ ID NO: 744 having 1, 2 or 3 point mutations that add, delete or substitute any of the amino acids of the polypeptide comprising SEQ ID NO: 744.

Preferably, the herein provided polypeptide fusion between SDC4 and NRG1 is oriented such that the part spanning the N-terminus to the fusion junction is polypeptide sequence from SDC4 and the part spanning the fusion junction to the C-terminus is NRG1 polypeptide sequence. Preferably, the NRG1 polypeptide sequence comprises or encodes an EGF-like domain. Said EGF-like domain containing SDC4-NRG1 polypeptide fusion is preferably comprised by an aberrant cell as mentioned herein.

Also provided is a polypeptide fusion encoded by a polynucleotide which comprises a portion of exon 4 of SDC4, or an allelic variant thereof, and a portion of exon 2 of NRG1, or an allelic variant thereof. The polypeptide encoded by exon 4 of SDC4 preferably comprises or consists of SEQ ID NO: 754, or an allelic variant of SEQ ID NO: 754. The polypeptide encoded by exon 2 of NRG1 preferably comprises or consists of SEQ ID NO: 140, or an allelic variant of SEQ ID NO: 140. Preferably, said polypeptide fusion further includes any one of SEQ IDs NO: 751-753 or SEQ ID NOs: 141-151, or any allelic variant of SEQ ID NO: 751-753 or SEQ ID NOs: 141-151. SEQ ID NO: 751-753 correspond to the individual polypeptide sequences encoded by exon 1-3 of SDC4. SEQ ID NOs: 141-151 correspond to the individual polypeptide sequences encoded by exons 3-13 of NRG1, respectively. Said portion of exon 2 of NRG1 may also be comprised by SEQ ID NO: 154, or an allelic variant of SEQ ID NO: 154, which sequence corresponds to the polypeptide sequence encoded by all of exons 2-13.

Preferably, the polypeptide encoded by the portion of exon 4 of SDC4 comprises or consists of 8, 9, 10, 11, 12, 13 or 14 contiguous amino acids from SEQ ID NO: 754, or from an allelic variant SEQ ID NO: 754. The allelic variant has at least 85% identity to SEQ ID NO: 754, preferably at least 90% identity, 92%, 94%, 96% or more preferably at least 98% sequence identity thereto. Also, the polypeptide encoded by the portion of exon 2 of NRG1 comprises or consists of 8, 9, 10, 11, 12, 13 or 14 contiguous amino acids from SEQ ID NO: 140, or from an allelic variant SEQ ID NO: 140. The allelic variant has at least 85% identity to SEQ ID NO: 140, preferably at least 90% identity, 92%, 94%, 96% or more preferably at least 98% sequence identity thereto.

Preferably, any SDC4-NRG1 polypeptide fusion of the present disclosure includes the polypeptide sequence according to SEQ ID NO: 825, or an allelic variant thereof. SEQ ID NO: 825 contains a fusion junction between SDC4 and NRG1, which fusion is located between amino acid at position 24 (which, together with amino acids at positions 1-23, stems from SDC4) and the amino acid at position 26 (which, together with amino acids at positions 27-49 stems from NRG1). At position 25, an alanine (A, ala) residue is present due to an unpredicted in-frame fusion of NRG1 with SDC4. Preferably, the SDC4-NRG1 polypeptide fusion includes 8, 9, 10, 11, 12, 13 or 14 contiguous amino acids from SEQ ID NO: 825, or of an allelic variant thereof, including at least the amino acids at position 24, 25 and 26 thereof.

In a preferred embodiment, there is provided a polypeptide sequence according to SEQ ID NO: 825, or a polypeptide which comprises 8, 9, 10, 11, 12, 13, 14 or all contiguous amino acids from SEQ ID NO: 825, or of an allelic variant thereof, including at least the amino acids at position 24, 25 and 26. Said polypeptide sequence has at least 85% identity to SEQ ID NO: 825, preferably at least 90% identity, 92%, 94%, 96% or more preferably at least 98% sequence identity thereto.

Preferably, any SDC4-NRG1 polypeptide fusion of the present disclosure includes the polypeptide sequence of SEQ ID NO: 825 having one or more (i.e. 1, 2, 3, 4, 5, 6, 7, 8, 9 or 10) point mutations that add, delete or substitute any of the amino acids of the polypeptide comprising SEQ ID NO: 825. Preferably, said polypeptide fusion includes the polypeptide sequence of SEQ ID NO: 825 having 1, 2, 3, 4, or 5 point mutations that add, delete or substitute any of the amino acids of the polypeptide comprising SEQ ID NO: 825. More preferably, said polynucleotide fusion includes the polypeptide sequence of SEQ ID NO: 825 having 1, 2 or 3 point mutations that add, delete or substitute any of the amino acids of the polypeptide comprising SEQ ID NO: 825.

Preferably, the herein provided polypeptide fusion between SDC4 and NRG1 is oriented such that the part spanning the N-terminus to the fusion junction is polypeptide sequence from SDC4 and the part spanning the fusion junction to the C-terminus is NRG1 polypeptide sequence. Preferably, the NRG1 polypeptide sequence comprises or encodes an EGF-like domain. Said EGF-like domain containing SDC4-NRG1 polypeptide fusion is preferably comprised by an aberrant cell as mentioned herein.

### SLC4A4-NRG1 Polypeptide Fusion

Also provided is a polypeptide fusion encoded by a polynucleotide which comprises a portion of exon 14 of SLC4A4, or an allelic variant thereof, and a portion of exon 6 of NRG1, or an allelic variant thereof. The polypeptide encoded by exon 14 of SLC4A4 preferably comprises or consists of SEQ ID NO: 806, or an allelic variant of SEQ ID NO: 806. The polypeptide encoded by exon 6 of NRG1 preferably comprises or consists of SEQ ID NO: 144, or an allelic variant of SEQ ID NO: 144. Preferably, said polypeptide fusion further includes any one of SEQ ID NOs: 794-805, or any allelic variant of SEQ ID NO: 794-805, and preferably further includes SEQ ID NOs: 145-151, or any allelic variant of SEQ ID NOs: 145-151. SEQ ID NOs: 794-805 correspond to the individual polypeptide sequences encoded by exons 2-13 of SLC4A4. SEQ ID NOs: 145-151 correspond to the individual polypeptide sequences encoded by exons 7-13 of NRG1, respectively. Said portion of exon 6 of NRG1 may also be comprised by SEQ ID NO: 156, or an allelic variant of SEQ ID NO: 156, which sequence corresponds to the polypeptide sequence encoded by all of exons 6-13.

Preferably, the polypeptide encoded by the portion of exon 14 of SLC4A4 comprises or consists of 8, 9, 10, 11, 12, 13 or 14 contiguous amino acids from SEQ ID NO: 806, or from an allelic variant SEQ ID NO: 806. The allelic variant has at least 85% identity to SEQ ID NO: 806, preferably at least 90% identity, 92%, 94%, 96% or more preferably at least 98% sequence identity thereto. Also, the polypeptide encoded by the portion of exon 6 of NRG1 comprises or consists of 8, 9, 10, 11, 12, 13 or 14 contiguous amino acids from SEQ ID NO: 144, or from an allelic variant SEQ ID NO: 144. The allelic variant has at least 85% identity to SEQ ID NO: 144, preferably at least 90% identity, 92%, 94%, 96% or more preferably at least 98% sequence identity thereto.

Preferably, any SLC4A4-NRG1 polypeptide fusion of the present disclosure includes the polypeptide sequence according to SEQ ID NO: 766, or an allelic variant thereof. SEQ ID NO: 766 contains a fusion junction between SLC4A4 and NRG1, which fusion is located between amino acid at position 24 (which, together with amino acids at positions 1-23, stems from SLC4A4) and the amino acid at position 26 (which, together with amino acids at positions 27-49 stems from NRG1). At position 25, an alanine (A, ala) residue is present due to an unpredicted in-frame fusion of NRG1 with SLC4A4. Preferably, the SLC4A4-NRG1 polypeptide fusion includes 8, 9, 10, 11, 12, 13 or 14 contiguous amino acids from SEQ ID NO: 766, or of an allelic variant thereof, including at least the amino acids at position 24, 25 and 26 thereof.

In a preferred embodiment, there is provided a polypeptide sequence according to SEQ ID NO: 766, or a polypeptide which comprises 8, 9, 10, 11, 12, 13, 14 or all contiguous amino acids from SEQ ID NO: 766, or of an allelic variant thereof, including at least the amino acids at position 24, 25 and 26. Said polypeptide sequence has at least 85% identity to SEQ ID NO: 766, preferably at least 90% identity, 92%, 94%, 96% or more preferably at least 98% sequence identity thereto.

Preferably, any SLC4A4-NRG1 polypeptide fusion of the present disclosure includes the polypeptide sequence of SEQ ID NO: 766 having one or more (i.e. 1, 2, 3, 4, 5, 6, 7, 8, 9 or 10) point mutations that add, delete or substitute any of the amino acids of the polypeptide comprising SEQ ID NO: 766. Preferably, said polypeptide fusion includes the polypeptide sequence of SEQ ID NO: 766 having 1, 2, 3, 4, or 5 point mutations that add, delete or substitute any of the amino acids of the polypeptide comprising SEQ ID NO: 766. More preferably, said polynucleotide fusion includes the polypeptide sequence of SEQ ID NO: 766 having 1, 2 or 3 point mutations that add, delete or substitute any of the amino acids of the polypeptide comprising SEQ ID NO: 766.

Preferably, the herein provided polypeptide fusion between SLC4A4 and NRG1 is oriented such that the part spanning the N-terminus to the fusion junction is polypeptide sequence from SLC4A4 and the part spanning the fusion junction to the C-terminus is NRG1 polypeptide sequence. Preferably, the NRG1 polypeptide sequence comprises or encodes an EGF-like domain. Said EGF-like domain containing SLC4A4-NRG1 polypeptide fusion is preferably comprised by an aberrant cell as mentioned herein.

### ZFAT-NRG1 Polypeptide Fusion

In accordance with the present disclosure, there is provided a polypeptide fusion encoded by a polynucleotide comprising a ZFAT nucleic acid sequence (or a portion of a ZFAT nucleic acid sequence) fused with an NRG1 nucleic acid sequence (or a portion of a NRG1 nucleic acid sequence). The ZFAT nucleic acid sequence, or portion thereof, preferably encodes a sequence comprising or consisting of any one of SEQ ID NOs: 847-863, or an allelic variant of any one of these SEQ ID NOs. The NRG1 nucleic acid sequence, or portion thereof, preferably encodes a sequence comprising or consisting of any one of SEQ ID NOs: 139-152, or an allelic variant of any one of these SEQ ID NOs.

The ZFAT allelic variant of any one of SEQ ID NOs: 847-863, preferably has at least 85% sequence identity therewith, more preferably 90%, 92%, 94%, 96% or even more preferably at least 98% sequence identity therewith. The NRG1 allelic variant of any one of SEQ ID NOs: 139-152, preferably has at least 85% sequence identity therewith, more preferably 90%, 92%, 94%, 96% or even more preferably at least 98% sequence identity therewith.

Preferably, the portion of the ZFAT nucleic acid sequence of said fusion encodes a polypeptide part of ZFAT which comprises or consists of 8, 9, 10, 11, 12, 13 or 14 contiguous amino acids from any one of SEQ ID NOs: 847-863, or an allelic variant of any one of SEQ ID NOs: 847-863. Preferably, the portion of the NRG1 nucleic acid sequence of said fusion encodes a polypeptide part of NRG1 which comprises or consists 8, 9, 10, 11, 12, 13 or 14 contiguous amino acids from of any one of SEQ ID NOs: 139-152, or an allelic variant of any one of SEQ ID NOs: 139-152.

Preferably, any ZFAT-NRG1 polypeptide fusion of the present disclosure includes a polypeptide sequence of any one of SEQ ID NOs: 847-863 having one or more (i.e. 1, 2, 3, 4, 5, 6, 7, 8, 9 or 10) point mutations that add, delete or substitute any of the amino acids of the polypeptides of SEQ ID NOs: 847-863. Preferably, said polypeptide fusion includes a polypeptide sequence of any one of SEQ ID NOs: 847-863 having 1, 2, 3, 4, or 5 point mutations that add, delete or substitute any of the amino acids of polypeptides of SEQ ID NOs: 847-863. More preferably, said polynucleotide fusion includes a polypeptide sequence of any one of SEQ ID NOs: 847-863 having 1, 2, or 3 point mutations that add, delete or substitute any of the amino acids of the polypeptides of SEQ ID NOs: 847-863.

In a preferred embodiment, there is provided a polypeptide fusion encoded by a polynucleotide which comprises a portion of exon 12 of ZFAT, or an allelic variant thereof, and a portion of exon 6 of NRG1, or an allelic variant thereof. The polypeptide encoded by exon 12 of ZFAT preferably comprises or consists of SEQ ID NO: 858, or an allelic variant of SEQ ID NO: 858. The polypeptide encoded by exon 6 of NRG1 preferably comprises or consists of SEQ ID NO: 144, or an allelic variant of SEQ ID NO: 144. Preferably, said polypeptide fusion further includes any one of SEQ ID NOs: 847-857, and any one of SEQ ID NOs: 145-151, or any allelic variant of SEQ ID NOs: 847-857 or SEQ ID NOs: 145-151. SEQ ID NOs: 847-857 correspond to the individual polypeptide sequences encoded by exons 1-11 of ZFAT and SEQ ID NOs: 145-151 correspond to the individual polypeptide sequences encoded by exons 7-13 of NRG1, respectively. Said portion of exon 6 of NRG1 may also be according to SEQ ID NO: 156, or an allelic variant of SEQ ID NO: 156, which sequence corresponds to the polypeptide sequence encoded by all of exons 6-13.

Preferably, the polypeptide encoded by the portion of exon 12 of ZFAT comprises or consists of 8, 9, 10, 11, 12, 13 or 14 contiguous amino acids from SEQ ID NO: 858, or from an allelic variant SEQ ID NO: 858. The allelic variant has at least 85% identity to SEQ ID NO: 858, preferably at least 90% identity, 92%, 94%, 96% or more preferably at least 98% sequence identity thereto. Also, the polypeptide encoded by the portion of exon 6 of NRG1 comprises or consists of 8, 9, 10, 11, 12, 13 or 14 contiguous amino acids from SEQ ID NO: 144, or from an allelic variant SEQ ID NO: 144. The allelic variant has at least 85% identity to SEQ ID NO: 144, preferably at least 90% identity, 92%, 94%, 96% or more preferably at least 98% sequence identity thereto.

Preferably, any ZFAT-NRG1 polypeptide fusion of the present disclosure includes the polypeptide sequence according to SEQ ID NO: 829, or an allelic variant thereof. SEQ ID NO: 829 contains a fusion junction between ZFAT and NRG1, which fusion is located between amino acid at position 24 (which, together with amino acids at positions 1-23, stems from ZFAT) and the amino acid at position 26 (which, together with amino acids at positions 27-49 stems from NRG1). At position 25, an alanine (A, Ala) residue is present due to an unpredicted in-frame fusion of NRG1 with ZFAT. Preferably, the ZFAT-NRG1 polypeptide fusion includes 8, 9, 10, 11, 12, 13 or 14 contiguous amino acids from SEQ ID NO: 829, or of an allelic variant thereof, including at least the amino acids at position 24, 25 and 26.

In a preferred embodiment, there is provided a polypeptide sequence according to SEQ ID NO: 829, or a polypeptide which comprises 8, 9, 10, 11, 12, 13, 14 or all contiguous amino acids from SEQ ID NO: 829, or of an allelic variant thereof, including at least the amino acids at position 24, 25 and 26. Said polypeptide sequence has at least 85% identity to SEQ ID NO: 829, preferably at least 90% identity, 92%, 94%, 96% or more preferably at least 98% sequence identity thereto.

Preferably, any ZFAT-NRG1 polypeptide fusion of the present disclosure includes the polypeptide sequence of SEQ ID NO: 829 having one or more (i.e. 1, 2, 3, 4, 5, 6, 7, 8, 9 or 10) point mutations that add, delete or substitute any of the amino acids of the polypeptide comprising SEQ ID NO: 829. Preferably, said polypeptide fusion includes the polypeptide sequence of SEQ ID NO: 829 having 1, 2, 3, 4, or 5 point mutations that add, delete or substitute any of the amino acids of the polypeptide comprising SEQ ID NO: 829. More preferably, said polynucleotide fusion includes the polypeptide sequence of SEQ ID NO: 829 polypeptide having 1, 2 or 3 point mutations that add, delete or substitute any of the amino acids of the polypeptide comprising SEQ ID NO: 829.

Preferably, the herein provided polypeptide fusion between NRG1 and ZFAT is oriented such that the part spanning the N-terminus to the fusion junction is polypeptide sequence from ZFAT and the part spanning the fusion junction to the C-terminus is NRG1 polypeptide sequence. Preferably, the NRG1 polypeptide sequence comprises or encodes an EGF-like domain. Said EGF-like domain containing ZFAT-NRG1 polypeptide fusion is preferably comprised by an aberrant cell as mentioned herein.

### DSCAML1-NRG1 Polypeptide Fusion

In accordance with the present disclosure, there is provided a polypeptide fusion encoded by a polynucleotide comprising a DSCAML1 nucleic acid sequence (or a portion of a DSCAML1 nucleic acid sequence) fused with an NRG1 nucleic acid sequence (or a portion of a NRG1 nucleic acid sequence). The DSCAML1 nucleic acid sequence, or portion thereof, preferably encodes a sequence comprising or consisting of any one of SEQ ID NOs: 904-937, or an allelic variant of any one of these SEQ ID NOs. The NRG1 nucleic acid sequence, or portion thereof, preferably encodes a sequence comprising or consisting of any one of SEQ ID NOs 139-152, or an allelic variant of any one of these SEQ ID NOs.

The DSCAML1 allelic variant of any one of SEQ ID NOs: 904-937, preferably has at least 85% sequence identity therewith, more preferably 90%, 92%, 94%, 96% or even more preferably at least 98% sequence identity therewith. The NRG1 allelic variant of any one of SEQ ID NOs: 139-152, preferably has at least 85% sequence identity therewith, more preferably 90%, 92%, 94%, 96% or even more preferably at least 98% sequence identity therewith.

Preferably, the portion of the DSCAML1 nucleic acid sequence of said fusion encodes a polypeptide part of DSCAML1 which comprises or consists of 8, 9, 10, 11, 12, 13 or 14 contiguous amino acids from any one of SEQ ID NOs: 904-937, or an allelic variant of any one of SEQ ID NOs: 904-937. Preferably, the portion of the NRG1 nucleic acid sequence of said fusion encodes a polypeptide part of NRG1 which comprises or consists 8, 9, 10, 11, 12, 13 or 14 contiguous amino acids from of any one of SEQ ID NOs: 139-152, or an allelic variant of any one of SEQ ID NOs: 139-152.

Preferably, any DSCAML1-NRG1 polypeptide fusion of the present disclosure includes a polypeptide sequence of any one of SEQ ID NOs: 904-937 having one or more (i.e. 1, 2, 3, 4, 5, 6, 7, 8, 9 or 10) point mutations that add, delete or substitute any of the amino acids of the polypeptides of SEQ ID NOs: 904-937. Preferably, said polypeptide fusion includes a polypeptide sequence of any one of SEQ ID NOs: 904-937 having 1, 2, 3, 4, or 5 point mutations that add, delete or substitute any of the amino acids of polypeptides of SEQ ID NOs: 904-937. More preferably, said polynucleotide fusion includes a polypeptide sequence of any one of SEQ ID NOs: 904-937 having 1, 2, or 3 point mutations that add, delete or substitute any of the amino acids of the polypeptides of SEQ ID NOs: 904-937.

In a preferred embodiment, there is provided a polypeptide fusion encoded by a polynucleotide which comprises a portion of exon 3 of DSCAML1, or an allelic variant thereof, and a portion of exon 2 of NRG1, or an allelic variant thereof. The polypeptide encoded by exon 3 of DSCAML1 preferably comprises or consists of SEQ ID NO: 906, or an allelic variant of SEQ ID NO: 906. The polypeptide encoded by exon 2 of NRG1 preferably comprises or consists of SEQ ID NO: 140, or an allelic variant of SEQ ID NO: 140. Preferably, said polypeptide fusion further includes any one of SEQ ID NOs: 904-905, and any one of SEQ ID NOs: 141-151, or any allelic variant of SEQ ID NOs: 904-905 or SEQ ID NOs: 141-151. SEQ ID NOs: 904-905 correspond to the individual polypeptide sequences encoded by exons 1-2 of DSCAML1 and SEQ ID NOs: 141-151 correspond to the individual polypeptide sequences encoded by exons 3-13 of NRG1, respectively. Said portion of exon 2 of NRG1 may also be according to SEQ ID NO: 154, or an allelic variant of SEQ ID NO: 154, which sequence corresponds to the polypeptide sequence encoded by all of exons 2-13.

Preferably, the polypeptide encoded by the portion of exon 3 of DSCAML1 comprises or consists of 8, 9, 10, 11, 12, 13 or 14 contiguous amino acids from SEQ ID NO: 906, or from an allelic variant SEQ ID NO: 906. The allelic variant has at least 85% identity to SEQ ID NO: 906, preferably at least 90% identity, 92%, 94%, 96% or more preferably at least 98% sequence identity thereto. Also, the polypeptide encoded by the portion of exon 2 of NRG1 comprises or consists of 8, 9, 10, 11, 12, 13 or 14 contiguous amino acids from SEQ ID NO: 140, or from an allelic variant SEQ ID NO: 140. The allelic variant has at least 85% identity to SEQ ID NO: 140, preferably at least 90% identity, 92%, 94%, 96% or more preferably at least 98% sequence identity thereto.

Preferably, any DSCAML1-NRG1 polypeptide fusion of the present disclosure includes the polypeptide sequence according to SEQ ID NO: 869, or an allelic variant thereof. SEQ ID NO: 869 contains a fusion junction between DSCAML1 and NRG1, which fusion is located between amino acid at position 24 (which, together with amino acids at positions 1-23, stems from DSCAML1) and the amino acid at position 26 (which, together with amino acids at positions 27-49 stems from NRG1). At position 25, an alanine (A, Ala) residue is present due to an unpredicted in-frame fusion of NRG1 with DSCAML1. Preferably, the DSCAML1-NRG1 polypeptide fusion includes 8, 9, 10, 11, 12, 13 or 14 contiguous amino acids from SEQ ID NO: 869, or of an allelic variant thereof, including at least the amino acids at position 24, 25 and 26.

In a preferred embodiment, there is provided a polypeptide sequence according to SEQ ID NO: 869, or a polypeptide which comprises 8, 9, 10, 11, 12, 13, 14 or all contiguous amino acids from SEQ ID NO: 869, or of an allelic variant thereof, including at least the amino acids at position 24, 25 and 26. Said polypeptide sequence has at least 85% identity to SEQ ID NO: 869, preferably at least 90% identity, 92%, 94%, 96% or more preferably at least 98% sequence identity thereto.

Preferably, any DSCAML1-NRG1 polypeptide fusion of the present disclosure includes the polypeptide sequence of SEQ ID NO: 869 having one or more (i.e. 1, 2, 3, 4, 5, 6, 7, 8, 9 or 10) point mutations that add, delete or substitute any of the amino acids of the polypeptide comprising SEQ ID NO: 869. Preferably, said polypeptide fusion includes the polypeptide sequence of SEQ ID NO: 869 having 1, 2, 3, 4, or 5 point mutations that add, delete or substitute any of the amino acids of the polypeptide comprising SEQ ID NO: 869. More preferably, said polynucleotide fusion includes the polypeptide sequence of SEQ ID NO: 869 polypeptide having 1, 2 or 3 point mutations that add, delete or substitute any of the amino acids of the polypeptide comprising SEQ ID NO: 869.

Preferably, the herein provided polypeptide fusion between NRG1 and DSCAML1 is oriented such that the part spanning the N-terminus to the fusion junction is polypeptide sequence from DSCAML1 and the part spanning the fusion junction to the C-terminus is NRG1 polypeptide sequence. Preferably, the NRG1 polypeptide sequence comprises or encodes an EGF-like domain. Said EGF-like domain containing DSCAML1-NRG1 polypeptide fusion is preferably comprised by an aberrant cell as mentioned herein.

Each of the herein mentioned polypeptide fusions comprising NRG1, including VAPB-NRG1, CADM1-NRG1, CD44-NRG1, SLC3A2-NRG1, VTCN1-NRG1, CDH1-NRG1, CXADR-NRG1, GTF2E2-NRG1, CSMD1-NRG1, PTN-NRG1, ST14-NRG1, THBS1-NRG1, AGRN-NRG1, PVALB-NRG1, APP-NRG1, WRN-NRG1, DAAM1-NRG1, ASPH-NRG1, NOTCH2-NRG1, CD74-NRG1, SDC4-NRG1, SLC4A4-NRG1, ZFAT-NRG1 and DSCAML1-NRG1 is preferably isolated. Any method of the invention preferably comprises isolating one or more polypeptide-containing components from a sample. The one or more polypeptide-containing components are typically isolated from any cells or cellular material in the sample.

### Assays for Detecting or Identifying NRG1 Fusions

Typically, any polynucleotide or polypeptide fusion to be detected is obtained or derived from an aberrant cell expressing a polynucleotide fusion that comprises an EGF-like domain of NRG1. A detection assay may not be directed at identifying the presence of an EGF-like domain as it may suffice to merely detect the actual fusion junction which is indicative for the presence of a polynucleotide or polypeptide that further contains an EGF-like domain of NRG1. The presence of an EGF-like domain can be inferred from specifically detecting or identifying an in frame polynucleotide fusion between NRG1 and any fusion partner as such fusion junction is found to be 5′ to the EGF-like domain of NRG1.

Various techniques are available for the skilled person to detect known polynucleotide or polypeptide fusions in samples obtained from human subjects, each of which typically includes a binding agent that specifically binds to a target of interest. Such a binding agent refers to an agent, such as a primer, a primer pair, a probe or an antibody, that allows selective binding to a target sequence with the aim of specifically detecting the target. Binding includes hybridizing or annealing to polynucleotide sequences, typically to amplify and/or detect the target, or to binding of an epitope by an antibody with high affinity and specificity to allow detection of the target.

Technologies based on the use of a primer, a primer pair or a probe that specifically binds to a polynucleotide fusion are well known in the art. Likewise, technologies wherein polypeptides are detected on the basis of binding agents are well known in the art, in particular antibodies that specifically bind a polypeptide are available.

Other ways to use primer, primer pair or probes of the present disclosure include next generation sequencing (NGS), or use of panels comprising multiplex assays to detect fusions (e.g. Archer FusionPlex™ Custom Solid Panel that utilizes the Anchored Multiplex PCR (AMP™) technology targeting known exons), or digital droplet PCR (ddPCR™) for instance using the QX200™ AutoDG™ Droplet Digital PCR System (by BioRad). Other means include the use of molecular beacons or quantitative PCR (Q-PCR) using a TaqMan™ probe or an intercalating fluorescent dye, such as SYBR™ Green, fluorescence in situ hybridization (FISH), next generation sequencing (NGS), ddPCR™, Anchored Multiplex PCR, semi-quantitative PCR or quantitative PCR.

Still other methods to detect a polynucleotide fusion of NRG1 with a fusion partner as mentioned herein using a probe of the present disclosure, is by IHC or FISH, such as break apart FISH wherein two ends of the gene of interest are labeled with different colors. To detect an NRG1 comprising fusion as mentioned herein, preferably the forward strand of hg38 chr8:31,639,222-32,764,405 is used to design suitable probes to label two ends of the NRG1 gene to allow detection of any fusion.

The present disclosure provides a nucleic acid probe, primer or primer pair for detection of any polynucleotide fusion as mentioned herein. Also provided is a detection assay comprising said nucleic acid probe, primer or primer pair for detection of the presence of any polynucleotide fusion as mentioned herein. Such a nucleic acid probe, primer or primer pair has a length to allow detection of a polynucleotide of interest, but a preferred length is about 10 to about 40 nucleotides.

Preferably, any nucleic acid probe, primer or primer pair used for detection of a polynucleotide fusion comprises a detectable label as mentioned herein.

Preferably, any nucleic acid probe, primer or primer pair is used in an assay to detect the presence of a polynucleotide fusion selected from VAPB-NRG1, CADM1-NRG1, CD44-NRG1, SLC3A2-NRG1, VTCN1-NRG1, CDH1-NRG1, CXADR-NRG1, GTF2E2-NRG1, CSMD1-NRG1, PTN-NRG1, ST14-NRG1, THBS1-NRG1, AGRN-NRG1, PVALB-NRG1, APP-NRG1, WRN-NRG1, DAAM1-NRG1, ASPH-NRG1, NOTCH2-NRG1, CD74-NRG1, SDC4-NRG1, SLC4A4-NRG1, ZFAT-NRG1 or DSCAML1-NRG1, in particular to detect the presence of fusion junctions as herein disclosed in said polynucleotide fusions.

Preferably, detecting the presence of any polynucleotide fusion mentioned herein is by using said probe, primer or primer pair that span the nucleic acid junction between NRG1 and its fusion partner allowing amplification of the fusion junction and detection thereof.

### Primer or Probes for Use in Assays for Detecting VAPB-NRG1 Fusions

The present disclosure provides a nucleic acid primer, primer pair or probe for detection of a polynucleotide fusion comprising a VAPB nucleic acid sequence (or a portion of a VAPB nucleic acid sequence fused with an NRG1 nucleic acid sequence (or a portion of a NRG1 nucleic acid sequence). Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 23, or to an allelic variant of SEQ ID NO: 23, and the polynucleotide according to SEQ ID NO: 138, or to an allelic variant of SEQ ID NO: 138.

Also, the present disclosure provides a nucleic acid primer, primer pair or probe for detection of a VAPB-NRG1 polynucleotide fusion comprising or consisting of SEQ ID NO: 3, which sequence includes the nucleic acids at position 43 and 44. Preferably, the nucleic acid probe, primer or primer pair specifically hybridizes to, or has 95%, 96%, 97%, 98%, 99% or preferably 100% sequence identity over a length of about 12 to about 40 nucleotides over a length of about 12 to about 40 nucleotides with, the polynucleotide according to SEQ ID NO: 3, or to an allelic variant of SEQ ID NO: 3, which sequence preferably includes the nucleic acids at position 43 and 44.

Preferably, the nucleic acid probe, primer or primer pair for detection of the fusion of VAPB with NRG1 specifically hybridizes to (or has 95%, 96%, 97%, 98%, 99% or preferably 100% sequence identity over a length of about 12 to about 40 nucleotides over a length of about 12 to about 40 nucleotides with) a sequence, such as used in a gain-of-allele detection assay, comprised by exon 1 from VAPB, or a sequence located 5′ of exon 1 and/or to a sequence comprised by exon 2 from NRG1, or a sequence located 3′ of exon 2, such as a gene sequence of NRG1. Preferably, the nucleic acid probe, primer or primer pair for detection of the fusion of VAPB with NRG1 specifically hybridizes to or has 95%, 96%, 97%, 98%, 99% or preferably 100% sequence identity over a length of about 12 to about 40 nucleotides with a sequence comprised by SEQ ID NO: 17, or to an allelic variant of SEQ ID NO: 17; and/or to a sequence comprised by SEQ ID NO: 153, or to an allelic variant of SEQ ID NO: 153. Also provided are nucleic acid probe, primer or primer pair for detection of the fusions involving allelic variants of any one of said exons.

Preferably, exon 1 from VAPB comprises or consists of SEQ ID NO: 17 or an allelic variant thereof.

### Primer or Probes for Use in Assays for Detecting CADM1-NRG1 Fusions

The present disclosure provides a nucleic acid primer, primer pair or probe for detection of a CADM1-NRG1 polynucleotide fusion comprising or consisting of SEQ ID NO: 7, which sequence includes the nucleic acids at position 53 and 54. Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 7, or to an allelic variant of SEQ ID NO: 7, which sequence preferably includes the nucleic acids at position 53 and 54.

Preferably, the nucleic acid probe, primer or primer pair for detection of the fusion of CADM1 with NRG1 specifically hybridizes to (or has 95%, 96%, 97%, 98%, 99% or preferably 100% sequence identity over a length of about 12 to about 40 nucleotides with) a sequence, such as used in a gain-of-allele detection assay, comprised by exon 7 from CADM1, or a sequence located 5′ of exon 7 and/or to a sequence comprised by exon 6 from NRG1, or a sequence located 3′ of exon 6, such as a gene sequence of NRG1. Preferably, the nucleic acid probe, primer or primer pair for detection of the fusion of CADM1 with NRG1 specifically hybridizes to or has 95%, 96%, 97%, 98%, 99% or preferably 100% sequence identity over a length of about 12 to about 40 nucleotides with a sequence comprised by SEQ ID NO: 57, or to an allelic variant of SEQ ID NO: 57; and/or to a sequence comprised by SEQ ID NO: 155, or to an allelic variant of SEQ ID NO: 155. Also provided are nucleic acid probe, primer or primer pair for detection of the fusions involving allelic variants of any one of said exons.

Preferably, exon 7 from CADM1 comprises or consists of SEQ ID NO: 39 or an allelic variant thereof.

### Primer or Probes for Use in Assays for Detecting CD44-NRG1 Fusions

The present disclosure provides a nucleic acid primer, primer pair or probe for detection of a CD44-NRG1 polynucleotide fusion comprising or consisting of SEQ ID NO: 11, which sequence includes the nucleic acids at position 52 and 53. Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 11, or to an allelic variant of SEQ ID NO: 11, which sequence preferably includes the nucleic acids at position 52 and 53.

Preferably, the nucleic acid probe, primer or primer pair for detection of the fusion of CD44 with NRG1 specifically hybridizes to (or has 95%, 96%, 97%, 98%, 99% or preferably 100% sequence identity over a length of about 12 to about 40 nucleotides with) a sequence, such as used in a gain of allele detection assay, comprised by exon 5 from CD44, or a sequence located 5′ of exon 5 and/or to a sequence comprised by exon 2 from NRG1, or a sequence located 3′ of exon 2, such as a gene sequence of NRG1. Preferably, the nucleic acid probe, primer or primer pair for detection of the fusion of CD44 with NRG1 specifically hybridizes to or has 95%, 96%, 97%, 98%, 99% or preferably 100% sequence identity over a length of about 12 to about 40 nucleotides with a sequence comprised by SEQ ID NO: 99, or to an allelic variant of SEQ ID NO: 99; and/or to a sequence comprised by SEQ ID NO: 153, or to an allelic variant of SEQ ID NO: 153. Also provided are nucleic acid probe, primer or primer pair for detection of the fusions involving allelic variants of any one of said exons.

Preferably, exon 5 from CD44 comprises or consists of SEQ ID NO: 65 or an allelic variant thereof.

The present disclosure also provides a nucleic acid primer, primer pair or probe for detection of a CD44-NRG1 polynucleotide fusion comprising or consisting of SEQ ID NO: 761, which sequence includes the nucleic acids at position 75 and 76. Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 761, or to an allelic variant of SEQ ID NO: 761, which sequence preferably includes the nucleic acids at position 75 and 76.

Preferably, the nucleic acid probe, primer or primer pair for detection of the fusion of CD44 with NRG1 specifically hybridizes to (or has 95%, 96%, 97%, 98%, 99% or preferably 100% sequence identity over a length of about 12 to about 40 nucleotides with) a sequence, such as used in a gain of allele detection assay, comprised by exon 5 from CD44, or a sequence located 5′ of exon 5 and/or to a sequence comprised by exon 6 from NRG1, or a sequence located 3′ of exon 6, such as a gene sequence of NRG1. Preferably, the nucleic acid probe, primer or primer pair for detection of the fusion of CD44 with NRG1 specifically hybridizes to or has 95%, 96%, 97%, 98%, 99% or preferably 100% sequence identity over a length of about 12 to about 40 nucleotides with a sequence comprised by SEQ ID NO: 99, or to an allelic variant of SEQ ID NO: 99; and/or to a sequence comprised by SEQ ID NO: 155, or to an allelic variant of SEQ ID NO: 155. Also provided are nucleic acid probe, primer or primer pair for detection of the fusions involving allelic variants of any one of said exons.

Preferably, exon 5 from CD44 comprises or consists of SEQ ID NO: 65 or an allelic variant thereof.

### Primer or Probes for Use in Assays for Detecting SLC3A2-NRG1 Fusions

The present disclosure provides a nucleic acid primer, primer pair or probe for detection of a SLC3A2 transcript version 6-NRG1 polynucleotide fusion comprising or consisting of SEQ ID NO: 15, which sequence includes the nucleic acids at position 53 and 54. Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 15, or to an allelic variant of SEQ ID NO: 15, which sequence preferably includes the nucleic acids at position 53 and 54.

Preferably, the nucleic acid probe, primer or primer pair for detection of the fusion of transcript version 6 of SLC3A2 with NRG1 specifically hybridizes to (or has 95%, 96%, 97%, 98%, 99% or preferably 100% sequence identity over a length of about 12 to about 40 nucleotides with) a sequence, such as used in a gain-of-allele detection assay, comprised by exon 1 from said SLC3A2 or a sequence located 5′ of exon 1 and/or to a sequence comprised by exon 5 from NRG1, or a sequence located 3′ of exon 5, such as a gene sequence of NRG1. Preferably, the nucleic acid probe, primer or primer pair for detection of the fusion of said SLC3A2 with NRG1 specifically hybridizes to, or has 95%, 96%, 97%, 98%, 99% or preferably 100% sequence identity over a length of about 12 to about 40 nucleotides with, a sequence comprised by SEQ ID NO: 103, or to an allelic variant of SEQ ID NO: 103; and/or to a sequence comprised by SEQ ID NO: 157, or to an allelic variant of SEQ ID NO: 157. Also provided are nucleic acid probe, primer or primer pair for detection of the fusions involving allelic variants of any one of said exons.

Preferably, exon 1 from transcript version 6 of SLC3A2 comprises or consists of SEQ ID NO: 103, or an allelic variant thereof.

The present disclosure also provides a nucleic acid primer, primer pair or probe for detection of a SLC3A2 transcript version 3-NRG1 polynucleotide fusion comprising or consisting of SEQ ID NO: 454, which sequence includes the nucleic acids at position 93 and 94. Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 454, or to an allelic variant of SEQ ID NO: 454, which sequence preferably includes the nucleic acids at position 93 and 94.

Preferably, the nucleic acid probe, primer or primer pair for detection of the fusion of transcript version 3 of SLC3A2 with NRG1 specifically hybridizes to (or has 95%, 96%, 97%, 98%, 99% or preferably 100% sequence identity over a length of about 12 to about 40 nucleotides with) a sequence, such as used in a gain of allele detection assay, comprised by exon 2 from said SLC3A2 or a sequence located 5′ of exon 2 and/or to a sequence comprised by exon 6 from NRG1, or a sequence located 3′ of exon 6, such as a gene sequence of NRG1. Preferably, the nucleic acid probe, primer or primer pair for detection of the fusion of said SLC3A2 with NRG1 specifically hybridizes to, or has 95%, 96%, 97%, 98%, 99% or preferably 100% sequence identity over a length of about 12 to about 40 nucleotides with, a sequence comprised by SEQ ID NO: 482, or to an allelic variant of SEQ ID NO: 482; and/or to a sequence comprised by SEQ ID NO: 155, or to an allelic variant of SEQ ID NO: 155. Also provided are nucleic acid probe, primer or primer pair for detection of the fusions involving allelic variants of any one of said exons.

Preferably, exon 2 from transcript version 3 of SLC3A2 comprises or consists of SEQ ID NO: 457, or an allelic variant thereof.

### Primer or Probes for Use in Assays for Detecting VTCN1-NRG1 Fusions

The present disclosure provides a nucleic acid primer, primer pair or probe for detection of a VTCN1-NRG1 polynucleotide fusion comprising or consisting of SEQ ID NO: 166, which sequence includes the nucleic acids at position 65 and 66. Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 166, or to an allelic variant of SEQ ID NO: 166, which sequence preferably includes the nucleic acids at position 65 and 66.

Preferably, the nucleic acid probe, primer or primer pair for detection of the fusion of VTCN1 with NRG1 specifically hybridizes to (or has 95%, 96%, 97%, 98%, 99% or preferably 100% sequence identity over a length of about 12 to about 40 nucleotides with) a sequence, such as used in a gain-of-allele detection assay, comprised by exon 2 from VTCN1 or a sequence located 5′ of exon 2 and/or to a sequence comprised by exon 2 from NRG1, or a sequence located 3′ of exon 2, such as a gene sequence of NRG1. Preferably, the nucleic acid probe, primer or primer pair for detection of the fusion of VTCN1 with NRG1 specifically hybridizes to, or has 95%, 96%, 97%, 98%, 99% or preferably 100% sequence identity over a length of about 12 to about 40 nucleotides with, a sequence comprised by SEQ ID NO: 181, or to an allelic variant of SEQ ID NO: 181; and/or to a sequence comprised by SEQ ID NO: 153, or to an allelic variant of SEQ ID NO: 153. Also provided are nucleic acid probe, primer or primer pair for detection of the fusions involving allelic variants of any one of said exons.

Preferably, exon 2 from VTCN1 comprises or consists of SEQ ID NO: 169, or an allelic variant thereof.

### Primer or Probes for Use in Assays for Detecting CDH1-NRG1 Fusions

The present disclosure provides a nucleic acid primer, primer pair or probe for detection of a CDH1-NRG1 polynucleotide fusion comprising or consisting of SEQ ID NO: 186, which sequence includes the nucleic acids at position 119 and 120. Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 186, or to an allelic variant of SEQ ID NO: 186, which sequence preferably includes the nucleic acids at position 119 and 120.

Preferably, the nucleic acid probe, primer or primer pair for detection of the fusion of CDH1 with NRG1 specifically hybridizes to (or has 95%, 96%, 97%, 98%, 99% or preferably 100% sequence identity over a length of about 12 to about 40 nucleotides with) a sequence, such as used in a gain-of-allele detection assay, comprised by exon 11 from CDH1 or a sequence located 5′ of exon 11 and/or to a sequence comprised by exon 2 from NRG1, or a sequence located 3′ of exon 2, such as a gene sequence of NRG1. Preferably, the nucleic acid probe, primer or primer pair for detection of the fusion of CDH1 with NRG1 specifically hybridizes to, or has 95%, 96%, 97%, 98%, 99% or preferably 95%, 96%, 97%, 98%, 99% or preferably 100% sequence identity over a length of about 12 to about 40 nucleotides with, a sequence comprised by SEQ ID NO: 213, or to an allelic variant of SEQ ID NO: 213; and/or to a sequence comprised by SEQ ID NO: 153, or to an allelic variant of SEQ ID NO: 153. Also provided are nucleic acid probe, primer or primer pair for detection of the fusions involving allelic variants of any one of said exons.

Preferably, exon 11 from CDH1 comprises or consists of SEQ ID NO: 198, or an allelic variant thereof.

### Primer or Probes for Use in Assays for Detecting CXADR-NRG1 Fusions

The present disclosure provides a nucleic acid primer, primer pair or probe for detection of a CXADR-NRG1 polynucleotide fusion comprising or consisting of SEQ ID NO: 217, which sequence includes the nucleic acids at position 43 and 44. Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 217, or to an allelic variant of SEQ ID NO: 217, which sequence preferably includes the nucleic acids at position 43 and 44.

Preferably, the nucleic acid probe, primer or primer pair for detection of the fusion of CXADR with NRG1 specifically hybridizes to (or has 95%, 96%, 97%, 98%, 99% or preferably 100% sequence identity over a length of about 12 to about 40 nucleotides with) a sequence, such as used in a gain-of-allele detection assay, comprised by exon 1 from CXADR, or a sequence located 5′ of exon 1 and/or to a sequence comprised by exon 2 from NRG1, or a sequence located 3′ of exon 2, such as a gene sequence of NRG1. Preferably, the nucleic acid probe, primer or primer pair for detection of the fusion of CXADR with NRG1 specifically hybridizes to or has 95%, 96%, 97%, 98%, 99% or preferably 100% sequence identity over a length of about 12 to about 40 nucleotides with a sequence comprised by SEQ ID NO: 219, or to an allelic variant of SEQ ID NO: 219; and/or to a sequence comprised by SEQ ID NO: 153, or to an allelic variant of SEQ ID NO: 153. Also provided are nucleic acid probe, primer or primer pair for detection of the fusions involving allelic variants of any one of said exons.

Preferably, exon 1 from CXADR comprises or consists of SEQ ID NO: 219 or an allelic variant thereof.

### Primer or Probes for Use in Assays for Detecting GTF2E2-NRG1 Fusions

The present disclosure provides a nucleic acid primer, primer pair or probe for detection of a GTF2E2-NRG1 polynucleotide fusion comprising or consisting of SEQ ID NO: 233, which sequence includes the nucleic acids at position 141 and 142. Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 233, or to an allelic variant of SEQ ID NO: 233, which sequence preferably includes the nucleic acids at position 141 and 142.

Preferably, the nucleic acid probe, primer or primer pair for detection of the fusion of GTF2E2 with NRG1 specifically hybridizes to (or has 95%, 96%, 97%, 98%, 99% or preferably 100% sequence identity over a length of about 12 to about 40 nucleotides with) a sequence, such as used in a gain-of-allele detection assay, comprised by exon 2 from GTF2E2, or a sequence located 5′ of exon 2 and/or to a sequence comprised by exon 2 from NRG1, or a sequence located 3′ of exon 2, such as a gene sequence of NRG1. Preferably, the nucleic acid probe, primer or primer pair for detection of the fusion of GTF2E2 with NRG1 specifically hybridizes to or has 95%, 96%, 97%, 98%, 99% or preferably 100% sequence identity over a length of about 12 to about 40 nucleotides with a sequence comprised by SEQ ID NO: 252, or to an allelic variant of SEQ ID NO: 252; and/or to a sequence comprised by SEQ ID NO: 153, or to an allelic variant of SEQ ID NO: 153. Also provided are nucleic acid probe, primer or primer pair for detection of the fusions involving allelic variants of any one of said exons.

Preferably, exon 2 from GTF2E2 comprises or consists of SEQ ID NO: 236 or an allelic variant thereof.

### Primer or Probes for Use in Assays for Detecting CSMD1-NRG1 Fusions

The present disclosure provides a nucleic acid primer, primer pair or probe for detection of a CSMD1-NRG1 polynucleotide fusion comprising or consisting of SEQ ID NO: 255, which sequence includes the nucleic acids at position 88 and 89. Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 255, or to an allelic variant of SEQ ID NO: 255, which sequence preferably includes the nucleic acids at position 88 and 89.

Preferably, the nucleic acid probe, primer or primer pair for detection of the fusion of CSMD1 with NRG1 specifically hybridizes to (or has 95%, 96%, 97%, 98%, 99% or preferably 100% sequence identity over a length of about 12 to about 40 nucleotides with) a sequence, such as used in a gain-of-allele detection assay, comprised by exon 23 from CSMD1, or a sequence located 5′ of exon 23 and/or to a sequence comprised by exon 6 from NRG1, or a sequence located 3′ of exon 6, such as a gene sequence of NRG1. Preferably, the nucleic acid probe, primer or primer pair for detection of the fusion of CSMD1 with NRG1 specifically hybridizes to or has 95%, 96%, 97%, 98%, 99% or preferably 100% sequence identity over a length of about 12 to about 40 nucleotides with a sequence comprised by SEQ ID NO: 309, or to an allelic variant of SEQ ID NO: 309; and/or to a sequence comprised by SEQ ID NO: 155, or to an allelic variant of SEQ ID NO: 155. Also provided are nucleic acid probe, primer or primer pair for detection of the fusions involving allelic variants of any one of said exons.

Preferably, exon 23 from CSMD1 comprises or consists of SEQ ID NO: 279 or an allelic variant thereof.

### Primer or Probes for Use in Assays for Detecting PTN-NRG1 Fusions

The present disclosure provides a nucleic acid primer, primer pair or probe for detection of a PTN-NRG1 polynucleotide fusion comprising or consisting of SEQ ID NO: 313, which sequence includes the nucleic acids at position 102 and 103. Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 313, or to an allelic variant of SEQ ID NO: 313, which sequence preferably includes the nucleic acids at position 102 and 103.

Preferably, the nucleic acid probe, primer or primer pair for detection of the fusion of PTN with NRG1 specifically hybridizes to (or has 95%, 96%, 97%, 98%, 99% or preferably 100% sequence identity over a length of about 12 to about 40 nucleotides with) a sequence, such as used in a gain-of-allele detection assay, comprised by exon 4 from PTN, or a sequence located 5′ of exon 4 and/or to a sequence comprised by exon 2 from NRG1, or a sequence located 3′ of exon 2, such as a gene sequence of NRG1. Preferably, the nucleic acid probe, primer or primer pair for detection of the fusion of PTN with NRG1 specifically hybridizes to or has 95%, 96%, 97%, 98%, 99% or preferably 100% sequence identity over a length of about 12 to about 40 nucleotides with a sequence comprised by SEQ ID NO: 326, or to an allelic variant of SEQ ID NO: 326; and/or to a sequence comprised by SEQ ID NO: 153, or to an allelic variant of SEQ ID NO: 153. Also provided are nucleic acid probe, primer or primer pair for detection of the fusions involving allelic variants of any one of said exons.

Preferably, exon 4 from PTN comprises or consists of SEQ ID NO: 318 or an allelic variant thereof.

### Primer or Probes for Use in Assays for Detecting ST14-NRG1 Fusions

The present disclosure provides a nucleic acid primer, primer pair or probe for detection of a ST14-NRG1 polynucleotide fusion comprising or consisting of SEQ ID NO: 330, which sequence includes the nucleic acids at position 95 and 96. Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 330, or to an allelic variant of SEQ ID NO: 330, which sequence preferably includes the nucleic acids at position 95 and 96.

Preferably, the nucleic acid probe, primer or primer pair for detection of the fusion of ST14 with NRG1 specifically hybridizes to (or has 95%, 96%, 97%, 98%, 99% or preferably 100% sequence identity over a length of about 12 to about 40 nucleotides with) a sequence, such as used in a gain of allele detection assay, comprised by exon 11 from ST14, or a sequence located 5′ of exon 11 and/or to a sequence comprised by exon 6 from NRG1, or a sequence located 3′ of exon 6, such as a gene sequence of NRG1. Preferably, the nucleic acid probe, primer or primer pair for detection of the fusion of ST14 with NRG1 specifically hybridizes to or has 95%, 96%, 97%, 98%, 99% or preferably 100% sequence identity over a length of about 12 to about 40 nucleotides with a sequence comprised by SEQ ID NO: 372, or to an allelic variant of SEQ ID NO: 372; and/or to a sequence comprised by SEQ ID NO: 155, or to an allelic variant of SEQ ID NO: 155. Also provided are nucleic acid probe, primer or primer pair for detection of the fusions involving allelic variants of any one of said exons.

Preferably, exon 11 from ST14 comprises or consists of SEQ ID NO: 362 or an allelic variant thereof.

### Primer or Probes for Use in Assays for Detecting THBS1-NRG1 Fusions

The present disclosure provides a nucleic acid primer, primer pair or probe for detection of a THBS1-NRG1 polynucleotide fusion comprising or consisting of SEQ ID NO: 376, which sequence includes the nucleic acids at position 56 and 57. Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 376, or to an allelic variant of SEQ ID NO: 376, which sequence preferably includes the nucleic acids at position 56 and 57.

Preferably, the nucleic acid probe, primer or primer pair for detection of the fusion of THBS1 with NRG1 specifically hybridizes to (or has 95%, 96%, 97%, 98%, 99% or preferably 100% sequence identity over a length of about 12 to about 40 nucleotides with) a sequence, such as used in a gain of allele detection assay, comprised by exon 9 from THBS1, or a sequence located 5′ of exon 9 and/or to a sequence comprised by exon 6 from NRG1, or a sequence located 3′ of exon 6, such as a gene sequence of NRG1. Preferably, the nucleic acid probe, primer or primer pair for detection of the fusion of THBS1 with NRG1 specifically hybridizes to or has 95%, 96%, 97%, 98%, 99% or preferably 100% sequence identity over a length of about 12 to about 40 nucleotides with a sequence comprised by SEQ ID NO: 399, or to an allelic variant of SEQ ID NO: 399; and/or to a sequence comprised by SEQ ID NO: 155, or to an allelic variant of SEQ ID NO: 155. Also provided are nucleic acid probe, primer or primer pair for detection of the fusions involving allelic variants of any one of said exons.

Preferably, exon 9 from THBS1 comprises or consists of SEQ ID NO: 386 or an allelic variant thereof.

### Primer or Probes for Use in Assays for Detecting AGRN-NRG1 Fusions

The present disclosure provides a nucleic acid primer, primer pair or probe for detection of a AGRN-NRG1 polynucleotide fusion comprising or consisting of SEQ ID NO: 403, which sequence includes the nucleic acids at position 106 and 107. Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 403, or to an allelic variant of SEQ ID NO: 403, which sequence preferably includes the nucleic acids at position 106 and 107.

Preferably, the nucleic acid probe, primer or primer pair for detection of the fusion of AGRN with NRG1 specifically hybridizes to (or has 95%, 96%, 97%, 98%, 99% or preferably 100% sequence identity over a length of about 12 to about 40 nucleotides with) a sequence, such as used in a gain of allele detection assay, comprised by exon 12 from AGRN, or a sequence located 5′ of exon 12 and/or to a sequence comprised by exon 6 from NRG1, or a sequence located 3′ of exon 6, such as a gene sequence of NRG1. Preferably, the nucleic acid probe, primer or primer pair for detection of the fusion of AGRN with NRG1 specifically hybridizes to or has 95%, 96%, 97%, 98%, 99% or preferably 100% sequence identity over a length of about 12 to about 40 nucleotides with a sequence comprised by SEQ ID NO: 433, or to an allelic variant of SEQ ID NO: 433; and/or to a sequence comprised by SEQ ID NO: 155, or to an allelic variant of SEQ ID NO: 155. Also provided are nucleic acid probe, primer or primer pair for detection of the fusions involving allelic variants of any one of said exons.

Preferably, exon 12 from AGRN comprises or consists of SEQ ID NO: 416 or an allelic variant thereof.

### Primer or Probes for Use in Assays for Detecting PVALB-NRG1 Fusions

The present disclosure provides a nucleic acid primer, primer pair or probe for detection of a polynucleotide fusion comprising a PVALB nucleic acid sequence (or a portion of a PVALB nucleic acid sequence fused with an NRG1 nucleic acid sequence (or a portion of a NRG1 nucleic acid sequence). Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 444, or to an allelic variant of SEQ ID NO: 444, and the polynucleotide according to SEQ ID NO: 138, or to an allelic variant of SEQ ID NO: 138.

Also, the present disclosure provides a nucleic acid primer, primer pair or probe for detection of a PVALB-NRG1 polynucleotide fusion comprising or consisting of SEQ ID NO: 437, which sequence includes the nucleic acids at position 102 and 103. Preferably, the nucleic acid probe, primer or primer pair specifically hybridizes to, or has 95%, 96%, 97%, 98%, 99% or preferably 100% sequence identity over a length of about 12 to about 40 nucleotides over a length of about 12 to about 40 nucleotides with, the polynucleotide according to SEQ ID NO: 437, or to an allelic variant of SEQ ID NO: 437, which sequence preferably includes the nucleic acids at position 102 and 103.

Preferably, the nucleic acid probe, primer or primer pair for detection of the fusion of PVALB with NRG1 specifically hybridizes to (or has 95%, 96%, 97%, 98%, 99% or preferably 100% sequence identity over a length of about 12 to about 40 nucleotides with) a sequence, such as used in a gain-of-allele detection assay, comprised by exon 4 from PVALB, or a sequence located 5′ of exon 4 and/or to a sequence comprised by exon 6 from NRG1, or a sequence located 3′ of exon 6, such as a gene sequence of NRG1. Preferably, the nucleic acid probe, primer or primer pair for detection of the fusion of PVALB with NRG1 specifically hybridizes to or has 95%, 96%, 97%, 98%, 99% or preferably 100% sequence identity over a length of about 12 to about 40 nucleotides with a sequence comprised by SEQ ID NO: 450, or to an allelic variant of SEQ ID NO: 450; and/or to a sequence comprised by SEQ ID NO: 155, or to an allelic variant of SEQ ID NO: 155. Also provided are nucleic acid probe, primer or primer pair for detection of the fusions involving allelic variants of any one of said exons.

Preferably, exon 4 from PVALB comprises or consists of SEQ ID NO: 442 or an allelic variant thereof.

### Primer or Probes for Use in Assays for Detecting APP-NRG1 Fusions

The present disclosure provides a nucleic acid primer, primer pair or probe for detection of a APP-NRG1 polynucleotide fusion comprising or consisting of SEQ ID NO: 486, which sequence includes the nucleic acids at position 54 and 55. Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 486, or to an allelic variant of SEQ ID NO: 486, which sequence preferably includes the nucleic acids at position 54 and 55.

Preferably, the nucleic acid probe, primer or primer pair for detection of the fusion of APP with NRG1 specifically hybridizes to (or has 95%, 96%, 97%, 98%, 99% or preferably 100% sequence identity over a length of about 12 to about 40 nucleotides with) a sequence, such as used in a gain of allele detection assay, comprised by exon 14 from APP, or a sequence located 5′ of exon 14 and/or to a sequence comprised by exon 6 from NRG1, or a sequence located 3′ of exon 6, such as a gene sequence of NRG1. Preferably, the nucleic acid probe, primer or primer pair for detection of the fusion of APP with NRG1 specifically hybridizes to or has 95%, 96%, 97%, 98%, 99% or preferably 100% sequence identity over a length of about 12 to about 40 nucleotides with a sequence comprised by SEQ ID NO: 524, or to an allelic variant of SEQ ID NO: 524; and/or to a sequence comprised by SEQ ID NO: 155, or to an allelic variant of SEQ ID NO: 155. Also provided are nucleic acid probe, primer or primer pair for detection of the fusions involving allelic variants of any one of said exons.

Preferably, exon 14 from APP comprises or consists of SEQ ID NO: 501 or an allelic variant thereof.

### Primer or Probes for Use in Assays for Detecting WRN-NRG1 Fusions

The present disclosure provides a nucleic acid primer, primer pair or probe for detection of a WRN-NRG1 polynucleotide fusion comprising or consisting of SEQ ID NO: 528, which sequence includes the nucleic acids at position 96 and 97. Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 528, or to an allelic variant of SEQ ID NO: 528, which sequence preferably includes the nucleic acids at position 96 and 97.

Preferably, the nucleic acid probe, primer or primer pair for detection of the fusion of WRN with NRG1 specifically hybridizes to (or has 95%, 96%, 97%, 98%, 99% or preferably 100% sequence identity over a length of about 12 to about 40 nucleotides with) a sequence, such as used in a gain of allele detection assay, comprised by exon 33 from WRN, or a sequence located 5′ of exon 33 and/or to a sequence comprised by exon 6 from NRG1, or a sequence located 3′ of exon 6, such as a gene sequence of NRG1. Preferably, the nucleic acid probe, primer or primer pair for detection of the fusion of WRN with NRG1 specifically hybridizes to or has 95%, 96%, 97%, 98%, 99% or preferably 100% sequence identity over a length of about 12 to about 40 nucleotides with a sequence comprised by SEQ ID NO: 601, or to an allelic variant of SEQ ID NO: 601; and/or to a sequence comprised by SEQ ID NO: 155, or to an allelic variant of SEQ ID NO: 155. Also provided are nucleic acid probe, primer or primer pair for detection of the fusions involving allelic variants of any one of said exons.

Preferably, exon 33 from WRN comprises or consists of SEQ ID NO: 562 or an allelic variant thereof.

### Primer or Probes for Use in Assays for Detecting DAAM1-NRG1 Fusions

The present disclosure provides a nucleic acid primer, primer pair or probe for detection of a DAAM1-NRG1 polynucleotide fusion comprising or consisting of SEQ ID NO: 605, which sequence includes the nucleic acids at position 75 and 76. Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 605, or to an allelic variant of SEQ ID NO: 605, which sequence preferably includes the nucleic acids at position 75 and 76.

Preferably, the nucleic acid probe, primer or primer pair for detection of the fusion of DAAM1 with NRG1 specifically hybridizes to (or has 95%, 96%, 97%, 98%, 99% or preferably 100% sequence identity over a length of about 12 to about 40 nucleotides with) a sequence, such as used in a gain-of-allele detection assay, comprised by exon 1 from DAAM1 and/or to a sequence comprised by exon 1 from NRG1, or a sequence located 3′ of exon 1, such as a gene sequence of NRG1. Preferably, the nucleic acid probe, primer or primer pair for detection of the fusion of DAAM1 with NRG1 specifically hybridizes to or has 95%, 96%, 97%, 98%, 99% or preferably 100% sequence identity over a length of about 12 to about 40 nucleotides with a sequence comprised by SEQ ID NO: 606, or to an allelic variant of SEQ ID NO: 606; and/or to a sequence comprised by SEQ ID NO: 153, or to an allelic variant of SEQ ID NO: 153. Also provided are nucleic acid probe, primer or primer pair for detection of the fusions involving allelic variants of any one of said exons.

Preferably, exon 1 from DAAM1 comprises or consists of SEQ ID NO: 606 or an allelic variant thereof.

### Primer or Probes for Use in Assays for Detecting ASPH-NRG1 Fusions

The present disclosure provides a nucleic acid primer, primer pair or probe for detection of a polynucleotide fusion comprising a ASPH nucleic acid sequence (or a portion of a ASPH nucleic acid sequence fused with an NRG1 nucleic acid sequence (or a portion of a NRG1 nucleic acid sequence). Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 662, or to an allelic variant of SEQ ID NO: 662, and the polynucleotide according to SEQ ID NO: 138, or to an allelic variant of SEQ ID NO: 138.

Also, the present disclosure provides a nucleic acid primer, primer pair or probe for detection of a ASPH-NRG1 polynucleotide fusion comprising or consisting of SEQ ID NO: 635, which sequence includes the nucleic acids at position 75 and 76. Preferably, the nucleic acid probe, primer or primer pair specifically hybridizes to, or has 95%, 96%, 97%, 98%, 99% or preferably 100% sequence identity over a length of about 12 to about 40 nucleotides over a length of about 12 to about 40 nucleotides with, the polynucleotide according to SEQ ID NO: 635, or to an allelic variant of SEQ ID NO: 635, which sequence preferably includes the nucleic acids at position 75 and 76.

Preferably, the nucleic acid probe, primer or primer pair for detection of the fusion of ASPH with NRG1 specifically hybridizes to (or has 95%, 96%, 97%, 98%, 99% or preferably 100% sequence identity over a length of about 12 to about 40 nucleotides with) a sequence, such as used in a gain-of-allele detection assay, comprised by exon 22 from ASPH, or a sequence located 5′ of exon 22 and/or to a sequence comprised by exon 2 from NRG1, or a sequence located 3′ of exon 2, such as a gene sequence of NRG1. Preferably, the nucleic acid probe, primer or primer pair for detection of the fusion of ASPH with NRG1 specifically hybridizes to or has 95%, 96%, 97%, 98%, 99% or preferably 100% sequence identity over a length of about 12 to about 40 nucleotides with a sequence comprised by SEQ ID NO: 689, or to an allelic variant of SEQ ID NO: 689; and/or to a sequence comprised by SEQ ID NO: 153, or to an allelic variant of SEQ ID NO: 153. Also provided are nucleic acid probe, primer or primer pair for detection of the fusions involving allelic variants of any one of said exons.

Preferably, exon 22 from ASPH comprises or consists of SEQ ID NO: 658 or an allelic variant thereof.

### Primer or Probes for Use in Assays for Detecting NOTCH2-NRG1 Fusions

The present disclosure provides a nucleic acid primer, primer pair or probe for detection of a NOTCH2-NRG1 polynucleotide fusion comprising or consisting of SEQ ID NO: 693, which sequence includes the nucleic acids at position 75 and 76. Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 693, or to an allelic variant of SEQ ID NO: 693, which sequence preferably includes the nucleic acids at position 75 and 76.

Preferably, the nucleic acid probe, primer or primer pair for detection of the fusion of NOTCH2 with NRG1 specifically hybridizes to (or has 95%, 96%, 97%, 98%, 99% or preferably 100% sequence identity over a length of about 12 to about 40 nucleotides with) a sequence, such as used in a gain-of-allele detection assay, comprised by exon 6 from NOTCH2, or a sequence located 5′ of exon 6 and/or to a sequence comprised by exon 6 from NRG1, or a sequence located 3′ of exon 6, such as a gene sequence of NRG1. Preferably, the nucleic acid probe, primer or primer pair for detection of the fusion of NOTCH2 with NRG1 specifically hybridizes to or has 95%, 96%, 97%, 98%, 99% or preferably 100% sequence identity over a length of about 12 to about 40 nucleotides with a sequence comprised by SEQ ID NO: 713, or to an allelic variant of SEQ ID NO: 713; and/or to a sequence comprised by SEQ ID NO: 155, or to an allelic variant of SEQ ID NO: 155. Also provided are nucleic acid probe, primer or primer pair for detection of the fusions involving allelic variants of any one of said exons.

Preferably, exon 6 from NOTCH2 comprises or consists of SEQ ID NO: 700 or an allelic variant thereof.

### Primer or Probes for Use in Assays for Detecting CD74-NRG1 Fusions

The present disclosure provides a nucleic acid primer, primer pair or probe for detection of a CD74-NRG1 polynucleotide fusion comprising or consisting of SEQ ID NO: 717, which sequence includes the nucleic acids at position 75 and 76. Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 717, or to an allelic variant of SEQ ID NO: 717, which sequence preferably includes the nucleic acids at position 75 and 76.

Preferably, the nucleic acid probe, primer or primer pair for detection of the fusion of CD74 with NRG1 specifically hybridizes to (or has 95%, 96%, 97%, 98%, 99% or preferably 100% sequence identity over a length of about 12 to about 40 nucleotides with) a sequence, such as used in a gain-of-allele detection assay, comprised by exon 2 from CD74, or a sequence located 5′ of exon 2 and/or to a sequence comprised by exon 2 from NRG1, or a sequence located 3′ of exon 2, such as a gene sequence of NRG1. Preferably, the nucleic acid probe, primer or primer pair for detection of the fusion of CD74 with NRG1 specifically hybridizes to or has 95%, 96%, 97%, 98%, 99% or preferably 100% sequence identity over a length of about 12 to about 40 nucleotides with a sequence comprised by SEQ ID NO: 739, or to an allelic variant of SEQ ID NO: 739; and/or to a sequence comprised by SEQ ID NO: 153, or to an allelic variant of SEQ ID NO: 153. Also provided are nucleic acid probe, primer or primer pair for detection of the fusions involving allelic variants of any one of said exons.

Preferably, exon 2 from CD74 comprises or consists of SEQ ID NO: 720 or an allelic variant thereof.

### Primer or Probes for Use in Assays for Detecting SDC4-NRG1 Fusions

The present disclosure provides a nucleic acid primer, primer pair or probe for detection of a SDC4-NRG1 polynucleotide fusion comprising or consisting of SEQ ID NO: 743, which sequence includes the nucleic acids at position 75 and 76. Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 743, or to an allelic variant of SEQ ID NO: 743, which sequence preferably includes the nucleic acids at position 75 and 76.

Preferably, the nucleic acid probe, primer or primer pair for detection of the fusion of SDC4 with NRG1 specifically hybridizes to (or has 95%, 96%, 97%, 98%, 99% or preferably 100% sequence identity over a length of about 12 to about 40 nucleotides with) a sequence, such as used in a gain of allele detection assay, comprised by exon 2 from SDC4, or a sequence located 5′ of exon 2 and/or to a sequence comprised by exon 2 from NRG1, or a sequence located 3′ of exon 2, such as a gene sequence of NRG1. Preferably, the nucleic acid probe, primer or primer pair for detection of the fusion of SDC4 with NRG1 specifically hybridizes to or has 95%, 96%, 97%, 98%, 99% or preferably 100% sequence identity over a length of about 12 to about 40 nucleotides with a sequence comprised by SEQ ID NO: 757, or to an allelic variant of SEQ ID NO: 757; and/or to a sequence comprised by SEQ ID NO: 153, or to an allelic variant of SEQ ID NO: 153. Also provided are nucleic acid probe, primer or primer pair for detection of the fusions involving allelic variants of any one of said exons.

Preferably, exon 2 from SDC4 comprises or consists of SEQ ID NO: 746 or an allelic variant thereof.

The present disclosure provides a nucleic acid primer, primer pair or probe for detection of a SDC4-NRG1 polynucleotide fusion comprising or consisting of SEQ ID NO: 824, which sequence includes the nucleic acids at position 75 and 76. Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 824, or to an allelic variant of SEQ ID NO: 824, which sequence preferably includes the nucleic acids at position 75 and 76.

Preferably, the nucleic acid probe, primer or primer pair for detection of the fusion of SDC4 with NRG1 specifically hybridizes to (or has 95%, 96%, 97%, 98%, 99% or preferably 100% sequence identity over a length of about 12 to about 40 nucleotides with) a sequence, such as used in a gain of allele detection assay, comprised by exon 4 from SDC4, or a sequence located 5′ of exon 4 and/or to a sequence comprised by exon 2 from NRG1, or a sequence located 3′ of exon 2, such as a gene sequence of NRG1. Preferably, the nucleic acid probe, primer or primer pair for detection of the fusion of SDC4 with NRG1 specifically hybridizes to or has 95%, 96%, 97%, 98%, 99% or preferably 100% sequence identity over a length of about 12 to about 40 nucleotides with a sequence comprised by SEQ ID NO: 940, or to an allelic variant of SEQ ID NO: 940; and/or to a sequence comprised by SEQ ID NO: 153, or to an allelic variant of SEQ ID NO: 153. Also provided are nucleic acid probe, primer or primer pair for detection of the fusions involving allelic variants of any one of said exons.

Preferably, exon 2 from SDC4 comprises or consists of SEQ ID NO: 748 or an allelic variant thereof.

### Primer or Probes for Use in Assays for Detecting SLC4A4-NRG1 Fusions

The present disclosure provides a nucleic acid primer, primer pair or probe for detection of a SLC4A4-NRG1 polynucleotide fusion comprising or consisting of SEQ ID NO: 765, which sequence includes the nucleic acids at position 75 and 76. Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 765, or to an allelic variant of SEQ ID NO: 765, which sequence preferably includes the nucleic acids at position 75 and 76.

Preferably, the nucleic acid probe, primer or primer pair for detection of the fusion of SLC4A4 with NRG1 specifically hybridizes to (or has 95%, 96%, 97%, 98%, 99% or preferably 100% sequence identity over a length of about 12 to about 40 nucleotides with) a sequence, such as used in a gain-of-allele detection assay, comprised by exon 14 from SLC4A4, or a sequence located 5′ of exon 14 and/or to a sequence comprised by exon 6 from NRG1, or a sequence located 3′ of exon 6, such as a gene sequence of NRG1. Preferably, the nucleic acid probe, primer or primer pair for detection of the fusion of SLC4A4 with NRG1 specifically hybridizes to or has 95%, 96%, 97%, 98%, 99% or preferably 100% sequence identity over a length of about 12 to about 40 nucleotides with a sequence comprised by SEQ ID NO: 820, or to an allelic variant of SEQ ID NO: 820; and/or to a sequence comprised by SEQ ID NO: 155, or to an allelic variant of SEQ ID NO: 155. Also provided are nucleic acid probe, primer or primer pair for detection of the fusions involving allelic variants of any one of said exons.

Preferably, exon 14 from SLC4A4 comprises or consists of SEQ ID NO: 780 or an allelic variant thereof.

### Primer or Probes for Use in Assays for Detecting ZFAT-NRG1 Fusions

The present disclosure provides a nucleic acid primer, primer pair or probe for detection of a polynucleotide fusion comprising a ZFAT nucleic acid sequence (or a portion of a ZFAT nucleic acid sequence fused with an NRG1 nucleic acid sequence (or a portion of a NRG1 nucleic acid sequence). Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 846, or to an allelic variant of SEQ ID NO: 846, and the polynucleotide according to SEQ ID NO: 138, or to an allelic variant of SEQ ID NO: 138.

Also, the present disclosure provides a nucleic acid primer, primer pair or probe for detection of a ZFAT-NRG1 polynucleotide fusion comprising or consisting of SEQ ID NO: 828, which sequence includes the nucleic acids at position 75 and 76. Preferably, the nucleic acid probe, primer or primer pair specifically hybridizes to, or has 95%, 96%, 97%, 98%, 99% or preferably 100% sequence identity over a length of about 12 to about 40 nucleotides over a length of about 12 to about 40 nucleotides with, the polynucleotide according to SEQ ID NO: 828, or to an allelic variant of SEQ ID NO: 828, which sequence preferably includes the nucleic acids at position 75 and 76.

Preferably, the nucleic acid probe, primer or primer pair for detection of the fusion of ZFAT with NRG1 specifically hybridizes to (or has 95%, 96%, 97%, 98%, 99% or preferably 100% sequence identity over a length of about 12 to about 40 nucleotides with) a sequence, such as used in a gain of allele detection assay, comprised by exon 12 from ZFAT, or a sequence located 5′ of exon 12 and/or to a sequence comprised by exon 6 from NRG1, or a sequence located 3′ of exon 6, such as a gene sequence of NRG1. Preferably, the nucleic acid probe, primer or primer pair for detection of the fusion of ZFAT with NRG1 specifically hybridizes to or has 95%, 96%, 97%, 98%, 99% or preferably 100% sequence identity over a length of about 12 to about 40 nucleotides with a sequence comprised by SEQ ID NO: 864, or to an allelic variant of SEQ ID NO: 864; and/or to a sequence comprised by SEQ ID NO: 155, or to an allelic variant of SEQ ID NO: 155. Also provided are nucleic acid probe, primer or primer pair for detection of the fusions involving allelic variants of any one of said exons.

Preferably, exon 12 from ZFAT comprises or consists of SEQ ID NO: 841 or an allelic variant thereof.

### Primer or Probes for Use in Assays for Detecting DSCAML1-NRG1 Fusions

The present disclosure provides a nucleic acid primer, primer pair or probe for detection of a polynucleotide fusion comprising a DSCAML1 nucleic acid sequence (or a portion of a DSCAML1 nucleic acid sequence fused with an NRG1 nucleic acid sequence (or a portion of a NRG1 nucleic acid sequence). Preferably, the nucleic acid probe, primer or primer pair specifically hybridizes to, or has 95%, 96%, 97%, 98%, 99% or preferably 100% sequence identity over a length of about 12 to about 40 nucleotides with, the polynucleotide according to SEQ ID NO: 903, or to an allelic variant of SEQ ID NO: 903, and the polynucleotide according to SEQ ID NO: 138, or to an allelic variant of SEQ ID NO: 138.

Also, the present disclosure provides a nucleic acid primer, primer pair or probe for detection of a DSCAML1-NRG1 polynucleotide fusion comprising or consisting of SEQ ID NO: 868, which sequence includes the nucleic acids at position 75 and 76. Preferably, the nucleic acid probe, primer or primer pair specifically hybridizes to, or has 95%, 96%, 97%, 98%, 99% or preferably 100% sequence identity over a length of about 12 to about 40 nucleotides over a length of about 12 to about 40 nucleotides with, the polynucleotide according to SEQ ID NO: 868, or to an allelic variant of SEQ ID NO: 868, which sequence preferably includes the nucleic acids at position 75 and 76.

Preferably, the nucleic acid probe, primer or primer pair for detection of the fusion of DSCAML1 with NRG1 specifically hybridizes to (or has 95%, 96%, 97%, 98%, 99% or preferably 100% sequence identity over a length of about 12 to about 40 nucleotides with) a sequence, such as used in a gain-of-allele detection assay, comprised by exon 3 from DSCAML1, or a sequence located 5′ of exon 3 and/or to a sequence comprised by exon 2 from NRG1, or a sequence located 3′ of exon 2, such as a gene sequence of NRG1. Preferably, the nucleic acid probe, primer or primer pair for detection of the fusion of DSCAML1 with NRG1 specifically hybridizes to or has 95%, 96%, 97%, 98%, 99% or preferably 100% sequence identity over a length of about 12 to about 40 nucleotides with a sequence comprised by SEQ ID NO: 938, or to an allelic variant of SEQ ID NO: 938; and/or to a sequence comprised by SEQ ID NO: 153, or to an allelic variant of SEQ ID NO: 153. Also provided are nucleic acid probe, primer or primer pair for detection of the fusions involving allelic variants of any one of said exons.

Preferably, exon 3 from DSCAML1 comprises or consists of SEQ ID NO: 872 or an allelic variant thereof.

### Probes for Use in In-Situ Hybridization

Also provided are probes for use in an in-situ hybridization (ISH) assay to detect any genetic rearrangements involving VAPB, CADM1, CD44, SLC3A2, VTCN1, CDH1, CXADR, GTF2E2, CSMD1, PTN, ST14, THBS1, AGRN, PVALB, APP, WRN, DAAM1, ASPH, NOTCH2, CD74, SDC4, SLC4A4, ZFAT or DSCAML1. In particular, the probes are for use in a fluorescence in-situ hybridization (FISH) or break apart FISH. The genetic rearrangement are preferably genetic fusions of VAPB, CADM1, CD44, SLC3A2, VTCN1 CDH1, CXADR, GTF2E2, CSMD1, PTN, ST14, THBS1, AGRN, PVALB, APP, WRN, DAAM1, ASPH, NOTCH2, CD74, SDC4, SLC4A4, ZFAT or DSCAML1 with NRG1, but since these ISH assays target the 5′ and 3′ sides of the herein disclosed fusion junctions, the ISH assays can be used to detect any genetic rearrangement that involve said genes.

Thus, in particular are provided a first and a second nucleic acid probe for use in an in-situ hybridization assay to detect a genetic rearrangement of VAPB, CADM1, CD44, SLC3A2, VTCN1, CDH1, CXADR, GTF2E2, CSMD1, PTN, ST14, THBS1, AGRN, PVALB, APP, WRN, DAAM1, ASPH, NOTCH2, CD74, SDC4, SLC4A4, ZFAT or DSCAML1, wherein:
the first probe for detection of a genetic rearrangement of VAPB specifically hybridizes to a VAPB sequence which is located 5′ from the nucleic acid of position 42 or 43 of SEQ ID NO: 1, and the second probe specifically hybridizes to a VAPB sequence which is located 3′ from the nucleic acid of position 42 or 43 of SEQ ID NO: 1;
the first probe for detection of a genetic rearrangement of CADM1 specifically hybridizes to a CADM1 sequence which is located 5′ from the nucleic acid of position 53 of SEQ ID NO: 5, and the second probe specifically hybridizes to a CADM1 sequence which is located 3′ from the nucleic acid of position 53 of SEQ ID NO: 5;
the first probe for detection of a genetic rearrangement of CD44 specifically hybridizes to a CD44 sequence which is located 5′ from the nucleic acid of position 52 of SEQ ID NO: 9, and the second probe specifically hybridizes to a CD44 sequence which is located 3′ from the nucleic acid of position 52 of SEQ ID NO: 9;
the first probe for detection of a genetic rearrangement of CD44 specifically hybridizes to a CD44 sequence which is located 5′ from the nucleic acid of position 75 of SEQ ID NO: 759, and the second probe specifically hybridizes to a CD44 sequence which is located 3′ from the nucleic acid of position 75 of SEQ ID NO: 759;
the first probe for detection of a genetic rearrangement of SLC3A2 specifically hybridizes to a SLC3A2 sequence which is located 5′ from the nucleic acid of position 53 of SEQ ID NO: 13, and the second probe specifically hybridizes to a SLC3A2 sequence which is located 3′ from the nucleic acid of position 53 of SEQ ID NO: 13;
the first probe for detection of a genetic rearrangement of VTCN1 specifically hybridizes to a VTCN1 sequence which is located 5′ from the nucleic acid of position 65 of SEQ ID NO: 164, and the second probe specifically hybridizes to a VTCN1 sequence which is located 3′ from the nucleic acid of position 65 of SEQ ID NO: 164;
the first probe for detection of a genetic rearrangement of CDH1 specifically hybridizes to a CDH1 sequence which is located 5′ from the nucleic acid of position 119 of SEQ ID NO: 184, and the second probe specifically hybridizes to a CDH1 sequence which is located 3′ from the nucleic acid of position 119 of SEQ ID NO: 184;
the first probe for detection of a genetic rearrangement of CXADR specifically hybridizes to a CXADR sequence which is located 5′ from the nucleic acid of position 43 of SEQ ID NO: 215, and the second probe specifically hybridizes to a CXADR sequence which is located 3′ from the nucleic acid of position 43 of SEQ ID NO: 215;
the first probe for detection of a genetic rearrangement of GTF2E2 specifically hybridizes to a GTF2E2 sequence which is located 5′ from the nucleic acid of position 141 of SEQ ID NO: 231, and the second probe specifically hybridizes to a GTF2E2 sequence which is located 3′ from the nucleic acid of position 141 of SEQ ID NO: 231;
the first probe for detection of a genetic rearrangement of CSMD1 specifically hybridizes to a CSMD1 sequence which is located 5′ from the nucleic acid of position 88 of SEQ ID NO: 253, and the second probe specifically hybridizes to a CSMD1 sequence which is located 3′ from the nucleic acid of position 88 of SEQ ID NO: 253;
the first probe for detection of a genetic rearrangement of PTN specifically hybridizes to a PTN sequence which is located 5′ from the nucleic acid of position 102 of SEQ ID NO: 311, and the second probe specifically hybridizes to a PTN sequence which is located 3′ from the nucleic acid of position 102 of SEQ ID NO: 311;
the first probe for detection of a genetic rearrangement of ST14 specifically hybridizes to a ST14 sequence which is located 5′ from the nucleic acid of position 95 of SEQ ID NO: 328, and the second probe specifically hybridizes to a ST14 sequence which is located 3′ from the nucleic acid of position 95 of SEQ ID NO: 328;
the first probe for detection of a genetic rearrangement of THBS1 specifically hybridizes to a THBS1 sequence which is located 5′ from the nucleic acid of position 56 of SEQ ID NO: 374, and the second probe specifically hybridizes to a THBS1 sequence which is located 3′ from the nucleic acid of position 56 of SEQ ID NO: 374;
the first probe for detection of a genetic rearrangement of AGRN specifically hybridizes to a AGRN sequence which is located 5′ from the nucleic acid of position 106 of SEQ ID NO: 401, and the second probe specifically hybridizes to a AGRN sequence which is located 3′ from the nucleic acid of position 106 of SEQ ID NO: 401;
the first probe for detection of a genetic rearrangement of PVALB specifically hybridizes to a PVALB sequence which is located 5′ from the nucleic acid of position 102 of SEQ ID NO: 435, and the second probe specifically hybridizes to a PVALB sequence which is located 3′ from the nucleic acid of position 102 of SEQ ID NO: 435;
the first probe for detection of a genetic rearrangement of SLC3A2 specifically hybridizes to a SLC3A2 sequence which is located 5′ from the nucleic acid of position 93 of SEQ ID NO: 452, and the second probe specifically hybridizes to a SLC3A2 sequence which is located 3′ from the nucleic acid of position 93 of SEQ ID NO: 452;
the first probe for detection of a genetic rearrangement of APP specifically hybridizes to a APP sequence which is located 5′ from the nucleic acid of position 54 of SEQ ID NO: 484, and the second probe specifically hybridizes to a APP sequence which is located 3′ from the nucleic acid of position 54 of SEQ ID NO: 484;
the first probe for detection of a genetic rearrangement of WRN specifically hybridizes to a WRN sequence which is located 5′ from the nucleic acid of position 96 of SEQ ID NO: 526, and the second probe specifically hybridizes to a WRN sequence which is located 3′ from the nucleic acid of position 96 of SEQ ID NO: 526;
the first probe for detection of a genetic rearrangement of DAAM1 specifically hybridizes to a DAAM1 sequence which is located 5′ from the nucleic acid of position 75 of SEQ ID NO: 603, and the second probe specifically hybridizes to a DAAM1 sequence which is located 3′ from the nucleic acid of position 75 of SEQ ID NO: 603;
the first probe for detection of a genetic rearrangement of ASPH specifically hybridizes to a ASPH sequence which is located 5′ from the nucleic acid of position 75 of SEQ ID NO: 633, and the second probe specifically hybridizes to a ASPH sequence which is located 3′ from the nucleic acid of position 75 of SEQ ID NO: 633;
the first probe for detection of a genetic rearrangement of NOTCH2 specifically hybridizes to a NOTCH2 sequence which is located 5′ from the nucleic acid of position 75 of SEQ ID NO: 691, and the second probe specifically hybridizes to a NOTCH2 sequence which is located 3′ from the nucleic acid of position 75 of SEQ ID NO: 691;
the first probe for detection of a genetic rearrangement of CD74 specifically hybridizes to a CD74 sequence which is located 5′ from the nucleic acid of position 75 of SEQ ID NO: 715, and the second probe specifically hybridizes to a CD74 sequence which is located 3′ from the nucleic acid of position 75 of SEQ ID NO: 715;
the first probe for detection of a genetic rearrangement of SDC4 specifically hybridizes to a SDC4 sequence which is located 5′ from the nucleic acid of position 75 of SEQ ID NO: 741, and the second probe specifically hybridizes to a SDC4 sequence which is located 3′ from the nucleic acid of position 75 of SEQ ID NO: 741;
the first probe for detection of a genetic rearrangement of SDC4 specifically hybridizes to a SDC4 sequence which is located 5′ from the nucleic acid of position 75 of SEQ ID NO: 822, and the second probe specifically hybridizes to a SDC4 sequence which is located 3′ from the nucleic acid of position 75 of SEQ ID NO: 822;
the first probe for detection of a genetic rearrangement of SLC4A4 specifically hybridizes to a SLC4A4 sequence which is located 5′ from the nucleic acid of position 75 of SEQ ID NO: 763, and the second probe specifically hybridizes to a SLC4A4 sequence which is located 3′ from the nucleic acid of position 75 of SEQ ID NO: 763;
the first probe for detection of a genetic rearrangement of ZFAT specifically hybridizes to a ZFAT sequence which is located 5′ from the nucleic acid of position 75 of SEQ ID NO: 826, and the second probe specifically hybridizes to a ZFAT sequence which is located 3′ from the nucleic acid of position 75 of SEQ ID NO: 826 or
the first probe for detection of a genetic rearrangement of DSCAML1 specifically hybridizes to a DSCAML1 sequence which is located 5′ from the nucleic acid of position 75 of SEQ ID NO: 866, and the second probe specifically hybridizes to a DSCAML1 sequence which is located 3′ from the nucleic acid of position 75 of SEQ ID NO: 866.

Alternatively, there is provided a preferred ISH assay wherein any polynucleotide fusion selected from VAPB-NRG1, CADM1-NRG1, CD44-NRG1, SLC3A2-NRG1, VTCN1. NRG1, CDH1-NRG1, CXADR-NRG1, GTF2E2-NRG1, CSMD1-NRG1, PTN-NRG1, ST14-NRG1, THBS1-NRG1, AGRN-NRG1, PVALB-NRG1, APP-NRG1, WRN-NRG1, DAAM1-NRG1, ASPH-NRG1, NOTCH2-NRG1, CD74-NRG1, SDC4-NRG1, SLC4A4-NRG1, ZFAT-NRG1 or DSCAML1-NRG1 is detected. Said assay makes use of a first and second probe, wherein if the first probe hybridizes to a sequence of the 5′ side of the fusion junction, the second probe hybridizes to a sequence of the other side, or if the first probe hybridizes to a sequence of the 3′ side of the fusion junction, the second probe hybridizes to a sequence of the other side, and wherein the first probe hybridizes to a sequence selected from VAPB, CADM1, CD44, SLC3A2, VTCN1, CDH1, CXADR, GTF2E2, CSMD1, PTN, ST14, THBS1, AGRN, PVALB, APP, WRN, DAAM1, ASPH, NOTCH2, CD74, SDC4, SLC4A4, ZFAT or DSCAML1, and the second probe hybridizes to an NRG1 sequence, preferably a sequence of the EGF-like domain such as mentioned herein. Including a probe that hybridizes to an EGF-like domain places said domain in the vicinity of the NRG1 fusion partner sequence.

In particular is provided, a first and a second nucleic acid probe for use in an in-situ hybridization assay to detect a VAPB-NRG1 fusion, wherein the first probe specifically hybridizes to a VAPB sequence which is located 5′ from the nucleic acid of position 42 or 43 of SEQ ID NO: 3, and the second probe specifically hybridizes to a NRG1 sequence which is located 3′ from the nucleic acid of position 43 or 44 of SEQ ID NO: 3, such as a sequence comprised by the EGF-like domain of the present disclosure, in particular that of SEQ ID NO: 163.

In particular is also provided, a first and a second nucleic acid probe for use in an in-situ hybridization assay to detect a CADM1-NRG1 fusion, wherein the first probe specifically hybridizes to a CADM1 sequence which is located 5′ from the nucleic acid of position 53 of SEQ ID NO: 7, and the second probe specifically hybridizes to a NRG1 sequence which is located 3′ from the nucleic acid of position 54 of SEQ ID NO: 7, such as a sequence comprised by the EGF-like domain of the present disclosure, in particular that of SEQ ID NO: 163.

In particular is also provided, a first and a second nucleic acid probe for use in an in-situ hybridization assay to detect a CD44-NRG1 fusion, wherein the first probe specifically hybridizes to a CD44 sequence which is located 5′ from the nucleic acid of position 52 of SEQ ID NO: 11, and the second probe specifically hybridizes to a NRG1 sequence which is located 3′ from the nucleic acid of position 53 of SEQ ID NO: 11, such as a sequence comprised by the EGF-like domain of the present disclosure, in particular that of SEQ ID NO: 163.

In particular is also provided, a first and a second nucleic acid probe for use in an in-situ hybridization assay to detect a CD44-NRG1 fusion, wherein the first probe specifically hybridizes to a CD44 sequence which is located 5′ from the nucleic acid of position 75 of SEQ ID NO: 761, and the second probe specifically hybridizes to a NRG1 sequence which is located 3′ from the nucleic acid of position 76 of SEQ ID NO: 761, such as a sequence comprised by the EGF-like domain of the present disclosure, in particular that of SEQ ID NO: 163.

In particular is also provided, a first and a second nucleic acid probe for use in an in-situ hybridization assay to detect a SLC3A2-NRG1 fusion, wherein the first probe specifically hybridizes to a SLC3A2 sequence which is located 5′ from the nucleic acid of position 53 of SEQ ID NO: 15, and the second probe specifically hybridizes to a NRG1 sequence which is located 3′ from the nucleic acid of position 54 of SEQ ID NO: 15, such as a sequence comprised by the EGF-like domain of the present disclosure, in particular that of SEQ ID NO: 163.

In particular is also provided, a first and a second nucleic acid probe for use in an in-situ hybridization assay to detect a VTCN1-NRG1 fusion, wherein the first probe specifically hybridizes to a VTCN1 sequence which is located 5′ from the nucleic acid of position 65 of SEQ ID NO: 166, and the second probe specifically hybridizes to a VTCN1 sequence which is located 3′ from the nucleic acid of position 66 of SEQ ID NO: 166, such as a sequence comprised by the EGF-like domain of the present disclosure, in particular that of SEQ ID NO: 163.

In particular is also provided, a first and a second nucleic acid probe for use in an in-situ hybridization assay to detect a CDH1-NRG1 fusion, wherein the first probe specifically hybridizes to a CDH1 sequence which is located 5′ from the nucleic acid of position 119 of SEQ ID NO: 186, and the second probe specifically hybridizes to a CDH1 sequence which is located 3′ from the nucleic acid of position 120 of SEQ ID NO: 186, such as a sequence comprised by the EGF-like domain of the present disclosure, in particular that of SEQ ID NO: 163.

In particular is also provided, a first and a second nucleic acid probe for use in an in-situ hybridization assay to detect a CXADR-NRG1 fusion, wherein the first probe specifically hybridizes to a CXADR sequence which is located 5′ from the nucleic acid of position 43 of SEQ ID NO: 217, and the second probe specifically hybridizes to a CXADR sequence which is located 3′ from the nucleic acid of position 44 of SEQ ID NO: 217, such as a sequence comprised by the EGF-like domain of the present disclosure, in particular that of SEQ ID NO: 163.

In particular is also provided, a first and a second nucleic acid probe for use in an in-situ hybridization assay to detect a GTF2E2-NRG1 fusion, wherein the first probe specifically hybridizes to a GTF2E2 sequence which is located 5′ from the nucleic acid of position 141 of SEQ ID NO: 233, and the second probe specifically hybridizes to a GTF2E2 sequence which is located 3′ from the nucleic acid of position 142 of SEQ ID NO: 233, such as a sequence comprised by the EGF-like domain of the present disclosure, in particular that of SEQ ID NO: 163.

In particular is also provided, a first and a second nucleic acid probe for use in an in-situ hybridization assay to detect a CSMD1-NRG1 fusion, wherein the first probe specifically hybridizes to a CSMD1 sequence which is located 5′ from the nucleic acid of position 88 of SEQ ID NO: 255, and the second probe specifically hybridizes to a CSMD1 sequence which is located 3′ from the nucleic acid of position 89 of SEQ ID NO: 255, such as a sequence comprised by the EGF-like domain of the present disclosure, in particular that of SEQ ID NO: 163.

In particular is also provided, a first and a second nucleic acid probe for use in an in-situ hybridization assay to detect a PTN-NRG1 fusion, wherein the first probe specifically hybridizes to a PTN sequence which is located 5′ from the nucleic acid of position 102 of SEQ ID NO: 313, and the second probe specifically hybridizes to a PTN sequence which is located 3′ from the nucleic acid of position 103 of SEQ ID NO: 313, such as a sequence comprised by the EGF-like domain of the present disclosure, in particular that of SEQ ID NO: 163.

In particular is also provided, a first and a second nucleic acid probe for use in an in-situ hybridization assay to detect a ST14-NRG1 fusion, wherein the first probe specifically hybridizes to a ST14 sequence which is located 5′ from the nucleic acid of position 95 of SEQ ID NO: 330, and the second probe specifically hybridizes to a ST14 sequence which is located 3′ from the nucleic acid of position 96 of SEQ ID NO: 330, such as a sequence comprised by the EGF-like domain of the present disclosure, in particular that of SEQ ID NO: 163.

In particular is also provided, a first and a second nucleic acid probe for use in an in-situ hybridization assay to detect a THBS1-NRG1 fusion, wherein the first probe specifically hybridizes to a THBS1 sequence which is located 5′ from the nucleic acid of position 56 of SEQ ID NO: 376, and the second probe specifically hybridizes to a THBS1 sequence which is located 3′ from the nucleic acid of position 57 of SEQ ID NO: 376, such as a sequence comprised by the EGF-like domain of the present disclosure, in particular that of SEQ ID NO: 163.

In particular is also provided, a first and a second nucleic acid probe for use in an in-situ hybridization assay to detect a AGRN-NRG1 fusion, wherein the first probe specifically hybridizes to a AGRN sequence which is located 5′ from the nucleic acid of position 106 of SEQ ID NO: 403, and the second probe specifically hybridizes to a AGRN sequence which is located 3′ from the nucleic acid of position 107 of SEQ ID NO: 403, such as a sequence comprised by the EGF-like domain of the present disclosure, in particular that of SEQ ID NO: 163.

In particular is also provided, a first and a second nucleic acid probe for use in an in-situ hybridization assay to detect a PVALB-NRG1 fusion, wherein the first probe specifically hybridizes to a PVALB sequence which is located 5′ from the nucleic acid of position 102 of SEQ ID NO: 437, and the second probe specifically hybridizes to a PVALB sequence which is located 3′ from the nucleic acid of position 103 of SEQ ID NO: 437, such as a sequence comprised by the EGF-like domain of the present disclosure, in particular that of SEQ ID NO: 163.

In particular is also provided, a first and a second nucleic acid probe for use in an in-situ hybridization assay to detect a SLC3A2-NRG1 fusion, wherein the first probe specifically hybridizes to a SLC3A2 sequence which is located 5′ from the nucleic acid of position 93 of SEQ ID NO: 454, and the second probe specifically hybridizes to a SLC3A2 sequence which is located 3′ from the nucleic acid of position 94 of SEQ ID NO: 454, such as a sequence comprised by the EGF-like domain of the present disclosure, in particular that of SEQ ID NO: 163.

In particular is also provided, a first and a second nucleic acid probe for use in an in-situ hybridization assay to detect a APP-NRG1 fusion, wherein the first probe specifically hybridizes to a APP sequence which is located 5′ from the nucleic acid of position 54 of SEQ ID NO: 486, and the second probe specifically hybridizes to a NRG1 sequence which is located 3′ from the nucleic acid of position 55 of SEQ ID NO: 486, such as a sequence comprised by the EGF-like domain of the present disclosure, in particular that of SEQ ID NO: 163.

In particular is also provided, a first and a second nucleic acid probe for use in an in-situ hybridization assay to detect a WRN-NRG1 fusion, wherein the first probe specifically hybridizes to a WRN sequence which is located 5′ from the nucleic acid of position 96 of SEQ ID NO: 528, and the second probe specifically hybridizes to a NRG1 sequence which is located 3′ from the nucleic acid of position 97 of SEQ ID NO: 528, such as a sequence comprised by the EGF-like domain of the present disclosure, in particular that of SEQ ID NO: 163.

In particular is also provided, a first and a second nucleic acid probe for use in an in-situ hybridization assay to detect a DAAM1-NRG1 fusion, wherein the first probe specifically hybridizes to a DAAM1 sequence which is located 5′ from the nucleic acid of position 75 of SEQ ID NO: 605, and the second probe specifically hybridizes to a NRG1 sequence which is located 3′ from the nucleic acid of position 76 of SEQ ID NO: 605, such as a sequence comprised by the EGF-like domain of the present disclosure, in particular that of SEQ ID NO: 163.

In particular is also provided, a first and a second nucleic acid probe for use in an in-situ hybridization assay to detect a ASPH-NRG1 fusion, wherein the first probe specifically hybridizes to a ASPH sequence which is located 5′ from the nucleic acid of position 75 of SEQ ID NO: 635, and the second probe specifically hybridizes to a NRG1 sequence which is located 3′ from the nucleic acid of position 76 of SEQ ID NO: 635, such as a sequence comprised by the EGF-like domain of the present disclosure, in particular that of SEQ ID NO: 163.

In particular is also provided, a first and a second nucleic acid probe for use in an in-situ hybridization assay to detect a NOTCH2-NRG1 fusion, wherein the first probe specifically hybridizes to a NOTCH2 sequence which is located 5′ from the nucleic acid of position 75 of SEQ ID NO: 693, and the second probe specifically hybridizes to a NRG1 sequence which is located 3′ from the nucleic acid of position 76 of SEQ ID NO: 693, such as a sequence comprised by the EGF-like domain of the present disclosure, in particular that of SEQ ID NO: 163.

In particular is also provided, a first and a second nucleic acid probe for use in an in-situ hybridization assay to detect a CD74-NRG1 fusion, wherein the first probe specifically hybridizes to a CD74 sequence which is located 5′ from the nucleic acid of position 75 of SEQ ID NO: 717, and the second probe specifically hybridizes to a NRG1 sequence which is located 3′ from the nucleic acid of position 76 of SEQ ID NO: 717, such as a sequence comprised by the EGF-like domain of the present disclosure, in particular that of SEQ ID NO: 163.

In particular is also provided, a first and a second nucleic acid probe for use in an in-situ hybridization assay to detect a SDC4-NRG1 fusion, wherein the first probe specifically hybridizes to a SDC4 sequence which is located 5′ from the nucleic acid of position 75 of SEQ ID NO: 743, and the second probe specifically hybridizes to a NRG1 sequence which is located 3′ from the nucleic acid of position 76 of SEQ ID NO: 743, such as a sequence comprised by the EGF-like domain of the present disclosure, in particular that of SEQ ID NO: 163.

In particular is also provided, a first and a second nucleic acid probe for use in an in-situ hybridization assay to detect a SDC4-NRG1 fusion, wherein the first probe specifically hybridizes to a SDC4 sequence which is located 5′ from the nucleic acid of position 75 of SEQ ID NO: 824, and the second probe specifically hybridizes to a NRG1 sequence which is located 3′ from the nucleic acid of position 76 of SEQ ID NO: 824, such as a sequence comprised by the EGF-like domain of the present disclosure, in particular that of SEQ ID NO: 163.

In particular is also provided, a first and a second nucleic acid probe for use in an in-situ hybridization assay to detect a SLC4A4-NRG1 fusion, wherein the first probe specifically hybridizes to a SLC4A4 sequence which is located 5′ from the nucleic acid of position 75 of SEQ ID NO: 765, and the second probe specifically hybridizes to a NRG1 sequence which is located 3′ from the nucleic acid of position 76 of SEQ ID NO: 765, such as a sequence comprised by the EGF-like domain of the present disclosure, in particular that of SEQ ID NO: 163.

In particular is also provided, a first and a second nucleic acid probe for use in an in-situ hybridization assay to detect a ZFAT-NRG1 fusion, wherein the first probe specifically hybridizes to a ZFAT sequence which is located 5′ from the nucleic acid of position 75 of SEQ ID NO: 828, and the second probe specifically hybridizes to a NRG1 sequence which is located 3′ from the nucleic acid of position 76 of SEQ ID NO: 828, such as a sequence comprised by the EGF-like domain of the present disclosure, in particular that of SEQ ID NO: 163.

In particular is also provided, a first and a second nucleic acid probe for use in an in-situ hybridization assay to detect a DSCAML1-NRG1 fusion, wherein the first probe specifically hybridizes to a DSCAML1 sequence which is located 5′ from the nucleic acid of position 75 of SEQ ID NO: 868, and the second probe specifically hybridizes to a NRG1 sequence which is located 3′ from the nucleic acid of position 76 of SEQ ID NO: 868, such as a sequence comprised by the EGF-like domain of the present disclosure, in particular that of SEQ ID NO: 163.

Any of the herein mentioned nucleic acid probe, primer or primer pair is preferably used in the following identification or detection methods for detection of any one of the polynucleotide, polypeptide fusions, portions or allelic variants thereof, selected from VAPB-NRG1, CADM1-NRG1 CD44-NRG1, SLC3A2-NRG1, VTCN1-NRG1, CDH1-NRG1, CXADR-NRG1, GTF2E2-NRG1, CSMD1-NRG1, PTN-NRG1, ST14-NRG1, THBS1-NRG1, AGRN-NRG1, PVALB-NRG1, APP-NRG1, WRN-NRG1, DAAM1-NRG1, ASPH-NRG1, NOTCH2-NRG1, CD74-NRG1, SDC4-NRG1, SLC4A4-NRG1, ZFAT-NRG1 and DSCAML1-NRG1.

### Assays for Detecting NRG Polynucleotide Fusions

According to the present disclosure, there is provided a method for identifying in a sample any polynucleotide fusion as mentioned herein, or a polypeptide encoded therefrom, said method comprising testing a sample obtained from a subject to detect the presence of the fusion in the sample.

Also provided is a method for detecting in a sample the presence of a polynucleotide fusion, or a polypeptide encoded therefrom, said method comprising testing a sample obtained from a subject to detect the presence of the fusion in the sample.

Also provided is a method for establishing whether an aberrant cell from a subject comprises a polynucleotide fusion, or a polypeptide encoded therefrom, said method comprising testing the polynucleotide or polypeptide contents of an aberrant cell obtained from the subject for the presence of the fusion in the sample.

Also provided is a method for identifying a subject as carrying a polynucleotide fusion, or a polypeptide encoded therefrom said method comprising testing a sample obtained from a subject and detecting the presence of the fusion in the sample. The testing is preferably followed by a step of associating the detected fusion in the sample with the subject as carrying the fusion.

Preferably, said testing comprises detecting any polynucleotide or polypeptide fusion as mentioned herein by utilizing a binding agent that specifically binds the polynucleotide, such as any nucleic acid probe, primer or primer pair as mentioned herein, or that specifically binds a polypeptide encoded from any polynucleotide fusion as mentioned herein, or alternatively utilizing a binding agent that binds a polynucleotide that comprises the polynucleotide fusion. The binding agent is utilized as part of a detection step or to allow detection is a subsequent step. Thus, the testing in any one of the methods of the disclosure preferably comprises utilizing a binding agent that specifically binds any polynucleotide fusion, or any polypeptide encoded therefrom, to detect such fusion. Said binding agent preferably comprises or consists of a primer, a primer pair, a probe or an antibody. Also, the binding agent preferably comprises a detectable label.

Alternatively, the testing comprises utilizing a binding agent that binds a polynucleotide that comprises the polynucleotide fusion to detect the fusion. In this alternative, the binding agent binds a polynucleotide that is located 5′ and/or 3′ of the polynucleotide fusion that contains the actual junction between NRG1 and its fusion partner. The polynucleotide to which the binding agent anneals or hybridizes, functions as an adaptor which is ligated, or otherwise attached, to the polynucleotide fusion after which the fusion is detected. Such an adaptor preferably comprises a tag or molecular barcode, such as used in sequencing methodologies that include Next Generation Sequencing. Such an adaptor, preferably comprising a barcode or tag, is attached to the polynucleotide fusion which enables detection of said fusion after amplification. In particular, the binding agent can be used in Anchored Multiplex PCR (or AMP), wherein after cDNA generation from mRNA, a target-enriched library for NGS is obtained.

Preferably, said binding agent specifically binds a polynucleotide sequence according to any of SEQ ID NO: 1, SEQ ID NO: 2, SEQ ID NO: 3, SEQ ID NO: 5, SEQ ID NO: 6, SEQ ID NO: 7, SEQ ID NO: 9, SEQ ID NO: 10, SEQ ID NO: 11, SEQ ID NO: 13, SEQ ID NO: 14, SEQ ID NO: 15, SEQ ID NO: 164, SEQ ID NO: 165, SEQ ID NO: 166, SEQ ID NO: 184, SEQ ID NO: 185, SEQ ID NO: 186, SEQ ID NO: 215, SEQ ID NO: 216, SEQ ID NO:217, SEQ ID NO:231, SEQ ID NO:232, SEQ ID NO:233, SEQ ID NO:253, SEQ ID NO:254, SEQ ID NO: 255, SEQ ID NO:311, SEQ ID NO:312, SEQ ID NO:313, SEQ ID NO:328, SEQ ID NO: 329, SEQ ID NO:330, SEQ ID NO:374, SEQ ID NO:375, SEQ ID NO:376, SEQ ID NO: 401, SEQ ID NO:402, SEQ ID NO:403, SEQ ID NO:435, SEQ ID NO:436, SEQ ID NO: 437, SEQ ID NO:452, SEQ ID NO:453, SEQ ID NO:454; SEQ ID NO: 484, SEQ ID NO: 485, SEQ ID NO: 486, SEQ ID NO: 526, SEQ ID NO: 527, SEQ ID NO: 528, SEQ ID NO: 603, SEQ ID NO: 604, SEQ ID NO: 605, SEQ ID NO: 633, SEQ ID NO: 634, SEQ ID NO: 635, SEQ ID NO: 691, SEQ ID NO: 692, SEQ ID NO: 693, SEQ ID NO: 715, SEQ ID NO: 716, SEQ ID NO: 717, SEQ ID NO: 741, SEQ ID NO: 742, SEQ ID NO: 743, SEQ ID NO: 759, SEQ ID NO: 760, SEQ ID NO: 761, SEQ ID NO: 763, SEQ ID NO: 764, SEQ ID NO: 765, SEQ ID NO: 822, SEQ ID NO: 823, SEQ ID NO: 824, SEQ ID NO: 826, SEQ ID NO: 827, SEQ ID NO: 828, SEQ ID NO: 866, SEQ ID NO: 867 or SEQ ID NO: 868. These sequences allow for specific hybridization in close proximity of the fusion junction of interest, or even contain the fusion junction, making for good candidate sequences for binding by said binding agent.

Alternatively, the binding agent specifically binds a polynucleotide sequence that comprises a sequence according to any of SEQ ID NO: 1, SEQ ID NO: 2, SEQ ID NO: 3, SEQ ID NO: 5, SEQ ID NO: 6, SEQ ID NO: 7, SEQ ID NO: 9, SEQ ID NO: 10, SEQ ID NO: 11, SEQ ID NO: 13, SEQ ID NO: 14, SEQ ID NO: 15, SEQ ID NO: 164, SEQ ID NO: 165, SEQ ID NO: 166, SEQ ID NO: 184, SEQ ID NO: 185, SEQ ID NO: 186, SEQ ID NO: 215, SEQ ID NO: 216, SEQ ID NO:217, SEQ ID NO:231, SEQ ID NO:232, SEQ ID NO:233, SEQ ID NO: 253, SEQ ID NO:254, SEQ ID NO:255, SEQ ID NO:311, SEQ ID NO:312, SEQ ID NO: 313, SEQ ID NO:328, SEQ ID NO:329, SEQ ID NO:330, SEQ ID NO:374, SEQ ID NO: 375, SEQ ID NO:376, SEQ ID NO:401, SEQ ID NO:402, SEQ ID NO:403, SEQ ID NO: 435, SEQ ID NO:436, SEQ ID NO:437, SEQ ID NO:452, SEQ ID NO:453, SEQ ID NO: 454, SEQ ID NO: 484, SEQ ID NO: 485, SEQ ID NO: 486, SEQ ID NO: 526, SEQ ID NO: 527, SEQ ID NO: 528, SEQ ID NO: 603, SEQ ID NO: 604, SEQ ID NO: 605, SEQ ID NO: 633, SEQ ID NO: 634, SEQ ID NO: 635, SEQ ID NO: 691, SEQ ID NO: 692, SEQ ID NO: 693, SEQ ID NO: 715, SEQ ID NO: 716, SEQ ID NO: 717, SEQ ID NO: 741, SEQ ID NO: 742, SEQ ID NO: 743, SEQ ID NO: 759, SEQ ID NO: 760, SEQ ID NO: 761, SEQ ID NO: 763, SEQ ID NO: 764, SEQ ID NO: 765, SEQ ID NO: 822, SEQ ID NO: 823, SEQ ID NO: 824, SEQ ID NO: 826, SEQ ID NO: 827, SEQ ID NO: 828, SEQ ID NO: 866, SEQ ID NO: 867 or SEQ ID NO: 868. In a preferred aspect of this alternative, the binding agent preferably anneals or hybridizes to an adaptor sequence which is attached, preferably by ligation, to the polynucleotide fusion sequence. Such an adaptor, preferably comprising a barcode or tag, is attached to the polynucleotide fusion which enables detection of said fusion after amplification. Additionally or alternatively, the binding agent comprises a gene-specific primer, a primer pair, or probe, any of which binds to a nucleotide sequence which is located 5′ or 3′ from a fusion breakpoint to allow amplification of the junction of NRG1 with VAPB, CADM1, CD44, SLC3A2, VTCN1, CDH1, CXADR, GTF2E2, CSMD1, PTN, ST14, THBS1, AGRN, PVALB, APP, WRN, DAAM1, ASPH, NOTCH2, CD74, SDC4, SLC4A4, ZFAT or DSCAML1.

Also, the testing for the presence of any polynucleotide fusion of the present disclosure preferably comprises amplification of a polynucleotide carrying an NRG1 fusion, or a portion thereof, preferably using a DNA polymerase, more preferably a thermostable DNA polymerase or Taq polymerase.

Preferably, the binding agent comprises a first and a second primer for amplifying a nucleotide sequence comprising an NRG1 sequence and a VAPB, CADM1, CD44, SLC3A2, VTCN1, CDH1, CXADR, GTF2E2, CSMD1, PTN, ST14, THBS1, AGRN, PVALB, APP, WRN, DAAM1, ASPH, NOTCH2, CD74, SDC4, SLC4A4, ZFAT or DSCAML1 sequence to detect any polynucleotide fusions mentioned herein. More preferably, provided is a first primer hybridizing or annealing to a nucleotide sequence specific for the NRG1 sequence part of the fusion and a second primer hybridizing or annealing to a nucleotide sequence specific for a fusion partner selected from VAPB, CADM1, CD44, SLC3A2, VTCN1, CDH1, CXADR, GTF2E2, CSMD1, PTN, ST14, THBS1, AGRN, PVALB APP, WRN, DAAM1, ASPH, NOTCH2, CD74, SDC4, SLC4A4, ZFAT or DSCAML1. Preferably, the first and second primers bind in close proximity to a fusion junction to amplify a sequence comprising a part of the NRG1 sequence and a part of the sequence of VAPB, CADM1, CD44, SLC3A2, VTCN1, CDH1, CXADR, GTF2E2, CSMD1, PTN, ST14, THBS1, AGRN, PVALB, APP, WRN, DAAM1, ASPH, NOTCH2, CD74, SDC4, SLC4A4, ZFAT or DSCAML1. The first or second primer may also bind a position spanning the fusion junction. Typically, the amplified sequence comprises up to a 1000, 900, 800, 700, 600, 500, 400, or 300 base pairs. Typically, the amplified sequence spans the NRG1 fusion junction.

Also provided is thus a binding agent which is or comprises a nucleic acid probe or primer of 10-40 nucleotides in length for detection of a polynucleotide fusion of VAPB, CADM1, CD44, SLC3A2, VTCN1, CDH1, CXADR, GTF2E2, CSMD1, PTN, ST14, THBS1, AGRN, PVALB, APP, WRN, DAAM1, ASPH, NOTCH2, CD74, SDC4, SLC4A4, ZFAT or DSCAML1 with NRG1 of the present disclosure, wherein the fusion comprises the nucleic acids at position 43 and 44 of SEQ ID NO: 3, the nucleic acids at position 53 and 54 of SEQ ID NO: 7, the nucleic acids at position 52 and 53 of SEQ ID NO: 11, the nucleic acids at position 53 and 54 of SEQ ID NO: 15, the nucleic acids at position 65 and 66 of SEQ ID NO: 166, the nucleic acids at position 119 and 120 of SEQ ID NO: 186, the nucleic acids at position 43 and 44 of SEQ ID NO: 217, the nucleic acid at position 141 and 142 of SEQ ID NO: 233, the nucleic acids at position 88 and 89 of SEQ ID NO: 255, the nucleic acids at position 102 and 103 of SEQ ID NO: 313, the nucleic acids at position 95 and 96 of SEQ ID NO: 330, the nucleic acids at position 56 and 57 of SEQ ID NO: 376, the nucleic acids at position 106 and 107 of SEQ ID NO: 403, the nucleic acids at position 102 and 103 of SEQ ID NO: 437 or the nucleic acids at position 93 and 94 of SEQ ID NO: 454, the nucleic acids at position 54 and 55 of SEQ ID NO: 486, the nucleic acids at position 96 and 97 of SEQ ID NO: 528, the nucleic acids at position 75 and 76 of SEQ ID NO: 605, the nucleic acids at position 75 and 76 of SEQ ID NO: 635, the nucleic acids at position 75 and 76 of SEQ ID NO: 693, the nucleic acids at position 75 and 76 of SEQ ID NO: 717, the nucleic acids at position 75 and 76 of SEQ ID NO: 743, the nucleic acids at position 75 and 76 of SEQ ID NO: 761, the nucleic acids at position 75 and 76 of SEQ ID NO: 765, the nucleic acids at position 75 and 76 of SEQ ID NO: 824, the nucleic acids at position 75 and 76 of SEQ ID NO: 828, the nucleic acids at position 75 and 76 of SEQ ID NO: 868, respectively. The nucleic acids of these positions define the break point or fusion junction between NRG1 and its fusions partner.

Preferably, said testing as mentioned herein comprises amplifying or detecting a sequence that discriminates between the presence and absence of the polynucleotide fusion, or polypeptide encoded therefrom.

In a preferred embodiment, the binding agent is a polypeptide and detecting the presence thereof is preferably by flow-cytometry (FC), fluorescence in situ hybridization (FISH), immunocytochemistry (ICC), immuno histochemistry (IHC) or immunofluorescence (IF). Various techniques are available for the skilled person to detect an NRG1 fusion of the present disclosure in a sample using FC, IHC, FISH, ICC or IF. For instance, ICC can be performed by using a first antibody targeting NRG1 and a second antibody targeting an NRG1 fusion partner selected from VAPB, CADM1, CD44, SLC3A2, VTCN1, CDH1, CXADR, GTF2E2, CSMD1, PTN, ST14, THBS1, AGRN, PVALB, APP, WRN, DAAM1, ASPH, NOTCH2, CD74, SDC4, SLC4A4, ZFAT or DSCAML1. Co-localization of the signal of the first and second antibody or detecting the presence of a polypeptide product of the expected size using a protein size-based discrimination assay reveals the presence of the respective NRG1 fusion product. Preferably, the anti-NRG1 antibody targets the EGF-like domain of NRG1 and the second antibody targets an extracellular domain of the fusion partner, such as VAPB, CADM1, CD44, SLC3A2, VTCN1, CDH1, CXADR, GTF2E2, CSMD1, PTN, ST14, THBS1, AGRN, PVALB, APP, WRN, DAAM1, ASPH, NOTCH2, CD74, SDC4, SLC4A4, ZFAT or DSCAML1. One example of a FISH protocol is provided by Frithiof et al. in OncoTargets and therapy vol. 9, 7095-7103, 16 Nov. 2016. In addition, antibodies can be detected using two different fluorescence detection systems in combination with the use of two different channels in a fluorescence microscope. Results are interpreted based on the overlapping or non-overlapping appearance of the two fluorescence signals. It lies well within the ambit of the skilled person to develop antibodies against polypeptides of known structure and sequence.

Preferably, the polynucleotide or polypeptide fusion to be detected is obtained from an aberrant cell that expresses an NRG1 EGF-like domain comprising polynucleotide fusion. Preferably, any detection method of the present disclosure comprises a step of obtaining the sample from a subject, followed by a step of isolating the polynucleotide or polypeptide encoded therefrom, from the sample. Also, said method preferably comprises a step of purifying or isolating the polynucleotide or polypeptide from the sample.

Preferably, the sample is obtained from a subject suffering from, or suspect of suffering from, having an aberrant cell, a cancer or a tumor. The sample may be a liquid biopsy sample or a sample taken from a solid cancer or a solid tumor. The solid sample preferably is a formalin embedded paraffin fixed sample containing any one of aberrant cells, a section of a cancer or of a tumor. Taking a biopsy from a solid cancer or solid tumor involves impactful disruption of the physical integrity of the skin, and possibly internal organs, to reach the tumor under investigation. Using liquid biopsy, such as selected from blood, blood serum, blood plasma, pleural effusion, saliva, urine, semen, sputum, vaginal fluid, amniotic fluid, peritoneal fluid, cerebrospinal fluid, bone marrow, cell free lavage or another biofluid, allows for more affordable, quick, reliable and less invasive sampling over such methods. Thus, preferably, the sample is a liquid biopsy sample.

### Antibody-Based Assays for Detecting or Identifying NRG1 Fusions

Also provided is a polypeptide-based detection assay and antibodies for use therein. The detection assay aims to detect any polypeptide NRG1 fusion of the disclosure, in particular a VAPB-NRG1, CADM1-NRG1, CD44-NRG1, SLC3A2-NRG1, VTCN1-NRG1, CDH1-NRG1, CXADR-NRG1, GTF2E2-NRG1, CSMD1-NRG1, PTN-NRG1, ST14-NRG1, THBS1-NRG1, AGRN-NRG1, PVALB-NRG1, APP-NRG1, WRN-NRG1, ASPH-NRG1, NOTCH2-NRG1, CD74-NRG1, SDC4-NRG1, SLC4A4-NRG1, ZFAT-NRG1 and DSCAML1-NRG1 fusion of the disclosure.

In particular is provided, a first antibody, or a set of a first and a second antibody, suitable for detection of any polypeptide fusion as mentioned herein, preferably any polypeptide fusion encoded by any polynucleotide fusion as mentioned herein. It lies well within the abilities of the skilled person to design, develop and produce antibodies that are capable of and suitable for specific binding to polypeptide fusions as disclosed herein.

In case a first antibody is used without the second antibody, the first antibody specifically detects an epitope that is unique for the NRG1 polypeptide fusion. Such an epitope includes an epitope that spans the polypeptide fusion between NRG1 and its fusion partner. Alternatively, in case a first antibody is used without the second antibody, and the first antibody is used in a detection assay, such as discussed further below, said assay includes a step to distinguish from a non-fused polypeptide (e.g. a separation step based on size or charge, such as CIEX or HPLC). In case a first and second antibody is used, the first antibody detects one part of the polypeptide fusion (i.e. VAPB, CADM1, CD44, SLC3A2, VTCN1, CDH1, CXADR, GTF2E2, CSMD1, PTN, ST14, THBS1, AGRN, PVALB, APP, WRN, ASPH, NOTCH2, CD74, SDC4, SLC4A4, ZFAT or DSCAML1) and the second antibody detects the NRG1-comprising part of the polypeptide fusion.

More in particular, the present disclosure provides a first antibody, or a set of a first and a second antibody, for detection of a polypeptide encoded by a polynucleotide fusion comprising a nucleic acid encoding a protein sequence of VAPB, or an allelic variant of VAPB, fused with a nucleic acid encoding a protein sequence of NRG1, or an allelic variant of NRG1.

Preferably, the first antibody, or set of a first and a second antibody, is for detection of a polypeptide fusion comprising a portion of the polypeptide encoded by exon 1 of VAPB, or an allelic variant of exon 1, fused with a portion of the polypeptide encoded by exon 2 of NRG1, or an allelic variant of exon 2. Also provided is a detection assay comprising a first antibody or a set of a first and second antibodies for detection of the presence of a polypeptide encoded by a polynucleotide fusion involving VAPB and NRG1, wherein the first antibody preferably binds the VAPB-NRG1 polypeptide fusion and the set of first and second antibodies preferably binds VAPB and NRG1.

Also provided is a first antibody, or a set of a first and a second antibody, for detection of a polypeptide fusion comprising a portion of the polypeptide encoded by exon 7 of CADM1, or an allelic variant of exon 7, fused with a portion of the polypeptide encoded by of exon 6 of NRG1, or an allelic variant of exon 6. Also provided is a detection assay comprising a first antibody or a set of a first and second antibodies for detection of the presence of a polypeptide fusion comprising a portion of the polypeptide encoded by of exon 7 of CADM1, or an allelic variant of exon 7, fused with a portion of the polypeptide encoded by of exon 6 of NRG1, or an allelic variant of exon 6 wherein the first antibody preferably binds the CADM1-NRG1 polypeptide fusion and the set of first and second antibodies preferably binds CADM1 and NRG1, respectively.

Also provided is a first antibody or a set of a first and a second antibody pair for detection of a polypeptide fusion comprising a portion of the polypeptide encoded by exon 5 of CD44, or an allelic variant of exon 5, fused with a portion of the polypeptide encoded by exon 2 of NRG1, or an allelic variant of exon 2. Also provided is a detection assay comprising a first antibody or a set of a first and second antibodies for detection of the presence of a polypeptide fusion comprising a portion of the polypeptide encoded by exon 5 of CD44, or an allelic variant of exon 5, fused with a portion of the polypeptide encoded by exon 2 of NRG1, or an allelic variant of exon 2, wherein the first antibody preferably binds the CD44-NRG1 polypeptide fusion and the set of first and second antibodies preferably binds CD44 and NRG1, respectively.

Also provided is a first antibody, or a set of a first and a second antibody, for detection of a polypeptide fusion comprising a portion of the polypeptide encoded by exon 5 of CD44, or an allelic variant of exon 5, fused with a portion of the polypeptide encoded by of exon 6 of NRG1, or an allelic variant of exon 6. Also provided is a detection assay comprising a first antibody or a set of a first and second antibodies for detection of the presence of a polypeptide fusion comprising a portion of the polypeptide encoded by of exon 5 of CD44, or an allelic variant of exon 5, fused with a portion of the polypeptide encoded by of exon 6 of NRG1, or an allelic variant of exon 6 wherein the first antibody preferably binds the CD44-NRG1 polypeptide fusion and the set of first and second antibodies preferably binds CD44 and NRG1, respectively.

Also provided is a first antibody or a set of a first and a second antibody for detection of a polypeptide fusion comprising a portion of the polypeptide encoded by exon 1 of transcript version 6 of SLC3A2, or an allelic variant of exon 1, fused with a portion of the polypeptide encoded by exon 5 of NRG1, or an allelic variant of exon 5. Also provided is a detection assay comprising a first antibody or a set of a first and second antibodies for detection of the presence of a polypeptide comprising a portion of the polypeptide encoded by exon 1 of said SLC3A2, or an allelic variant of exon 1, fused with a portion of the polypeptide encoded by exon 5 of NRG1, or an allelic variant of exon 5, wherein the first antibody preferably binds the SLC3A2-NRG1 polypeptide fusion of SLC3A2 transcript version 6 and the set of first and second antibodies preferably binds said SLC3A2 and NRG1, respectively.

Also provided is a first antibody or a set of a first and a second antibody pair for detection of a polypeptide fusion comprising a portion of the polypeptide encoded by exon 2 of VTCN1, or an allelic variant of exon 2, fused with a portion of the polypeptide encoded by exon 2 of NRG1, or an allelic variant of exon 2. Also provided is a detection assay comprising a first antibody or a set of a first and second antibodies for detection of the presence of a polypeptide fusion comprising a portion of the polypeptide encoded by exon 2 of VTCN1, or an allelic variant of exon 2, fused with a portion of the polypeptide encoded by exon 2 of NRG1, or an allelic variant of exon 2, wherein the first antibody preferably binds the VTCN1-NRG1 polypeptide fusion and the set of first and second antibodies preferably binds VTCN1 and NRG1, respectively.

Also provided is a first antibody or a set of a first and a second antibody pair for detection of a polypeptide fusion comprising a portion of the polypeptide encoded by exon 11 of CDH1, or an allelic variant of exon 11, fused with a portion of the polypeptide encoded by exon 2 of NRG1, or an allelic variant of exon 2. Also provided is a detection assay comprising a first antibody or a set of a first and second antibodies for detection of the presence of a polypeptide fusion comprising a portion of the polypeptide encoded by exon 11 of CDH1, or an allelic variant of exon 11, fused with a portion of the polypeptide encoded by exon 2 of NRG1, or an allelic variant of exon 2, wherein the first antibody preferably binds the CDH1-NRG1 polypeptide fusion and the set of first and second antibodies preferably binds CDH1 and NRG1, respectively.

Also provided is a first antibody or a set of a first and a second antibody pair for detection of a polypeptide fusion comprising a portion of the polypeptide encoded by exon 1 of CXADR, or an allelic variant of exon 1, fused with a portion of the polypeptide encoded by exon 2 of NRG1, or an allelic variant of exon 2. Also provided is a detection assay comprising a first antibody or a set of a first and second antibodies for detection of the presence of a polypeptide fusion comprising a portion of the polypeptide encoded by exon 1 of CXADR, or an allelic variant of exon 1, fused with a portion of the polypeptide encoded by exon 2 of NRG1, or an allelic variant of exon 2, wherein the first antibody preferably binds the CXADR-NRG1 polypeptide fusion and the set of first and second antibodies preferably binds CXADR and NRG1, respectively.

Also provided is a first antibody or a set of a first and a second antibody pair for detection of a polypeptide fusion comprising a portion of the polypeptide encoded by exon 2 of GFT2E2, or an allelic variant of exon 2, fused with a portion of the polypeptide encoded by exon 2 of NRG1, or an allelic variant of exon 2. Also provided is a detection assay comprising a first antibody or a set of a first and second antibodies for detection of the presence of a polypeptide fusion comprising a portion of the polypeptide encoded by exon 2 of GFT2E2, or an allelic variant of exon 2, fused with a portion of the polypeptide encoded by exon 2 of NRG1, or an allelic variant of exon 2, wherein the first antibody preferably binds the GFT2E2-NRG1 polypeptide fusion and the set of first and second antibodies preferably binds GFT2E2 and NRG1, respectively.

Also provided is a first antibody or a set of a first and a second antibody pair for detection of a polypeptide fusion comprising a portion of the polypeptide encoded by exon 23 of CSMD1, or an allelic variant of exon 23, fused with a portion of the polypeptide encoded by exon 6 of NRG1, or an allelic variant of exon 6. Also provided is a detection assay comprising a first antibody or a set of a first and second antibodies for detection of the presence of a polypeptide fusion comprising a portion of the polypeptide encoded by exon 23 of CSMD1, or an allelic variant of exon 23, fused with a portion of the polypeptide encoded by exon 6 of NRG1, or an allelic variant of exon 6, wherein the first antibody preferably binds the CSMD1-NRG1 polypeptide fusion and the set of first and second antibodies preferably binds CSMD1 and NRG1, respectively.

Also provided is a first antibody or a set of a first and a second antibody pair for detection of a polypeptide fusion comprising a portion of the polypeptide encoded by exon 4 of PTN, or an allelic variant of exon 4, fused with a portion of the polypeptide encoded by exon 2 of NRG1, or an allelic variant of exon 2. Also provided is a detection assay comprising a first antibody or a set of a first and second antibodies for detection of the presence of a polypeptide fusion comprising a portion of the polypeptide encoded by exon 4 of PTN, or an allelic variant of exon 4, fused with a portion of the polypeptide encoded by exon 2 of NRG1, or an allelic variant of exon 2, wherein the first antibody preferably binds the PTN-NRG1 polypeptide fusion and the set of first and second antibodies preferably binds PTN and NRG1, respectively.

Also provided is a first antibody or a set of a first and a second antibody pair for detection of a polypeptide fusion comprising a portion of the polypeptide encoded by exon 11 of ST14, or an allelic variant of exon 11, fused with a portion of the polypeptide encoded by exon 6 of NRG1, or an allelic variant of exon 6. Also provided is a detection assay comprising a first antibody or a set of a first and second antibodies for detection of the presence of a polypeptide fusion comprising a portion of the polypeptide encoded by exon 11 of ST14, or an allelic variant of exon 11, fused with a portion of the polypeptide encoded by exon 6 of NRG1, or an allelic variant of exon 6, wherein the first antibody preferably binds the ST14-NRG1 polypeptide fusion and the set of first and second antibodies preferably binds ST14 and NRG1, respectively.

Also provided is a first antibody or a set of a first and a second antibody pair for detection of a polypeptide fusion comprising a portion of the polypeptide encoded by exon 9 of THBS1, or an allelic variant of exon 9, fused with a portion of the polypeptide encoded by exon 6 of NRG1, or an allelic variant of exon 6. Also provided is a detection assay comprising a first antibody or a set of a first and second antibodies for detection of the presence of a polypeptide fusion comprising a portion of the polypeptide encoded by exon 9 of THBS1, or an allelic variant of exon 9, fused with a portion of the polypeptide encoded by exon 6 of NRG1, or an allelic variant of exon 6, wherein the first antibody preferably binds the THBS1-NRG1 polypeptide fusion and the set of first and second antibodies preferably binds THBS1 and NRG1, respectively.

Also provided is a first antibody or a set of a first and a second antibody pair for detection of a polypeptide fusion comprising a portion of the polypeptide encoded by exon 12 of AGRN, or an allelic variant of exon 12, fused with a portion of the polypeptide encoded by exon 6 of NRG1, or an allelic variant of exon 6. Also provided is a detection assay comprising a first antibody or a set of a first and second antibodies for detection of the presence of a polypeptide fusion comprising a portion of the polypeptide encoded by exon 12 of AGRN, or an allelic variant of exon 12, fused with a portion of the polypeptide encoded by exon 6 of NRG1, or an allelic variant of exon 6, wherein the first antibody preferably binds the AGRN-NRG1 polypeptide fusion and the set of first and second antibodies preferably binds AGRN and NRG1, respectively.

Also provided is a first antibody or a set of a first and a second antibody pair for detection of a polypeptide fusion comprising a portion of the polypeptide encoded by exon 4 of PVALB, or an allelic variant of exon 4, fused with a portion of the polypeptide encoded by exon 6 of NRG1, or an allelic variant of exon 6. Also provided is a detection assay comprising a first antibody or a set of a first and second antibodies for detection of the presence of a polypeptide fusion comprising a portion of the polypeptide encoded by exon 4 of PVALB, or an allelic variant of exon 4, fused with a portion of the polypeptide encoded by exon 6 of NRG1, or an allelic variant of exon 6, wherein the first antibody preferably binds the PVALB-NRG1 polypeptide fusion and the set of first and second antibodies preferably binds PVALB and NRG1, respectively.

Also provided is a first antibody or a set of a first and a second antibody pair for detection of a polypeptide fusion comprising a portion of the polypeptide encoded by exon 2 of transcript version 3 of SLC3A2, or an allelic variant of exon 2, fused with a portion of the polypeptide encoded by exon 6 of NRG1, or an allelic variant of exon 6. Also provided is a detection assay comprising a first antibody or a set of a first and second antibodies for detection of the presence of a polypeptide fusion comprising a portion of the polypeptide encoded by exon 2 of said SLC3A2, or an allelic variant of exon 2, fused with a portion of the polypeptide encoded by exon 6 of NRG1, or an allelic variant of exon 6, wherein the first antibody preferably binds the SLC3A2-NRG1 polypeptide fusion of transcript version 3 and the set of first and second antibodies preferably binds said SLC3A2 and NRG1, respectively.

Also provided is a first antibody, or a set of a first and a second antibody, for detection of a polypeptide fusion comprising a portion of the polypeptide encoded by exon 14 of APP, or an allelic variant of exon 14, fused with a portion of the polypeptide encoded by of exon 6 of NRG1, or an allelic variant of exon 6. Also provided is a detection assay comprising a first antibody or a set of a first and second antibodies for detection of the presence of a polypeptide fusion comprising a portion of the polypeptide encoded by of exon 14 of APP, or an allelic variant of exon 14, fused with a portion of the polypeptide encoded by of exon 6 of NRG1, or an allelic variant of exon 6 wherein the first antibody preferably binds the APP-NRG1 polypeptide fusion and the set of first and second antibodies preferably binds APP and NRG1, respectively.

Also provided is a first antibody, or a set of a first and a second antibody, for detection of a polypeptide fusion comprising a portion of the polypeptide encoded by exon 33 of WRN, or an allelic variant of exon 33, fused with a portion of the polypeptide encoded by of exon 6 of NRG1, or an allelic variant of exon 6. Also provided is a detection assay comprising a first antibody or a set of a first and second antibodies for detection of the presence of a polypeptide fusion comprising a portion of the polypeptide encoded by of exon 33 of WRN, or an allelic variant of exon 33, fused with a portion of the polypeptide encoded by of exon 6 of NRG1, or an allelic variant of exon 6 wherein the first antibody preferably binds the WRN-NRG1 polypeptide fusion and the set of first and second antibodies preferably binds WRN and NRG1, respectively.

The present disclosure also provides a first antibody, or a set of a first and a second antibody, for detection of a polypeptide encoded by a polynucleotide fusion comprising a nucleic acid encoding a protein sequence of ASPH, or an allelic variant of ASPH, fused with a nucleic acid encoding a protein sequence of NRG1, or an allelic variant of NRG1.

More in particular is provided a first antibody, or a set of a first and a second antibody, for detection of a polypeptide fusion comprising a portion of the polypeptide encoded by exon 22 of ASPH, or an allelic variant of exon 22, fused with a portion of the polypeptide encoded by of exon 2 of NRG1, or an allelic variant of exon 2. Also provided is a detection assay comprising a first antibody or a set of a first and second antibodies for detection of the presence of a polypeptide fusion comprising a portion of the polypeptide encoded by of exon 22 of ASPH, or an allelic variant of exon 22, fused with a portion of the polypeptide encoded by of exon 2 of NRG1, or an allelic variant of exon 2 wherein the first antibody preferably binds the ASPH-NRG1 polypeptide fusion and the set of first and second antibodies preferably binds ASPH and NRG1, respectively.

Also provided is a first antibody, or a set of a first and a second antibody, for detection of a polypeptide fusion comprising a portion of the polypeptide encoded by exon 6 of NOTCH2, or an allelic variant of exon 6, fused with a portion of the polypeptide encoded by of exon 6 of NRG1, or an allelic variant of exon 6. Also provided is a detection assay comprising a first antibody or a set of a first and second antibodies for detection of the presence of a polypeptide fusion comprising a portion of the polypeptide encoded by of exon 6 of NOTCH2, or an allelic variant of exon 6, fused with a portion of the polypeptide encoded by of exon 6 of NRG1, or an allelic variant of exon 6 wherein the first antibody preferably binds the NOTCH2-NRG1 polypeptide fusion and the set of first and second antibodies preferably binds NOTCH2 and NRG1, respectively.

Also provided is a first antibody, or a set of a first and a second antibody, for detection of a polypeptide fusion comprising a portion of the polypeptide encoded by exon 2 of CD74, or an allelic variant of exon 2, fused with a portion of the polypeptide encoded by of exon 2 of NRG1, or an allelic variant of exon 2. Also provided is a detection assay comprising a first antibody or a set of a first and second antibodies for detection of the presence of a polypeptide fusion comprising a portion of the polypeptide encoded by of exon 2 of CD74, or an allelic variant of exon 2, fused with a portion of the polypeptide encoded by of exon 2 of NRG1, or an allelic variant of exon 2 wherein the first antibody preferably binds the CD74-NRG1 polypeptide fusion and the set of first and second antibodies preferably binds CD74 and NRG1, respectively.

Also provided is a first antibody, or a set of a first and a second antibody, for detection of a polypeptide fusion comprising a portion of the polypeptide encoded by exon 2 of SDC4, or an allelic variant of exon 2, fused with a portion of the polypeptide encoded by of exon 2 of NRG1, or an allelic variant of exon 2. Also provided is a detection assay comprising a first antibody or a set of a first and second antibodies for detection of the presence of a polypeptide fusion comprising a portion of the polypeptide encoded by of exon 2 of SDC4, or an allelic variant of exon 2, fused with a portion of the polypeptide encoded by of exon 2 of NRG1, or an allelic variant of exon 2 wherein the first antibody preferably binds the SDC4-NRG1 polypeptide fusion and the set of first and second antibodies preferably binds SDC4 and NRG1, respectively.

Also provided is a first antibody, or a set of a first and a second antibody, for detection of a polypeptide fusion comprising a portion of the polypeptide encoded by exon 4 of SDC4, or an allelic variant of exon 4, fused with a portion of the polypeptide encoded by of exon 2 of NRG1, or an allelic variant of exon 2. Also provided is a detection assay comprising a first antibody or a set of a first and second antibodies for detection of the presence of a polypeptide fusion comprising a portion of the polypeptide encoded by of exon 4 of SDC4, or an allelic variant of exon 4, fused with a portion of the polypeptide encoded by of exon 2 of NRG1, or an allelic variant of exon 2 wherein the first antibody preferably binds the SDC4-NRG1 polypeptide fusion and the set of first and second antibodies preferably binds SDC4 and NRG1, respectively.

Also provided is a first antibody, or a set of a first and a second antibody, for detection of a polypeptide fusion comprising a portion of the polypeptide encoded by exon 14 of SLC4A4, or an allelic variant of exon 14, fused with a portion of the polypeptide encoded by of exon 6 of NRG1, or an allelic variant of exon 6. Also provided is a detection assay comprising a first antibody or a set of a first and second antibodies for detection of the presence of a polypeptide fusion comprising a portion of the polypeptide encoded by of exon 14 of SLC4A4, or an allelic variant of exon 14, fused with a portion of the polypeptide encoded by of exon 6 of NRG1, or an allelic variant of exon 6 wherein the first antibody preferably binds the SLC4A4-NRG1 polypeptide fusion and the set of first and second antibodies preferably binds SLC4A4 and NRG1, respectively.

The present disclosure also provides a first antibody, or a set of a first and a second antibody, for detection of a polypeptide encoded by a polynucleotide fusion comprising a nucleic acid encoding a protein sequence of ZFAT, or an allelic variant of ZFAT, fused with a nucleic acid encoding a protein sequence of NRG1, or an allelic variant of NRG1.

More in particular is provided a first antibody, or a set of a first and a second antibody, for detection of a polypeptide fusion comprising a portion of the polypeptide encoded by exon 12 of ZFAT, or an allelic variant of exon 12, fused with a portion of the polypeptide encoded by of exon 6 of NRG1, or an allelic variant of exon 6. Also provided is a detection assay comprising a first antibody or a set of a first and second antibodies for detection of the presence of a polypeptide fusion comprising a portion of the polypeptide encoded by of exon 12 of ZFAT, or an allelic variant of exon 12, fused with a portion of the polypeptide encoded by of exon 6 of NRG1, or an allelic variant of exon 6 wherein the first antibody preferably binds the ZFAT-NRG1 polypeptide fusion and the set of first and second antibodies preferably binds ZFAT and NRG1, respectively.

The present disclosure also provides a first antibody, or a set of a first and a second antibody, for detection of a polypeptide encoded by a polynucleotide fusion comprising a nucleic acid encoding a protein sequence of DSCAML1, or an allelic variant of DSCAML1, fused with a nucleic acid encoding a protein sequence of NRG1, or an allelic variant of NRG1.

More in particular is provided a first antibody, or a set of a first and a second antibody, for detection of a polypeptide fusion comprising a portion of the polypeptide encoded by exon 3 of DSCAML1, or an allelic variant of exon 3, fused with a portion of the polypeptide encoded by of exon 2 of NRG1, or an allelic variant of exon 2. Also provided is a detection assay comprising a first antibody or a set of a first and second antibodies for detection of the presence of a polypeptide fusion comprising a portion of the polypeptide encoded by of exon 3 of DSCAML1, or an allelic variant of exon 3, fused with a portion of the polypeptide encoded by of exon 2 of NRG1, or an allelic variant of exon 2 wherein the first antibody preferably binds the DSCAML1-NRG1 polypeptide fusion and the set of first and second antibodies preferably binds DSCAML1 and NRG1, respectively.

Preferably, the anti-NRG1 antibody preferably binds to the EGF-like domain of NRG1. Detection of the presence of an EGF-like domain in a polypeptide fusion of the present disclosure is advantageous since it shows the EGF-like domain is translated and thus part of an in-frame fusion with VAPB, CADM1, CD44, SLC3A2, VTCN1, CDH1, CXADR, GTF2E2, CSMD1, PTN, ST14, THBS1, AGRN, PVALB, APP, WRN, ASPH, NOTCH2, CD74, SDC4, SLC4A4, ZFAT or DSCAML1 as a fusion partner.

The herein mentioned antibodies against NRG1, VAPB, CADM1, CD44, SLC3A2, VTCN1, CDH1, CXADR, GTF2E2, CSMD1, PTN, ST14, THBS1, AGRN, PVALB, APP, WRN, ASPH, NOTCH2, CD74, SDC4, SLC4A4, ZFAT or DSCAML1 can be used in flow-cytometry (FC), fluorescence in situ hybridization (FISH), immunocytochemistry (ICC), immuno histochemistry (IHC) or immunofluorescence (IF).

### Methods of Treatment

The fusions of the present disclosure are present or have been identified in human patients having aberrant cells, in particular patient that have been diagnosed with cancer. The cancer of the present disclosure in particular is a pancreatic cancer, more in particular a pancreatic ductal adenocarcinoma, a sarcoma, a bladder cancer, a colon cancer, a rectal cancer, a colorectal cancer, a gallbladder cancer, a head and neck cancer, a prostate cancer, a uterus cancer, a breast cancer, an ovarian cancer, a liver cancer, an endometrial cancer, a lung cancer, in particular a non-small cell lung cancer or an invasive mucinous adenocarcinoma.

In general terms, the present disclosure also provides methods of treatment of subjects having an aberrant cell, like a cancer, a tumor, wherein said cancer, tumor or aberrant cell, which cell, cancer or tumor comprises an NRG1 polynucleotide fusion of the present disclosure or expresses an NRG1 polypeptide fusion according to the present disclosure.

The present NRG1 fusions have been identified as part of a clinical trial referred to as the eNRGy study and the Early Access Program (NCT02912949 and NCT04100694, respectively). As of the cut-off date of Apr. 12, 2022, a total of about 70 percent of enrolled, non-excluded patients with an identified NRG1 fusion, exhibited investigator-assessed responses observed according to RECIST 1.1 criteria across fusion partners and in multiple different cancer types. Investigator-assessed responses were observed in each category of Table 1. The indications for which responses were observed include pancreatic ductal adenocarcinoma, non-small cell lung cancer, breast cancer and cholangiocarcinoma.

### Prevalence of NRG1 Fusion Partners

TABLE 1
overview of the percentage of NRG1 fusion partners found expressed as
percentage of the indicated fusion partner from the total number of
fusions identified in individuals in clinical trial NCT02912949 with cut-off
of Apr. 12^{th}, 2022. Other comprises NRG1 fusions identified at less
than 2% of the total identified as of the cut-off date.

| NRG1 fusion partner category | Percentage of total |
|---|---|
| CD74 | 31% |
| SLC3A2 | 16% |
| SDC4 | 9% |
| RBPMS | 4% |
| CDH1 | 2% |
| VTCN1 | 2% |
| Others | 23% |

Preferably, the aberrant cell, cancer (or cancer cell) or tumor (or tumor cell) comprises a polynucleotide fusion of the present disclosure which further comprises an in-frame fusion of a coding sequence that codes for EGF-like domain of NRG1.

The disclosure also provides a method of treating a subject having an ErbB-2 and/or ErbB-3 positive aberrant cell, cancer cell or tumor cell that comprises an NRG1 polynucleotide fusion or expresses an NRG1 polypeptide fusion as mentioned herein, said method comprising detecting the presence of any polynucleotide or polypeptide fusion mentioned herein, followed by administering to the subject an effective amount of an ErbB-2 and/or ErbB-3 targeting agent, thereby treating the cancer in the subject. The presence of any one of the NRG1 fusions of the present disclosure, be it a polynucleotide or a polypeptide translated therefrom, is indicative of cancer.

Also provided is a method for inhibiting the progression in a subject of an ErbB-2 and ErbB-3 positive cancer that comprises an NRG1 polynucleotide fusion or expresses an NRG1 polypeptide fusion as mentioned herein, said method comprising detecting the presence of any polynucleotide or polypeptide fusion mentioned herein, followed by administering to the subject an effective amount of an ErbB-2 and/or ErbB-3 targeting agent.

Also provided is an ErbB-2 and/or ErbB-3 targeting agent for use in the treatment of a subject that has an ErbB-2 and ErbB-3 positive cancer that comprises an NRG1 polynucleotide fusion or expresses an NRG1 polypeptide fusion as mentioned herein, said treatment comprising detecting the presence of any polynucleotide or polypeptide fusion mentioned herein, followed by administering an effective amount of the ErbB-2 and/or ErbB-3 targeting agent to the subject.

Also provided is an ErbB-2 and/or ErbB-3 targeting agent for use in the manufacture of a medicament for the treatment of a subject that carries an ErbB-2 and ErbB-3 positive cancer that comprises an NRG1 polynucleotide fusion or expresses an NRG1 polypeptide fusion as mentioned herein, said treatment comprising detecting the presence of any polynucleotide or polypeptide fusion mentioned herein, followed by administering an effective amount of the ErbB-2 and/or ErbB-3 targeting agent to the subject.

Furthermore provided is a method for assessing whether a subject suffers from a cancer or is prone to suffering from a cancer, the method comprising testing a sample obtained from a subject to detect the presence of an NRG1 polynucleotide fusion or an NRG1 polypeptide fusion as mentioned herein in the sample, and assessing that said subject suffers from said cancer or is prone to suffering therefrom, by identifying the presence of said fusion. The testing preferably comprises detecting the fusion by utilizing a binding agent that specifically binds said polynucleotide or utilizing a binding agent that binds a polynucleotide that comprises said polynucleotide fusion. More details on a binding agent that binds a polynucleotide that comprises said polynucleotide fusion are given below herein.

Preferably, the method of diagnosis as mentioned herein comprises the step of associating the detection of an NRG1 fusion in the sample with a cancer expressing an NRG1 polypeptide as mentioned herein to diagnose the subject as having a cancer expressing said NRG1 polypeptide fusion. Detection of the NRG1 fusion molecule, allows to identify a subject as having a cancer that expresses the NRG1 fusion polypeptide and to diagnose said subject with such a cancer. Having knowledge about the underlying oncogenic driver of a cancer allows improved or better tailored treatment to be selected and prescribed. Thus, detection in a sample from a subject of fusion molecules, such as fusion polypeptides or polynucleotides, allows to improve the chances of survival of a subject having an NRG1 associated cancer.

The present disclosure thus provides a method to treat cancer. The cancer is preferably a recurrent cancer or a metastasized cancer. Recurrence typically refers to local recurrence and means that the cancer is in the same place as the original cancer or very close to it. A tumor is typically said to be a metastasized tumor when the tumor has migrated to lymph nodes or tissues near the original cancer or spread to more distant organs or tissues far from the original cancer. In such cases both indications can be used.

The cancer in particular is a pancreatic cancer, pancreatic adenocarcinoma, pancreatic ductal adenocarcinoma, sarcoma, bladder, colorectal, gallbladder, head and neck cancer, prostate, uterus, breast cancer, an ovarian cancer, a liver cancer, an endometrial cancer, a lung cancer such as non-small cell lung cancer, in particular a non-small cell lung cancer, more in particular invasive mucinous adenocarcinoma. Preferably, the tumor genome shows presence or absence of mutations in one or more genes selected from the group consisting of EGFR, KRAS, cKIT-BRCA1-2, MET, ROS, RET, ALK, preferably KRAS.

The present disclosure makes use of an ErbB-2 and/or ErbB-3 targeting agent for treatment of a cancer that expresses an NRG1 fusion polypeptide, wherein the cancer is preferably an ErbB-2 and/or ErbB-3 positive cancer. Said targeting agent is selected from the group consisting of a multispecific antibody comprising a first antigen-binding site that binds an extracellular part of ErbB-2 and a second antigen-binding site that binds an extracellular part of ErbB-3, a tyrosine kinase inhibitor of ErbB-2, a monospecific bivalent antibody comprising an antigen-binding site that bind an extracellular part of ErbB-2, a monospecific bivalent antibody comprising an antigen-binding site that bind an extracellular part of ErbB-3, or any combination thereof.

Also included for the treatment of a cancer is the administration of one or more compounds selected from the group consisting of an inhibitor of a component of the PI3Kinase pathway, an inhibitor of a component of the MAPK pathway, a microtubule disrupting drug, and an inhibitor of a histone deacetylase (HDAC). Said inhibitor preferably comprises a tyrosine kinase inhibitor, a PI3Ka inhibitor, an Akt inhibitor, an mTOR inhibitor or an Src inhibitor. Said tyrosine kinase inhibitor is preferably afatinib, lapatinib and/or neratinib. Said PI3Ka inhibitor is preferably BYL719. In one embodiment, said Akt inhibitor is MK-2206. In one preferred embodiment, said mTOR inhibitor is everolimus. In one preferred embodiment, said Src inhibitor is saracatinib. In one preferred embodiment, said microtubule disrupting drug is paclitaxel. In one preferred embodiment, said HDAC inhibitor is vorinostat. In one embodiment, said binding compound that is specific for ErbB 2 and ErbB 3 is MM 111.

The ErbB-2 targeting agent is preferably with a monospecific bivalent antibody comprising antigen-binding sites that bind an extracellular part of ErbB-2. Such antibody is preferably trastuzumab, pertuzumab, or trastuzumab-emtansine. The ErbB-2 targeted treatment is preferably an ErbB-2 TKI. The ErbB-2 TKI is preferably one or more of lapatinib, canertinib, neratinib, tucatinib (or irbinitinib), CP-724714, tarloxitinib, mubritinib, afatinib, varlitinib, and dacomitinib, preferably afatinib. An ErbB-2 TKI may also affect ErbB-1 signaling but is different from an ErbB-1 TKI in that it has significant activity on ErbB-2. The ErbB-3 targeted treatment is preferably with a monospecific bivalent antibody comprising antigen-binding sites that bind an extracellular part of ErbB-3. Such antibody is preferably patritumab, seribantumab, lumretuzumab, elgemtumab, GSK2849330, KTN3379 or AV-203.

Preferably, the monospecific bivalent antibody comprising antigen-binding sites that bind an extracellular part of ErbB-3 comprises patritumab, seribantumab, lumretuzumab, elgemtumab, GSK2849330, KTN3379 or AV-203.

The tyrosine kinase inhibitor is preferably afatinib, lapatinib and/or neratinib.

More preferably, the ErbB-2 and/or ErbB-3 targeting agent is a multispecific, or more preferably a bispecific antibody, comprising a first antigen-binding site that binds an extracellular part of ErbB-2 and a second antigen-binding site that binds an extracellular part of ErbB-3. In particular, said bispecific antibody is zenocutuzumab.

The antibody can preferably reduce a ligand-induced receptor function of ErbB-3 on a ErbB-2 and ErbB-3 positive cell. Also, the antibody can preferably reduce ligand-induced growth of an ErbB-2 and ErbB-3 positive cell. The antibody is preferably a multispecific or bispecific antibody comprising a first antigen-binding site that binds an extracellular part of ErbB-2 and a second antigen-binding site that binds an extracellular part of ErbB-3.

The subject is preferably a human subject. The subject is preferably a subject eligible for monoclonal antibody therapy using an ErbB-2 specific antibody such as trastuzumab. In a preferred embodiment the subject comprises a tumor/cancer, preferably an ErbB-2/ErbB-3 positive cancer, preferably a tumor/cancer with an ErbB-2 therapy resistant phenotype and/or a heregulin resistance phenotype, preferably a monoclonal antibody resistant phenotype. A tumor involving such phenotype can escape treatment with a current anti-HER2 regimen, such as (but not limited to) monoclonal antibody therapy against ErbB-2.

In particular, the cancer comprises a tumor that is a metastasized tumor, preferably wherein the tumor has migrated to lymph nodes or tissues near the original cancer or spread to more distant organs or tissues far from the original cancer.

The present disclosure also includes in vivo models, such as xenograft or transgenic animal models expressing within their genome or engrafted aberrant cells comprising a polynucleotide fusion as mentioned herein, and/or expressing a polypeptide fusion encoded therefrom, and treatment of such models with an ERB2 and/or Erb3 targeting agent or other targeting agent for evaluation of therapeutic activity of such agent. Preferably, the animal model is a non-human animal model.

In disclosed embodiments, said in vivo animal model comprises a polynucleotide fusion according to the present disclosure and/or expresses a polypeptide fusion encoded therefrom, wherein preferably the polynucleotide fusion or polypeptide fusion comprised by the animal model is comprised by an engrafted aberrant cell present in the animal model or comprised by the genome of the animal model.

In disclosed embodiments, the present disclosure provides a method of treatment of said in vivo animal model with an Erb2 and/or Erb3 targeting agent selected from the group consisting of a multispecific antibody comprising a first antigen-binding site that binds an extracellular part of ErbB-2 and a second antigen-binding site that binds an extracellular part of ErbB-3, a tyrosine kinase inhibitor of ErbB-2, a monospecific bivalent antibody comprising an antigen-binding site that bind an extracellular part of ErbB-2, a monospecific bivalent antibody comprising an antigen-binding site that bind an extracellular part of ErbB-3, or any combination thereof, said method comprising administering to the animal said Erb2 and/or Erb3 targeting agent.

In disclosed embodiments, the present disclosure provides an Erb2 and/or Erb3 targeting agent selected from the group consisting of a multispecific antibody comprising a first antigen-binding site that binds an extracellular part of ErbB-2 and a second antigen-binding site that binds an extracellular part of ErbB-3, a tyrosine kinase inhibitor of ErbB-2, a monospecific bivalent antibody comprising an antigen-binding site that bind an extracellular part of ErbB-2, a monospecific bivalent antibody comprising an antigen-binding site that bind an extracellular part of ErbB-3, or any combination thereof, for use in treatment of said in vivo animal model. Said method preferably comprises administering to the animal said Erb2 and/or Erb3 targeting agent.

### Dosing and Administration

Actual dosage levels of the active ingredients in the pharmaceutical compositions of the present disclosure or as administered in any method of treatment may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. Said active ingredient is preferably any ErbB-2 and/or ErbB-3 targeting agent of the present disclosure. Any approved active ingredient or drug undergoing a clinical trial, in particular once entered a phase two of a clinical trial, can be considered by a physician to be administered in accordance with the approved dosage regime or clinical trial dosage regime.

The amount of any ErbB-2 and/or ErbB-3 targeting agent as mentioned herein to be administered to a patient is typically in the therapeutic window, meaning that a sufficient quantity is used for obtaining a therapeutic effect, while the amount does not exceed a threshold value leading to an unacceptable extent of side-effects. The lower the amount of the therapeutic substance needed for obtaining a desired therapeutic effect, the larger the therapeutic window will typically be. The selected dosage level will depend upon a variety of factors including the route of administration, the time of administration, the rate of excretion of the particular compound being employed, the duration of the treatment, other drugs, compounds and/or materials used in combination, the age, sex, weight, condition, general health and prior medical history of the subject being treated, and like factors well known in the medical arts.

With respect to zenocutuzumab, it has a good safety profile at relatively high doses, thus providing a large therapeutic window compared to other targeted or cytotoxic, chemotherapy agents. Dosing of the bispecific antibody of the present disclosure follows a weekly, biweekly or tri-weekly administration regimen of 750 mg, preferably a bi-weekly dose of 750 mg. The dosing is preferably in subjects with pancreatic cancer, NSCLC or a solid tumor, and includes any subject having a solid tumor harboring an NRG1-fusion, wherein such subject has progressed upon the administration of chemotherapy, standard of care or an ErbB-2 or ErbB-3 targeting agent or TKI. Alternatively, a dosing regimen is followed comprising a weekly flat dose administration of 400 mg, preferably commenced after a single administration of 800 mg. Following this alternative dosing regimen, the bispecific antibody of the disclosure is preferably administered in a weekly dose of 400 mg for 3 weeks followed by 1 week without dosing. Next, one or more cycles of a period of four weeks, consisting of three weekly flat dosages of 400 mg, followed by a week without administration is followed. This is preferably followed until a therapeutic effect is observed.

Dosing preferably involves intravenous injections of two infusions of the bispecific antibody of the disclosure to arrive at the complete dose, preferably when dosing >360 mg antibody. Alternatively, a single infusion of the complete dose may be given for lower dosages, for instance when dosing ≤360 mg antibody. Pre-medication maybe included in the dosing regimen to mitigate infusion-related reactions.

### GENERAL

The articles “a” and “an” are used herein to refer to one or more than one (i.e. to one or at least one) of the grammatical object of the article.

Throughout the present specification and the accompanying claims, the words “comprise”, “include” and “having” and variations such as “comprises”, “comprising”, “includes” and “including” are to be interpreted inclusively. That is, these words are intended to convey the possible inclusion of other elements or integers not specifically recited, where the context allows.

“Percent (%) identity” as referring to nucleic acid or amino acid sequences is defined as the percentage of residues in a candidate sequence that are identical with the residues in a selected sequence, after aligning the sequences for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/based or amino acids. The sequence identity is the percentage of identical matches between the two sequences over the reported aligned region.

A comparison of sequences and determination of percentage of sequence identity between two sequences can be accomplished using a mathematical algorithm. The skilled person will be aware of the fact that several different computer programs are available to align two sequences and determine the identity between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley). The percent sequence identity between two amino acid sequences or nucleic acid sequences may be determined using the Needleman and Wunsch algorithm for the alignment of two sequences. (Needleman, S. B. and Wunsch, C. D. (1970) J. Mol. Biol. 48, 443-453). The Needleman-Wunsch algorithm has been implemented in the computer program NEEDLE. For the purpose of this disclosure the NEEDLE program from the EMBOSS package is used to determine percent identity of amino acid and nucleic acid sequences (version 2.8.0 or higher, EMBOSS: The European Molecular Biology Open Software Suite (2000) Rice, P. Longden J. and Bleasby, A. Trends in Genetics 16, (6) pp 276-277, http://emboss.bioinformatics.nl/). For protein sequences, EBLOSUM62 is used for the substitution matrix. For DNA sequences, DNAFULL is used. The parameters used are a gap-open penalty of 10 and a gap extension penalty of 0.5.

After alignment by the program NEEDLE as described above the percentage of sequence identity between a query sequence and a sequence of the disclosure is calculated as follows: Number of corresponding positions in the alignment showing an identical amino acid or identical nucleotide in both sequences divided by the total length of the alignment after subtraction of the total number of gaps in the alignment.

“Allelic variant” means a naturally occurring allele of the specific sequence as identified in a patient derived sample, which sequence as identified has been assigned a SEQ ID NO, and which variant falls within a defined range of variability. The variability is defined as having at least 85% sequence identity to the sequence it is indicated to be an allelic variant of, preferably at least 90% identity, 92%, 94%, 95%, 96% or more preferably at least 98% sequence identity thereto. Such variants are considered an allele of the sequence it is compared with and therefore still qualify as the gene it is a variant of. If not expressly mentioned in relation to a particular allelic variant herein, these sequence identity percentages are still applicable to the allelic variant referred to.

A “bispecific antibody” means an antibody having one variable domain of the antibody that binds to a first antigen whereas a second variable domain of the antibody binds to a second antigen, wherein said first and second antigens are not identical. The term “bispecific antibody” also encompasses biparatopic antibodies, wherein one variable domain of the antibody binds to a first epitope on an antigen whereas a second variable domain of the antibody binds a second epitope on the same antigen. The term further includes antibodies wherein at least one VH is capable of specifically recognizing a first antigen and a VL, paired with the at least one VH in an immunoglobulin variable domain, is capable of specifically recognizing a second antigen. The resulting VH/VL pair will bind either antigen 1 or antigen 2, and are called “two-in-one antibodies”, described in for instance WO 2008/027236, WO 2010/108127 and Schaefer et al (Cancer Cell 20, 472-486, October 2011). A bispecific antibody according to the present disclosure is not limited to any particular bispecific format or method of producing it.

A “detectable label” means a chemical, biological, or other modification, including but not limited to fluorescence, mass, residue, dye, radioisotope, label, or tag modifications, etc., which allows the presence of the molecule, herein preferably a polynucleotide or polypeptide, of interest to be detected. Preferably, the detectable label is a visible label, a fluorescent dye, a quencher, a UV-detectable label, a chromogenic label, a radioactive label, an electrochemical label, a tag, an enzyme that produces a detectable label when in contact with a substrate specific for said enzyme or a molecular barcode. Exemplary fluorescent dyes include water-soluble rhodamine dyes, fluoresceins, 4,7-dichlorofluoresceins, benzoxanthene dyes and energy transfer dyes, as disclosed in the following references: Handbook of Molecular Probes and Research Reagents, 8th ed. (2002), Molecular Probes, Eugene, Oreg.; WO 2001/32783; U.S. Pat. Publ. Nos. US 2002-0081616, US 2002-0086985; and Lee et al., 1997, Nucleic Acids Research 25:2816-2822.

“Isolated” or “purified” means a nucleic acid or amino acid sequence that is removed from its natural environment, isolated or separated. An “isolated” nucleic acid molecule includes one which is separated from other nucleic acid molecules which are present in the natural source of the nucleic acid molecule. Also, an “isolated” nucleic acid molecule, such as a cDNA molecule, includes the state of being free of other cellular material or culture medium when produced by recombinant techniques, or substantially free of chemical precursors or other chemicals when chemically synthesized.

“Sample” herein means a sample obtained from a subject or patient. Such a sample is a biological sample or a patient sample, which terms are exchangeably used herein as the samples mentioned herein are biological samples obtained from patients. The term “sample” includes a liquid biopsy sample, a sample that is taken from a solid cancer or tumor and will contain polynucleotides fusions and/or polypeptides fusions before any processing is applied to it. In particular, the sample comprises an aberrant cell, such as a tumor cell or a cancer cell.

The language “free of other cellular material or culture medium” includes preparations of nucleic acid molecule in which the molecule is separated from cellular components of the cells from which it is isolated or recombinantly produced.

“Primer” means a single-stranded nucleotide molecule of sufficient length that specifically hybridizes to a nucleotide sequence of interest to allow specific binding, also referred to herein as hybridization or annealing, to a targeted or selected nucleotide sequence that, together with a second primer, defines a region of interest that is amplified in a polymerase chain reaction, or PCR. Herein, the first and second primers, or primer pairs, are specifically designed to amplify the nucleotide region spanning the NRG1 fusion junction. In particular, a combination of a first primer hybridizing to a nucleotide sequence specific for the NRG1 sequence part at one side of the fusion junction and a second primer hybridizing to a nucleotide sequence specific for a fusion partner selected from VAPB, CADM1, CD44, SLC3A2, VTCN1, CDH1, CXADR, GTF2E2, CSMD1, PTN, ST14, THBS1, AGRN, PVALB, APP, WRN, DAAM1, ASPH, NOTCH2, CD74, SDC4, SLC4A4, ZFAT or DSCAML1 at the other side of the fusion junction is used. Typically, a primer itself does not comprise a detectable label. Over the entire length of its target sequence in an NRG1 fusion polynucleotide, a primer typically has at least about 80%, at least about 85%, at least about 90%, at least about 95%, at least about 97%, at least about 98% or at least about 99% sequence identity to the target sequence. The polynucleotide primer is most preferably complementary (100% sequence identity over the entire length) to its target sequence. In all instances herein, sequence identity is typically measured over the entire length of the non-variant/target sequence.

“Probe” or “nucleic acid probe” means a single-stranded nucleotide molecule of sufficient length that specifically hybridizes to a nucleotide sequence of interest. In particular, in the context of the present disclosure, the probe allows detection of fusions occurring in VAPB, CADM1, CD44, SLC3A2, VTCN1, CDH1, CXADR, GTF2E2, CSMD1, PTN, ST14, THBS1, AGRN, PVALB, APP, WRN, DAAM1, ASPH, NOTCH2, CD74, SDC4, SLC4A4, ZFAT or DSCAML1, in particular a fusion selected from VAPB-NRG1, CADM1-NRG1, CD44-NRG1, SLC3A2-NRG1, VTCN1-NRG1, CDH1-NRG1, CXADR-NRG1, GTF2E2-NRG1, CSMD1-NRG1, PTN-NRG1, ST14-NRG1, THBS1-NRG1, AGRN-NRG1, PVALB-NRG1, APP-NRG1, WRN-NRG1, DAAM1-NRG1, ASPH-NRG1, NOTCH2-NRG1, CD74-NRG1, SDC4-NRG1, SLC4A4-NRG1, ZFAT-NRG1 and DSCAML1-NRG1. A probe typically comprises one or two detectable labels. Over the entire length of its target sequence in the NRG1 fusion polynucleotide, a polynucleotide probe typically has at least about 80%, at least about 85%, at least about 90%, at least about 95%, at least about 97%, at least about 98% or at least about 99% sequence identity to the target sequence. The polynucleotide primer is most preferably complementary (100% sequence identity over the entire length) to its target sequence. In all instances herein, sequence identity is typically measured over the entire length of the non-variant/target sequence.

The polynucleotide probe is preferably a DNA probe, a TaqMan probe, a molecular beacon, a scorpion probe or probe as used in FISH. DNA probes hybridise to the, typically complementary, target sequence and then can be detected, for instance using a detectable label. TaqMan probes are known in the art and are polynucleotides that have a fluorescent dye attached to the 5′ end and a quencher to the 3′ end. The polymerase used in PCR cleaves hybridised probes freeing the fluorescent dye from quenching such it can be detected. Molecular beacon probes are known in the art and are similar to TaqMan probes except (rather than using cleavage to separate the dye from the quencher) hybridisation to the target sequence separates the dye from the quencher. Scorpion probes are known the art and are similar to molecular beacons except the 3′ end also contains a sequence that is complementary to the extension product of the primer on the 5′ end which opens the probe on hybridisation and allows the dye to be detected. The polynucleotide probe is preferably a TaqMan probe. The polynucleotide probe is preferably a TaqMan probe and is used in any of the PCR methods discussed above.

A probe as used in a technique like FISH, or break apart FISH, has a suitable length and comprises a single detectable label. FISH involves detection of highly specific DNA probes which have been hybridized to either interphase or metaphase chromosomes that are made visible to allow detection using fluorescence microscopy. Break apart FISH is particularly suitable to detect fusion breakpoints. In that case, two ends of the gene of interest are labeled with different colors, and when a translocation occurs that gives rise to a fusion, the two individual colors do not co-localize and two primary colors are observed. In normal cells with normal copies of the labeled gene of interest, the two colors co-localize giving rise to a fused signal.

A probe or primer for identification of a fusion polynucleotide comprised by a cell, can be isolated or purified using standard molecular biology techniques or synthesized based on the sequence information in the database records described herein. Such nucleic acid molecules as described herein can be isolated using standard hybridization and cloning techniques (e.g. as described in Sambrook et al., ed., Molecular Cloning: A Laboratory Manual, 2nd ed., Cold Spring Harbor Laboratory Press, Cold Spring Harbor, NY, 1989). Also, a fusion polynucleotide can be isolated, purified or synthesized using any suitable standard molecular biological technique. In some embodiments, the primers or probe are specific for cDNA. For example, in some embodiments the primers or probes are specific for an exon-exon junction in cDNA.

“Polynucleotide fusion” means a covalently bonded connection between two polynucleotides, wherein on one side of the fusion junction a nucleotide sequence from one gene is present and on the other side a nucleotide sequence from another gene is present. In the context of polynucleotide fusions, the connection is operably linked in a manner that permits transcription into a protein. On one side, said nucleotide sequences are preferably from VAPB, CADM1, CD44, SLC3A2, VTCN1, CDH1, CXADR, GTF2E2, CSMD1, PTN, ST14, THBS1, AGRN, PVALB, APP, WRN, DAAM1, ASPH, NOTCH2, CD74, SDC4, SLC4A4, ZFAT or DSCAML1 and preferably from NRG1 on the other side of the fusion junction.

“Polypeptide fusion” means a covalently bonded connection between two amino acids, wherein on one side of the fusion junction an amino acid sequence from one polypeptide is present and on the other side an amino acid sequence from another polypeptide is present. On one side, said amino acid sequences are preferably from VAPB, CADM1, CD44, SLC3A2, VTCN1, CDH1, CXADR, GTF2E2, CSMD1, PTN, ST14, THBS1, AGRN, PVALB, APP, WRN, ASPH, NOTCH2, CD74, SDC4, SLC4A4, ZFAT or DSCAML1 and preferably from NRG1 on the other side of the fusion junction.

“Fusion partner” means a gene or polypeptide with which a fusion with the NRG1 gene or polypeptide has occurred due to a genetic rearrangement. In the present disclosure, the fusion partner is one of VAPB, CADM1, CD44, SLC3A2, VTCN1, CDH1, CXADR, GTF2E2, CSMD1, PTN, ST14, THBS1, AGRN, PVALB, APP, WRN, DAAM1, ASPH, NOTCH2, CD74, SDC4, SLC4A4, ZFAT or DSCAML1. Typically, the fusion partner is located upstream, (at the 5′ end of the sense strand) of the NRG1 gene, and is fused in-frame leading to translation into a chimeric protein which comprises a part of the fusion partner and an NRG1 part. A fusion gene results from joining parts of two different genes and a fusion polypeptide is the translational product from such fusion genes.

“Fusion point” means the position in an amino acid or nucleotide sequence where on one side of the position an NRG1 sequence is present and on the other side an NRG1 fusion partner sequence is present. It is the junction or position where genetic rearrangement occurs forming a chimeric gene, mRNA and/or protein sequence. The term “fusion point” herein is exchangeable with “junction” or “fusion junction”.

A “3′ located gene sequence” means any element, of regulatory nature or not, that is located downstream or 3′ of the gene referred to and considered to be part of said gene. A regulatory element herein includes an enhancer, silencer, response element, intron, exon, embedded promoter, which element is specific to the gene involved to allow amplification and identification of the fusion in a unique fashion due to the proximity to the fusion junction. A 3′ located gene sequence in this context also includes any gene sequences positioned in between any regulatory elements that can be used to uniquely identify a fusion as involved.

A “5′ located gene sequence” means any element, of regulatory nature or not, that is located upstream or 5′ of the gene referred to and considered to be part of said gene. A regulatory element herein includes a promoter, enhancer, silencer, response element, intron, exon, embedded promoter, which element is specific to the gene involved to allow amplification and identification of the fusion in a unique fashion due to the proximity to the fusion junction. A 5′ located gene sequence in this context also includes any gene sequences positioned in between any regulatory elements that can be used to uniquely identify a fusion as involved.

The term ‘ErbB-2’ as used herein refers to the protein that in humans is encoded by the ERBB-2 gene. Alternative names for the gene or protein include CD340; HER-2; HER-2/neu; MLN 19; NEU; NGL; TKR1. The ERBB-2 gene is frequently called HER2 (from human epidermal growth factor receptor 2). Where reference is made herein to ErbB-2, the reference refers to human ErbB-2. An antibody comprising an antigen-binding site that binds ErbB-2, binds human ErbB-2. The ErbB-2 antigen-binding site may, due to sequence and tertiary structure similarity between human and other mammalian orthologs, also bind such an ortholog but not necessarily so. Database accession numbers for the human ErbB-2 protein and the gene encoding it are (NP_001005862.1, NP_004439.2 NC_000017.10 NT_010783.15 NC_018928.2). The accession numbers are primarily given to provide a further method of identification of ErbB-2 as a target, the actual sequence of the ErbB-2 protein bound the antibody may vary, for instance because of a mutation in the encoding gene such as those occurring in some cancers or the like. The ErbB-2 antigen binding site binds ErbB-2 and a variety of variants thereof, such as those expressed by some ErbB-2 positive tumor cells. The antigen-binding site that binds ErbB-2 preferably binds domain I of ErbB-2.

The term ‘ErbB-3’ as used herein refers to the protein that in humans is encoded by the ERBB3 gene. Alternative names for the gene or protein are HER3; LCCS2; MDA-BF-1; c-ErbB-3; c-ErbB3; ErbB3-S; p180-ErbB3; p45-sErbB3; and p85-sErbB3. Where reference is made herein to ErbB-3, the reference refers to human ErbB-3. An antibody comprising an antigen-binding site that binds ErbB-3, binds human ErbB-3. The ErbB-3 antigen-binding site may, due to sequence and tertiary structure similarity between human and other mammalian orthologs, also bind such an ortholog but not necessarily so. Database accession numbers for the human ErbB-3 protein and the gene encoding it are (NP_001005915.1, NP_001973.2, NC_000012.11, NC_018923.2, NT_029419.12). The accession numbers are primarily given to provide a further method of identification of ErbB-3 as a target, the actual sequence of the ErbB-3 protein bound by an antibody may vary, for instance because of a mutation in the encoding gene such as those occurring in some cancers or the like. The ErbB-3 antigen binding site binds ErbB-3 and a variety of variants thereof, such as those expressed by some ErbB-3 positive tumor cells. The antigen-binding site that binds ErbB-3 preferably binds domain III of ErbB-3.

When reference is made to ErbB-2 or ErbB-3 or an alternative name for the same, the reference is to human ErbB-2 or ErbB-3. Antibodies as referred to herein bind to ErbB-2 or ErbB-3 and many mutated ErbB-2 or ErbB-3 proteins as can be found in cancers.

Any range of numerical SEQ ID NOs as mentioned herein expressly includes all the individual SEQ ID NOs falling within the relevant range and includes the end points thereof. Thus, for the avoidance of doubt, if for instance reference is made to SEQ ID NOs: 17-23, it is intended to mention and disclose SEQ ID NO: 17, SEQ ID NO: 18, SEQ ID NO: 19, SEQ ID NO: 20, SEQ ID NO: 21, SEQ ID NO: 22 and SEQ ID NO: 23 in the context the range of SEQ ID NOs is mentioned.

### CLAUSES

The following clauses describe exemplary embodiments.

1. A polynucleotide comprising a VAPB nucleic acid sequence, or an allelic variant of the VAPB sequence, fused with an NRG1 nucleic acid sequence, or an allelic variant of the NRG1 sequence.

2. The polynucleotide according to clause 1, wherein the VAPB nucleic acid sequence comprises or consists of any one of SEQ ID NOs: 17-23, or an allelic variant of any one of SEQ ID NOs: 17-23, and the NRG1 nucleic acid sequence comprises or consists of any one of SEQ ID NOs: 125-138.

3. The polynucleotide according to clauses 1 or 2, wherein the VAPB nucleic acid sequence (or the allelic variant thereof), is 5′ to the NRG1 nucleic acid sequence (or the allelic variant thereof).

4. The polynucleotide according to any one of the previous clauses, wherein the allelic variant of the VAPB nucleic acid sequences has at least 85% identity to any one of SEQ ID NOs: 17-23, preferably at least 90% identity, more preferably at least 95% sequence identity; and the allelic variant of the NRG1 nucleic acid sequences has at least 85% identity to any one of SEQ ID NOs: 125-138, preferably at least 90% identity, more preferably at least 95% sequence identity.

5. The polynucleotide according to any one of the previous clauses, wherein the fusion of the VAPB nucleic acid with the NRG1 nucleic acid comprises 2 to about 40 contiguous nucleic acids from SEQ ID NO: 3, preferably including the nucleic acids at positions 43 and 44.

6. The polynucleotide according to any one of the previous clauses, wherein the nucleic acid encoding the NRG1 protein sequence (or the allelic variant thereof), comprises or encodes an EGF-like domain of NRG1, preferably the EGF-like domain according to SEQ ID NO: 163.

7. A polynucleotide comprising
a PVALB nucleic acid sequence, or an allelic variant of the PVALB sequence, fused with an NRG1 nucleic acid sequence, or an allelic variant of the NRG1 sequence or
a ASPH nucleic acid sequence, or an allelic variant of the ASPH sequence, fused with an NRG1 nucleic acid sequence, or an allelic variant of the NRG1 sequence or
a DAAM1 nucleic acid sequence, or an allelic variant of the DAAM1 sequence, fused with an NRG1 nucleic acid sequence, or an allelic variant of the NRG1 sequence or
a ZFAT nucleic acid sequence, or an allelic variant of the ZFAT sequence, fused with an NRG1 nucleic acid sequence, or an allelic variant of the NRG1 sequence or
a DSCAML1 nucleic acid sequence, or an allelic variant of the DSCAML1 sequence, fused with an NRG1 nucleic acid sequence, or an allelic variant of the NRG1 sequence.

8. The polynucleotide according to clause 7, wherein
the PVALB nucleic acid sequence comprises or consists of any one of SEQ ID NOs: 439-444, or an allelic variant of any one of SEQ ID NOs: 439-444, and the NRG1 nucleic acid sequence comprises or consists of any one of SEQ ID NOs: 125-138;
the DAAM1 nucleic acid sequence comprises or consists of any one of SEQ ID NOs: 606-631, or an allelic variant of any one of SEQ ID NOs: 606-631, and the NRG1 nucleic acid sequence comprises or consists of any one of SEQ ID NOs: 125-138;
the ZFAT nucleic acid sequence comprises or consists of any one of SEQ ID NOs: 830-846, or an allelic variant of any one of SEQ ID NOs: 830-846, and the NRG1 nucleic acid sequence comprises or consists of any one of SEQ ID NOs: 125-138; or
the DSCAML1 nucleic acid sequence comprises or consists of any one of SEQ ID NOs: 870-903, or an allelic variant of any one of SEQ ID NOs: 870-903, and the NRG1 nucleic acid sequence comprises or consists of any one of SEQ ID NOs: 125-138.

9. The polynucleotide according to clauses 7 or 8, wherein the PVALB, DAAM1, ZFAT or DSCAML1 nucleic acid sequence (or the allelic variant thereof), is 5′ to the NRG1 nucleic acid sequence (or the allelic variant thereof).

10. The polynucleotide according to any one of clauses 7-9, wherein
the allelic variant of the PVALB nucleic acid sequences has at least 85% identity to any one of SEQ ID NOs: 439-444, preferably at least 90% identity, more preferably at least 95% sequence identity; and the allelic variant of the NRG1 nucleic acid sequences has at least 85% identity to any one of SEQ ID NOs: 125-138, preferably at least 90% identity, more preferably at least 95% sequence identity;
the allelic variant of the DAAM1 nucleic acid sequences has at least 85% identity to any one of SEQ ID NOs: 606-631, preferably at least 90% identity, more preferably at least 95% sequence identity; and the allelic variant of the NRG1 nucleic acid sequences has at least 85% identity to any one of SEQ ID NOs: 125-138, preferably at least 90% identity, more preferably at least 95% sequence identity;
the allelic variant of the ZFAT nucleic acid sequences has at least 85% identity to any one of SEQ ID NOs: 830-846, preferably at least 90% identity, more preferably at least 95% sequence identity; and the allelic variant of the NRG1 nucleic acid sequences has at least 85% identity to any one of SEQ ID NOs: 125-138, preferably at least 90% identity, more preferably at least 95% sequence identity; or
the allelic variant of the DSCAML1 nucleic acid sequences has at least 85% identity to any one of SEQ ID NOs: 870-903, preferably at least 90% identity, more preferably at least 95% sequence identity; and the allelic variant of the NRG1 nucleic acid sequences has at least 85% identity to any one of SEQ ID NOs: 125-138, preferably at least 90% identity, more preferably at least 95% sequence identity.

11. The polynucleotide according to any one of clauses 7-10, wherein
the fusion of the PVALB nucleic acid with the NRG1 nucleic acid comprises 2 to about 40 contiguous nucleic acids from SEQ ID NO: 437, preferably including the nucleic acids at positions 102 and 103;
the fusion of the DAAM1 nucleic acid with the NRG1 nucleic acid comprises 2 to about 40 contiguous nucleic acids from SEQ ID NO: 605, preferably including the nucleic acids at positions 75 and 76;
the fusion of the ZFAT nucleic acid with the NRG1 nucleic acid comprises 2 to about 40 contiguous nucleic acids from SEQ ID NO: 828, preferably including the nucleic acids at positions 75 and 76; or
the fusion of the DSCAML1 nucleic acid with the NRG1 nucleic acid comprises 2 to about 40 contiguous nucleic acids from SEQ ID NO: 868, preferably including the nucleic acids at positions 75 and 76.

12. The polynucleotide according to any one of clauses 7-11, wherein the nucleic acid encoding the NRG1 protein sequence (or the allelic variant thereof), comprises or encodes an EGF-like domain of NRG1, preferably the EGF-like domain according to SEQ ID NO: 163.

13. A polynucleotide comprising:
a portion of exon 1 of VAPB, or of an allelic variant of exon 1, fused with a portion of exon 2 of NRG1, or an allelic variant of exon 2;
a portion of exon 7 of CADM1, or of an allelic variant of exon 7, and a portion of exon 6 of NRG1, or an allelic variant of exon 6;
a portion of exon 5 of CD44, or of an allelic variant of exon 5, and a portion of exon 2 of NRG1, or an allelic variant of exon 2;
a portion of exon 1 of transcript version 6 of SLC3A2, or of an allelic variant of exon 1, and a portion of exon 5 of NRG1, or an allelic variant of exon 5;
a portion of exon 2 of VTCN1, or of an allelic variant of exon 2, and a portion of exon 2 of NRG1, or an allelic variant of exon 2;
a portion of exon 11 of CDH1, or of an allelic variant of exon 11, and a portion of exon 2 of NRG1, or an allelic variant of exon 2;
a portion of exon 1 of CXADR, or of an allelic variant of exon 1, and a portion of exon 2 of NRG1, or an allelic variant of exon 2;
a portion of exon 2 of GTF2E2, or of an allelic variant of exon 2, and a portion of exon 2 of NRG1, or an allelic variant of exon 2;
a portion of exon 23 of CSMD1, or of an allelic variant of exon 23, and a portion of exon 6 of NRG1, or an allelic variant of exon 6;
a portion of exon 4 of PTN, or of an allelic variant of exon 4, and a portion of exon 2 of NRG1, or an allelic variant of exon 2;
a portion of exon 11 of ST14, or of an allelic variant of exon 11, and a portion of exon 6 of NRG1, or an allelic variant of exon 6;
a portion of exon 9 of THBS1, or of an allelic variant of exon 9, and a portion of exon 6 of NRG1, or an allelic variant of exon 6;
a portion of exon 12 of AGRN, or of an allelic variant of exon 12, and a portion of exon 6 of NRG1, or an allelic variant of exon 6;
a portion of exon 4 of PVALB, or of an allelic variant of exon 4, and a portion of exon 6 of NRG1, or an allelic variant of exon 6;
a portion of exon 2 of transcript version 3 of SLC3A2, or of an allelic variant of exon 2, and a portion of exon 6 of NRG1, or an allelic variant of exon 6;
a portion of exon 14 of APP, or of an allelic variant of exon 14, and a portion of exon 6 of NRG1, or an allelic variant of exon 6;
a portion of exon 33 of WRN, or of an allelic variant of exon 33, and a portion of exon 6 of NRG1, or an allelic variant of exon 6;
a portion of exon 1 of DAAM1, or of an allelic variant of exon 1, and a portion of exon 1 of NRG1, or an allelic variant of exon 1;
a portion of exon 22 of ASPH, or of an allelic variant of exon 22, and a portion of exon 2 of NRG1, or an allelic variant of exon 2;
a portion of exon 6 of NOTCH2, or of an allelic variant of exon 6, and a portion of exon 6 of NRG1, or an allelic variant of exon 6;
a portion of exon 2 of CD74, or of an allelic variant of exon 2, and a portion of exon 2 of NRG1, or an allelic variant of exon 2;
a portion of exon 2 of SDC4, or of an allelic variant of exon 2, and a portion of exon 2 of NRG1, or an allelic variant of exon 2;
a portion of exon 5 of CD44, or of an allelic variant of exon 5, and a portion of exon 6 of NRG1, or an allelic variant of exon 6;
a portion of exon 14 of SLC4A4, or of an allelic variant of exon 14, and a portion of exon 6 of NRG1, or an allelic variant of exon 6;
a portion of exon 4 of SDC4, or of an allelic variant of exon 4, and a portion of exon 2 of NRG1, or an allelic variant of exon 2;
a portion of exon 12 of ZFAT, or of an allelic variant of exon 12, and a portion of exon 6 of NRG1, or an allelic variant of exon 6, or
a portion of exon 3 of DSCAML1, or of an allelic variant of exon 3, and a portion of exon 2 of NRG1, or an allelic variant of exon 2.

14. The polynucleotide according to clause 13, wherein exon 1 of VAPB is that of SEQ ID NO: 17; exon 7 of CADM1 is that of SEQ ID NO: 39; exon 5 of CD44 is that of SEQ ID NO: 65; exon 1 of SLC3A2 is that of SEQ ID NO: 103; exon 2 of VTCN1 is that of SEQ ID NO: 169; exon 11 of CDH1 is that of SEQ ID NO: 198; exon 1 of CXADR is that of SEQ ID NO: 219; exon 2 of GTF2E2 is that of SEQ ID NO: 236; exon 23 of CSMD1 is that of SEQ ID NO: 279; exon 4 of PTN is that of SEQ ID NO: 318; exon 11 of ST14 is that of SEQ ID NO: 342; exon 9 of THBS1 is that of SEQ ID NO: 386; exon 12 of AGRN is that of SEQ ID NO: 416; exon 4 of PVALB is that of SEQ ID NO: 442; exon 2 of SLC3A2 is that of SEQ ID NO: 457; exon 14 of APP is that of SEQ ID NO: 501; exon 33 of WRN is that of SEQ ID NO: 562; exon 1 of DAAM1 is that of SEQ ID NO: 606; exon 22 of ASPH is that of SEQ ID NO: 658; exon 6 of NOTCH2 is that of SEQ ID NO: 700; exon 2 of CD74 is that of SEQ ID NO: 720; exon 2 of SDC4 is that of SEQ ID NO: 746; exon 5 of CD44 is that of SEQ ID NO: 65; exon 14 of SLC4A4 is that of SEQ ID NO: 780; exon 4 of SDC4 is that of SEQ ID NO: 748; exon 12 of ZFAT is that of SEQ ID NO: 841; exon 3 of DSCAML1 is that of SEQ ID NO: 872 and exons 1, 2, 5 and 6 of NRG1 are those of SEQ ID NOs: 125, 126, 129 and 130, respectively.

15. The polynucleotide according to any one of clauses 13 or 14, wherein: —the portion of exon 1 of VAPB, or the allelic variant thereof, is 5′ to the portion of exon 2 of NRG1, or the allelic variant thereof;
the portion of exon 7 of CADM1, or the allelic variant thereof, is 5′ to the portion of exon 6 of NRG1, or the allelic variant thereof;
the portion of exon 5 of CD44, or the allelic variant thereof, is 5′ to the portion of exon 2 of NRG1, or the allelic variant thereof;
the portion of exon 1 of SLC3A2, or the allelic variant thereof, is 5′ to the portion of exon 5 of NRG1, or the allelic variant thereof;
the portion of exon 2 of VTCN1, or the allelic variant thereof, is 5′ to the portion of exon 2 of NRG1, or the allelic variant thereof;
the portion of exon 11 of CDH1, or the allelic variant thereof, is 5′ to the portion of exon 2 of NRG1, or the allelic variant thereof;
   the portion of exon 1 of CXADR, or the allelic variant thereof, is 5′ to the portion of exon 2 of NRG1, or the allelic variant thereof;
   the portion of exon 2 of GTF2E2, or the allelic variant thereof, is 5′ to the portion of exon 2 of NRG1, or the allelic variant thereof;
   the portion of exon 23 CSMD1, or the allelic variant thereof, is 5′ to the portion of exon 6 of NRG1, or the allelic variant thereof;
   the portion of exon 4 PTN, or the allelic variant thereof, is 5′ to the portion of exon 2 of NRG1, or the allelic variant thereof;
   the portion of exon 11 ST14, or the allelic variant thereof, is 5′ to the portion of exon 6 of NRG1, or the allelic variant thereof;
   the portion of exon 9 THBS1, or the allelic variant thereof, is 5′ to the portion of exon 6 of NRG1, or the allelic variant thereof;
   the portion of exon 12 AGRN, or the allelic variant thereof, is 5′ to the portion of exon 6 of NRG1, or the allelic variant thereof;
   the portion of exon 4 PVALB, or the allelic variant thereof, is 5′ to the portion of exon 6 of NRG1, or the allelic variant thereof;
   the portion of exon 2 SCL3A2, or the allelic variant thereof, is 5′ to the portion of exon 6 of NRG1, or the allelic variant thereof;
   the portion of exon 14 of APP, or the allelic variant thereof, is 5′ to the portion of exon 6 of NRG1, or the allelic variant of exon 6;
   the portion of exon 33 of WRN, or the allelic variant thereof, is 5′ to the portion of exon 6 of NRG1, or the allelic variant of exon 6;
   the portion of exon 1 of DAAM1, or the allelic variant thereof, is 5′ to the portion of exon 1 of NRG1, or the allelic variant of exon 1;
   the portion of exon 22 of ASPH, or the allelic variant thereof, is 5′ to the portion of exon 2 of NRG1, or the allelic variant of exon 2;
   the portion of exon 6 of NOTCH2, or the allelic variant thereof, is 5′ to the portion of exon 6 of NRG1, or the allelic variant of exon 6;
   the portion of exon 2 of CD74, or the allelic variant thereof, is 5′ to the portion of exon 2 of NRG1, or the allelic variant of exon 2;
   the portion of exon 2 of SDC4, or the allelic variant thereof, is 5′ to the portion of exon 2 of NRG1, or the allelic variant of exon 2;
   the portion of exon 5 of CD44, or the allelic variant thereof, is 5′ to the portion of exon 6 of NRG1, or the allelic variant of exon 6;
   the portion of exon 14 of SLC4A4, or the allelic variant thereof, is 5′ to the portion of exon 6 of NRG1, or the allelic variant of exon 6;
   a portion of exon 4 of SDC4, or the allelic variant thereof, is 5′ to the portion of exon 2 of NRG1, or the allelic variant of exon 2;
   the portion of exon 12 of ZFAT, or the allelic variant thereof, is 5′ to the portion of exon 6 of NRG1, or the allelic variant of exon 6, and
   the portion of exon 3 of DSCAML1, or the allelic variant thereof, is 5′ to the portion of exon 2 of NRG1, or the allelic variant of exon 2.

16. The polynucleotide according to any one of clauses 13-15, wherein:
the allelic variant of exon 1 of VAPB has at least 85% identity to SEQ ID NO: 17, preferably at least 90%, 92%, 94%, 96% or even 98% identity thereto;
the allelic variant of exon 7 of CADM1 has at least 85% identity to SEQ ID NO: 39, preferably at least 90%, 92%, 94%, 96% or even 98% identity thereto;
the allelic variant of exon 5 of CD44 has at least 85% identity to SEQ ID NO: 65, preferably at least 90% identity, more preferably at least 95% sequence identity, or preferably at least 90%, 92%, 94%, 96% or even 98% identity thereto;
the allelic variant of exon 1 of SLC3A2 has at least 85% identity to SEQ ID NO: 103, preferably at least 90%, 92%, 94%, 96% or even 98% identity thereto;
the allelic variant of exon 2 of VTCN1 has at least 85% identity to SEQ ID NO: 169, preferably at least 90%, 92%, 94%, 96% or even 98% identity thereto;
the allelic variant of exon 11 of CDH1 has at least 85% identity to SEQ ID NO: 198, preferably at least 90%, 92%, 94%, 96% or even 98% identity thereto;
the allelic variant of exon 2 of NRG1 has at least 85% identity to SEQ ID NO: 126, preferably at least 90%, 92%, 94%, 96% or even 98% identity thereto;
the allelic variant of exon 5 of NRG1 has at least 85% identity to SEQ ID NO: 129, preferably at least 90%, 92%, 94%, 96% or even 98% identity thereto;
the allelic variant of exon 6 of NRG1 has at least 85% identity to SEQ ID NO: 130, preferably at least 90%, 92%, 94%, 96% or even 98% identity thereto;
   the allelic variant of exon 1 of CXADR has at least 85% identity to SEQ ID NO: 219, preferably at least 90%, 92%, 94%, 96% or even 98% identity thereto;
   the allelic variant of exon 2 of GTF2E2 has at least 85% identity to SEQ ID NO: 236, preferably at least 90%, 92%, 94%, 96% or even 98% identity thereto;
   the allelic variant of exon 23 of CSMD1 has at least 85% identity to SEQ ID NO: 279, preferably at least 90%, 92%, 94%, 96% or even 98% identity thereto;
   the allelic variant of exon 4 of PTN has at least 85% identity to SEQ ID NO: 318, preferably at least 90%, 92%, 94%, 96% or even 98% identity thereto;
   the allelic variant of exon 11 of ST14 has at least 85% identity to SEQ ID NO: 342, preferably at least 90%, 92%, 94%, 96% or even 98% identity thereto;
   the allelic variant of exon 9 of THBS1 has at least 85% identity to SEQ ID NO: 386, preferably at least 90%, 92%, 94%, 96% or even 98% identity thereto;
   the allelic variant of exon 12 of AGRN has at least 85% identity to SEQ ID NO: 416, preferably at least 90%, 92%, 94%, 96% or even 98% identity thereto;
   the allelic variant of exon 4 of PVALB has at least 85% identity to SEQ ID NO: 442, preferably at least 90%, 92%, 94%, 96% or even 98% identity thereto;
   the allelic variant of exon 2 of SCL3A2 has at least 85% identity to SEQ ID NO: 457, preferably at least 90%, 92%, 94%, 96% or even 98% identity thereto;
   the allelic variant of exon 14 of APP has at least 85% identity to SEQ ID NO: 501, preferably at least 90%, 92%, 94%, 96% or even 98% identity thereto;
   the allelic variant of exon 33 of WRN has at least 85% identity to SEQ ID NO: 562, preferably at least 90%, 92%, 94%, 96% or even 98% identity thereto;
   the allelic variant of exon 1 of DAAM1 has at least 85% identity to SEQ ID NO: 606, preferably at least 90%, 92%, 94%, 96% or even 98% identity thereto;
   the allelic variant of exon 1 of NRG1 has at least 85% identity to SEQ ID NO: 125, preferably at least 90%, 92%, 94%, 96% or even 98% identity thereto;
   the allelic variant of exon 22 of ASPH has at least 85% identity to SEQ ID NO: 658, preferably at least 90%, 92%, 94%, 96% or even 98% identity thereto;
   the allelic variant of exon 6 of NOTCH2 has at least 85% identity to SEQ ID NO: 700, preferably at least 90%, 92%, 94%, 96% or even 98% identity thereto;
   the allelic variant of exon 2 of CD74 has at least 85% identity to SEQ ID NO: 720, preferably at least 90%, 92%, 94%, 96% or even 98% identity thereto;
   the allelic variant of exon 2 of SDC4 has at least 85% identity to SEQ ID NO: 746, preferably at least 90%, 92%, 94%, 96% or even 98% identity thereto;
   the allelic variant of exon 5 of CD44 has at least 85% identity to SEQ ID NO: 65, preferably at least 90%, 92%, 94%, 96% or even 98% identity thereto;
   the allelic variant of exon 14 of SLC4A4 has at least 85% identity to SEQ ID NO: 780, preferably at least 90%, 92%, 94%, 96% or even 98% identity thereto;
   the allelic variant of exon 4 of SDC4 has at least 85% identity to SEQ ID NO: 748, preferably at least 90%, 92%, 94%, 96% or even 98% identity thereto;
   the allelic variant of exon 12 of ZFAT has at least 85% identity to SEQ ID NO: 841, preferably at least 90%, 92%, 94%, 96% or even 98% identity thereto; and
   the allelic variant of exon 3 of DSCAML1 has at least 85% identity to SEQ ID NO: 872, preferably at least 90%, 92%, 94%, 96% or even 98% identity thereto.

17. The polynucleotide according to any one of clauses 13-16, wherein:
the fusion of VAPB with NRG1 comprises 2 to about 40 contiguous nucleic acids from SEQ ID NO: 3, including the nucleic acids at position 43 and 44;
the fusion of CADM1 with NRG1 comprises 2 to about 40 contiguous nucleic acids from SEQ ID NO: 7, including the nucleic acids at position 53 and 54;
the fusion of CD44 with NRG1 comprises 2 to about 40 contiguous nucleic acids from SEQ ID NO: 11, including the nucleic acids at position 52 and 53;
the fusion of SLC3A2 with NRG1 comprises 2 to about 40 contiguous nucleic acids from SEQ ID NO: 15, including the nucleic acids at position 53 and 54;
the fusion of VTCN1 with NRG1 comprises 2 to about 40 contiguous nucleic acids from SEQ ID NO: 166, including the nucleic acids at position 65 and 66;
the fusion of CDH1 with NRG1 comprises 2 to about 40 contiguous nucleic acids from SEQ ID NO: 186, including the nucleic acids at position 119 and 120;
the fusion of CXADR with NRG1 comprises 2 to about 40 contiguous nucleic acids from SEQ ID NO: 217, including the nucleic acids at position 43 and 44;
the fusion of GTF2E2 with NRG1 comprises 2 to about 40 contiguous nucleic acids from SEQ ID NO: 233, including the nucleic acids at position 141 and 142;
the fusion of CSMD1 with NRG1 comprises 2 to about 40 contiguous nucleic acids from SEQ ID NO: 255, including the nucleic acids at position 88 and 89;
the fusion of PTN with NRG1 comprises 2 to about 40 contiguous nucleic acids from SEQ ID NO: 313, including the nucleic acids at position 102 and 103;
the fusion of ST14 with NRG1 comprises 2 to about 40 contiguous nucleic acids from SEQ ID NO: 330, including the nucleic acids at position 95 and 96;
the fusion of THBS1 with NRG1 comprises 2 to about 40 contiguous nucleic acids from SEQ ID NO: 376, including the nucleic acids at position 56 and 57;
the fusion of AGRN with NRG1 comprises 2 to about 40 contiguous nucleic acids from SEQ ID NO: 403, including the nucleic acids at position 106 and 107;
the fusion of PVALB with NRG1 comprises 2 to about 40 contiguous nucleic acids from SEQ ID NO: 437, including the nucleic acids at position 102 and 103;
the fusion of SLC3A2 with NRG1 comprises 2 to about 40 contiguous nucleic acids from SEQ ID NO: 454, including the nucleic acids at position 93 and 94;
the fusion of APP with NRG1 comprises 2 to about 40 contiguous nucleic acids from SEQ ID NO: 486, including the nucleic acids at position 54 and 55;
the fusion of WRN with NRG1 comprises 2 to about 40 contiguous nucleic acids from SEQ ID NO: 528, including the nucleic acids at position 96 and 97;
the fusion of DAAM1 with NRG1 comprises 2 to about 40 contiguous nucleic acids from SEQ ID NO: 605, including the nucleic acids at position 75 and 76;
the fusion of ASPH with NRG1 comprises 2 to about 40 contiguous nucleic acids from SEQ ID NO: 635, including the nucleic acids at position 75 and 76;
the fusion of NOTCH2 with NRG1 comprises 2 to about 40 contiguous nucleic acids from SEQ ID NO: 693, including the nucleic acids at position 75 and 76;
the fusion of CD74 with NRG1 comprises 2 to about 40 contiguous nucleic acids from SEQ ID NO: 717, including the nucleic acids at position 75 and 76;
the fusion of SDC4 with NRG1 comprises 2 to about 40 contiguous nucleic acids from SEQ ID NO: 743, including the nucleic acids at position 75 and 76;
the fusion of CD44 with NRG1 comprises 2 to about 40 contiguous nucleic acids from SEQ ID NO: 761, including the nucleic acids at position 75 and 76;
the fusion of SLC4A4 with NRG1 comprises 2 to about 40 contiguous nucleic acids from SEQ ID NO: 765, including the nucleic acids at position 75 and 76;
the fusion of SDC4 with NRG1 comprises 2 to about 40 contiguous nucleic acids from SEQ ID NO: 824, including the nucleic acids at position 75 and 76;
the fusion of ZFAT with NRG1 comprises 2 to about 40 contiguous nucleic acids from SEQ ID NO: 828, including the nucleic acids at position 75 and 76; and
the fusion of DSCAML1 with NRG1 comprises 2 to about 40 contiguous nucleic acids from SEQ ID NO: 868, including the nucleic acids at position 75 and 76.

18. The polynucleotide according to any one of clauses 13-17, wherein:
the fusion of VAPB with NRG1 comprises SEQ ID NO: 3, or an allelic variant thereof;
the fusion of CADM1 with NRG1 comprises SEQ ID NO: 7, or an allelic variant thereof;
the fusion of CD44 with NRG1 comprises SEQ ID NO: 11, or an allelic variant thereof;
the fusion of SLC3A2 with NRG1 comprises SEQ ID NO: 15, or an allelic variant thereof;
the fusion of VTCN1 with NRG1 comprises SEQ ID NO: 166, or an allelic variant thereof;
the fusion of CDH1 with NRG1 comprises SEQ ID NO: 186, or an allelic variant thereof;
the fusion of CXADR with NRG1 comprises SEQ ID NO: 217, or an allelic variant thereof;
the fusion of GTF2E2 with NRG1 comprises SEQ ID NO: 233, or an allelic variant thereof;
the fusion of CSMD1 with NRG1 comprises SEQ ID NO: 255, or an allelic variant thereof;
the fusion of PTN with NRG1 comprises SEQ ID NO: 313, or an allelic variant thereof;
the fusion of ST14 with NRG1 comprises SEQ ID NO: 330, or an allelic variant thereof;
the fusion of THBS1 with NRG1 comprises SEQ ID NO: 376, or an allelic variant thereof;
the fusion of AGRN with NRG1 comprises SEQ ID NO: 403, or an allelic variant thereof;
the fusion of PVALB with NRG1 comprises SEQ ID NO: 437, or an allelic variant thereof;
the fusion of SLC3A2 with NRG1 comprises SEQ ID NO: 454, or an allelic variant thereof;
the fusion of APP with NRG1 comprises SEQ ID NO: 486, or an allelic variant thereof;
the fusion of WRN with NRG1 comprises SEQ ID NO: 528, or an allelic variant thereof;
the fusion of DAAM1 with NRG1 comprises SEQ ID NO: 605, or an allelic variant thereof;
the fusion of ASPH with NRG1 comprises SEQ ID NO: 635, or an allelic variant thereof;
the fusion of NOTCH2 with NRG1 comprises SEQ ID NO: 693, or an allelic variant thereof;
the fusion of CD74 with NRG1 comprises SEQ ID NO: 717, or an allelic variant thereof;
the fusion of SDC4 with NRG1 comprises SEQ ID NO: 743, or an allelic variant thereof;
the fusion of CD44 with NRG1 comprises SEQ ID NO: 761, or an allelic variant thereof;
the fusion of SLC4A4 with NRG1 comprises SEQ ID NO: 765, or an allelic variant thereof;
the fusion of SDC4 with NRG1 comprises SEQ ID NO: 824, or an allelic variant thereof;
the fusion of ZFAT with NRG1 comprises SEQ ID NO: 828, or an allelic variant thereof; and
the fusion of DSCAML1 with NRG1 comprises SEQ ID NO: 868, or an allelic variant thereof.

19. The polynucleotide according to any one of clauses 13-18, wherein:
the portion of exon 1 of VAPB is or comprises SEQ ID NO: 1, or an allelic variant SEQ ID NO: 1;
the portion of exon 7 of CADM1 is or comprises SEQ ID NO: 5, or an allelic variant of SEQ ID NO: 5;
the portion of exon 5 of CD44 is or comprises SEQ ID NO: 9, or an allelic variant SEQ ID NO: 9;
the portion of exon 1 of SLC3A2 is or comprises SEQ ID NO: 13, or an allelic variant SEQ ID NO: 13;
the portion of exon 2 of VTCN1 is or comprises SEQ ID NO: 164, or an allelic variant SEQ ID NO: 164;
   the portion of exon 11 of CDH1 is or comprises SEQ ID NO: 184, or an allelic variant SEQ ID NO: 184;
   the portion of exon 1 of CXADR is or comprises SEQ ID NO: 219, or an allelic variant SEQ ID NO: 219;
   the portion of exon 2 of GTF2E2 is or comprises SEQ ID NO: 236, or an allelic variant SEQ ID NO: 236;
   the portion of exon 23 of CXADR is or comprises SEQ ID NO: 279, or an allelic variant SEQ ID NO: 279;
   the portion of exon 4 of PTN is or comprises SEQ ID NO: 318, or an allelic variant SEQ ID NO: 318;
   the portion of exon 11 of ST14 is or comprises SEQ ID NO:342, or an allelic variant SEQ ID NO: 342;
   the portion of exon 9 of THBS1 is or comprises SEQ ID NO: 385, or an allelic variant SEQ ID NO: 386;
   the portion of exon 12 of AGRN is or comprises SEQ ID NO: 416, or an allelic variant SEQ ID NO: 416;
   the portion of exon 4 of PVALB is or comprises SEQ ID NO: 442, or an allelic variant SEQ ID NO: 442;
   the portion of exon 2 of SLC3A2 is or comprises SEQ ID NO: 457, or an allelic variant SEQ ID NO: 457;
the portion of exon 2 of NRG1 is or comprises SEQ ID NO: 165, or an allelic variant SEQ ID NO: 165;
the portion of exon 5 of NRG1 is or comprises SEQ ID NO: 14, or an allelic variant SEQ ID NO: 14;
the portion of exon 6 of NRG1 is or comprises SEQ ID NO: 6, or an allelic variant thereof;
   the portion of exon 14 of APP is or comprises SEQ ID NO: 484, or an allelic variant thereof;
   the portion of exon 33 of WRN is or comprises SEQ ID NO: 526, or an allelic variant thereof;
   the portion of exon 1 of DAAM1 is or comprises SEQ ID NO: 603, or an allelic variant thereof;
   the portion of exon 22 of ASPH is or comprises SEQ ID NO: 633, or an allelic variant thereof;
   the portion of exon 6 of NOTCH2 is or comprises SEQ ID NO: 691, or an allelic variant thereof;
   the portion of exon 2 of CD74 is or comprises SEQ ID NO: 715, or an allelic variant thereof;
   the portion of exon 2 of SDC4 is or comprises SEQ ID NO: 741, or an allelic variant thereof;
   the portion of exon 5 of CD44 is or comprises SEQ ID NO: 759, or an allelic variant thereof;
   the portion of exon 14 of SLC4A4 is or comprises SEQ ID NO: 763, or an allelic variant thereof;
   a portion of exon 4 of SDC4 is or comprises SEQ ID NO: 822, or an allelic variant thereof;
   the portion of exon 12 of ZFAT is or comprises SEQ ID NO: 826, or an allelic variant thereof,
   the portion of exon 3 of DSCAML1 is or comprises SEQ ID NO: 866, or an allelic variant thereof; and
   the portion of exon 1 of NRG1 is or comprises SEQ ID NO: 604, or an allelic variant thereof.

20. The polynucleotide according to any one of clauses 13-18, wherein:
the fusion of VAPB with NRG1 comprises a fusion junction between exon 1 of VAPB and exon 2 of NRG1, preferably the junction between the nucleic acids at position 43 of VAPB and at position 44 of NRG1 of SEQ ID NO: 3;
the fusion of CADM1 with NRG1 comprises a fusion junction between exon 7 of CADM1 and exon 6 of NRG1, preferably the junction between the nucleic acids at position 53 of CADM1 and at position 54 of NRG1 of SEQ ID NO: 7;
the fusion of CD44 with NRG1 comprises a fusion junction between exon 5 of CD44 and exon 2 of NRG1, preferably the junction between the nucleic acids at position 52 of CD44 and at position 53 of NRG1 of SEQ ID NO: 11;
the fusion of SLC3A2 with NRG1 comprises a fusion junction between exon 1 of SLC3A2 and exon 5 of NRG1, preferably the junction between the nucleic acids at position 53 of SLC3A2 and at position 54 of NRG1 of SEQ ID NO: 15;
the fusion of VTCN1 with NRG1 comprises a fusion junction between exon 2 of VTCN1 and exon 2 of NRG1, preferably the junction between the nucleic acids at position 65 of VTCN1 and at position 66 of NRG1 of SEQ ID NO: 166;
the fusion of CDH1 with NRG1 comprises a fusion junction between exon 11 of CDH1 and exon 2 of NRG1, preferably the junction between the nucleic acids at position 119 of CDH1 and at position 120 of NRG1 of SEQ ID NO: 186
the fusion of CXADR with NRG1 comprises a fusion junction between exon 1 of CXADR and exon 2 of NRG1, preferably the junction between the nucleic acids at position 43 of CXADR and at position 44 of NRG1 of SEQ ID NO: 217;
the fusion of GTF2E2 with NRG1 comprises a fusion junction between exon 2 of GTF2E2 and exon 2 of NRG1, preferably the junction between the nucleic acids at position 141 of GTF2E2 and at position 142 of NRG1 of SEQ ID NO: 233;
the fusion of CSMD1 with NRG1 comprises a fusion junction between exon 23 of CSMD1 and exon 6 of NRG1, preferably the junction between the nucleic acids at position 88 of CSMD1 and at position 89 of NRG1 of SEQ ID NO: 255;
the fusion of PTN with NRG1 comprises a fusion junction between exon 4 of PTN and exon 2 of NRG1, preferably the junction between the nucleic acids at position 102 of PTN and at position 103 of NRG1 of SEQ ID NO: 313;
the fusion of ST14 with NRG1 comprises a fusion junction between exon 11 of ST14 and exon 6 of NRG1, preferably the junction between the nucleic acids at position 95 of ST14 and at position 96 of NRG1 of SEQ ID NO: 330;
the fusion of THBS1 with NRG1 comprises a fusion junction between exon 9 of THBS1 and exon 6 of NRG1, preferably the junction between the nucleic acids at position 56 of THBS1 and at position 57 of NRG1 of SEQ ID NO: 376;
the fusion of AGRN with NRG1 comprises a fusion junction between exon 12 of AGRN and exon 6 of NRG1, preferably the junction between the nucleic acids at position 106 of AGRN and at position 107 of NRG1 of SEQ ID NO: 403;
the fusion of PVALB with NRG1 comprises a fusion junction between exon 4 of PVALB and exon 6 of NRG1, preferably the junction between the nucleic acids at position 102 of PVALB and at position 103 of NRG1 of SEQ ID NO: 437;
the fusion of SLC3A2 with NRG1 comprises a fusion junction between exon 2 of SLC3A2 and exon 6 of NRG1, preferably the junction between the nucleic acids at position 93 of SLC3A2 and at position 94 of NRG1 of SEQ ID NO: 454;
the fusion of APP with NRG1 comprises a fusion junction between exon 14 of APP and exon 6 of NRG1, preferably the junction between the nucleic acids at position 54 of APP and at position 55 of NRG1 of SEQ ID NO: 486;
the fusion of WRN with NRG1 comprises a fusion junction between exon 33 of WRN and exon 6 of NRG1, preferably the junction between the nucleic acids at position 96 of WRN and at position 97 of NRG1 of SEQ ID NO: 528;
the fusion of DAAM1 with NRG1 comprises a fusion junction between exon 1 of DAAM1 and exon 1 of NRG1, preferably the junction between the nucleic acids at position 75 of DAAM1 and at position 76 of NRG1 of SEQ ID NO: 605;
the fusion of ASPH with NRG1 comprises a fusion junction between exon 22 of ASPH and exon 2 of NRG1, preferably the junction between the nucleic acids at position 75 of ASPH and at position 76 of NRG1 of SEQ ID NO: 635;
the fusion of NOTCH2 with NRG1 comprises a fusion junction between exon 6 of NOTCH2 and exon 6 of NRG1, preferably the junction between the nucleic acids at position 75 of NOTCH2 and at position 76 of NRG1 of SEQ ID NO: 693;
the fusion of CD74 with NRG1 comprises a fusion junction between exon 2 of CD74 and exon 2 of NRG1, preferably the junction between the nucleic acids at position 75 of CD74 and at position 76 of NRG1 of SEQ ID NO: 717;
the fusion of SDC4 with NRG1 comprises a fusion junction between exon 2 of SDC4 and exon 2 of NRG1, preferably the junction between the nucleic acids at position 75 of SDC4 and at position 76 of NRG1 of SEQ ID NO: 743;
the fusion of CD44 with NRG1 comprises a fusion junction between exon 5 of CD44 and exon 6 of NRG1, preferably the junction between the nucleic acids at position 75 of CD44 and at position 76 of NRG1 of SEQ ID NO: 761;
the fusion of SLC4A4 with NRG1 comprises a fusion junction between exon 14 of SLC4A4 and exon 6 of NRG1, preferably the junction between the nucleic acids at position 75 of SLC4A4 and at position 76 of NRG1 of SEQ ID NO: 765;
the fusion of SDC4 with NRG1 comprises a fusion junction between exon 4 of SDC4 and exon 2 of NRG1, preferably the junction between the nucleic acids at position 75 of SDC4 and at position 76 of NRG1 of SEQ ID NO: 824;
the fusion of ZFAT with NRG1 comprises a fusion junction between exon 12 of ZFAT and exon 6 of NRG1, preferably the junction between the nucleic acids at position 75 of ZFAT and at position 76 of NRG1 of SEQ ID NO: 828; and
the fusion of DSCAML1 with NRG1 comprises a fusion junction between exon 3 of DSCAML1 and exon 2 of NRG1, preferably the junction between the nucleic acids at position 75 of DSCAML1 and at position 76 of NRG1 of SEQ ID NO: 868.

21. The polynucleotide according to any one of the previous clauses, wherein the polynucleotide is isolated or purified.

22. The polynucleotide according to any one of the previous clauses, wherein any one of the fusions is an in-frame fusion.

23. The polynucleotide according to any one of the previous clauses, wherein the polynucleotide is a mammalian polynucleotide, preferably a human polynucleotide.

24. A polypeptide fusion encoded by the polynucleotide according to any one of the previous clauses.

25. A vector comprising the polynucleotide according to any one of clauses 1-23.

26. A recombinant host cell comprising the polynucleotide of any of clauses 1-23 or the vector of clause 25.

27. A method of making the polypeptide fusion of clause 24, comprising maintaining the host cell of clause 26 under conditions suitable for expression of the polynucleotide comprised by the host cell, whereby the polynucleotide is expressed and a polypeptide fusion is produced, followed by isolating or purifying the polypeptide fusion.

28. A method for making a recombinant host cell comprising introducing the vector of clause 25 into a host cell.

29. A detection assay comprising a nucleic acid probe, primer or primer pair for detection of the presence of a polynucleotide fusion according to any one of clauses 1-23.

30. A nucleic acid probe, primer or primer pair for detection of a polynucleotide fusion according to any of the clauses 1-23.

31. The nucleic acid probe, primer or primer pair of clause 30, having a length of 10-40 nucleotides.

32. The nucleic acid probe, primer or primer pair of clauses 30 or 31, wherein the fusion as detected comprises:
the fusion of VAPB with NRG1 comprising or consisting of SEQ ID NO: 3, and preferably includes the nucleic acids at position 43 and 44;
the fusion of CADM1 with NRG1 comprising or consisting of SEQ ID NO: 7, and preferably includes the nucleic acids at position 53 and 54;
the fusion of CD44 with NRG1 comprising or consisting of SEQ ID NO: 11, and preferably includes the nucleic acids at position 52 and 53;
the fusion of SLC3A2 with NRG1 comprising or consisting of SEQ ID NO: 15, and preferably includes the nucleic acids at position 53 and 54;
the fusion of VTCN1 with NRG1 comprising or consisting of SEQ ID NO: 166, and preferably includes the nucleic acids at position 65 and 66;
the fusion of CDH1 with NRG1 comprising or consisting of SEQ ID NO: 186, and preferably includes the nucleic acids at position 119 and 120;
the fusion of CXADR with NRG1 comprising or consisting of SEQ ID NO: 217, and preferably includes the nucleic acids at position 43 and 44;
the fusion of GTF2E2 with NRG1 comprising or consisting of SEQ ID NO: 233, and preferably includes the nucleic acids at position 141 and 142;
the fusion of CSMD1 with NRG1 comprising or consisting of SEQ ID NO: 255, and preferably includes the nucleic acids at position 88 and 89;
the fusion of PTN with NRG1 comprising or consisting of SEQ ID NO: 313, and preferably includes the nucleic acids at position 102 and 103;
the fusion of ST14 with NRG1 comprising or consisting of SEQ ID NO: 330, and preferably includes the nucleic acids at position 95 and 96;
the fusion of THBS1 with NRG1 comprising or consisting of SEQ ID NO: 376, and preferably includes the nucleic acids at position 56 and 57;
the fusion of AGRN with NRG1 comprising or consisting of SEQ ID NO: 403, and preferably includes the nucleic acids at position 106 and 107;
the fusion of PVALB with NRG1 comprising or consisting of SEQ ID NO: 437, and preferably includes the nucleic acids at position 102 and 103,
the fusion of SLC3A2 with NRG1 comprising or consisting of SEQ ID NO: 454, and preferably includes the nucleic acids at position 93 and 94;
the fusion of APP with NRG1 comprising or consisting of SEQ ID NO: 486, and preferably includes the nucleic acids at position 54 and 55;
the fusion of WRN with NRG1 comprising or consisting of SEQ ID NO: 528, and preferably includes the nucleic acids at position 96 and 97;
the fusion of DAAM1 with NRG1 comprising or consisting of SEQ ID NO: 605, and preferably includes the nucleic acids at position 75 and 76;
the fusion of ASPH with NRG1 comprising or consisting of SEQ ID NO: 635, and preferably includes the nucleic acids at position 75 and 76;
the fusion of NOTCH2 with NRG1 comprising or consisting of SEQ ID NO: 693, and preferably includes the nucleic acids at position 75 and 76;
the fusion of CD74 with NRG1 comprising or consisting of SEQ ID NO: 717, and preferably includes the nucleic acids at position 75 and 76;
the fusion of SDC4 with NRG1 comprising or consisting of SEQ ID NO: 743, and preferably includes the nucleic acids at position 75 and 76;
the fusion of CD44 with NRG1 comprising or consisting of SEQ ID NO: 761, and preferably includes the nucleic acids at position 75 and 76;
the fusion of SLC4A4 with NRG1 comprising or consisting of SEQ ID NO: 765, and preferably includes the nucleic acids at position 75 and 76;
the fusion of SDC4 with NRG1 comprising or consisting of SEQ ID NO: 824, and preferably includes the nucleic acids at position 75 and 76;
the fusion of ZFAT with NRG1 comprising or consisting of SEQ ID NO: 828, and preferably includes the nucleic acids at position 75 and 76; and
the fusion of DSCAML1 with NRG1 comprising or consisting of SEQ ID NO: 868, and preferably includes the nucleic acids at position 75 and 76.

33. The nucleic acid probe, primer or primer pair of any one of clauses 30-32, wherein:
the probe, primer or primer pair for detection of the fusion of VAPB with NRG1 specifically hybridizes to, or has 95% or more complementary sequence identity with, a sequence comprised by exon 1 from VAPB or a sequence located 5′ of exon 1 and/or to a sequence comprised by exon 2 from NRG1, or a sequence located 3′ of exon 2;
the probe, primer or primer pair for detection of the fusion of CADM1 with NRG1 specifically hybridizes to, or has 95% or more complementary sequence identity with, a sequence comprised by exon 7 from CADM1, or a sequence located 5′ of exon 7 and/or to a sequence comprised by exon 6 from NRG1, or a sequence located 3′ of exon 6;
the probe, primer or primer pair for detection of the fusion of CD44 with NRG1 specifically hybridizes to, or has 95% or more complementary sequence identity with, a sequence comprised by exon 5 from CD44, or a sequence located 5′ of exon 5 and/or to a sequence comprised by exon 2 from NRG1, or a sequence located 3′ of exon 2;
the probe, primer or primer pair for detection of the fusion of transcript version 6 of SLC3A2 with NRG1 specifically hybridizes to, or has 95% or more complementary sequence identity with, a sequence comprised by exon 1 from said SLC3A2 or a sequence located 5′ of exon 1 and/or to a sequence comprised by exon 5 from NRG1, or a sequence located 3′ of exon 5;
the probe, primer or primer pair for detection of the fusion of VTCN1 with NRG1 specifically hybridizes to, or has 95% or more complementary sequence identity with, a sequence comprised by exon 2 from VTCN1, or a sequence located 5′ of exon 2 and/or to a sequence comprised by exon 2 from NRG1, or a sequence located 3′ of exon 2;
the probe, primer or primer pair for detection of the fusion of CDH1 with NRG1 specifically hybridizes to, or has 95% or more complementary sequence identity with, a sequence comprised by exon 11 from CDH1, or a sequence located 5′ of exon 11 and/or to a sequence comprised by exon 2 from NRG1, or a sequence located 3′ of exon 2;
   the probe, primer or primer pair for detection of the fusion of CXADR with NRG1 specifically hybridizes to, or has 95% or more complementary sequence identity with, a sequence comprised by exon 1 from CXADR, or a sequence located 5′ of exon 1 and/or to a sequence comprised by exon 2 from NRG1, or a sequence located 3′ of exon 2;
   the probe, primer or primer pair for detection of the fusion of GTF2E2 with NRG1 specifically hybridizes to, or has 95% or more complementary sequence identity with, a sequence comprised by exon 2 from GTF2E2, or a sequence located 5′ of exon 2 and/or to a sequence comprised by exon 2 from NRG1, or a sequence located 3′ of exon 2;
   the probe, primer or primer pair for detection of the fusion of CSMD1 with NRG1 specifically hybridizes to, or has 95% or more complementary sequence identity with, a sequence comprised by exon 23 from CSMD1, or a sequence located 5′ of exon 23 and/or to a sequence comprised by exon 6 from NRG1, or a sequence located 3′ of exon 6;
   the probe, primer or primer pair for detection of the fusion of PTN with NRG1 specifically hybridizes to, or has 95% or more complementary sequence identity with, a sequence comprised by exon 4 from PTN, or a sequence located 5′ of exon 4 and/or to a sequence comprised by exon 2 from NRG1, or a sequence located 3′ of exon 2;
   the probe, primer or primer pair for detection of the fusion of ST14 with NRG1 specifically hybridizes to, or has 95% or more complementary sequence identity with, a sequence comprised by exon 11 from ST14, or a sequence located 5′ of exon 11 and/or to a sequence comprised by exon 6 from NRG1, or a sequence located 3′ of exon 6;
   the probe, primer or primer pair for detection of the fusion of THBS1 with NRG1 specifically hybridizes to, or has 95% or more complementary sequence identity with, a sequence comprised by exon 9 from THBS1, or a sequence located 5′ of exon 9 and/or to a sequence comprised by exon 6 from NRG1, or a sequence located 3′ of exon 6;
   the probe, primer or primer pair for detection of the fusion of AGRN with NRG1 specifically hybridizes to, or has 95% or more complementary sequence identity with, a sequence comprised by exon 12 from AGRN, or a sequence located 5′ of exon 12 and/or to a sequence comprised by exon 6 from NRG1, or a sequence located 3′ of exon 6;
   the probe, primer or primer pair for detection of the fusion of PVALB with NRG1 specifically hybridizes to, or has 95% or more complementary sequence identity with, a sequence comprised by exon 4 from PVALB, or a sequence located 5′ of exon 4 and/or to a sequence comprised by exon 6 from NRG1, or a sequence located 3′ of exon 6;
   the probe, primer or primer pair for detection of the fusion of transcript version 3 of SLC3A2 with NRG1 specifically hybridizes to, or has 95% or more complementary sequence identity with, a sequence comprised by exon 2 from said SLC3A2, or a sequence located 5′ of exon 2 and/or to a sequence comprised by exon 6 from NRG1, or a sequence located 3′ of exon 6;
   the probe, primer or primer pair for detection of the fusion of APP with NRG1 specifically hybridizes to, or has 95% or more complementary sequence identity with, a sequence comprised by exon 14 from APP, or a sequence located 5′ of exon 14 and/or to a sequence comprised by exon 6 from NRG1, or a sequence located 3′ of exon 6;
   the probe, primer or primer pair for detection of the fusion of WRN with NRG1 specifically hybridizes to, or has 95% or more complementary sequence identity with, a sequence comprised by exon 33 from WRN, or a sequence located 5′ of exon 33 and/or to a sequence comprised by exon 6 from NRG1, or a sequence located 3′ of exon 6;
   the probe, primer or primer pair for detection of the fusion of DAAM1 with NRG1 specifically hybridizes to, or has 95% or more complementary sequence identity with, a sequence comprised by exon 1 from DAAM1, or a sequence located 5′ of exon 1 and/or to a sequence comprised by exon 1 from NRG1, or a sequence located 3′ of exon 1;
   the probe, primer or primer pair for detection of the fusion of ASPH with NRG1 specifically hybridizes to, or has 95% or more complementary sequence identity with, a sequence comprised by exon 22 from ASPH, or a sequence located 5′ of exon 22 and/or to a sequence comprised by exon 2 from NRG1, or a sequence located 3′ of exon 2;
   the probe, primer or primer pair for detection of the fusion of NOTCH2 with NRG1 specifically hybridizes to, or has 95% or more complementary sequence identity with, a sequence comprised by exon 6 from NOTCH2, or a sequence located 5′ of exon 6 and/or to a sequence comprised by exon 6 from NRG1, or a sequence located 3′ of exon 6;
   the probe, primer or primer pair for detection of the fusion of CD74 with NRG1 specifically hybridizes to, or has 95% or more complementary sequence identity with, a sequence comprised by exon 2 from CD74, or a sequence located 5′ of exon 2 and/or to a sequence comprised by exon 2 from NRG1, or a sequence located 3′ of exon 2;
   the probe, primer or primer pair for detection of the fusion of SDC4 with NRG1 specifically hybridizes to, or has 95% or more complementary sequence identity with, a sequence comprised by exon 2 from SDC4, or a sequence located 5′ of exon 2 and/or to a sequence comprised by exon 2 from NRG1, or a sequence located 3′ of exon 2;
   the probe, primer or primer pair for detection of the fusion of CD44 with NRG1 specifically hybridizes to, or has 95% or more complementary sequence identity with, a sequence comprised by exon 5 from CD44, or a sequence located 5′ of exon 5 and/or to a sequence comprised by exon 6 from NRG1, or a sequence located 3′ of exon 6;
   the probe, primer or primer pair for detection of the fusion of SLC4A4 with NRG1 specifically hybridizes to, or has 95% or more complementary sequence identity with, a sequence comprised by exon 14 from SLC4A4, or a sequence located 5′ of exon 14 and/or to a sequence comprised by exon 6 from NRG1, or a sequence located 3′ of exon 6;
   the probe, primer or primer pair for detection of the fusion of SDC4 with NRG1 specifically hybridizes to, or has 95% or more complementary sequence identity with, a sequence comprised by exon 4 from SDC4, or a sequence located 5′ of exon 4 and/or to a sequence comprised by exon 2 from NRG1, or a sequence located 3′ of exon 2;
   the probe, primer or primer pair for detection of the fusion of ZFAT with NRG1 specifically hybridizes to, or has 95% or more complementary sequence identity with, a sequence comprised by exon 12 from ZFAT, or a sequence located 5′ of exon 12 and/or to a sequence comprised by exon 6 from NRG1, or a sequence located 3′ of exon 6; or
   the probe, primer or primer pair for detection of the fusion of DSCAML1 with NRG1 specifically hybridizes to, or has 95% or more complementary sequence identity with, a sequence comprised by exon 3 from DSCAML1, or a sequence located 5′ of exon 3 and/or to a sequence comprised by exon 2 from NRG1, or a sequence located 3′ of exon 2.

34. The nucleic acid probe, primer or primer pair of clause 33, wherein:
exon 1 from VAPB comprises or consists of SEQ ID NO: 17 or an allelic variant thereof;
exon 7 from CADM1 comprises or consists of SEQ ID NO: 39 or an allelic variant thereof;
exon 5 from CD44 comprises or consists of SEQ ID NO: 65 or an allelic variant thereof;
exon 1 from SLC3A2 comprises or consists of SEQ ID NO: 103 or an allelic variant thereof;
exon 2 from VTCN1 comprises or consists of SEQ ID NO: 169 or an allelic variant thereof;
exon 11 from CDH1 comprises or consists of SEQ ID NO: 198 or an allelic variant thereof;
exon 1 from CXADR comprises or consists of SEQ ID NO: 219 or an allelic variant thereof;
exon 2 from GTF2E2 comprises or consists of SEQ ID NO: 236 or an allelic variant thereof;
exon 23 from CSMD1 comprises or consists of SEQ ID NO: 279 or an allelic variant thereof;
exon 4 from PTN comprises or consists of SEQ ID NO: 318 or an allelic variant thereof;
exon 11 from ST14 comprises or consists of SEQ ID NO: 342 or an allelic variant thereof;
exon 9 from THBS1 comprises or consists of SEQ ID NO: 386 or an allelic variant thereof;
exon 12 from AGRN comprises or consists of SEQ ID NO: 416 or an allelic variant thereof;
exon 4 from PVALB comprises or consists of SEQ ID NO: 442 or an allelic variant thereof;
exon 2 from SLC3A2 comprises or consists of SEQ ID NO: 457 or an allelic variant thereof;
exon 14 from APP comprises or consists of SEQ ID NO: 501 or an allelic variant thereof;
exon 33 from WRN comprises or consists of SEQ ID NO: 562 or an allelic variant thereof;
exon 1 from DAAM1 comprises or consists of SEQ ID NO: 606 or an allelic variant thereof;
exon 22 from ASPH comprises or consists of SEQ ID NO: 658 or an allelic variant thereof;
exon 6 from NOTCH2 comprises or consists of SEQ ID NO: 700 or an allelic variant thereof;
exon 2 from CD74 comprises or consists of SEQ ID NO: 720 or an allelic variant thereof;
exon 2 from SDC4 comprises or consists of SEQ ID NO: 746 or an allelic variant thereof;
exon 5 from CD44 comprises or consists of SEQ ID NO: 65 or an allelic variant thereof;
exon 14 from SLC4A4 comprises or consists of SEQ ID NO: 780 or an allelic variant thereof;
exon 4 from SDC4 comprises or consists of SEQ ID NO: 748 or an allelic variant thereof;
exon 12 from ZFAT comprises or consists of SEQ ID NO: 841 or an allelic variant thereof;
exon 3 from DSCAML1 comprises or consists of SEQ ID NO: 872 or an allelic variant thereof and
exons 1, 2, 5 and 6 from NRG1 comprise or consist of SEQ ID NO: 125, 126, 129 and 130, respectively, or an allelic variant thereof.

35. The nucleic acid probe, primer or primer pair of clause 33, wherein:
the probe, primer or primer pair for detection of the fusion of VAPB with NRG1 specifically hybridizes to, or has 95% or more complementary sequence identity with, a sequence comprised by SEQ ID NO: 17, or to an allelic variant thereof, and/or to a sequence comprised by SEQ ID NO: 153;
the probe, primer or primer pair for detection of the fusion of CADM1 with NRG1 specifically hybridizes to, or has 95% or more complementary sequence identity with, a sequence comprised by SEQ ID NO: 57 and/or to a sequence comprised by SEQ ID NO: 155;
the probe, primer or primer pair for detection of the fusion of CD44 with NRG1 specifically hybridizes to, or has 95% or more complementary sequence identity with, a sequence comprised by SEQ ID NO: 99 and/or to a sequence comprised by SEQ ID NO: 153;
the probe, primer or primer pair for detection of the fusion of SLC3A2 with NRG1 specifically hybridizes to, or has 95% or more complementary sequence identity with, a sequence comprised by SEQ ID NO: 103 and/or to a sequence comprised by SEQ ID NO: 157;
the probe, primer or primer pair for detection of the fusion of VTCN1 with NRG1 specifically hybridizes to, or has 95% or more complementary sequence identity with, a sequence comprised by SEQ ID NO: 181 and/or to a sequence comprised by SEQ ID NO: 153;
the probe, primer or primer pair for detection of the fusion of CDH1 with NRG1 specifically hybridizes to, or has 95% or more complementary sequence identity with, a sequence comprised by SEQ ID NO: 213 and/or to a sequence comprised by SEQ ID NO: 153;
the probe, primer or primer pair for detection of the fusion of CXADR with NRG1 specifically hybridizes to, or has 95% or more complementary sequence identity with, a sequence comprised by SEQ ID NO: 219 and/or to a sequence comprised by SEQ ID NO: 153;
the probe, primer or primer pair for detection of the fusion of GTF2E2 with NRG1 specifically hybridizes to, or has 95% or more complementary sequence identity with, a sequence comprised by SEQ ID NO: 252 and/or to a sequence comprised by SEQ ID NO: 153;
the probe, primer or primer pair for detection of the fusion of CSMD1 with NRG1 specifically hybridizes to, or has 95% or more complementary sequence identity with, a sequence comprised by SEQ ID NO: 309 and/or to a sequence comprised by SEQ ID NO: 155;
the probe, primer or primer pair for detection of the fusion of PTN with NRG1 specifically hybridizes to, or has 95% or more complementary sequence identity with, a sequence comprised by SEQ ID NO: 326 and/or to a sequence comprised by SEQ ID NO: 153;
the probe, primer or primer pair for detection of the fusion of ST14 with NRG1 specifically hybridizes to, or has 95% or more complementary sequence identity with, a sequence comprised by SEQ ID NO: 372 and/or to a sequence comprised by SEQ ID NO: 155;
the probe, primer or primer pair for detection of the fusion of THBS1 with NRG1 specifically hybridizes to, or has 95% or more complementary sequence identity with, a sequence comprised by SEQ ID NO: 399 and/or to a sequence comprised by SEQ ID NO: 155;
the probe, primer or primer pair for detection of the fusion of AGRN with NRG1 specifically hybridizes to, or has 95% or more complementary sequence identity with, a sequence comprised by SEQ ID NO: 433 and/or to a sequence comprised by SEQ ID NO: 155;
the probe, primer or primer pair for detection of the fusion of PVALB with NRG1 specifically hybridizes to, or has 95% or more complementary sequence identity with, a sequence comprised by SEQ ID NO: 450 and/or to a sequence comprised by SEQ ID NO: 155;
the probe, primer or primer pair for detection of the fusion of SLC3A2 with NRG1 specifically hybridizes to, or has 95% or more complementary sequence identity with, a sequence comprised by SEQ ID NO: 482 and/or to a sequence comprised by SEQ ID NO: 155;
the probe, primer or primer pair for detection of the fusion of APP with NRG1 specifically hybridizes to, or has 95% or more complementary sequence identity with, a sequence comprised by SEQ ID NO: 524 and/or to a sequence comprised by SEQ ID NO: 155;
the probe, primer or primer pair for detection of the fusion of WRN with NRG1 specifically hybridizes to, or has 95% or more complementary sequence identity with, a sequence comprised by SEQ ID NO: 601 and/or to a sequence comprised by SEQ ID NO: 155;
the probe, primer or primer pair for detection of the fusion of DAAM1 with NRG1 specifically hybridizes to, or has 95% or more complementary sequence identity with, a sequence comprised by SEQ ID NO: 606 and/or to a sequence comprised by SEQ ID NO: 138;
the probe, primer or primer pair for detection of the fusion of ASPH with NRG1 specifically hybridizes to, or has 95% or more complementary sequence identity with, a sequence comprised by SEQ ID NO: 689 and/or to a sequence comprised by SEQ ID NO: 153;
the probe, primer or primer pair for detection of the fusion of NOTCH2 with NRG1 specifically hybridizes to, or has 95% or more complementary sequence identity with, a sequence comprised by SEQ ID NO: 713 and/or to a sequence comprised by SEQ ID NO: 155;
the probe, primer or primer pair for detection of the fusion of CD74 with NRG1 specifically hybridizes to, or has 95% or more complementary sequence identity with, a sequence comprised by SEQ ID NO: 739 and/or to a sequence comprised by SEQ ID NO: 153;
the probe, primer or primer pair for detection of the fusion of SDC4 with NRG1 specifically hybridizes to, or has 95% or more complementary sequence identity with, a sequence comprised by SEQ ID NO: 757 and/or to a sequence comprised by SEQ ID NO: 153;
the probe, primer or primer pair for detection of the fusion of CD44 with NRG1 specifically hybridizes to, or has 95% or more complementary sequence identity with, a sequence comprised by SEQ ID NO: 99 and/or to a sequence comprised by SEQ ID NO: 155;
the probe, primer or primer pair for detection of the fusion of SLC4A4 with NRG1 specifically hybridizes to, or has 95% or more complementary sequence identity with, a sequence comprised by SEQ ID NO: 820 and/or to a sequence comprised by SEQ ID NO: 155;
the probe, primer or primer pair for detection of the fusion of SDC4 with NRG1 specifically hybridizes to, or has 95% or more complementary sequence identity with, a sequence comprised by SEQ ID NO: 940 and/or to a sequence comprised by SEQ ID NO: 153;
the probe, primer or primer pair for detection of the fusion of ZFAT with NRG1 specifically hybridizes to, or has 95% or more complementary sequence identity with, a sequence comprised by SEQ ID NO: 864 and/or to a sequence comprised by SEQ ID NO: 155; and
the probe, primer or primer pair for detection of the fusion of DSCAML1 with NRG1 specifically hybridizes to, or has 95% or more complementary sequence identity with, a sequence comprised by SEQ ID NO: 938 and/or to a sequence comprised by SEQ ID NO: 153.

36. A first and a second nucleic acid probe for use in an in-situ hybridization assay to detect a polynucleotide fusion of any one of clauses 1-23,
wherein the first probe specifically hybridizes to a VAPB sequence which is located 5′ from the nucleic acid of position 43 of SEQ ID NO: 3, and the second probe specifically hybridizes to a NRG1 sequence which is located 3′ from the nucleic acid of position 44 of SEQ ID NO: 3;
wherein the first probe specifically hybridizes to a CADM1 sequence which is located 5′ from the nucleic acid of position 53 of SEQ ID NO: 7, and the second probe specifically hybridizes to a NRG1 sequence which is located 3′ from the nucleic acid of position 54 of SEQ ID NO: 7;
wherein the first probe specifically hybridizes to a CD44 sequence which is located 5′ from the nucleic acid of position 52 of SEQ ID NO: 11, and the second probe specifically hybridizes to a NRG1 sequence which is located 3′ from the nucleic acid of position 53 of SEQ ID NO: 11;
wherein the first probe specifically hybridizes to a SLC3A2 sequence which is located 5′ from the nucleic acid of position 53 of SEQ ID NO: 15, and the second probe specifically hybridizes to a NRG1 sequence which is located 3′ from the nucleic acid of position 54 of SEQ ID NO: 15;
wherein the first probe specifically hybridizes to a VTCN1 sequence which is located 5′ from the nucleic acid of position 65 of SEQ ID NO: 166, and the second probe specifically hybridizes to a NRG1 sequence which is located 3′ from the nucleic acid of position 66 of SEQ ID NO: 166;
wherein the first probe specifically hybridizes to a CDH1 sequence which is located 5′ from the nucleic acid of position 119 of SEQ ID NO: 186, and the second probe specifically hybridizes to a NRG1 sequence which is located 3′ from the nucleic acid of position 120 of SEQ ID NO: 186;
wherein the first probe specifically hybridizes to a CXADR sequence which is located 5′ from the nucleic acid of position 43 of SEQ ID NO: 217, and the second probe specifically hybridizes to a NRG1 sequence which is located 3′ from the nucleic acid of position 44 of SEQ ID NO: 217;
wherein the first probe specifically hybridizes to a GTF2E2 sequence which is located 5′ from the nucleic acid of position 141 of SEQ ID NO: 233, and the second probe specifically hybridizes to a NRG1 sequence which is located 3′ from the nucleic acid of position 142 of SEQ ID NO: 233;
wherein the first probe specifically hybridizes to a CSMD1 sequence which is located 5′ from the nucleic acid of position 88 of SEQ ID NO: 255, and the second probe specifically hybridizes to a NRG1 sequence which is located 3′ from the nucleic acid of position 89 of SEQ ID NO: 255;
wherein the first probe specifically hybridizes to a PTN sequence which is located 5′ from the nucleic acid of position 102 of SEQ ID NO: 313, and the second probe specifically hybridizes to a NRG1 sequence which is located 3′ from the nucleic acid of position 103 of SEQ ID NO: 313;
wherein the first probe specifically hybridizes to a ST14 sequence which is located 5′ from the nucleic acid of position 95 of SEQ ID NO: 330, and the second probe specifically hybridizes to a NRG1 sequence which is located 3′ from the nucleic acid of position 96 of SEQ ID NO: 330;
wherein the first probe specifically hybridizes to a THBS1 sequence which is located 5′ from the nucleic acid of position 56 of SEQ ID NO: 376, and the second probe specifically hybridizes to a NRG1 sequence which is located 3′ from the nucleic acid of position 57 of SEQ ID NO: 376;
wherein the first probe specifically hybridizes to a AGRN sequence which is located 5′ from the nucleic acid of position 106 of SEQ ID NO: 403, and the second probe specifically hybridizes to a NRG1 sequence which is located 3′ from the nucleic acid of position 107 of SEQ ID NO: 403;
wherein the first probe specifically hybridizes to a PVALB sequence which is located 5′ from the nucleic acid of position 102 of SEQ ID NO: 437, and the second probe specifically hybridizes to a NRG1 sequence which is located 3′ from the nucleic acid of position 103 of SEQ ID NO: 437;
wherein the first probe specifically hybridizes to a SLC3A2 sequence which is located 5′ from the nucleic acid of position 93 of SEQ ID NO: 454, and the second probe specifically hybridizes to a NRG1 sequence which is located 3′ from the nucleic acid of position 94 of SEQ ID NO: 454;
wherein the first probe specifically hybridizes to a APP sequence which is located 5′ from the nucleic acid of position 54 of SEQ ID NO: 486, and the second probe specifically hybridizes to a NRG1 sequence which is located 3′ from the nucleic acid of position 55 of SEQ ID NO: 486;
wherein the first probe specifically hybridizes to a WRN sequence which is located 5′ from the nucleic acid of position 96 of SEQ ID NO: 528, and the second probe specifically hybridizes to a NRG1 sequence which is located 3′ from the nucleic acid of position 97 of SEQ ID NO: 528;
wherein the first probe specifically hybridizes to a DAAM1 sequence which is located 5′ from the nucleic acid of position 75 of SEQ ID NO: 605, and the second probe specifically hybridizes to a NRG1 sequence which is located 3′ from the nucleic acid of position 76 of SEQ ID NO: 605;
wherein the first probe specifically hybridizes to a ASPH sequence which is located 5′ from the nucleic acid of position 75 of SEQ ID NO: 635, and the second probe specifically hybridizes to a NRG1 sequence which is located 3′ from the nucleic acid of position 76 of SEQ ID NO: 635;
wherein the first probe specifically hybridizes to a NOTCH2 sequence which is located 5′ from the nucleic acid of position 75 of SEQ ID NO: 693, and the second probe specifically hybridizes to a NRG1 sequence which is located 3′ from the nucleic acid of position 76 of SEQ ID NO: 693;
wherein the first probe specifically hybridizes to a CD74 sequence which is located 5′ from the nucleic acid of position 75 of SEQ ID NO: 717, and the second probe specifically hybridizes to a NRG1 sequence which is located 3′ from the nucleic acid of position 76 of SEQ ID NO: 717;
wherein the first probe specifically hybridizes to a SDC4 sequence which is located 5′ from the nucleic acid of position 75 of SEQ ID NO: 743, and the second probe specifically hybridizes to a NRG1 sequence which is located 3′ from the nucleic acid of position 76 of SEQ ID NO: 743;
wherein the first probe specifically hybridizes to a CD44 sequence which is located 5′ from the nucleic acid of position 75 of SEQ ID NO: 761, and the second probe specifically hybridizes to a NRG1 sequence which is located 3′ from the nucleic acid of position 76 of SEQ ID NO: 761;
wherein the first probe specifically hybridizes to a SLC4A4 sequence which is located 5′ from the nucleic acid of position 75 of SEQ ID NO: 765, and the second probe specifically hybridizes to a NRG1 sequence which is located 3′ from the nucleic acid of position 76 of SEQ ID NO: 765;
wherein the first probe specifically hybridizes to a SDC4 sequence which is located 5′ from the nucleic acid of position 75 of SEQ ID NO: 824, and the second probe specifically hybridizes to a NRG1 sequence which is located 3′ from the nucleic acid of position 76 of SEQ ID NO: 824;
wherein the first probe specifically hybridizes to a ZFAT sequence which is located 5′ from the nucleic acid of position 75 of SEQ ID NO: 828, and the second probe specifically hybridizes to a NRG1 sequence which is located 3′ from the nucleic acid of position 76 of SEQ ID NO: 828; or
wherein the first probe specifically hybridizes to a DSCAML1 sequence which is located 5′ from the nucleic acid of position 75 of SEQ ID NO: 868, and the second probe specifically hybridizes to a NRG1 sequence which is located 3′ from the nucleic acid of position 76 of SEQ ID NO: 868.

37. A first antibody or a set of a first and a second antibody pair for detection of a polypeptide encoded by a polynucleotide fusion according to any of the clauses 1-23.

38. A detection assay comprising a first antibody or a set of a first and second antibodies for detection of the presence of a polypeptide encoded by a polynucleotide fusion according to any one of clauses 1-23, wherein the first antibody or set of a first and second antibodies preferably is the first antibody or a set of a first and second antibodies of clause 34.

39. The first antibody or set of first and second antibodies of clause 38, or the detection assay according to clause 32, wherein the first antibody binds a polypeptide fusion selected from VAPB-NRG1, CADM1-NRG1, CD44-NRG1, SLC3A2-NRG1, CDH1-NRG1, CXADR-NRG1, GTF2E2-NRG1, CSMD1-NRG1, PTN-NRG1, ST14-NRG1, THBS1-NRG1, AGRN-NRG1, PVALB-NRG1, APP-NRG1, WRN-NRG1, ASPH-NRG1, NOTCH2-NRG1, CD74-NRG1, SDC4-NRG1, SLC4A4-NRG1, ZFAT-NRG1 or DSCAML1-NRG1 and the set of first and second antibodies binds VAPB and NRG1 or CADM1 and NRG1, or CD44 and NRG1, SLC3A2 and NRG1, CDH1 and NRG1, CXADR and NRG1, GTF2E2 and NRG1, CSMD1 and NRG1, PTN and NRG1, ST14 and NRG1, THBS1 and NRG1, AGRN and NRG1, PVALB and NRG1, APP and NRG1, WRN and NRG1, ASPH and NRG1, NOTCH2 and NRG1, CD74 and NRG1, SDC4 and NRG1, SLC4A4 and NRG1, ZFAT and NRG1, or DSCAML1 and NRG1 respectively.

40. A method for identifying a polynucleotide fusion, or a polypeptide encoded therefrom, according to any one of the clauses 1-23 in a sample, said method comprising testing a sample obtained from a subject to detect the presence of the fusion in the sample.

41. A method for detecting the presence of a polynucleotide fusion, or a polypeptide encoded therefrom, according to any one of the clauses 1-23 in a sample, said method comprising testing a sample obtained from a subject to detect the presence of the fusion in the sample.

42. A method for establishing whether an aberrant cell from a subject comprises a fusion, or a polypeptide encoded therefrom, according to any one of clauses 1-23, said method comprising testing the polynucleotide or polypeptide contents of the aberrant cell obtained from the subject for the presence of the fusion in the sample.

43. A method for identifying a subject as carrying a polynucleotide fusion, or a polypeptide encoded therefrom, according to any one of clauses 1-23, said method comprising testing a sample obtained from a subject to detect the presence of the fusion in the sample.

44. The method of any one of clauses 37-40, wherein the testing comprises detecting the fusion by utilizing a binding agent that specifically binds the polynucleotide, such as the nucleic acid probe, primer or primer pair of clause 31-36, or a polypeptide encoded therefrom, or utilizing a binding agent that binds a polynucleotide that comprises the polynucleotide fusion.

45. The method of any one of clauses 40-44, wherein the testing comprises amplifying or detecting a sequence that discriminates between the presence and absence of the polynucleotide fusion, or polypeptide encoded therefrom.

46. The method of any one of clauses 41-45, wherein the polynucleotide fusion is obtained from an aberrant cell expressing a polynucleotide fusion that comprises an EGF-like domain of NRG1.

47. The method of any one of clauses 40-46, wherein the method comprises a step of obtaining the sample from a subject, followed by a step of isolating the polynucleotide or polypeptide encoded therefrom, from the sample.

48. The method of any one of clauses 40-47, wherein the method comprises a step of purifying or isolating the polynucleotide or polypeptide from the sample.

49. The method of any one of clauses 40-48, wherein the binding agent is or comprises a primer, a primer pair, a probe or an antibody.

50. The method of any one of clauses 40-49, wherein the testing is an ex vivo method, preferably an in vitro method.

51. The method of any one of clauses 40-50, wherein the binding agent comprises or is associated with a detectable label.

52. The method of any one of clauses 40-51, wherein the sample is a liquid biopsy sample or a solid sample, such as a formalin fixed paraffin embedded tissue (FFPE) sample.

53. The method of any one of clauses 40-52, wherein the sample comprises blood, serum, plasma, pleural liquid, urine, semen, amniotic fluid or peritoneal fluid.

54. The method of any one of clauses 40-53, wherein the sample comprises an aberrant cell, such as a tumor cell or a cancer cell, or the polynucleotide or polypeptide contents thereof.

55. A method of treating a subject having an ErbB-2 and/or ErbB-3 positive cancer or tumor comprising a polynucleotide fusion and/or expressing a fusion polypeptide encoded therefrom, said method comprising administering to the subject an effective amount of an ErbB-2 and/or ErbB-3 targeting agent, wherein the fusion is a fusion according to any one of clauses 1-23.

56. A method for inhibiting the progression in a subject of an ErbB-2 and ErbB-3 positive cancer or tumor comprising a polynucleotide fusion and/or expressing a fusion polypeptide encoded therefrom, said method comprising administering to the subject an effective amount of an ErbB-2 and/or ErbB-3 targeting agent, wherein said fusion is a fusion according to any one of clauses 1-23.

57. An ErbB-2 and/or ErbB-3 targeting agent for use in the treatment of a subject that has an ErbB-2 and ErbB-3 positive cancer or tumor comprising a polynucleotide fusion and/or expressing a fusion polypeptide encoded therefrom, said treatment comprising administering an effective amount of the ErbB-2 and/or ErbB-3 targeting agent to the subject, wherein said fusion is a fusion according to any one of clauses 1-23.

58. A method for diagnosing a subject for an aberrant cell that comprises a polynucleotide fusion, or polypeptide encoded therefrom according to any one of clauses 1-23, the method comprising testing a sample obtained from a subject to detect the presence of the fusion in the sample.

59. The method of clause 58, wherein the testing comprises detecting the fusion by utilizing a binding agent that specifically binds the polynucleotide, such as the nucleic acid probe, primer or primer pair of any one of clauses 30-35, or a polypeptide encoded therefrom, or utilizing a binding agent that binds a polynucleotide that comprises the polynucleotide fusion.

60. A method for assessing whether a subject suffers from a cancer or tumor or is prone to suffering from a cancer or tumor, the method comprising testing a sample obtained from a subject to detect the presence of a polynucleotide fusion, or polypeptide encoded therefrom according to any one of clauses 1-23 in the sample, and assessing that said subject suffers from said cancer or tumor or is prone to suffering from said cancer or tumor, by identifying the presence of said polynucleotide or polypeptide fusion.

61. The method or use according to any one of clauses 55-58, wherein the ErbB-2 and/or ErbB-3 targeting agent is selected from the group consisting of a multispecific antibody comprising a first antigen-binding site that binds an extracellular part of ErbB-2 and a second antigen-binding site that binds an extracellular part of ErbB-3, a tyrosine kinase inhibitor of ErbB-2, a monospecific bivalent antibody comprising an antigen-binding site that bind an extracellular part of ErbB-2, a monospecific bivalent antibody comprising an antigen-binding site that bind an extracellular part of ErbB-3, or any combination thereof.

62. The method or use according to any one of clauses 55-58, or 61, wherein the ErbB-2 and/or ErbB-3 targeting agent is zenocutuzumab.

63. The method or use according to any one of clauses 41-62, wherein the aberrant cell, cancer cell or tumor cell comprises the polynucleotide fusion of clauses 1-23, or a polypeptide encoded thereby, and wherein the polynucleotide fusion comprised by the cell further comprises an in-frame fusion of a coding sequence that codes for EGF-like domain of NRG1.

64. The method or use according to any one of clauses 41-63, wherein the aberrant cell is from a cancer, in particular said cancer is an adenocarcinoma, more in particular a mucinous adenocarcinoma, a pancreatic cancer, in particular a pancreatic adenocarcinoma, more in particular a pancreatic ductal adenocarcinoma, a renal cell carcinoma, a sarcoma, a bladder, a colon, a rectal, colorectal, a gallbladder, a head and neck cancer, a prostrate, a uterus, a breast cancer, an ovarian cancer, a liver cancer, an endometrial cancer, a lung cancer, preferably a non-small cell lung cancer, preferably, more preferably invasive mucinous adenocarcinoma, or a primary or metastatic cancer.

65. An in vivo animal model comprising a polynucleotide fusion according to any one of clauses 1-23 and/or expressing a polypeptide fusion encoded therefrom, wherein preferably the polynucleotide fusion or polypeptide fusion comprised by the animal model is comprised by an engrafted aberrant cell present in the animal model or comprised by the genome of the animal model.

66. A method of treatment of the in vivo animal model of claim 66 with an Erb2 and/or Erb3 targeting agent selected from the group consisting of a multispecific antibody comprising a first antigen-binding site that binds an extracellular part of ErbB-2 and a second antigen-binding site that binds an extracellular part of ErbB-3, a tyrosine kinase inhibitor of ErbB-2, a monospecific bivalent antibody comprising an antigen-binding site that bind an extracellular part of ErbB-2, a monospecific bivalent antibody comprising an antigen-binding site that bind an extracellular part of ErbB-3, or any combination thereof, said method comprising administering to the animal said Erb2 and/or Erb3 targeting agent.

67. A first and a second nucleic acid probe for use in an in-situ hybridization assay to detect a genetic rearrangement of VAPB, CADM1, CD44, SLC3A2, VTCN1, CDH1 CXADR, GTF2E2, CSMD1, PTN, ST14, THBS1, AGRN, PVALB, APP, WRN, DAAM1, ASPH, NOTCH2, CD74, SDC4, SLC4A4, ZFAT or DSCAML1 wherein:
the first probe for detection of a genetic rearrangement of VAPB specifically hybridizes to a VAPB sequence which is 5′ from the nucleic acid of position 42 or 43 of SEQ ID NO: 1, and the second probe specifically hybridizes to a VAPB sequence which is 3′ from the nucleic acid of position 42 or 43 of SEQ ID NO: 1;
the first probe for detection of a genetic rearrangement of CADM1 specifically hybridizes to a CADM1 sequence which is 5′ from the nucleic acid of position 53 of SEQ ID NO: 5, and the second probe specifically hybridizes to a CADM1 sequence which is 3′ from the nucleic acid of position 53 of SEQ ID NO: 5;
the first probe for detection of a genetic rearrangement of CD44 specifically hybridizes to a CD44 sequence which is 5′ from the nucleic acid of position 52 of SEQ ID NO: 9, and the second probe specifically hybridizes to a CD44 sequence which is 3′ from the nucleic acid of position 52 of SEQ ID NO: 9;
the first probe for detection of a genetic rearrangement of SLC3A2 specifically hybridizes to a SLC3A2 sequence which is 5′ from the nucleic acid of position 53 of SEQ ID NO: 13, and the second probe specifically hybridizes to a SLC3A2 sequence which is 3′ from the nucleic acid of position 53 of SEQ ID NO: 13;
the first probe for detection of a genetic rearrangement of VTCN1 specifically hybridizes to a VTCN1 sequence which is 5′ from the nucleic acid of position 65 of SEQ ID NO: 164, and the second probe specifically hybridizes to a VTCN1 sequence which is 3′ from the nucleic acid of position 65 of SEQ ID NO: 164;
the first probe for detection of a genetic rearrangement of CDH1 specifically hybridizes to a CDH1 sequence which is 5′ from the nucleic acid of position 119 of SEQ ID NO: 184, and the second probe specifically hybridizes to a CDH1 sequence which is 3′ from the nucleic acid of position 119 of SEQ ID NO: 184;
the first probe for detection of a genetic rearrangement of CXADR specifically hybridizes to a CXADR sequence which is 5′ from the nucleic acid of position 43 of SEQ ID NO: 215, and the second probe specifically hybridizes to a CXADR sequence which is 3′ from the nucleic acid of position 43 of SEQ ID NO: 215;
the first probe for detection of a genetic rearrangement of GTF2E2 specifically hybridizes to a GTF2E2 sequence which is 5′ from the nucleic acid of position 141 of SEQ ID NO: 231, and the second probe specifically hybridizes to a GTF2E2 sequence which is 3′ from the nucleic acid of position 141 of SEQ ID NO: 231;
the first probe for detection of a genetic rearrangement of CSMD1 specifically hybridizes to a CSMD1 sequence which is 5′ from the nucleic acid of position 88 of SEQ ID NO: 253, and the second probe specifically hybridizes to a CSMD1 sequence which is 3′ from the nucleic acid of position 88 of SEQ ID NO:253;
the first probe for detection of a genetic rearrangement of PTN specifically hybridizes to a PTN sequence which is 5′ from the nucleic acid of position 102 of SEQ ID NO: 311, and the second probe specifically hybridizes to a PTN sequence which is 3′ from the nucleic acid of position 102 of SEQ ID NO: 311;
the first probe for detection of a genetic rearrangement of ST14 specifically hybridizes to a ST14 sequence which is 5′ from the nucleic acid of position 95 of SEQ ID NO: 328, and the second probe specifically hybridizes to a ST14 sequence which is 3′ from the nucleic acid of position 95 of SEQ ID NO: 328;
the first probe for detection of a genetic rearrangement of AGRN specifically hybridizes to a AGRN sequence which is 5′ from the nucleic acid of position 106 of SEQ ID NO: 401, and the second probe specifically hybridizes to a AGRN sequence which is 3′ from the nucleic acid of position 106 of SEQ ID NO: 401;
the first probe for detection of a genetic rearrangement of THBS1 specifically hybridizes to a THBS1 sequence which is 5′ from the nucleic acid of position 56 of SEQ ID NO: 374, and the second probe specifically hybridizes to a THBS1 sequence which is 3′ from the nucleic acid of position 56 of SEQ ID NO: 374;
the first probe for detection of a genetic rearrangement of PVALB specifically hybridizes to a PVALB sequence which is 5′ from the nucleic acid of position 102 of SEQ ID NO: 435, and the second probe specifically hybridizes to a PVALB sequence which is 3′ from the nucleic acid of position 102 of SEQ ID NO: 435;
the first probe for detection of a genetic rearrangement of SLC3A2 specifically hybridizes to a SLC3A2 sequence which is 5′ from the nucleic acid of position 93 of SEQ ID NO: 452, and the second probe specifically hybridizes to a SLC3A2 sequence which is 3′ from the nucleic acid of position 93 of SEQ ID NO: 452;
the first probe for detection of a genetic rearrangement of APP specifically hybridizes to a APP sequence which is 5′ from the nucleic acid of position 54 of SEQ ID NO: 484, and the second probe specifically hybridizes to a APP sequence which is 3′ from the nucleic acid of position 54 of SEQ ID NO: 484;
the first probe for detection of a genetic rearrangement of WRN specifically hybridizes to a WRN sequence which is 5′ from the nucleic acid of position 96 of SEQ ID NO: 526, and the second probe specifically hybridizes to a WRN sequence which is 3′ from the nucleic acid of position 96 of SEQ ID NO: 526;
the first probe for detection of a genetic rearrangement of DAAM1 specifically hybridizes to a DAAM1 sequence which is 5′ from the nucleic acid of position 75 of SEQ ID NO: 603, and the second probe specifically hybridizes to a DAAM1 sequence which is 3′ from the nucleic acid of position 75 of SEQ ID NO: 603;
the first probe for detection of a genetic rearrangement of ASPH specifically hybridizes to a ASPH sequence which is 5′ from the nucleic acid of position 75 of SEQ ID NO: 633, and the second probe specifically hybridizes to a ASPH sequence which is 3′ from the nucleic acid of position 75 of SEQ ID NO: 633;
the first probe for detection of a genetic rearrangement of NOTCH2 specifically hybridizes to a NOTCH2 sequence which is 5′ from the nucleic acid of position 75 of SEQ ID NO: 691, and the second probe specifically hybridizes to a NOTCH2 sequence which is 3′ from the nucleic acid of position 75 of SEQ ID NO: 691;
the first probe for detection of a genetic rearrangement of CD74 specifically hybridizes to a CD74 sequence which is 5′ from the nucleic acid of position 75 of SEQ ID NO: 715, and the second probe specifically hybridizes to a CD74 sequence which is 3′ from the nucleic acid of position 75 of SEQ ID NO: 715;
the first probe for detection of a genetic rearrangement of SDC4 specifically hybridizes to a SDC4 sequence which is 5′ from the nucleic acid of position 75 of SEQ ID NO: 741, and the second probe specifically hybridizes to a SDC4 sequence which is 3′ from the nucleic acid of position 75 of SEQ ID NO: 741;
the first probe for detection of a genetic rearrangement of CD44 specifically hybridizes to a CD44 sequence which is 5′ from the nucleic acid of position 75 of SEQ ID NO: 759, and the second probe specifically hybridizes to a CD44 sequence which is 3′ from the nucleic acid of position 75 of SEQ ID NO: 759;
the first probe for detection of a genetic rearrangement of SLC4A4 specifically hybridizes to a SLC4A4 sequence which is 5′ from the nucleic acid of position 75 of SEQ ID NO: 763, and the second probe specifically hybridizes to a SLC4A4 sequence which is 3′ from the nucleic acid of position 75 of SEQ ID NO: 763;
the first probe for detection of a genetic rearrangement of SDC4 specifically hybridizes to a SDC4 sequence which is 5′ from the nucleic acid of position 75 of SEQ ID NO: 822, and the second probe specifically hybridizes to a SDC4 sequence which is 3′ from the nucleic acid of position 75 of SEQ ID NO: 822;
the first probe for detection of a genetic rearrangement of ZFAT specifically hybridizes to a ZFAT sequence which is 5′ from the nucleic acid of position 75 of SEQ ID NO: 826, and the second probe specifically hybridizes to a ZFAT sequence which is 3′ from the nucleic acid of position 75 of SEQ ID NO: 826; or
the first probe for detection of a genetic rearrangement of DSCAML1 specifically hybridizes to a DSCAML1 sequence which is 5′ from the nucleic acid of position 75 of SEQ ID NO: 866, and the second probe specifically hybridizes to a DSCAML1 sequence which is 3′ from the nucleic acid of position 75 of SEQ ID NO: 866.

### EXAMPLES

### Example 1: Test Sample Preparation

Liquid samples or biopsies include blood, blood serum, blood plasma, pleural effusion, urine, semen, vaginal swab, amniotic fluid, peritoneal fluid, cell free lavage or other biofluids.

When blood is used as starting material, the blood can be collected in tubes treated with cell stabilizing agents such as Cell-Free DNA BCT tubes (Streck) which prevents lysis of blood cells post-collection and minimized contamination of wild-type DNA/RNA from normal cells.

### Example 2: Purification of Circulating DNA from Plasma

Although the protocol of this example is described in relation to the isolation of cfDNA from a plasma sample of 1 ml, the protocol also suitably allows for isolation from other volumes when higher yields are desired and other sample types, like urine and serum. The protocol is based on the QIAamp® circulating Nucleic Acid Kit (CNA) (Qiagen, Cat #55114) and Handbook dated October 2019 (HB-0202-006).

Before starting, plasma samples from a human subject are allowed to equilibrate to room temperature, either from storage at a suitable refrigerated temperature or directly after drawing from a patient. All centrifugation steps are carried out at room temperature. Where necessary, samples with a volume of below 1 ml are adjusted with phosphate buffered saline (PBS) to a total of 1 ml.

### Preparation of Buffers and Reagents.

Buffers ACB, ACW1 and ACW2 are prepared according to the manufacturer's instructions. Briefly, before use, 200 ml isopropanol (100 v/v %) is added to 300 ml buffer ACB concentrate to obtain 500 ml Buffer ACB. After adding isopropanol, the obtained buffer is mixed well. Before use, 25 ml ethanol 96-100 (v/v %) is added to 19 ml buffer ACW1 concentrate to obtain 44 ml buffer ACW1. The obtained liquid is mixed well after adding ethanol. Before use, 30 ml ethanol (96-100%) is added to 13 ml buffer ACW2 concentrate to obtain 43 ml buffer, which is subsequently well mixed. For instance, to process 12 samples of 1 ml plasma, 10.6 ml buffer ACL is mixed with 67.5 μl carrier RNA-containing AVE buffer.

Isolation of cfDNA from Plasma

Procedure: 100 μl QIAGEN Proteinase K is pipetted into a 50 ml centrifuge tube, 1 ml of plasma is added to the 50 ml tube. 0.8 ml ACL buffer (containing 1.0 μg carrier RNA) is added after which caps are closed and mixed by pulse-vortexing for 30 s while a visible vortex is formed in the tube. Sample and ACL buffer are mixed thoroughly to yield a homogeneous solution. The samples are immediately lysed by incubating at 60° C. for 30 minutes. 1.8 ml ACB buffer is added to the lysate in the tube after which the cap is closed and mix thoroughly by pulse-vortexing for 15-30 s. The lysate-ACB buffer mixture is incubated in the tube for 5 min on ice. QIAamp Mini columns are inserted into a VacConnector on a QIAvac 24 Plus which is set-up according the manufacturer's instructions. A 20 ml tube extender is firmly inserted into the open QIAamp Mini column.

After incubation on ice, the lysate-ACB buffer mixture is applied into the tube extender of the QIAamp Mini column. The vacuum pump is switched on and when all lysates are drawn through the columns completely, the vacuum pump is switched off and pressure is released to 0 mbar. The tube extender is carefully removed and discarded. If part of the QIAvac Connecting System, a Vacuum Regulator can be used. Cross-contamination is avoided by not moving the tube extenders over neighboring QIAamp Mini Columns. 600 μl ACW1 buffer is applied to the QIAamp Mini column. Lids of the column are left open and the vacuum pump switched on. After all of the ACW1 buffer has been drawn through the QIAamp Mini column, the vacuum pump is switched off and pressure is released to 0 mbar. 750 μl ACW2 buffer is applied to the QIAamp Mini column. The lid of the column is left open, and the vacuum pump switched on. After all of ACW2 buffer is drawn through the QIAamp Mini column, the vacuum pump is switched off and pressure is released to 0 mbar. 750 μl of ethanol (96 v/v %) is applied to the QIAamp Mini column. The lid of the column is left open, and the vacuum pump switched on. After all of ethanol is drawn through the spin column, the vacuum pump is switched off and pressure is released to 0 mbar. The lid of the QIAamp Mini column is closed and the column is removed from the vacuum manifold after which the VacConnector is discarded. The QIAamp Mini column is placed in a clean 2 ml collection tube, and centrifuged at full speed (20,000×g; 14,000 rpm) for 3 min. The QIAamp Mini Column is placed in a new 2 ml collection tube. The lid is opened and the assembly is incubated at 56° C. for 10 min to completely dry the membrane. The QIAamp Mini column is placed in a clean 1.5 ml elution tube and the lastly used 2 ml collection tube is discarded. 20 μl of AVE buffer is carefully applied to the center of the QIAamp Mini membrane. The lid is closed and incubated at room temperature for 3 min. Elution buffer AVE is equilibrated to room temperature. The recovered eluate volume is about 5 μl less than the elution volume applied to the QIAamp Mini column. The columns are centrifuged in a microcentrifuge at full speed (20,000×g; 14,000 rpm) for 1 min to elute the nucleic acids such that cell free DNA is obtained.

Free-circulating cell-free DNA is eluted in AVE buffer, ready for use in amplification reactions or storage at −30 to −15° C. Purified nucleic acids are free of proteins, nucleases, and other impurities.

Next, the concentration of the nucleic acids is determined prior to NRG1 fusion amplification and detection analysis according to the manufacturer's recommendation, such as quantitative amplification assays.

List of materials for the QIAvac 24 Plus System for vacuum processing of 1-24 QIAGEN spin columns. QIAvac 24 Plus: Vacuum Manifold for processing 1-24 spin columns which includes QIAvac 24 Plus Vacuum Manifold, Luer Plugs and Quick Couplings (Cat. No. 19413). VacConnectors (500): 500 disposable connectors for use with QIAamp Mini columns on luer slots or VacValves (Cat. No. 19407). VacValves (24): 24 valves for use with the QIAvac 24 Plus (Cat. No. 19408). Vacuum Regulator: For use with QIAvac manifolds (Cat. No. 19530). Vacuum Pump (230 V, 50 Hz): Universal vacuum pump (capacity 34 L/min, 8 mbar vacuum abs.) (Cat. No. 84020). QIAvac Connecting System: System to connect vacuum manifold with vacuum pump: includes tray, waste bottles, tubing, couplings, valve, gauge, 24 VacValves (Cat. No. 19419).

### Example 3: Circulating Tumor RNA (cfRNA)

Purification of Circulating RNA from Serum

Although the protocol of this example is described in relation to the isolation of circulating RNA from a serum sample of 4 ml, the protocol also suitably allows for isolation from other volumes when higher yields are desired and other sample types, like urine or plasma. The protocol is based on the QIAamp® circulating Nucleic Acid Kit (CNA) (Qiagen, Cat #55114) and Handbook dated October 2019 (HB-0202-006).

Before starting, serum samples from a human subject are allowed to equilibrate to room temperature, either from storage at a suitable refrigerated temperature or directly after drawing from a patient. All centrifugation steps are carried out at room temperature. Where necessary, samples with a volume of below 4 ml are adjusted with phosphate buffered saline (PBS) to a total of 4 ml.

### Preparation of Buffers and Reagents.

Buffers ACB, ACW1 and ACW2 are prepared according to the manufacturer's instructions. Briefly, before use, 200 ml isopropanol (100 v/v %) is added to 300 ml buffer ACB concentrate to obtain 500 ml Buffer ACB. After adding isopropanol, the obtained buffer is mixed well. Before use, 25 ml ethanol 96-100 (v/v %) is added to 19 ml buffer ACW1 concentrate to obtain 44 ml buffer ACW1. The obtained liquid is mixed well after adding ethanol. Before use, 30 ml ethanol (96-100%) is added to 13 ml buffer ACW2 concentrate to obtain 43 ml buffer, which is subsequently well mixed. For instance, to process 12 samples of 4 ml serum, 42.2 ml buffer ACL is mixed with 67.5 μl carrier RNA-containing AVE buffer.

Isolation of cfRNA from Serum

Procedure: 400 μl QIAGEN Proteinase K is pipetted into a 50 ml centrifuge tube, 4 ml of serum is added to the 50 ml tube. 3.2 ml ACL buffer (containing 1.0 μg carrier RNA) is added after which caps are closed and mixed by pulse-vortexing for 30 s while a visible vortex is formed in the tube. Sample and ACL buffer are mixed thoroughly to yield a homogeneous solution. The samples are immediately lysed by incubating at 60° C. for 30 minutes. 7.2 ml ACB buffer is added to the lysate in the tube after which the cap is closed and mix thoroughly by pulse-vortexing for 15-30 s. The lysate-ACB buffer mixture is incubated in the tube for 5 min on ice. QIAamp Mini columns are inserted into a VacConnector on a QIAvac 24 Plus which is set-up according the manufacturer's instructions. A 20 ml tube extender is firmly inserted into the open QIAamp Mini column.

After incubation on ice, the lysate-ACB buffer mixture is applied into the tube extender of the QIAamp Mini column. The vacuum pump is switched on and when all lysates are drawn through the columns completely, the vacuum pump is switched off and pressure is released to 0 mbar. The tube extender is carefully removed and discarded. If part of the QIAvac Connecting System, a Vacuum Regulator can be used. Cross-contamination is avoided by not moving the tube extenders over neighboring QIAamp Mini Columns. 600 μl ACW1 buffer is applied to the QIAamp Mini column. Lids of the column are left open and the vacuum pump switched on. After all of the ACW1 buffer has been drawn through the QIAamp Mini column, the vacuum pump is switched off and pressure is released to 0 mbar. 750 μl ACW2 buffer is applied to the QIAamp Mini column. The lid of the column is left open, and the vacuum pump switched on. After all of ACW2 buffer is drawn through the QIAamp Mini column, the vacuum pump is switched off and pressure is released to 0 mbar. 750 μl of ethanol (96 v/v %) is applied to the QIAamp Mini column. The lid of the column is left open, and the vacuum pump switched on. After all of ethanol is drawn through the spin column, the vacuum pump is switched off and pressure is released to 0 mbar. The lid of the QIAamp Mini column is closed and the column is removed from the vacuum manifold after which the VacConnector is discarded. The QIAamp Mini column is placed in a clean 2 ml collection tube, and centrifuged at full speed (20,000×g; 14,000 rpm) for 3 min. The QIAamp Mini Column is placed in a new 2 ml collection tube. The lid is opened and the assembly is incubated at 56° C. for 10 min to completely dry the membrane. The QIAamp Mini column is placed in a clean 1.5 ml elution tube and the lastly used 2 ml collection tube is discarded. 20 μl of AVE buffer is carefully applied to the center of the QIAamp Mini membrane. The lid is closed and incubated at room temperature for 3 min. Elution buffer AVE is equilibrated to room temperature. The recovered eluate volume is about 5 μl less than the elution volume applied to the QIAamp Mini column. The columns are centrifuged in a microcentrifuge at full speed (20,000×g; 14,000 rpm) for 1 min to elute the nucleic acids such that cell free RNA is obtained.

Free-circulating cell-free RNA is eluted in AVE buffer, ready for use in amplification reactions or storage at −30 to −15° C. Purified nucleic acids are free of proteins, nucleases, and other impurities.

Next, the concentration of the nucleic acids is determined prior to NRG1 fusion amplification and detection analysis according to the manufacturer's recommendation, such as quantitative amplification assays.

List of materials for the QIAvac 24 Plus System for vacuum processing of 1-24 QIAGEN spin columns. QIAvac 24 Plus: Vacuum Manifold for processing 1-24 spin columns which includes QIAvac 24 Plus Vacuum Manifold, Luer Plugs and Quick Couplings (Cat. No. 19413). VacConnectors (500): 500 disposable connectors for use with QIAamp Mini columns on luer slots or VacValves (Cat. No. 19407). VacValves (24): 24 valves for use with the QIAvac 24 Plus (Cat. No. 19408). Vacuum Regulator: For use with QIAvac manifolds (Cat. No. 19530). Vacuum Pump (230 V, 50 Hz): Universal vacuum pump (capacity 34 L/min, 8 mbar vacuum abs.) (Cat. No. 84020). QIAvac Connecting System: System to connect vacuum manifold with vacuum pump: includes tray, waste bottles, tubing, couplings, valve, gauge, 24 VacValves (Cat. No. 19419).

To avoid loss of RNA from biological material during and after purification, any plasticware or glassware is first thoroughly cleaned to eliminate possible RNase contamination. Inadvertent introduction of RNases into the isolation procedure is avoided by creating and maintaining an RNase-free environment.

### Example 4: Isolation of Circulating Tumor Cells

This protocol describes isolation of circulating tumor cells from whole blood using the AdnaTest BreastCancerSelect kit (ADNAGEN, catalog #T-1-508), which enables immunomagnetic enrichment of tumor cells via epithelial and tumor-associated antigens. Antibodies against epithelial and tumor associated antigens are conjugated to magnetic beads (Dynabeads) for labeling tumor cells in peripheral blood. Labeled cells are extracted by a magnetic particle concentrator (AdnaMag-L and AdnaMag-S) and are subsequently lysed. mRNA is isolated from the resulting lysate and used for NRG1 fusion detection.

Whole blood (at least 5 ml, but 10 ml is also possible) is collected in tubes containing EDTA ((e. g. ‘S Monovette® Kalium EDTA’, Sarstedt; ‘BD Vacutainer® K3EDTA’, Becton Dickinson) and is immediately placed on ice following the draw and processed within 4 hours.

Preparation of Select Beads: The BreastSelect Beads (provided in the kit) are resuspended thoroughly by pipetting. The volume of BreastSelect Beads required for all samples is calculated and the calculated volume transferred to 1.5 ml reaction tubes. The tubes are placed in AdnaMag-S Magnetic particle concentrator (AdnaGen GmbH, cat. No. T-1-800). Supernatant is removed after 1 min. Beads are washed with 1 ml PBS (Phosphate buffer saline; pH 7.0-7.3) three times. The tubes are removed from the AdnaMag-S and resuspended in 100 μl PBS.

Selection of Tumor cells: 5 ml of blood sample was first pipetted into a 15 ml tube. 100 μl of resuspended BreastSelect Beads are added to each blood sample. The tubes are rotated slowly (approx. 5 rpm) for 30 min at room temperature on a device allowing both tilting and rotation. The tubes are then placed into the AdnaMag-L (AdnaGen GmbH, cat. No. T-1-700) without magnetic slider, which is swung downwards to release blood drops captured in the cap. The magnetic slider is then inserted and the tubes incubated for 3 min at room temperature. The blood supernatant is removed completely with a 10 ml pipet without touching the beads. Washing is done three times by first removing the magnetic slider from the AdnaMag-L, adding 5 ml of PBS to the tubes and gentle resuspension of the magnetic bead/cell complex gently. The magnetic slider is placed back into the AdnaMag-L and tubes are incubated for 1 min at room temperature, followed by removal of the supernatant with a pipet. After washing, the magnetic slider is removed and bead/cell complexes are resuspended in 1 ml PBS and transferred to a 1.5 ml reaction tube. The reaction tube is placed into the AdnaMag-S with an inserted magnetic slider. After 1 min, the supernatant is completely removed for the subsequent cell lysis.

The magnetic slider is removed from the AdnaMag-S and 200 μl of room temperature equilibrated Lysis/Binding buffer (provided in the kit) is added to each reaction tube followed by resuspension by pipetting five times. The magnetic slider is inserted into the AdnaMag-S and incubated for 1 min. The supernatants containing the cell lysates are transferred to a new 1.5 ml reaction tubes and the tubes containing the beads are discarded. The cell lysates are suitable for mRNA-isolation or stored at −20° C. mRNA of the lysate is isolated using Oligo (dT) primers in a reverse transcription (RT-PCR) reaction and transcribed into cDNA template. NRG1 fusions are subsequently detected by PCR mediated amplification.

### Example 5: Extracellular Vesicles and Exosomes

Total vesicular RNA is obtained from blood using the ExoRNeasy Maxi kit (cat. No. 77023, QIAGEN GmbH, Hilden, Germany) which is based on membrane-based affinity binding step to isolate exosomes and other EVs from cell-free biofluids.

Step 1 includes plasma separation and storage. Whole blood is collected in BD Vacutainer® Venous Blood Collection Tubes (cat. no. 367525) containing EDTA (or PAXgene® Blood ccfDNA Tubes [cat. no. 768115] and Cell-Free DNA BCT®—but not RNA BCT—tubes from Streck). The tubes are stored at room temperature or 4° C. and processed within 1 hour. Blood samples are centrifuged in primary blood collection tubes for 10 min at 1900×g (3000 rpm) and 4° C. using a swinging bucket rotor. The upper (yellow) plasma phase is carefully transferred to a new tube (with conical bottom) without disturbing the intermediate buffy coat layer (containing white blood cells and platelets). Plasma samples are centrifuged in conical tubes for 15 min at 3000×g (or 10 min at 16,000×g—see above) and 4° C. or passed through a 0.8 μm filter (Sartorius® Minisart® NML [Sartorius cat. no. 16592]). The cleared supernatant is carefully transferred to a new tube without disturbing the pellet. The plasma is stored at 2-8° C. for up to 6 h and processed on the same day.

Step 2 describes capture and lysis of exosomes and other extracellular vesicles (EVs) from 4 ml of plasma. 4 ml of binding buffer XBP is added to 4 ml of plasma sample and mixed well immediately by gently inverting the tube 5 times. The plasma/XBP mix is added onto the exoEasy spin column (membrane affinity column which binds the EVs to the membrane) and centrifuged for 1 min at 500×g. The flow through is discarded and the column is placed back into the same collection tube. 10 ml wash buffer XWP is added to the exoEasy Maxi spin column, and centrifuged for 5 min at 5000×g. The flow through is discarded together with the collection tube. The spin column is transferred to a fresh collection tube. 700 μl QIAzol is added to the membrane for lysis of vesicles, centrifuged for 5 min at 5000×g to collect the lysate, and transferred completely to a 2 ml tube.

Step 3 describes isolation of total RNA from the lysate. Buffers RWT and RPE are prepared according to the manufacturer's instructions. The tube containing the lysate is briefly vortexed and incubated at room temperature for 5 min. 90 μl chloroform is then added to the tube and shaken vigorously for 15 s. After incubation at room temperature for 2-3 min, the tube is centrifuged for 15 min at 12,000×g at 4° C. The upper aqueous phase is transferred to a new collection tube, 2 volumes of 100% ethanol added and mixed thoroughly. 700 μl sample is pipetted into an RNeasy MinElute spin column in a 2 ml collection tube and centrifuged at ≥8000×g (≥10,000 rpm) for 15 s at room temperature. After discarding the flow through, this step is repeated using the remainder of the sample. 700 μl Buffer RWT is added to the RNeasy MinElute spin column and centrifuged for 15 s at ≥8000×g (≥10,000 rpm), followed by 500 μl Buffer RPE and centrifuged for 15 s at ≥8000×g (≥10,000 rpm). Flow-through is discarded at each step. Finally 500 μl Buffer RPE is added onto the RNeasy MinElute spin column and centrifuged for 2 min at ≥8000×g (≥10,000 rpm). The collection tube is discarded with the flow-through.

The RNeasy MinElute spin column is placed into a new 2 ml collection tube and centrifuge at full speed for 5 min with an open lid to dry the membrane. The collection tube is discarded with the flow through. RNA is eluted by adding 14 μl RNase-free water, let column stand for 1 min, and centrifuged for 1 min at full speed.

### Example 6: Tumor-Educated Platelets (TEPs)

Tumor cells are known to transfer (mutant) RNA into blood platelets, which are subsequently used to detect tumor-associated RNA markers. This protocol minimizes degradation of platelet mRNAs and minimizes the contamination by other cell types like red blood cells and leukocytes (Amisten S, Methods Mol Biol. 2012). RNA obtained from blood platelets is then used to amplify and detect NRG1 fusions.

Preparation of Antibody Conjugated Magnetic Beads: 1 ml Dynabead wash buffer (PBS with 0.1% (w/v) BSA, pH 7.4) is mixed with 250 μl Dynabead slurry (Dynabeads® Pan Mouse IgG, Invitrogen) and placed in the Dynamag magnet (DynaMag™ 2 or DynaMag™ 15, Invitrogen) for 1 min followed by removal of the liquid. After another wash, beads are removed from the magnet and resuspended in 250 μL wash buffer. 15 μl of anti-CD235a antibody (Mouse, anti-human CD235a, red blood cell surface marker, BD Inc, NJ, USA) and 15 μl anti-CD45 antibody (Mouse anti-human CD45, leukocyte surface marker, BD Inc, NJ, USA) are added to the beads, mixed and incubated for at least 30 min with gentle tilting and rotation. Supernatant is removed by placing the tube in the magnet for 1 min and beads are resuspended in 250 μl wash buffer before storing at 4° C.

Blood Collection and purification of platelets: A platelet inhibition cocktail is prepared with 18 ml ACD (sterile anticoagulant: citrate-dextrose solution, Sigma Aldrich), 12 μl of 1 mM PGE1 (Prostaglandin E1, 1 mM stock in ethanol, Sigma Aldrich), 120 μl of 30 mM acetylsalicylic acid (30 mM stock in ethanol, Sigma Aldrich), and 480 μl of 0.5 M EDTA (Sigma Aldrich). 8.5 ml of blood is collected in tubes of 15 ml tubes further containing 1.5 ml platelet inhibition cocktail. After centrifugation at room temperature at 200 g, 20 min, top 85% of the upper layer, the platelet rich plasma (PRP), is transferred to a new 15 ml tube. After another round of centrifugation (200 g, 10 min, room temperature) to further remove leukocytes and red blood cells, 85% of PRP is transferred to a 50 ml tube. The PRP is then filtered with the AutoStop™ BC high efficiency filter for leukocyte removal (Pall Inc, Cat. No. ATSBC1E), the filtrate is mixed with antibody conjugated magnetic beads and incubated while tilting and rotating at room temperature for 45 min. The PRP/bead mix is placed in the DynaMag™-2 for 2 min and supernatant transferred to a new tube to obtain depleted PRP. After repetition of PRP depletion step, supernatant is centrifuged to harvest platelets at 800 g, 10 min at room temperature. Platelet pellets are weighed and dissolved using 1 ml TRIzol (Invitrogen) per 100 mg of pellet, with a minimum of 1 mL TRIzol for samples weighing less than 100 mg.

Isolation of platelet RNA: Platelet samples dissolved in TRIzol are incubated at room temperature for 5 min and 200 μl chloroform (Sigma Aldrich) added to each tube. After shaking vigorously for 15-30 times, tubes are incubated for 2-3 min at room temperature and centrifuged at 12,000 g for 15 min at 4° C. 10 μg ultra-pure glycogen (UltraPure™ Glycogen, Invitrogen, Cat. No. 10814-010) is added to new RNase-free tubes. The aqueous phase of the TRIzol/chloroform mix is carefully added to glycogen containing tubes followed by 500 μl of cooled isopropanol (Sigma Aldrich). The tubes are mixed and left overnight at −20° C. After centrifugation at 12,000 g for 15 min at 4° C., the supernatant is removed and RNA pellet washed in 1 ml of 75% ethanol. Dried RNA pellets are dissolved in RNase-free water and can be further used for reverse transcription into cDNA.

### Example 7: ddPCR Nucleic Acid Amplification and Fusion Detection

Droplet Digital Polymerase Chain reaction (ddPCR) is used to detect target nucleic acid fusions from liquid biopsy cfDNA and cfRNA samples.

Droplet Digital Polymerase Chain reaction (ddPCR) performed with Bio-Rad's QX100™ or QX200™ Droplet Digital PCR system allows to detect target nucleic acid fusions from liquid biopsy DNA and/or RNA (cDNA) samples.

Primers and Probes: Primers are designed around the fusion junctions, or breakpoints, to amplify an NRG1 fusion product of 60-200 bp in size. Primer3 program (Whitehead Institute for Biomedical Research, Massachusetts Institute of Technology) is used to design primers with a GC content of 50-60%, Tₘ between 5° and 65° C. (at 50 mM salt concentration and 300 nM oligonucleotide concentration), avoiding secondary structures and primer dimers.

TaqMan™ hydrolysis probes are used which are sequence-specific and fluorescently labeled with FAM, HEX or VIC dyes. The probe sequence is chosen to be in between the two primers of the amplicon. Tₘ of the hydrolysis probe is 3-10° C. higher than that of the primers and the GC content is 30-80%. The probes are <30 nucleotides long.

Sample preparation: Concentration of input DNA/RNA is assessed using A260 spectroscopy, to ensure that the target DNA/RNA concentration that is being loaded is within the dynamic range of detection. The recommended DNA dynamic range of the QX100 or QX200 System is from 1 to 120,000 copies/20 μl reaction. Restriction digestion is also performed for genomic DNA using a 4-cutter or a 6-cutter enzyme and 10 Units enzyme/μg DNA according to the manufacturer's recommendations. Digested DNA is stored at −20° C. until further use.

When the experiment is performed with cfDNA as the starting material, synthetic DNA containing the sequences of the fusion variants is used as the positive control. The fusion sequence includes the fusion site plus sufficient length flanking on each side to cover the PCR amplicon. The resulting nucleotide sequence is between 50-250 ntd. A T7 promoter sequence (5′-CAGAGATGCATAATACGACTCACTATAGGGAGA-3′) is added to the 5′ end of the target sequence. The synthetic sequences are ordered from IDT (www.idtdna.com) as double stranded deoxyribonucleic acid (DNA) fragments and reconstituted in Tris-EDTA (TE) buffer to a final concentration of 10 ng/μl.

When RNA is used as the starting material, RT-PCR is used to first generate cDNA, for instance with Bio-Rad's iScript™ Select cDNA Synthesis Kit for oligo (dT) or gene-specific priming. cDNA synthesis is performed according to the manufacturer's manual. The concentration of cDNA is reduced to about 0.2 ng/μl RNA equivalent and 5 μl is used per Droplet Digital PCR reaction (total volume 20 μl).

For generation of positive control for cfRNA, 60 ng of synthetic DNA is converted into RNA using in vitro transcription and the resulting RNA transcripts are purified using phenol/guanidine based reagents. DNase I is added to remove residual template DNA. Quality of RNA is assessed by gel electrophoresis. The obtained RNA is diluted to concentration ranging from 0.25 to 2.5 fg, based on desired copy number output in the test sample. 10 μl aliquots of analytical RNA for use in the positive control are stored at −80° C. Nuclease-free water is used as negative control for both cfDNA and cfRNA.

ddPCR reaction: 20 μl PCR mix is prepared, containing the sample nucleic acid (1 μg of DNA or cDNA with a final concentration of 50 ng/μl), 2×ddPCR supermix for probes ((Bio-Rad, cat. no. 1863023; without 2′-deoxyuridine 5′-triphosphate)—10 μl, 20× fusion specific primers/probes set (450 nmol/L primers, 250 nmol/L FAM probe)—1 μl, 20× control target primers/probe set (450 nmol/L primers, 250 nmol/L HEX probe)—1 μl and nuclease-free water. An initial reaction pool slightly more than 20 μl (22-25 μl) is created to ensure that 20 μl of mixture is transferred to the DG8 cartridge (Bio-Rad, cat. no. 1864007). Reaction mixes are combined and mixed well in a separate tube and not in the droplet generator cartridge. Reaction mixes are then transferred to the DG8 Cartridge already preloaded in the DG8 Cartridge Holder (Bio-Rad, cat. no. 1863051).

After loading the 20 μl PCR reaction, 70 μl of Droplet Generation Oil (Bio-Rad, cat. no. 1863005) is loaded into the bottom wells of the DG8 Cartridge. A gasket is attached across the top of the DG8 Cartridge which is placed into the QX200 Droplet Generator. The Droplet Generator produces about 20,000 droplets per sample in about 2.5 min for eight samples. Droplets are then transferred to a 96-well plate by pipetting gently. The PCR plate is heat sealed using Bio-Rad's PX1™ PCR Plate Sealer and pierceable foil heat seal. The PCR plate is then placed in a C1000 Touch™ Thermal Cycler with 96-deep well reaction module for PCR. The thermal cycling conditions are as follows: Enzyme activation at 95° C. (10 min, 1 cycle); Denaturation (94° C., 30 sec) and Annealing/extension (55° C., 1 min) for 40 cycles; Enzyme deactivation at 98° C., 10 min, 1 cycle. A Ramp rate of 2° C./sec is used.

Following PCR amplification of the nucleic acid target in the droplets, the plate containing the droplets is placed in a QX100 or QX200 Droplet Reader, where using the QuantaSoft Software, each droplet is analyzed individually using a two-color detection system (set to detect FAM and HEX). Positive droplets, which contained at least one copy of the target DNA/RNA molecule, exhibit increased fluorescence compared to negative droplets. The concentration reported is in copies/μl of the final 1×ddPCR reaction. Thresholding tools are used to correctly designate the droplet populations as double negative (negative for both FAM and HEX), FAM positive, HEX positive, and double positive (positive for both FAM and HEX). Using the ABS analysis, absolute quantification of the target is obtained in copies/microliter and copies/droplet.

### Example 8: Anchored Multiplex PCR

Anchored Multiplex PCR (AMP) is used for the detection of gene fusions and to detect multiple fusions to the gene of interest. The following protocol is based on Archer FusionPlex Solid Tumor kit (ArcherDX, cat. no. AB0005) and to be performed using RNA obtained from liquid biopsy from e.g. example 6.

### Library Preparation:

A positive control is included which contains at least several confirmed gene fusions. A non-template control is included as an additional sample in every run.

Random Priming, First and Second strand cDNA synthesis: The desired input for the assay is 200 ng RNA, dilute in 10 mM Tris HCl pH8.0. 20 μl of the diluted RNA is added to the pre-chilled Random Priming reagent strip tubes (provided in the kit). Following mixing and a brief spin, the mixture is transferred to 96 well PCR plate, sealed with RT film (USA Scientific, cat. no. 2921-7800) and incubated in thermocycler block at 65° C. for 5 minutes (heated lid).

The random priming product is transferred into the First Strand reagent strip tubes (placed in pre-chilled aluminum block) and mixed, brief spin and transferred to a 96 well PCR plate. The following thermocycler program is used: 25° C. 10 min, 42° C. 30 min, 80° C. 20 min, 4° C. hold (heated lid).

1:10 dilution of the first strand product is made in a new set of PCR strip tubes in nuclease free water, to be used in Pre-Seq quality control (QC) assay. QC assay is primarily used for the verification of no cDNA synthesis from the no-template control. The QC assay is carried out according to the manufacturer's protocol.

For second strand cDNA synthesis, 21 μl nuclease free water is added to the remaining first strand product and 40 μl of this product is added to the Second Strand reagent strip tube (provided in the kit, placed in pre-chilled aluminum block). After mixing and a brief spin, the mixture is transferred to a 96 well PCR plate and sealed. The plate is inserted in thermocycler block, and the following thermocycler program used: 16° C. 60 min, 75° C. 20 min, 4° C. hold (heated lid).

End Repair: 40 μl of the second strand product is transferred into the End Repair reagent strip tube (provided in the kit, placed in pre-chilled aluminum block). After mixing and a brief spin, the strip tube is incubated in thermocycler block at 25° C. 30 min followed by 4° C. on hold. The End Repair product is added to purification beads (Agencourt AMPure XP Beads, Bechman Coulter, cat. no. A63881) at room temperature. After mixing, the mixture is incubated at room temperature for 5 minutes, followed by a 5-minute incubation on magnet (Alpaqua, cat. no. A32782). Supernatant is discarded, beads are washed twice with 200 μl 70% ethanol with 30 second incubations and air dried for 5 minutes. The beads are re-suspended in 22 μl 10 mM Tris HCl pH 8.0, incubated off the magnet for 3 minutes followed by a 2-minute incubation on the magnet.

Ligation Step 1: 20 μl from the End Repair bead purification plate is transferred into the Ligation Step 1 strip tubes (provided in the kit, placed in pre-chilled aluminum block). After mixing and a brief spin, the mixture is incubated in a 96 well PCR plate in thermocycler block for 37° C. 15 min and 4° C. on hold (heated lid). The entire volume of Ligation Step 1 product is added to 50 μl of room temperature equilibrated beads. After mixing, the mixture is incubated at room temperature for 5 minutes, followed by a 5-minute incubation on the magnet. The supernatant is discarded and beads washed twice with 200 μl 70% ethanol with 30 second incubations. After the final wash all 70% ethanol is removed and the beads air dried for 5 minutes. The beads are removed from magnet and re-suspended in 42 μl 10 mM Tris HCl pH 8.0, incubated off the magnet for 3 minutes followed by a 2-minute incubation on the magnet.

### Ligation Step 2:

MBC Adapter Strip tube reagents (provided in the kit) are removed from 4° C. storage and numbered properly. The sample-specific indexes are also recorded for sequencing purposes.

40 μl from the Ligation Step 1 bead purification plate is transferred to the MBC Adapter strip tubes (ArcherDX cat. no. AK0016-48, placed in pre-chilled aluminum block). After mixing and a brief spin, the mixture is transferred to Ligations Step 2 reagent strip tubes (provided in the kit, also placed in pre-chilled aluminum block). After mixing and a brief spin, the mixture is incubated in a thermocycler for 22° C. 5 min, and 4° C. on hold.

Ligation Cleanup beads (equilibrated at room temperature) are vortexed and 50 μl added to a new set of PCR strip tubes. After 1 minute incubation on magnet, the supernatant is discarded. Strip tubes are removed from magnet and re-suspended in 50 μl of Ligation Cleanup Buffer.

Ligation Step 2 product is added to the Ligation Cleanup bead strip tubes, mixed by vortexing and incubated at room temperature for 5 minutes. The mixing and incubation is repeated following which the samples are incubated on the magnet for 1 minute and supernatant discarded. 200 μl of Ligation Cleanup buffer is added for re-suspension and after a quick spin and the mixture is placed on magnet for 1 minute. Washing with the Ligation cleanup buffer is repeated. An identical wash is performed using ultrapure water, following which the beads are re-suspended in 20 μl of 5 mM NaOH and incubated in a PCR plate as follows in thermocycler block: 75° C. 10 min, 4° C. hold (heated lid). The PCR plate is placed on magnet for at least 3 minutes.

First PCR: 2 μl of the NRG1 specific primers (GSP1 primers) are added to each well of the First PCR reagent strip tubes (provided in the kit) and mixed with 18 μl of Ligation 2 cleanup product. After a brief spin, the mixture is incubated in a thermocycler with the following program: 95° C. 3 min, 15 cycles 95° C. 30 sec-65° C. 5 min (100% ramp rate), 72° C. 3 min, 4° C. hold (heated lid).

20 μl of First PCR product is added to 24 μl of room temperature-equilibrated beads, mixed and incubated at room temperature for 5 min and on magnet for 2 min. Following removal of supernatant, the beads are washed twice with 200 μl 70% ethanol with 30 second incubations, air dried and resuspended in 24 μl 10 mM Tris HCl pH8.0.

### Second PCR and Library Quantitation

2 μl of the additional NRG1 specific primers (GSP2 primers) are added to each well of the properly labeled Second PCR reagent strip tubes (provided in the kit) in a cold aluminum block, and mixed with 18 μl of the First PCR cleanup product. After a brief spin, the mixture is incubated in a thermocycler block as follows: 95° C. 3 min, 18 cycles 95° C. 30 sec-65° C. 5 min (100% ramp rate), 72° C. 3 min, 4° C. hold (heated lid).

20 μl of Second PCR product is added to 24 μl of room temperature-equilibrated beads, mixed and incubated at room temperature for 5 min and on magnet for 2 min. Following removal of supernatant, beads are washed twice with 200 μl of 70% ethanol with 30 second incubations, air dried and resuspended in 24 μl 10 mM Tris HCl pH8.0. After 2 minute incubation on the magnet, 20 μl of Second PCR cleanup product is transferred to a new PCR plate and quantified.

The concentration of each library is determined by qPCR using Kapa Biosystems Library Quantification Kit for Illumina no. KK4973, according to the manufacturer's instructions. The Second PCR product is serially diluted using 10 mM Tris HCl pH8.0 0.05% Tween. 6 μl of library quantitation master mix (Kapa Biosystems, cat. no. KK4973) is added per well to an optical 96-well plate followed by 4 μl of appropriate dilution or Standard (Kapa Biosystems, cat no. KK4906, KK4903). The following qPCR program is used: 1 cycle 95° C. 5 min, 35 cycles 95° C. 30 sec (4.4° C./sec ramp rate)-60° C. 45 sec (2.2° C./sec ramp rate). After library quantification is complete, all libraries are normalized and pooled by combining 10 μl of each normalized library into one 1.5 ml microcentrifuge tube.

### Sequencing of Libraries:

Sequencer reagent cartridge is removed from cold storage and placed in deionized water up to the fill line for at least one hour. The sequencer reagent kit (MiSeq Reagent Kit v3-600 cycle; Illumina; cat. no. MS-102-3003) is allowed to equilibrate with room temperature. 10 μl of the library pool is combined with 10 μl of 0.2N NaOH to make the Denatured Amplicon Library (DAL) pool and incubated at room temperature for 5 min. 10 μl of 200 mM Tris HCl pH 7.0 is added to the DAL, followed by 970 μl of HT1 buffer (provided in the kit).

The final load tube is made by combining 300 μl of HT1, 25 μl of 20 pM PhiX and 675 μl of the DAL pool. After mixing and a brief spin, the entire volume of the load tube is added to the sample well of the sequencer reagent cartridge and the cartridge is loaded into the sequencer running 2×151 bp reads with 2×8 index reads (MiSeqDx System—Illumina).

RNA Fusion data is analyzed using an appropriate bioinformatic data analysis software.

### Example 9: Next-Generation Sequencing

Next-generation sequencing is performed with DNA samples using MI-Exome test (Caris Molecular Intelligence®, Caris Life Sciences) that uses DNA and provides tumor mutational profiling using a custom human exome panel to detect variants including single nucleotide polymorphisms, insertions/deletions and DNA rearrangement and fusion events among others.

The following custom exome panel, which is a blend of five panels is used. The panel is validated for use with the Illumina NovaSeq 6000 instrument. The blend includes two off-the-shelf panels from Agilent and three panels developed and optimized by Caris:
Agilent Human All Exon V7 Panel (48 MB)
Agilent SNP Backbone Panel (3 MB panel with 1 MB resolution)
Caris 719-Gene Targeted Clinical Panel (1 MB)
Caris Intronic Fusion Panel (0.1 MB)
Caris Pathogenic Panel (0.1 MB)

For RNA as the starting material, next generation sequencing is performed using a whole transcriptome assay, for instance the MI Transcriptome™ assay (Caris Molecular Intelligence®, Caris Life Sciences). The assay detects gene fusions and splice variants using the Agilent SureSelect Human All Exon Panel when sequences on the Illumina NovaSeq instrument.

### Example 10: Fluorescence In Situ Hybridization (FISH)

FISH by break apart probes is carried out on circulating tumor cells (CTC) obtained from plasma or blood or other liquid biopsy samples. For instance, circulating tumor cells are purified from plasma, serum etc using either CellSearch® (Menarini silicon biosystems, Italy) or ISET® (Rarecells Diagnostics, Paris, France) systems and fixed on slides.

Briefly the CTCs enriched from whole blood are mounted on individual glass slides (Frithiof, Henrik et al. OncoTargets and therapy vol. 9, 7095-7103, 16 Nov. 2016). CTC-containing solution (˜900 μL) is transferred to a 1.5 mL Eppendorf tube and placed in a magnetic tray. Following 10 min incubation, the nonadherent solvent is removed. Cells are resuspended in 10 μL of 1× phosphate buffer saline and mounted on superfrost slides (ThermoScientific, Germany) which are incubated at 37° C. for 30 min. Fixation is accomplished by immersing the slides in 100% methanol for 5 min. Samples are stored at −20° C. until further evaluation.

Pretreatment of the cells is done using ZytoLight® FISH-Tissue Implementation Kit (Zyto Vision, Cat. No. Z-2028-5). Buffers PT1, ES1, WB1, WB2 and MT7 are provided in the kit.

Slides are incubated for 10 min at 70° C., followed by incubation for 2×10 min in xylene. The slides are then rehydrated using an ethanol series of 100%, 100%, 90%, 70% ethanol for 5 min each and finally washed 2×2 min in deionized or distilled water. Slides are incubated for 15 min in pre-warmed Heat Pretreatment Solution Citric (PT1) at 98° C. and transferred immediately to deionized or distilled water for washing 2×2 min. After blotting off the water, Pepsin Solution (ES1) is applied to the specimen and incubated for 15 min at 37° C. in a humidity chamber and washed for 5 min in Wash Buffer SSC (WB1). The slides are washed for 1 min in deionized water, dehydrated in an ethanol series with 70%, 90%, and 100% ethanol each for 1 min and air dried.

The ZytoLight SPEC NRG1 Dual Color Break Apart Probe (PL140) consists of ZyGreen and ZyOrange labeled polynucleotides. These probes target sequences that map to both the distal and the proximal sides of a breakpoint as occurred in a gene of interest, e.g. VAPB, CADM1, CD44, SLC3A2, VTCN1, CDH1, CXADR, GTF2E2, CSMD1, PTN, ST14, AGRN, THBS1 or PVALB. 10 μl probe is pipetted onto each pretreated specimen, covered with a 22 mm×22 mm coverslip and sealed. Denaturation is done by placing the slides in a hybridizer for 10 min at 75° C. following which the slides are transferred to a humidity chamber and hybridized overnight at 37° C.

On the next day, slides are submerged in 1× Wash Buffer A (WB2) at 37° C. for 1-3 min to remove the coverslip and washed again for 2×5 min at 37° with Wash Buffer A. Slides are incubated in 70%, 90% and 100% ethanol for 1 min each and air dried. 25 μl of DAPI/DuraTect-Solution (MT7) is pipetted onto the slides, covered with a coverlip and incubated in the dark for 15 min. Sample fluorescence is then evaluated by fluorescence microscopy using the appropriate filters for ZyGreen (excitation 503 nm/emission 528 nm) and for ZyOrange (excitation 580 nm/emission 599 nm). Results are interpreted based on the overlapping or non-overlapping appearance of green and orange signals.

### Example 11: Immunocytochemistry (ICC)

Immunocytochemistry is carried out on circulating tumor cells obtained from plasma or blood or other liquid biopsy samples to detect the expression of the NRG1 fusion protein. Circulating tumor cells are purified from plasma, serum etc using either CellSearch® (Menarini silicon biosystems, Italy) or ISET® (Rarecells Diagnostics, Paris, France) systems and fixed on slides.

Briefly the CTCs enriched from whole blood are mounted on individual glass slides (Frithiof, Henrik et al. OncoTargets and therapy vol. 9, 7095-7103, 16 Nov. 2016). The CTC-containing solution (˜900 μL) is transferred to a 1.5 mL Eppendorf tube and placed in a magnetic tray. Following 10 min incubation, the nonadherent solvent is removed. Cells are resuspended in 10 μL of 1× phosphate buffer saline and mounted on superfrost slides (ThermoScientific, Germany) which are incubated at 37° C. for 30 min. Fixation is accomplished by immersing the slides in 100% methanol for 5 min. Samples are stored at −20° C. until further evaluation.

Following fixation, detection of proteins on the slides is carried out using an appropriate immunostaining system (For example Ventana Discovery automated immunostaining system, Ventana Medical Systems, Tucson, AZ, USA). Briefly, cells mounted on superfrost slides are pretreated at pH 8.0 in an EDTA-based buffer. Two primary antibodies are applied to the NRG1 fusions of the present disclosure. For example, an anti-NRG1 antibody, targeting a region which is positioned at the C-terminal side of the fusion junction (such as the EGFR domain of NRG1) and another primary antibody targeting an epitope or domain of CD44 which is positioned N-terminal of the fusion junction. Antibody dilutions are performed with an appropriate diluent. The antibodies are detected using appropriate secondary antibodies and two different fluorescence detection systems. Immunostaining is visualized using different channels in a fluorescence microscope. Results are interpreted based on the overlapping or non-overlapping appearance of the two fluorescence signals.

### Example 12: Treatment Protocol

Treatment with zenocutuzumab in a patient diagnosed with each of the following fusion: VAPB-NRG1, CADM1-NRG1, CD44-NRG1, SLC3A2-NRG1, VTCN1-NRG1, CDH1-NRG1, CXADR-NRG1, GTF2E2-NRG1, CSMD1-NRG1, PTN-NRG1, ST14-NRG1, THBS1-NRG1, AGRN-NRG1, PVALB-NRG1, APP-NRG1, WRN-NRG1, DAAM1-NRG1, ASPH-NRG1, NOTCH2-NRG1, CD74-NRG1, SLC4A4-NRG1, SDC4-NRG1, ZFAT-NRG1 and DSCAML1-NRG1 is as follows.

A bi-weekly schedule is followed consisting of dosing 750 mg of zenocutuzumab over four hours for the first infusion followed by a two-hour dosing for each subsequent infusion every other week in a 4-week cycle. Also, premedication is included to manage IRRs (Infusion Related Reactions), which consists of antipyretics and antihistamines for all infusions. Corticosteroids are included prior to the Day 1 cycle 1 dose; thereafter they are administered according to the investigator's discretion to manage IRRs.

### Example 13: Patient-Derived VAPB-NRG1 Fusion Data

A paraffin fixed sample of tumorous material with 60% tumorous cells from the right parietal pleura obtained from a 68-year old male patient diagnosed with lung adenocarcinoma was obtained on which molecular biotechnological analyses were performed as follows.

An Archer FusionPlex™ panel that allows detection in a panel of 14 genes (ALK, BRAF, EGFR, FGFR1, FGFR2, FGFR3, KRAS, MET, NRG1, NTRK1, NTRK2, NTRK3, RET, ROS1) was used. Libraries were prepared according to the manufacturer's instructions and Next Generation Sequencing (NGS) was performed on an Illumina MiSeq machine and sample analysis was done using the Archer Analysis 6.0 site.

The sample tested positive for a VAPB-NRG1 fusion in this panel. Sequencing performed on the sample led to the identification of the sequence according to SEQ ID NO: 3. It revealed the presence of a fusion junction between exon 1 of VAPB and exon 2 of NRG1. The underlined sequence, nucleotides 1-43, corresponds to a portion of exon 1 of the gene coding for VAPB (NM_004738.4). Nucleotides 44-94 correspond to a portion of the gene coding for NRG1.

(SEQ ID NO: 3)
CAGGTCCTGAGCCTCGAGCCGCAGCACGAGCTCAAATTCCGAGCCTTGC
CTCCCCGATTGAAAGAGATGAAAAGCCAGGAATCGGCTGCAGGTT.

### Example 14: Patient-Derived CADM1-NRG1 Fusion Data

A formalin-fixed paraffin block with tumorous material from a male patient, diagnosed with metastatic lung adenocarcinoma NOS, was obtained on which molecular biotechnological analyses were performed as follows.

Molecular testing was performed after harvesting of metastatic brain tissue with an approved microdissection technique. Candidate slides were examined under a microscope and areas containing tumor cells (and separately normal cells, when necessary for testing) were circled. A laboratory technician harvested targeted tissue for extraction from the marked areas using a dissection microscope. The areas marked and extracted were microscopically reexamined on post microdissected slides and adequacy of microdissection was reviewed.

RNA-Seq performed on cDNA obtained from the sample led to the identification of the sequence according to SEQ ID NO: 7. It revealed the presence of an in-frame fusion with a junction occurring between exon 7 of the CADM1 gene and exon 6 of the NRG1 gene. The underlined sequence, nucleotides 1-53, corresponds to a portion of exon 7 of the CADM1 gene (NM_001301045.1). Nucleotides 54-85 correspond to a portion of the gene coding for NRG1 (NM_001159999.3).

(SEQ ID NO: 7)
AGCTTCAAACATAGTGGGGAAAGCTCACTCGGATTATATGCTGTATGTA
TACGCTACATCTACATCCACCACTGGGACAAGCCAT

A tumor mutational burden analysis was performed based on Next Generation Sequencing (NGS) analysis from genomic DNA isolated from the formalin-fixed paraffin embedded tumor sample using the Illumina NextSeq platform. Tumor mutational burden is calculated using only missense mutations that have not been previously reported as germline alterations. A high mutational burden is a potential indicator of immunotherapy response (Hellman et al., NEJM, 2018; Le et al., NEJM, 2015; Rizvi et al., Science, 2015; Rosenberg et al., Lancet 2016; Snyder et al., NEJM, 2014). Cut-off points in NSCLC are based on a large Phase 3 clinical trial which showed that patients with a TMB of 10 or more mutations per megabase had longer progression free survival when treated with immune checkpoint inhibitor combination therapy than those treated with chemotherapy (Hellman et al., NEJM, 2018). High: greater than or equal to 10 mutations/megabase, and Low: less than or equal to 9 mutations per megabase. The patient sample had a TMB score of 9.

Microsatellite Instability (MSI) analysis performed on the sample by NGS showed a stable MSI signature. MSI status by NGS (MSI-NGS) was measured by the direct analysis of known microsatellite regions sequenced in a panel of 592 known genes. To establish clinical threshold, MSI-NGS results were compared with results from over 2,000 matching clinical cases analyzed with traditional, PCR-based methods. Genomic variants in the microsatellite loci are detected using the same depth and frequency criteria as used for mutational detection. Only insertions and deletions resulting in a change in the number of tandem repeats were considered in this assay. The total number of microsatellite alterations in each sample were counted and grouped into three categories: High, Equivocal and Stable. MSI-low results are reported in the Stable category.

Immunohistochemistry performed on the sample revealed a negative result for ALK and PD-L1, and positive results for MLH1, MSH2, MSH6, PMS2 and PTEN.

Using NGS or RNA-Seq analysis, no fusions or mutations were detected in the obtained sample in the following cancer type relevant biomarkers: NTRK1, NTRK2, NTRK3, KRAS, ALK, DOR2, ErbB2, ErbB3, ErbB4, PIK3CA, TP53, MET, PD-L1, RET, ROS1, STKI1, TP53.

### Example 15: Patient-Derived CD44-NRG1 Fusion and Patient Data

A biological sample of tumorous material from the liver of a 46-year old male patient was obtained on which molecular biotechnological analyses were performed as follows.

An Archer FusionPlex™ Custom Solid Panel that utilizes the Anchored Multiplex PCR (AMP™) technology targeting 62 exons of previously reported chromosomal rearrangements was used to detect gene fusions in the tumor sample. Unidirectional gene specific primers (GSPs) were designed to 62 targeted exons. GPSs in combination with adapters-specific primers amplify known and novel fusion transcripts. Enriched amplicons were sequenced on an Illumina MiSeq instrument.

The sample tested positive for a CD44-NRG1 fusion in this investigational panel. Sequencing performed on the sample led to the identification of the sequence according to SEQ ID NO: 11. It revealed the presence of an in-frame fusion with a fusion junction occurring between exon 5 of CD44 and exon 2 of NRG1. The underlined sequence, nucleotides 1-52, corresponds to a portion from exon 5 of the CD44 gene (NM_000610). Nucleotides 53-110 correspond to a portion of exon 2 of the gene coding for NRG1.

(SEQ ID NO: 11)
GACGAAGACAGTCCCTGGATCACCGACAGCACAGACAGAATCCCTGCTA
CCACCTTGCCTCCCCGATTGAAAGAGATGAAAAGCCAGGAATCGGCTGC
AGGTTCCAAACT

The sample tested negative in this clinically validated panel with a total of 62 exon targets for exon 20 of ALK (NM_004304), exons 2,4,8 of ERG (NM_004449), exons 6,7,8,10,11 of EWSR1 (NM_005243), exons 17,18 of FGFR3 (NM_00142), exon 17 of FGFR2 (NM_00141.4), exon 2 of FOXO1 (NM_002015), exons 11,12,14,15 of NTRK3 (NM_002530), exon 12 of RET (NM_020975), exons 34,35 of ROS (NM_002944), exon 10 of SS18 (NM_001007559), exons 16,17,19 of STAT6 (NM_001178078), exon 6 of TAF15 (NM_139215), exon 6 of TFE3 (NM_006521), exons 7,9,11 of BRAF (NM_004333), exon 12 of NTRK1 (NM_002529), exons 5,8 of FUS (NM_004960), exon 20 of CIC (NM_015125).

### Example 16: Patient-Derived SLC3A2-NRG1 Fusion Data

Tumorous material showing adenocarcinoma histology of primary origin obtained via a left lower lobectomy from a female patient diagnosed with lung adenocarcinoma was obtained on which molecular biotechnological analyses were performed as follows.

Molecular testing was performed using Archer Lung Fusion Plex, Illumina MiniSEQ after harvesting of tumorous cells by sequencing of the following target genes: ALK, BRAF, FGFR1, FGFR2, FGFR3, KRAS, MET, NRG1, NTRK1, NTRK2, NTRK3, RET, ROS1. The reference genome was GRCh37/hg19.

RNA-Seq performed on cDNA obtained from the sample led to the identification of the sequence according to SEQ ID NO: 15. It revealed the presence of an in-frame fusion junction occurring between exon 1 of the SLC3A2 gene and exon 5 of the NRG1 gene. The underlined sequence, nucleotides 1-53, corresponds to a portion of exon 1 of the gene coding for SLC3A2 (NM_001013251). The remaining 35 nucleotides, nucleotides 54-88, correspond to a portion of exon 5 of the NRG1 gene.

(SEQ ID NO: 15)
CCGCATCGGCGACCTTCAGGCCTTCCAGGGCCACGGCGCGGGCAACCTG
GCGGCATCTACATCTACATCCACCACTGGGACAAGCCAT.

### Example 17: Patient-Derived VTCN1-NRG1 Fusion Data

Metastatic tumorous biopsy material from a selected tissue block comprising tumorous cells was obtained from the right parasternal site from a patient diagnosed with a lung adenocarcinoma on which molecular biotechnological analyses were performed as follows.

Molecular testing was performed using the Archer Fusion Plex RNA CTL_V6 (Archerdx) kit, on material obtained after harvesting of tumorous cells and TNA extraction by sequencing as realized via Nextseq pair end using flow cell N^{o}HFFJFAFX2, with Sophia DDM version 5.7.4-b127-31a209c, Pipeline ID ILL1AM1R2/v5.5.24.1/GEN1GN1FSQ2.

The indicated kit allows detection of the mutations in the following target regions (with exons in brackets): AKT1 (3), ALK (22, 23, 24, 25), AXL (5, 11, 15, 17), BRAF (11, 15), CTNNB1 (3), CYSLTR2 (6), DDR2 (17), EGFR (18, 19, 20, 21), ERBB2 (20), FGFR1 (2, 8, 9, 10, 17), FGFR2 (2, 5, 7, 8, 9, 10), FGFR3 (3, 5, 8, 9, 10), GNA11 (4,5), GNAS (8, 9), GNAQ (4,5), HRAS (2, 3, 4), IDH1 (4), IDH2 (4), KEAP1 (full), KIT (11, 13, 17), KRAS (2, 3, 4), MAP2K1 (2, 3), MET (13 à 19), NRAS (2, 3, 4), PIK3CA (9, 20), POLE (9 à 14), RAF1 (4, 5, 6, 7, 9, 10, 11, 12), RET (11, 13, 14, 15, 16), ROS1 (38), STK11 (full), TP53 (full). Detection of transcriptional fusions of: ALK, AXL, BRAF, CCND1, EGFR, FGFR1, FGFR2, FGFR3, MAP2K1, MET, NRG1, NTRK1, NTRK2, NTRK3, PPARG, RAF1, RET, ROS1. And expression of: ALK, CCND1, EGFR, ERBB2, FGFR1, FGFR2, FGFR3, MET, NTRK1, NTRK2, NTRK3, RET, ROS1.

No mutations were observed in exons 18-21 of the EGFR gene; no mutations were detected in exons 2 and 3 of the KRAS gene; no mutations were detected in exons 11 and 15 of the BRAF gene; no mutations were detected in exon 20 of the ERBB2 gene; no mutations were detected in exons 9 and 20 of the PIK3CA gene; no mutations were detected in exon 14 of the MET gene; mutation 395G>T was detected in exon 4 of the IDH1 gene, leading to an R132L amino acid change and a transcriptional fusion between exon 2 of VTCN1 and exon 2 of NRG1 was detected.

No transcriptional fusions implicating ALK, ROS1, RET, NTRK1, NTRK2, NTRK3, FGFR1, FGFR2 and FGFR3 genes were detected.

No overexpression of ERBB2 or MET was detected.

RNA-Seq performed on cDNA obtained from the sample led to the identification of the sequence according to SEQ ID NO: 166. It revealed the presence of an in-frame fusion junction occurring between exon 2 of the VTCN1 gene and exon 2 of NRG1. The underlined sequence, nucleotides 1-65, corresponds to a portion of exon 2 of the gene coding for VTCN1, accession number NM_024626.4. The remaining 28 nucleotides, nucleotides 66-93, correspond to a portion of exon 2 of NRG1.

(SEQ ID NO: 166)
CATAATTAGCATCATCATTATTCTGGCTGGAGCAATTGCACTCATCATT
GGCTTTGGTATTTCAGCCTTGCCTCCCCGATTGAAAGAGATGAA.

### Example 18: Patient-Derived CDH1-NRG1 Fusion Data

Tumorous material was obtained from a female patient diagnosed with pancreatic adenocarcinoma, on which molecular biotechnological analyses were performed as follows. Automated RNA extraction was performed with the Maxwell (Promega) FFPE LEV kit.

Molecular testing was performed using Archer Fusion Plex NGS. NGS was performed (S5XL—Life Technologies) on the Oncomine Comprehensive Assay V3 (OCAV3) panel that contains 161 genes, of which 81 are considered hotspot genes and 48 full-length genes, and 47 copy number genes. RNA-Seq performed on cDNA obtained from the sample led to the identification of the sequence according to SEQ ID NO: 186.

It revealed the presence of an in-frame fusion junction occurring between exon 11 of the CDH1 gene and exon 2 of NRG1. The underlined sequence, nucleotides 1-119, corresponds to a portion of exon 11 of the gene coding for CDH1 (NM_001317185.2). The remaining 30 nucleotides, nucleotides 120-149, correspond to a portion of exon 2 of the NRG1 gene. Also, NGS revealed the presence of mutation 1172C>T in exon 10 of the FGFR3 gene, giving rise to the amino acid change Ala391Val.

(SEQ ID NO: 186)
CTGGCTGGAGATTAATCCGGACACTGGTGCCATTTCCACTCGGGCTGAG
CTGGACAGGGAGGATTTTGAGCACGTGAAGAACAGCACGTACACAGCCC
TAATCATAGCTACAGACAATGCCTTGCCTCCCCGATTGAAAGAGATGAA
A.
CCTTGCCTCCCCGATTGAAAGAGATGAAAA

### Example 19: Patient-Derived CXADR-NRG1 Fusion Data

Tumorous material was obtained from a male patient diagnosed with colon cancer, on which molecular biotechnological analyses were performed as follows. Areas of tumor of submitted a tissue specimen or slides microdissected from surrounding tissue, and total RNA isolated using standard methods. The isolated RNA was subjected to reverse transcription, and double stranded DNA was constructed. The specimen was universal-tagged and barcoded, and subjected to hemi-nested multiplex PCR using gene-specific primers and universal tag specific primers (Archer FusionPlex chemistry). The products were prepared and sequenced using Illumina NextSeq v2 chemistry. Sequence data was analyzed using Archer Analysis Software, v4.1. The analytic sensitivity is approximately 5 RNA molecules encoding the fusion and is dependent on the specific fusion event present. Fusions are relative to the reported reference transcript.

Molecular analyses revealed the presence of an in frame fusion junction occurring between exon 1 of the CXADR gene and exon 2 of NRG1. The underlined sequence, nucleotides 1-43, corresponds to a portion of exon 1 of the gene coding for CXADR (NM_001207063.2). The remaining 58 nucleotides, nucleotides 44-101, correspond to a portion of exon 2 of the NRG1 gene.

(SEQ ID NO: 217)
ATGGCGCTCCTGCTGTGCTTCGTGCTCCTGTGCGGAGTAGTGGCCTTGC
CTCCCCGATTGAAAGAGATGAAAAGCCAGGAATCGGCTGCAGGTTCCAA
ACT.

### Example 20: Patient-Derived GTF2E2-NRG1 Fusion Data

A formalin-fixed paraffin block with tumorous material from a female patient, diagnosed with metastatic breast adenocarcinoma, NOS, was obtained on which molecular biotechnological analyses were performed as follows.

Molecular testing was performed after harvesting of tissue with an approved microdissection technique. Candidate slides were examined under a microscope and areas containing tumor cells (and separately normal cells, when necessary for testing) were circled. A laboratory technician harvested targeted tissue for extraction from the marked areas using a dissection microscope. The areas marked and extracted were microscopically reexamined on post-microdissected slides and adequacy of microdissection was reviewed.

Gene fusion and variant transcript detection were performed on mRNA isolated from a formalin-fixed paraffin embedded tumor sample using the Agilent SureSelectXT Low Input Library prep chemistry, optimized for FFPE tissue, in conjunction with the SureSelect Human All Exon V7 bait panel (48.2 Mb) and the Illumina NovaSeq. This assay is designed to detect fusions occurring at known and novel breakpoints within genes. This analysis led to the identification of the sequence according to SEQ ID NO: 233. It revealed the presence of an in frame fusion with a junction occurring between exon 2 of the GTF2E2 gene and exon 2 of the NRG1 gene. The underlined sequence, nucleotides 1-141, corresponds to a portion of exon 2 of the GTF2E2 gene (NM_002095.6). Nucleotides 142-268 correspond to a portion of the gene coding for NRG1 (NM_001159999.3).

(SEQ ID NO: 233)
GGGAGCTGTTCAAAAAACGAGCTCTTTCTACTCCTGTAGTAGAAAAACG
TTCAGCATCTTCTGAGTCATCATCATCATCGTCAAAGAAGAAGAAAACA
AAGGTAGAACATGGAGGATCGTCAGGCTCTAAACAAAATTCTGCCTTGC
CTCCCCGATTGAAAGAGATGAAAAGCCAGGAATCGGCTGCAGGTTCCAA
ACTAGTCCTTCGGTGTGAAACCAGTTCTGAATACTCCTCTCTCAGATTC
AAGTGGTTCAAGAATGGGAATGA

A tumor mutational burden (TMB) analysis was performed based on Next Generation Sequencing (NGS) analysis from genomic DNA isolated from the formalin-fixed paraffin embedded tumor sample using the Illumina NextSeq platform. Tumor Mutational Burden is calculated using only missense mutations that have not been previously reported as germline alterations. Defined threshold levels for Tumor Mutational Burden and establish cutoff points were used as follows:
High: greater than or equal to 17 mutations/Megabase (≥17 mutations/Mb).
Intermediate: greater than or equal to 7 but fewer than 17 mutations/Megabase (≥7 and <17 mutations/Mb).
Low: less than or equal to 6 mutations/Megabase (≤6 mutations/Mb). The patient sample indicated a TMB score of 9.

Using Microsatellite Instability (MSI) analysis by NGS, no microsatellite instability was detected.

Using NGS analysis, a frameshift mutation was detected in exon 6 of GATA3 (c.1305_1327del23, protein alteration P436fs) and loss-off function variant NTRK2 (protein alteration p.Q172 c.514C>T, (NM_006180.4).

### Example 21: Patient-Derived CSMD1-NRG1 Fusion Data

Tumorous material was obtained from a male patient diagnosed with colorectal cancer, on which the following molecular biotechnological analyses were performed.

A custom oncology testing panel consisting of 596 genes with single nucleotide variants, indels and translocations was using hybrid-capture next generation sequencing. Whole genome transcriptome RNA sequencing was performed using IDT xGen Exome research panel v1.0 hybridization probes allowing unbiased detection of expressed fusion transcripts from rearranged genes.

It revealed the presence of an in-frame fusion junction occurring between exon 23 of the CSMD1 gene and exon 6 of NRG1. The underlined sequence, nucleotides 1-88, corresponds to a portion of exon 23 of the gene coding for CSMD1 (NM_033225.6). The remaining nucleotides, nucleotides 89-150, correspond to a portion of exon 6 of the NRG1 gene. The relevant patient report mentioned Afatinib and Erlotinib as FDA approved therapies related to this CSMD1-NRG1 fusion, while GSK2849330 was mentioned as an investigational study. Also, clinical trial TAPUR (NCT02693535) was mentioned in relation to the CSMD1-NRG1 fusion.

No pathogenic germline mutations were identified in patient material, neither were pathogenic single nucleotide polymorphisms identified in KRAS or NRAS. The following four somatic genomic variants were identified in patient material: A Y220C missense mutation in TP53, a R564* stop gain mutation in APC, an N1455fs frame shift mutation in APC and a G469A missense mutation in BRAF. The MSI status was established as being stable and a tumor mutational burden of 4.6m/MB.

Identified variants of unknown significance included c.3332+1G>A splice region variant in APOB (NM_000384), c.425C>T p.S142L missense variant in GATA3 (NM_001002295), c.352C>T p.R118C missense variant in LZTR1 (NM_006767), c.227A>T p.H76L missense variant in CTC1 (NM_025099), c.239G>A p.R80H missense variant in CYP1B1 (NM_000104), c.1099C>T p.R367W missense variant in RECQL4 (NM_004260), c.871A>G p.R291G missense variant in SOX2 (NM_003106) and c85_87del pE29del in frame deletion in TCF7L2 (NM_001146274).

Prior treatment of the patient included Fluorouracil, Leucovorin, Irinotecan, Bevacizumab and Oxaliplatin.

(SEQ ID NO: 255)
ATCCTAAACAGCACATCCAATCACCTGTGGCTAGAGTTCAACACCAATG
GATCTGACACCGACCAAGGTTTTCAACTCACCTATACCACTACATCTAC
ATCCACCACTGGGACAAGCCATCTTGTAAAATGTGCGGAGAAGGAGAAA
ACT.

### Example 22: Patient-Derived PTN-NRG1 Fusion Data

A formalin fixed paraffin block with tumorous material from a female patient, diagnosed with metastatic breast carcinoma, NOS, was obtained on which molecular biotechnological analyses were performed as follows.

Molecular testing was performed after harvesting of tissue with an approved microdissection technique. Candidate slides were examined under a microscope and areas containing tumor cells (and separately normal cells, when necessary for testing) were circled. A laboratory technician harvested targeted tissue for extraction from the marked areas using a dissection microscope. The areas marked and extracted were microscopically reexamined on post-microdissected slides and adequacy of microdissection was reviewed.

Gene fusion and variant transcript detection were performed on mRNA isolated from a formalin-fixed paraffin embedded tumor sample using the Agilent SureSelectXT Low Input Library prep chemistry, optimized for FFPE tissue, in conjunction with the SureSelect Human All Exon V7 bait panel (48.2 Mb) and the Illumina NovaSeq. This assay is designed to detect fusions occurring at known and novel breakpoints within genes. This analysis led to the identification of the sequence according to SEQ ID NO: 313. It revealed the presence of an in frame fusion with a junction occurring between exon 4 of the PTN gene and exon 2 of the NRG1 gene. The underlined sequence, nucleotides 1-102, corresponds to a portion of exon 2 of the PTN gene (NM_001321386.2). Nucleotides 103-205 correspond to a portion of the gene coding for NRG1 (NM_001159999.3).

(SEQ ID NO: 313)
CCAGAACTGGAAGTCTGAAGCGAGCCCTGCACAATGCCGAATGCCAGAA
GACTGTCACCATCTCCAAGCCCTGTGGCAAACTGACCAAGCCCAAACCT
CAAGCCTTGCCTCCCCGATTGAAAGAGATGAAAAGCCAGGAATCGGCTG
CAGGTTCCAAACTAGTCCTTCGGTGTGAAACCAGTTCTGAATACTCCTC
TCTCAGATT

A tumor mutational burden (TMB) analysis was performed based on Next Generation Sequencing (NGS) analysis from genomic DNA isolated from the formalin-fixed paraffin embedded tumor sample using the Illumina NextSeq platform. Tumor Mutational Burden is calculated using only missense mutations that have not been previously reported as germline alterations. Defined threshold levels for Tumor Mutational Burden and establish cutoff points were used as follows:
High: greater than or equal to 17 mutations/Megabase (≥17 mutations/Mb).
Intermediate: greater than or equal to 7 but fewer than 17 mutations/Megabase (≥7 and <17 mutations/Mb).
Low: less than or equal to 6 mutations/Megabase (≤6 mutations/Mb). The patient sample indicated a low TMB score.

Using Microsatellite Instability (MSI) analysis by NGS, no microsatellite instability was detected.

Using NGS analysis, a p.E336*, c. 1006G>T mutation in exon 10 of TP53 was detected (NM_000546.5). No alterations were detected in NTRK1/2/3, AKT1, BRCA1/2, ERBB2, ESR1, PIK3CA, PTEN and negative IHC results were obtained for PD-L1 (based on 22c3 and SP142).

### Example 23: Patient-Derived ST14-NRG1 Fusion Data

Molecular analyses performed on tumorous material obtained from a patient diagnosed with non-small cell lung cancer revealed the presence of an in-frame fusion junction occurring between exon 11 of the ST14 gene and exon 6 of NRG1 comprised by the sequence according to SEQ ID NO: 330. The underlined sequence, nucleotides 1-95, corresponds to a portion of exon 11 of the gene coding for ST14 (NM_021978.4). The remaining 87 nucleotides, nucleotides 96-182, correspond to a portion of exon 6 of the NRG1 gene.

(SEQ ID NO: 330)
CAACAGCAACAAGATCACAGTTCGCTTCCACTCAGATCAGTCCTACACC
GACACCGGCTTCTTAGCTGAATACCTCTCCTACGACTCCAGTGACCCTA
CATCTACATCCACCACTGGGACAAGCCATCTTGTAAAATGTGCGGAGAA
GGAGAAAACTTTCTGTGTGAATGGAGGGGAGTGCT.

### Example 24: Patient-Derived THBS1-NRG1 Fusion Data

Fresh, frozen or a formalin-fixed paraffin block with tumorous material from which tumor DNA was extracted obtained from a patient diagnosed with pancreatic adenocarcinoma was obtained on which molecular biotechnological analyses were performed as follows.

Extracted DNA was surveyed for the presence of exonic DNA sequences of 447 cancer genes and 191 regions across 60 genes for detection of rearrangements. DNA was isolated from tissue containing at least 20% tumor nuclei and analyzed by massively parallel sequencing using a solution phase Agilent SureSelect hybrid capture kit and Illumina HiSeq 2500 sequencer.

Molecular analyses revealed the presence of an in-frame fusion junction occurring between exon 9 of the THBS1 gene and exon 6 of NRG1 comprised by the sequence according to SEQ ID NO: 376. The underlined sequence, nucleotides 1-56, corresponds to a portion of exon 9 of the gene coding for THBS1 (NM_003246.4). Nucleotides 57-145 correspond to a portion of exon 6 of the NRG1 gene.

(SEQ ID NO: 376)
ACCCTGTGAAGGCGAAGCGCGGGAGACCAAAGCCTGCAAGAAAGACGCC
TGCCCCACTACATCTACATCCACCACTGGGACAAGCCATCTTGTAAAAT
GTGCGGAGAAGGAGAAAACTTTCTGTGTGAATGGAGGGGAGTGCTTC.

Further molecular analyses did not reveal a fusion in ALK, NTRK1, NTRK2, NTRK3, ROS1 and no pathogenic mutations, copy number changes or structural variants involving BRCA1, BRCA2 or PALB2 were detected. The following mutations were identified: c.783-10_787delinsCCTG in TP53, a single copy deletion of TP53 at 17p13.1, c.* 178T>A, exon 3 of CDKN1B, c.1205A>C (p.N402T) in exon 12 of ERCC2, c. 1858T>C (p.C620R) in exon 21 of FANCD2, c.398A>C (p.N133T) in exon 5 of KIF1B, c. 1092-7T>C in NAB2, c.3245G>A (p.R1082H) in exon 20 of NOTCH1, c.1798C>T (p.R600W) in exon 13 of PTPN14 and c.1273C>A (p.H425N) in exon 9 of RASA1.

### Example 25: Patient-Derived AGRN-NRG1 Fusion Data

Tumorous material was obtained from a male patient diagnosed with pancreatic cancer (PDAC), on which the following molecular biotechnological analyses were performed.

A custom oncology testing panel consisting of 648 genes with single nucleotide variants, indels and translocations was using hybrid-capture next-generation sequencing. Whole-genome transcriptome RNA sequencing was performed allowing unbiased detection of expressed fusion transcripts from rearranged genes. The limit of detection of the assay is 5% variant allele fraction (VAF) with a sensitivity of 98.2% for single nucleotide variants, 5% VAF with sensitivity of 91.8% for indels and 91.7% sensitivity for translocations.

This analysis led to the identification of the sequence according to SEQ ID NO: 403. It revealed the presence of an in-frame fusion with a junction occurring between exon 12 of the AGRN gene and exon 6 of the NRG1 gene. The underlined sequence, nucleotides 1-106, corresponds to a portion of exon 12 of the AGRN gene (NM_001305275.2). Nucleotides 107-207 correspond to a portion of the gene coding for NRG1 (NM_001159999.3).

(SEQ ID NO: 403)
GTGTGCGGCTCAGATGGGGTCACCTACAGCACCGAGTGTGAGCTG
AAGAAGGCCAGGTGTGAGTCACAGCGAGGGCTCTACGTAGCGGCC
CAGGGAGCCTGCCGAGCTACATCTACATCCACCACTGGGACAAGC
CATCTTGTAAAATGTGCGGAGAAGGAGAAAACTTTCTGTGTGAAT
GGAGGGGAGTGCTTCATGGTGAAAGAC.

Also, overexpression of FGFR1 was detected.

### Example 26: Patient-Derived PVALB-NRG1 Fusion Data

Molecular analyses performed on tumorous material obtained from a patient diagnosed with non-small cell lung cancer revealed the presence of an in-frame fusion junction occurring between exon 4 of the PVALB gene and exon 6 of NRG1 comprised by the sequence according to SEQ ID NO: 437. The underlined sequence, nucleotides 1-102, corresponds to a portion of exon 4 of the gene coding for PVALB (NM_002854.3). Nucleotides 103-227 correspond to a portion of exon 6 of the NRG1 gene.

(SEQ ID NO: 437)
TAAAAGGCTTCTCCCCAGATGCCAGAGACCTGTCTGCTAAAGAAA
CCAAGATGCTGATGGCTGCTGGAGACAAAGATGGGGACGGCAAAA
TTGGGGTTGACGCTACATCTACATCCACCACTGGGACAAGCCATC
TTGTAAAATGTGCGGAGAAGGAGAAAACTTTCTGTGTGAATGGAG
GGGAGTGCTTCATGGTGAAAGACCTTTCAAACCCCTCGAGATACT
TG.

### Example 27: Patient-Derived SLC3A2-NRG1 Fusion Data

Tumorous material obtained from a patient diagnosed with a lung cancer on which molecular biotechnological analyses were performed as follows. Total RNA was extracted from a formalin-fixed paraffin block using a Maxwell RSC and ReliaPrep™ FFPE Total RNA Promega kit followed by Archer Dx CTL fusionplex. Bioinformatical analysis was performed using Archer Analysis Version 6.2.7.

This analysis revealed the presence of an in frame fusion junction occurring between exon 2 of the SLC3A2 gene and exon 6 of the NRG1 gene comprised by the sequence according to SEQ ID NO: 454. The underlined sequence, nucleotides 1-93, corresponds to a portion of exon 2 of the gene coding for SLC3A2 (NM_002394.6). Nucleotides 94-121 correspond to a portion of exon 6 of the NRG1 gene.

(SEQ ID NO: 454)
AGTTGGGGTCTCACTGTGTTGCCCAGACTGGTCTCGAACTCTTGG
CCTCAGGTGATCCTCTTCCCTCAGCTTCCCAGAATGCCGAGATGA
TAGCTACATCTACATCCACCACTGGGACAAG.

### Example 28: Patient-Derived APP-NRG1 Fusion Data

Tumorous material obtained from a patient diagnosed with a pancreatic cancer on which molecular biotechnological analyses were performed revealed the presence of an in-frame fusion junction occurring between exon 14 of the APP gene and exon 6 of the NRG1 gene comprised by the sequence according to SEQ ID NO: 486. The underlined sequence, nucleotides 1-54, corresponds to a portion of exon 14 of the gene coding for APP (NM_001136130.3). Nucleotides 55-141 correspond to a portion of exon 6 of the NRG1 gene.

(SEQ ID NO: 486)
TTGAGCCTGTTGATGCCCGCCCTGCTGCCGACCGAGGACTGACCA
CTCGACCAGCTACATCTACATCCACCACTGGGACAAGCCATCTTG
TAAAATGTGCGGAGAAGGAGAAAACTTTCTGTGTGAATGGAGGGG
AGTGCT

### Example 29: Patient-Derived WRN-NRG1 Fusion Data

Tumorous material obtained from a patient diagnosed with breast cancer on which molecular biotechnological analyses were performed revealed the presence of an in-frame fusion junction occurring between exon 33 of the WRN gene and exon 6 of the NRG1 gene comprised by the sequence according to SEQ ID NO: 528. The underlined sequence, nucleotides 1-96, corresponds to a portion of exon 33 of the gene coding for APP (NM_000553.6). Nucleotides 97-182 correspond to a portion of exon 6 of the NRG1 gene.

(SEQ ID NO: 528)
AAGCTGGCTGCCCCCTTGATTTGGAGCGAGCAGGCCTGACTCCAG
AGGTTCAGAAGATTATTGCTGATGTTATCCGAAACCCTCCCGTCA
ACTCAGCTACATCTACATCCACCACTGGGACAAGCCATCTTGTAA
AATGTGCGGAGAAGGAGAAAACTTTCTGTGTGAATGGAGGGGAGT
GC

### Example 30: Patient-Derived DAAM1-NRG1 Fusion Data

Tumorous material obtained from a patient diagnosed with a breast cancer on which molecular biotechnological analyses were performed revealed the presence of an in-frame fusion junction occurring between exon 1 of the DAAM1 gene and exon 1 of the NRG1 gene comprised by the sequence according to SEQ ID NO: 605. The underlined sequence, nucleotides 1-75, corresponds to a portion of exon 1 of the gene coding for DAAM1 (NM_001270520.2). Nucleotides 76-150 correspond to a portion of exon 1 of the NRG1 gene.

(SEQ ID NO: 605)
GAAGGAAACTGTTTAACCGGATCCCATTGTACCCAGAGTGCAGAG
CCGCCTTTCCAGCATGCAGGGGCTGCTCAGGACAGAGAGGGAGGA
GGCGCGCGGGGACGGGGACGCCCAGGAGGACCCACTCGCGGGTCC
CGCTCCGCTCCGGCA

### Example 31: Patient-Derived ASPH-NRG1 Fusion Data

Tumorous material obtained from a patient diagnosed with a colorectal adenocarcinoma on which molecular biotechnological analyses were performed revealed the presence of an in-frame fusion junction occurring between exon 22 of the ASPH gene and exon 2 of the NRG1 gene comprised by the sequence according to SEQ ID NO: 635. The underlined sequence, nucleotides 1-75, corresponds to a portion of exon 1 of the gene coding for ASPH (NM_001164750.2). Nucleotides 76-150 correspond to a portion of exon 2 of the NRG1 gene.

(SEQ ID NO: 635)
AAGGTCTCTTCCTGCCTGAGGATGAAAACCTGAGGGAAAAAGGGG
ACTGGAGCCAGTTCACGCTGTGGCAGCAAGCCTTGCCTCCCCGAT
TGAAAGAGATGAAAAGCCAGGAATCGGCTGCAGGTTCCAAACTAG
TCCTTCGGTGTGAAA

### Example 32: Patient-Derived NOTCH2-NRG1 Fusion Data

Tumorous material obtained from a patient diagnosed with a pancreatic cancer on which molecular biotechnological analyses were performed revealed the presence of an in-frame fusion junction occurring between exon 6 of the NOTCH2 gene and exon 6 of the NRG1 gene comprised by the sequence according to SEQ ID NO: 693. The underlined sequence, nucleotides 1-75, corresponds to a portion of exon 6 of the gene coding for NOTCH2 (NM_024408.4). Nucleotides 76-150 correspond to a portion of exon 6 of the NRG1 gene.

(SEQ ID NO: 693)
CCTCCTGTACTCCAGGCTCCACCTGCATCGACCGTGTGGCCTCCT
TCTCTTGCATGTGCCCAGAGGGGAAGGCAGCTACATCTACATCCA
CCACTGGGACAAGCCATCTTGTAAAATGTGCGGAGAAGGAGAAAA
CTTTCTGTGTGAATG

### Example 33: Patient-Derived CD74-NRG1 Fusion Data

Tumorous material obtained from a patient diagnosed with a lung cancer on which molecular biotechnological analyses were performed revealed the presence of an in-frame fusion junction occurring between exon 2 of the CD74 gene and exon 2 of the NRG1 gene comprised by the sequence according to SEQ ID NO: 717. The underlined sequence, nucleotides 1-75, corresponds to a portion of exon 2 of the gene coding for CD74 (NM_001025159.3). Nucleotides 76-150 correspond to a portion of exon 2 of the NRG1 gene.

(SEQ ID NO: 717)
AGGGCCGGCTGGACAAACTGACAGTCACCTCCCAGAACCTGCAGC
TGGAGAACCTGCGCATGAAGCTTCCCAAGCCCTTGCCTCCCCGAT
TGAAAGAGATGAAAAGCCAGGAATCGGCTGCAGGTTCCAAACTAG
TCCTTCGGTGTGAAA

### Example 34: Patient Derived SDC4-NRG1 Fusion Data

Tumorous material obtained from a patient diagnosed with a lung cancer on which molecular biotechnological analyses were performed revealed the presence of an in-frame fusion junction occurring between exon 2 of the SDC4 gene and exon 2 of the NRG1 gene comprised by the sequence according to SEQ ID NO: 743. The underlined sequence, nucleotides 1-75, corresponds to a portion of exon 2 of the gene coding for SDC4 (NM_002999.4). Nucleotides 76-150 correspond to a portion of exon 2 of the NRG1 gene.

(SEQ ID NO: 743)
TACCAGACGATGAGGATGTAGTGGGGCCCGGGCAGGAATCTGATG
ACTTTGAGCTGTCTGGCTCTGGAGATCTGGCCTTGCCTCCCCGAT
TGAAAGAGATGAAAAGCCAGGAATCGGCTGCAGGTTCCAAACTAG
TCCTTCGGTGTGAAA

### Example 35: Patient-Derived CD44-NRG1 Fusion Data

Tumorous material obtained from a patient diagnosed with a cancer on which molecular biotechnological analyses were performed revealed the presence of an in-frame fusion junction occurring between 5 exon of the CD44 gene and exon 6 of the NRG1 gene comprised by the sequence according to SEQ ID NO: 761. The underlined sequence, nucleotides 1-75, corresponds to a portion of exon 5 of the gene coding for CD44 (NM_000610.4). Nucleotides 76-150 correspond to a portion of exon 6 of the NRG1 gene.

(SEQ ID NO: 761)
TTTCTACTGTACACCCCATCCCAGACGAAGACAGTCCCTGGATCA
CCGACAGCACAGACAGAATCCCTGCTACCACTACATCTACATCCA
CCACTGGGACAAGCCATCTTGTAAAATGTGCGGAGAAGGAGAAAA
CTTTCTGTGTGAATG

### Example 36: Patient-Derived SLC4A4-NRG1 Fusion Data

Tumorous material obtained from a patient diagnosed with a pancreatic cancer on which molecular biotechnological analyses were performed revealed the presence of an in-frame fusion junction occurring between exon 14 of the SLC4A4 gene and exon 6 of the NRG1 gene comprised by the sequence according to SEQ ID NO: 765. The underlined sequence, nucleotides 1-75, corresponds to a portion of exon 14 of the gene coding for SLC4A4 (NM_001098484.3). Nucleotides 76-150 correspond to a portion of exon 6 of the NRG1 gene.

(SEQ ID NO: 765)
ACTACCCCATCAACTCCAACTTCAAAGTGGGCTACAACACTCTCT
TTTCCTGTACCTGTGTGCCACCTGACCCAGCTACATCTACATCCA
CCACTGGGACAAGCCATCTTGTAAAATGTGCGGAGAAGGAGAAAA
CTTTCTGTGTGAATG

### Example 37: Patient-Derived SDC4-NRG1 Fusion Data

Tumorous material obtained from a patient diagnosed with a lung cancer on which molecular biotechnological analyses were performed revealed the presence of an in-frame fusion junction occurring between exon 4 of the gene SDC4 and exon 2 of the NRG1 gene comprised by the sequence according to SEQ ID NO: 824. The underlined sequence, nucleotides 1-75, corresponds to a portion of exon 4 of the gene coding for SDC4 (NM_002999.4). Nucleotides 76-150 correspond to a portion of exon 2 of the NRG1 gene.

(SEQ ID NO: 824)
ATGTGTCCAACAAGGTGTCAATGTCCAGCACTGTGCAGGGCAGCA
ACATCTTTGAGAGAACGGAGGTCCTGGCAGCCTTGCCTCCCCGAT
TGAAAGAGATGAAAAGCCAGGAATCGGCTGCAGGTTCCAAACTAG
TCCTTCGGTGTGAAA

### Example 38: Patient-Derived ZFAT-NRG1 Fusion Data

Tumorous material obtained from a patient diagnosed with a lung cancer on which molecular biotechnological analyses were performed revealed the presence of an in-frame fusion junction occurring between exon 12 of the ZFAT gene and exon 6 of the NRG1 gene comprised by the sequence according to SEQ ID NO: 828. The underlined sequence, nucleotides 1-75, corresponds to a portion of exon 12 of the gene coding for ZFAT (NM_020863.4). Nucleotides 76-150 correspond to a portion of exon 6 of the NRG1 gene.

(SEQ ID NO: 828)
ACAGGAAGCACCCTAATGAGGAGTATGCCAACGTGGGCACCGGGG
AGCTGGCAGCGGAGGTGCTCATCCAGCAAGCTACATCTACATCCA
CCACTGGGACAAGCCATCTTGTAAAATGTGCGGAGAAGGAGAAAA
CTTTCTGTGTGAATG

### Example 39: Patient-Derived DSCAML1-NRG1 Fusion Data

Tumorous material obtained from a patient diagnosed with a pancreatic cancer on which molecular biotechnological analyses were performed revealed the presence of an in-frame fusion junction occurring between exon 3 of the gene DSCAML1 and exon 2 of the NRG1 gene comprised by the sequence according to SEQ ID NO: 868. The underlined sequence, nucleotides 1-75, corresponds to a portion of exon 3 of the gene coding for DSCAML1 (NM_020693.4). Nucleotides 76-150 correspond to a portion of exon 2 of the NRG1 gene.

(SEQ ID NO: 868)
GCCTCATCCCCTCTTCAGTGCAGGAATATGTTAGCGTTGTATCTT
GGGAGAAAGACACAGTCTCCATCATCCCAGCCTTGCCTCCCCGAT
TGAAAGAGATGAAAAGCCAGGAATCGGCTGCAGGTTCCAAACTAG
TCCTTCGGTGTGAAA

### Example 40: PDX Animal Model

A patient derived xenograft (PDX) animal model containing engrafted aberrant cells comprising a polynucleotide fusion and expressing a polypeptide fusion encoded therefrom is prepared for treatment with Zenocutuzumab as ERB2 and/or Erb3 targeting agent for evaluation of the therapeutic activity thereof.

PDX models can be generated from samples containing a cancer or tumor, such as in this example of a female patient diagnosed with pancreatic adenocarcinoma and a CDH1-NRG1 polynucleotide fusion (see example 18) essentially as described in Puig et al., A Personalized Preclinical Model to Evaluate the Metastatic Potential of Patient-Derived Colon Cancer Initiating Cells, Clin Cancer Res; 19(24), 6787-6801 (2013), which is incorporated in its entirety into this application.

For all mouse studies female NOD.CB17/AlhnRj-Prkdescid/Rj mice (Janvier Labs) aged between 6-8 weeks are used.

The models can be injected subcutaneously, or orthotopically in the pancreas, of immunodeficient mice. Orthotopic models generate local and distant metastases in lymph nodes, liver, lungs or carcinomatoses, reproducing the advance disease in PDAC patients.

At week 6, all mice treated with vehicle or zenocutuzumab are sacrificed and analysed for efficacy of the treatment.

A statistically relevant number of NOD-SCID mice can be given orthotopic pancreatic injections of 1*10\hat{ }6 tumor cells derived from said PDX model. From 15 days post-injection, weekly CT imaging is used to monitor mice and detect primary tumors in the pancreas. Treatments are initiated after at least 80% of animals have a primary tumor growing in the pancreas.

Mice are excluded from the study based on not complying with the following qualitative standards, including dying after surgery, being without primary tumor, mice with too large tumors, insufficient body weight, and general signs of illness. Dosing and treatment regime is according to Example 12, however, using standard procedures and calculations, care is taken to appropriately translate conditions relevant for mice from the human situation.

1. A polynucleotide comprising
a PVALB nucleic acid sequence, or an allelic variant of the PVALB sequence, fused with an NRG1 nucleic acid sequence, or an allelic variant of the NRG1 sequence or
a ASPH nucleic acid sequence, or an allelic variant of the ASPH sequence, fused with an NRG1 nucleic acid sequence, or an allelic variant of the NRG1 sequence or
a DAAM1 nucleic acid sequence, or an allelic variant of the DAAM1 sequence, fused with an NRG1 nucleic acid sequence, or an allelic variant of the NRG1 sequence or
a ZFAT nucleic acid sequence, or an allelic variant of the ZFAT sequence, fused with an NRG1 nucleic acid sequence, or an allelic variant of the NRG1 sequence or
a DSCAML1 nucleic acid sequence, or an allelic variant of the DSCAML1 sequence, fused with an NRG1 nucleic acid sequence, or an allelic variant of the NRG1 sequence.

2. The polynucleotide according to claim 1, wherein
the PVALB nucleic acid sequence comprises or consists of any one of SEQ ID NOs: 439-444, or an allelic variant of any one of SEQ ID NOs: 439-444, and the NRG1 nucleic acid sequence comprises or consists of any one of SEQ ID NOs: 125-138;
the DAAM1 nucleic acid sequence comprises or consists of any one of SEQ ID NOs: 606-631, or an allelic variant of any one of SEQ ID NOs: 606-631, and the NRG1 nucleic acid sequence comprises or consists of any one of SEQ ID NOs: 125-138;
the ZFAT nucleic acid sequence comprises or consists of any one of SEQ ID NOs: 830-846, or an allelic variant of any one of SEQ ID NOs: 830-846, and the NRG1 nucleic acid sequence comprises or consists of any one of SEQ ID NOs: 125-138; or
the DSCAML1 nucleic acid sequence comprises or consists of any one of SEQ ID NOs: 870-903, or an allelic variant of any one of SEQ ID NOs: 870-903, and the NRG1 nucleic acid sequence comprises or consists of any one of SEQ ID NOs: 125-138.

3. The polynucleotide according to claim 1 or 2, wherein the PVALB, DAAM1, ZFAT or DSCAML1 nucleic acid sequence (or the allelic variant thereof), is 5′ to the NRG1 nucleic acid sequence (or the allelic variant thereof).

4. The polynucleotide according to any one of claims 1-3, wherein
the allelic variant of the PVALB nucleic acid sequences has at least 85% identity to any one of SEQ ID NOs: 439-444, preferably at least 90% identity, more preferably at least 95% sequence identity; and the allelic variant of the NRG1 nucleic acid sequences has at least 85% identity to any one of SEQ ID NOs: 125-138, preferably at least 90% identity, more preferably at least 95% sequence identity;
the allelic variant of the DAAM1 nucleic acid sequences has at least 85% identity to any one of SEQ ID NOs: 606-631, preferably at least 90% identity, more preferably at least 95% sequence identity; and the allelic variant of the NRG1 nucleic acid sequences has at least 85% identity to any one of SEQ ID NOs: 125-138, preferably at least 90% identity, more preferably at least 95% sequence identity;
the allelic variant of the ZFAT nucleic acid sequences has at least 85% identity to any one of SEQ ID NOs: 830-846, preferably at least 90% identity, more preferably at least 95% sequence identity; and the allelic variant of the NRG1 nucleic acid sequences has at least 85% identity to any one of SEQ ID NOs: 125-138, preferably at least 90% identity, more preferably at least 95% sequence identity; or
the allelic variant of the DSCAML1 nucleic acid sequences has at least 85% identity to any one of SEQ ID NOs: 870-903, preferably at least 90% identity, more preferably at least 95% sequence identity; and the allelic variant of the NRG1 nucleic acid sequences has at least 85% identity to any one of SEQ ID NOs: 125-138, preferably at least 90% identity, more preferably at least 95% sequence identity.

5. The polynucleotide according to any one of claims 1-4, wherein,
the fusion of the PVALB nucleic acid with the NRG1 nucleic acid comprises 2 to about 40 contiguous nucleic acids from SEQ ID NO: 437, preferably including the nucleic acids at positions 102 and 103;
the fusion of the DAAM1 nucleic acid with the NRG1 nucleic acid comprises 2 to about 40 contiguous nucleic acids from SEQ ID NO: 605, preferably including the nucleic acids at positions 75 and 76;
the fusion of the ZFAT nucleic acid with the NRG1 nucleic acid comprises 2 to about 40 contiguous nucleic acids from SEQ ID NO: 828, preferably including the nucleic acids at positions 75 and 76; or
the fusion of the DSCAML1 nucleic acid with the NRG1 nucleic acid comprises 2 to about 40 contiguous nucleic acids from SEQ ID NO: 868, preferably including the nucleic acids at positions 75 and 76.

6. The polynucleotide according to any one of claims 1-5, wherein the nucleic acid encoding the NRG1 protein sequence (or the allelic variant thereof), comprises or encodes an EGF-like domain of NRG1, preferably the EGF-like domain according to SEQ ID NO: 163.

7. A polynucleotide comprising:
a portion of exon 1 of VAPB, or of an allelic variant of exon 1, fused with a portion of exon 2 of NRG1, or an allelic variant of exon 2;
a portion of exon 7 of CADM1, or of an allelic variant of exon 7, and a portion of exon 6 of NRG1, or an allelic variant of exon 6;
a portion of exon 5 of CD44, or of an allelic variant of exon 5, and a portion of exon 2 of NRG1, or an allelic variant of exon 2;
a portion of exon 1 of transcript version 6 of SLC3A2, or of an allelic variant of exon 1, and a portion of exon 5 of NRG1, or an allelic variant of exon 5;
a portion of exon 2 of VTCN1, or of an allelic variant of exon 2, and a portion of exon 2 of NRG1, or an allelic variant of exon 2;
a portion of exon 11 of CDH1, or of an allelic variant of exon 11, and a portion of exon 2 of NRG1, or an allelic variant of exon 2;
a portion of exon 1 of CXADR, or of an allelic variant of exon 1, and a portion of exon 2 of NRG1, or an allelic variant of exon 2;
a portion of exon 2 of GTF2E2, or of an allelic variant of exon 1, and a portion of exon 2 of NRG1, or an allelic variant of exon 2;
a portion of exon 23 of CSMD1, or of an allelic variant of exon 23, and a portion of exon 6 of NRG1, or an allelic variant of exon 6;
a portion of exon 4 of PTN, or of an allelic variant of exon 4, and a portion of exon 2 of NRG1, or an allelic variant of exon 2;
a portion of exon 11 of ST14, or of an allelic variant of exon 11, and a portion of exon 6 of NRG1, or an allelic variant of exon 6;
a portion of exon 9 of THBS1, or of an allelic variant of exon 9, and a portion of exon 6 of NRG1, or an allelic variant of exon 6;
· a portion of exon 12 of AGRN, or of an allelic variant of exon 12, and a portion of exon 6 of NRG1, or an allelic variant of exon 6;
a portion of exon 4 of PVALB, or of an allelic variant of exon 4, and a portion of exon 6 of NRG1, or an allelic variant of exon 6,
a portion of exon 2 of transcript version 3 of SLC3A2, or of an allelic variant of exon 2, and a portion of exon 6 of NRG1, or an allelic variant of exon 6;
a portion of exon 14 of APP, or of an allelic variant of exon 14, and a portion of exon 6 of NRG1, or an allelic variant of exon 6;
a portion of exon 33 of WRN, or of an allelic variant of exon 33, and a portion of exon 6 of NRG1, or an allelic variant of exon 6;
a portion of exon 1 of DAAM1, or of an allelic variant of exon 1, and a portion of exon 1 of NRG1, or an allelic variant of exon 1;
a portion of exon 22 of ASPH, or of an allelic variant of exon 22, and a portion of exon 2 of NRG1, or an allelic variant of exon 2;
a portion of exon 6 of NOTCH2, or of an allelic variant of exon 6, and a portion of exon 6 of NRG1, or an allelic variant of exon 6;
a portion of exon 2 of CD74, or of an allelic variant of exon 2, and a portion of exon 2 of NRG1, or an allelic variant of exon 2;
a portion of exon 2 of SDC4, or of an allelic variant of exon 2, and a portion of exon 2 of NRG1, or an allelic variant of exon 2;
a portion of exon 5 of CD44, or of an allelic variant of exon 5, and a portion of exon 6 of NRG1, or an allelic variant of exon 6;
a portion of exon 14 of SLC4A4, or of an allelic variant of exon 14, and a portion of exon 6 of NRG1, or an allelic variant of exon 6;
a portion of exon 4 of SDC4, or of an allelic variant of exon 4, and a portion of exon 2 of NRG1, or an allelic variant of exon 2;
a portion of exon 12 of ZFAT, or of an allelic variant of exon 12, and a portion of exon 6 of NRG1, or an allelic variant of exon 6, or
a portion of exon 3 of DSCAML1, or of an allelic variant of exon 3, and a portion of exon 2 of NRG1, or an allelic variant of exon 2.

8. The polynucleotide according to claim 7, wherein exon 1 of VAPB is that of SEQ ID NO: 17; exon 7 of CADM1 is that of SEQ ID NO: 39; exon 5 of CD44 is that of SEQ ID NO: 65; exon 1 of SLC3A2 is that of SEQ ID NO: 103; exon 2 of VTCN1 is that of SEQ ID NO: 169; exon 11 of CDH1 is that of SEQ ID NO: 198; exon 1 of CXADR is that of SEQ ID NO: 219; exon 2 of GTF2E2 is that of SEQ ID NO: 236; exon 23 of CSMD1 is that of SEQ ID NO: 279; exon 4 of PTN is that of SEQ ID NO: 318; exon 11 of ST14 is that of SEQ ID NO: 342; exon 9 of THBS1 is that of SEQ ID NO: 386; exon 12 of AGRN is that of SEQ ID NO: 416; exon 4 of PVALB is that of SEQ ID NO: 442; exon 2 of SLC3A2 is that of SEQ ID NO: 457; exon 14 of APP is that of SEQ ID NO: 501; exon 33 of WRN is that of SEQ ID NO: 562; exon 1 of DAAM1 is that of SEQ ID NO: 606; exon 22 of ASPH is that of SEQ ID NO: 658; exon 6 of NOTCH2 is that of SEQ ID NO: 700; exon 2 of CD74 is that of SEQ ID NO: 720; exon 2 of SDC4 is that of SEQ ID NO: 746; exon 5 of CD44 is that of SEQ ID NO: 65; exon 14 of SLC4A4 is that of SEQ ID NO: 780; exon 4 of SDC4 is that of SEQ ID NO: 748; exon 12 of ZFAT is that of SEQ ID NO: 841; exon 3 of DSCAML1 is that of SEQ ID NO: 872 and exons 1, 2, 5 and 6 of NRG1 are those of SEQ ID NOs: 125, 126, 129 and 130, respectively.

9. The polynucleotide according to any one of claim 7 or 8, wherein:
the portion of exon 1 of VAPB, or the allelic variant thereof, is 5′ to the portion of exon 2 of NRG1, or the allelic variant thereof;
the portion of exon 7 of CADM1, or the allelic variant thereof, is 5′ to the portion of exon 6 of NRG1, or the allelic variant thereof;
the portion of exon 5 of CD44, or the allelic variant thereof, is 5′ to the portion of exon 2 of NRG1, or the allelic variant thereof;
the portion of exon 1 of SLC3A2, or the allelic variant thereof, is 5′ to the portion of exon 5 of NRG1, or the allelic variant thereof;
the portion of exon 2 of VTCN1, or the allelic variant thereof, is 5′ to the portion of exon 2 of NRG1, or the allelic variant thereof;
the portion of exon 11 of CDH1, or the allelic variant thereof, is 5′ to the portion of exon 2 of NRG1, or the allelic variant thereof;
the portion of exon 1 of CXADR, or the allelic variant thereof, is 5′ to the portion of exon 2 of NRG1, or the allelic variant thereof;
the portion of exon 2 of GTF2E2, or the allelic variant thereof, is 5′ to the portion of exon 2 of NRG1, or the allelic variant thereof;
the portion of exon 23 CSMD1, or the allelic variant thereof, is 5′ to the portion of exon 6 of NRG1, or the allelic variant thereof;
the portion of exon 4 of PTN, or the allelic variant thereof, is 5′ to the portion of exon 2 of NRG1, or the allelic variant thereof;
the portion of exon 11 of ST14, or the allelic variant thereof, is 5′ to the portion of exon 6 of NRG1, or the allelic variant thereof;
the portion of exon 9 of THBS1, or the allelic variant thereof, is 5′ to the portion of exon 6 of NRG1, or the allelic variant thereof;
the portion of exon 12 of AGRN, or the allelic variant thereof, is 5′ to the portion of exon 6 of NRG1, or the allelic variant thereof;
the portion of exon 4 of PVALB, or the allelic variant thereof, is 5′ to the portion of exon 6 of NRG1, or the allelic variant thereof;
the portion of exon 2 of SCL3A2, or the allelic variant thereof, is 5′ to the portion of exon 6 of NRG1, or the allelic variant thereof;
the portion of exon 14 of APP, or the allelic variant thereof, is 5′ to the portion of exon 6 of NRG1, or the allelic variant of exon 6;
the portion of exon 33 of WRN, or the allelic variant thereof, is 5′ to the portion of exon 6 of NRG1, or the allelic variant of exon 6;
the portion of exon 1 of DAAM1, or the allelic variant thereof, is 5′ to the portion of exon 1 of NRG1, or the allelic variant of exon 1;
the portion of exon 22 of ASPH, or the allelic variant thereof, is 5′ to the portion of exon 2 of NRG1, or the allelic variant of exon 2;
the portion of exon 6 of NOTCH2, or the allelic variant thereof, is 5′ to the portion of exon 6 of NRG1, or the allelic variant of exon 6;
the portion of exon 2 of CD74, or the allelic variant thereof, is 5′ to the portion of exon 2 of NRG1, or the allelic variant of exon 2;
the portion of exon 2 of SDC4, or the allelic variant thereof, is 5′ to the portion of exon 2 of NRG1, or the allelic variant of exon 2;
the portion of exon 5 of CD44, or the allelic variant thereof, is 5′ to the portion of exon 6 of NRG1, or the allelic variant of exon 6;
the portion of exon 14 of SLC4A4, or the allelic variant thereof, is 5′ to the portion of exon 6 of NRG1, or the allelic variant of exon 6;
a portion of exon 4 of SDC4, or the allelic variant thereof, is 5′ to the portion of exon 2 of NRG1, or the allelic variant of exon 2;
the portion of exon 12 of ZFAT, or the allelic variant thereof, is 5′ to the portion of exon 6 of NRG1, or the allelic variant of exon 6, and
the portion of exon 3 of DSCAML1, or the allelic variant thereof, is 5′ to the portion of exon 2 of NRG1, or the allelic variant of exon 2.

10. The polynucleotide according to any one of claims 7-9, wherein:
the allelic variant of exon 1 of VAPB has at least 85% identity to SEQ ID NO: 17, preferably at least 90%, 92%, 94%, 96% or even 98% identity thereto;
the allelic variant of exon 7 of CADM1 has at least 85% identity to SEQ ID NO: 39, preferably at least 90%, 92%, 94%, 96% or even 98% identity thereto;
the allelic variant of exon 5 of CD44 has at least 85% identity to SEQ ID NO: 65, preferably at least 90%, 92%, 94%, 96% or even 98% identity thereto;
the allelic variant of exon 1 of SLC3A2 has at least 85% identity to SEQ ID NO: 103, preferably at least 90%, 92%, 94%, 96% or even 98% identity thereto;
the allelic variant of exon 2 of VTCN1 has at least 85% identity to SEQ ID NO: 169, preferably at least 90%, 92%, 94%, 96% or even 98% identity thereto;
the allelic variant of exon 11 of CDH1 has at least 85% identity to SEQ ID NO: 198, preferably at least 90%, 92%, 94%, 96% or even 98% identity thereto;
the allelic variant of exon 2 of NRG1 has at least 85% identity to SEQ ID NO: 126, preferably at least 90%, 92%, 94%, 96% or even 98% identity thereto;
the allelic variant of exon 5 of NRG1 has at least 85% identity to SEQ ID NO: 129, preferably at least 90%, 92%, 94%, 96% or even 98% identity thereto;
the allelic variant of exon 6 of NRG1 has at least 85% identity to SEQ ID NO: 130, preferably at least 90%, 92%, 94%, 96% or even 98% identity thereto;
the allelic variant of exon 1 of CXADR has at least 85% identity to SEQ ID NO: 219, preferably at least 90%, 92%, 94%, 96% or even 98% identity thereto;
the allelic variant of exon 2 of GTF2E2 has at least 85% identity to SEQ ID NO: 236, preferably at least 90%, 92%, 94%, 96% or even 98% identity thereto;
the allelic variant of exon 23 of CSMD1 has at least 85% identity to SEQ ID NO: 279, preferably at least 90%, 92%, 94%, 96% or even 98% identity thereto;
the allelic variant of exon 4 of PTN has at least 85% identity to SEQ ID NO: 318, preferably at least 90%, 92%, 94%, 96% or even 98% identity thereto;
the allelic variant of exon 11 of ST14 has at least 85% identity to SEQ ID NO: 342, preferably at least 90%, 92%, 94%, 96% or even 98% identity thereto;
the allelic variant of exon 9 of THBS1 has at least 85% identity to SEQ ID NO: 386, preferably at least 90%, 92%, 94%, 96% or even 98% identity thereto;
the allelic variant of exon 12 of AGRN has at least 85% identity to SEQ ID NO: 416, preferably at least 90%, 92%, 94%, 96% or even 98% identity thereto;
the allelic variant of exon 4 of PVALB has at least 85% identity to SEQ ID NO: 442, preferably at least 90%, 92%, 94%, 96% or even 98% identity thereto;
the allelic variant of exon 2 of SCL3A2 has at least 85% identity to SEQ ID NO: 457, preferably at least 90%, 92%, 94%, 96% or even 98% identity thereto;
the allelic variant of exon 14 of APP has at least 85% identity to SEQ ID NO: 501, preferably at least 90%, 92%, 94%, 96% or even 98% identity thereto;
the allelic variant of exon 33 of WRN has at least 85% identity to SEQ ID NO: 562, preferably at least 90%, 92%, 94%, 96% or even 98% identity thereto;
the allelic variant of exon 1 of DAAM1 has at least 85% identity to SEQ ID NO: 606, preferably at least 90%, 92%, 94%, 96% or even 98% identity thereto;
the allelic variant of exon 1 of NRG1 has at least 85% identity to SEQ ID NO: 125, preferably at least 90%, 92%, 94%, 96% or even 98% identity thereto;
the allelic variant of exon 22 of ASPH has at least 85% identity to SEQ ID NO: 658, preferably at least 90%, 92%, 94%, 96% or even 98% identity thereto;
the allelic variant of exon 6 of NOTCH2 has at least 85% identity to SEQ ID NO: 700, preferably at least 90%, 92%, 94%, 96% or even 98% identity thereto;
the allelic variant of exon 2 of CD74 has at least 85% identity to SEQ ID NO: 720, preferably at least 90%, 92%, 94%, 96% or even 98% identity thereto;
the allelic variant of exon 2 of SDC4 has at least 85% identity to SEQ ID NO: 746, preferably at least 90%, 92%, 94%, 96% or even 98% identity thereto;
the allelic variant of exon 5 of CD44 has at least 85% identity to SEQ ID NO: 65, preferably at least 90%, 92%, 94%, 96% or even 98% identity thereto;
the allelic variant of exon 14 of SLC4A4 has at least 85% identity to SEQ ID NO: 780, preferably at least 90%, 92%, 94%, 96% or even 98% identity thereto;
the allelic variant of exon 4 of SDC4 has at least 85% identity to SEQ ID NO: 748, preferably at least 90%, 92%, 94%, 96% or even 98% identity thereto;
the allelic variant of exon 12 of ZFAT has at least 85% identity to SEQ ID NO: 841, preferably at least 90%, 92%, 94%, 96% or even 98% identity thereto; and
the allelic variant of exon 3 of DSCAML1 has at least 85% identity to SEQ ID NO: 872, preferably at least 90%, 92%, 94%, 96% or even 98% identity thereto.

11. The polynucleotide according to any one of claims 7-10, wherein:
the fusion of VAPB with NRG1 comprises 2 to about 40 contiguous nucleic acids from SEQ ID NO: 3, including the nucleic acids at position 43 and 44;
the fusion of CADM1 with NRG1 comprises 2 to about 40 contiguous nucleic acids from SEQ ID NO: 7, including the nucleic acids at position 53 and 54;
the fusion of CD44 with NRG1 comprises 2 to about 40 contiguous nucleic acids from SEQ ID NO: 11, including the nucleic acids at position 52 and 53;
the fusion of SLC3A2 with NRG1 comprises 2 to about 40 contiguous nucleic acids from SEQ ID NO: 15, including the nucleic acids at position 53 and 54;
the fusion of VTCN1 with NRG1 comprises 2 to about 40 contiguous nucleic acids from SEQ ID NO: 166, including the nucleic acids at position 65 and 66;
the fusion of CDH1 with NRG1 comprises 2 to about 40 contiguous nucleic acids from SEQ ID NO: 186, including the nucleic acids at position 119 and 120;
the fusion of CXADR with NRG1 comprises 2 to about 40 contiguous nucleic acids from SEQ ID NO: 217, including the nucleic acids at position 43 and 44;
the fusion of GTF2E2 with NRG1 comprises 2 to about 40 contiguous nucleic acids from SEQ ID NO: 233, including the nucleic acids at position 141 and 142;
the fusion of CSMD1 with NRG1 comprises 2 to about 40 contiguous nucleic acids from SEQ ID NO: 255, including the nucleic acids at position 88 and 89;
the fusion of PTN with NRG1 comprises 2 to about 40 contiguous nucleic acids from SEQ ID NO: 313, including the nucleic acids at position 102 and 103;
the fusion of ST14 with NRG1 comprises 2 to about 40 contiguous nucleic acids from SEQ ID NO: 330, including the nucleic acids at position 95 and 96;
the fusion of THBS1 with NRG1 comprises 2 to about 40 contiguous nucleic acids from SEQ ID NO: 376, including the nucleic acids at position 56 and 57;
the fusion of AGRN with NRG1 comprises 2 to about 40 contiguous nucleic acids from SEQ ID NO: 403, including the nucleic acids at position 106 and 107;
the fusion of PVALB with NRG1 comprises 2 to about 40 contiguous nucleic acids from SEQ ID NO: 437, including the nucleic acids at position 102 and 103;
the fusion of SLC3A2 with NRG1 comprises 2 to about 40 contiguous nucleic acids from SEQ ID NO: 454, including the nucleic acids at position 93 and 94;
the fusion of APP with NRG1 comprises 2 to about 40 contiguous nucleic acids from SEQ ID NO: 486, including the nucleic acids at position 54 and 55;
the fusion of WRN with NRG1 comprises 2 to about 40 contiguous nucleic acids from SEQ ID NO: 528, including the nucleic acids at position 96 and 97;
the fusion of DAAM1 with NRG1 comprises 2 to about 40 contiguous nucleic acids from SEQ ID NO: 605, including the nucleic acids at position 75 and 76;
the fusion of ASPH with NRG1 comprises 2 to about 40 contiguous nucleic acids from SEQ ID NO: 635, including the nucleic acids at position 75 and 76;
the fusion of NOTCH2 with NRG1 comprises 2 to about 40 contiguous nucleic acids from SEQ ID NO: 693, including the nucleic acids at position 75 and 76;
the fusion of CD74 with NRG1 comprises 2 to about 40 contiguous nucleic acids from SEQ ID NO: 717, including the nucleic acids at position 75 and 76;
the fusion of SDC4 with NRG1 comprises 2 to about 40 contiguous nucleic acids from SEQ ID NO: 743, including the nucleic acids at position 75 and 76;
the fusion of CD44 with NRG1 comprises 2 to about 40 contiguous nucleic acids from SEQ ID NO: 761, including the nucleic acids at position 75 and 76;
the fusion of SLC4A4 with NRG1 comprises 2 to about 40 contiguous nucleic acids from SEQ ID NO: 765, including the nucleic acids at position 75 and 76;
the fusion of SDC4 with NRG1 comprises 2 to about 40 contiguous nucleic acids from SEQ ID NO: 824, including the nucleic acids at position 75 and 76;
the fusion of ZFAT with NRG1 comprises 2 to about 40 contiguous nucleic acids from SEQ ID NO: 828, including the nucleic acids at position 75 and 76; and
the fusion of DSCAML1 with NRG1 comprises 2 to about 40 contiguous nucleic acids from SEQ ID NO: 868, including the nucleic acids at position 75 and 76.

12. The polynucleotide according to any one of claims 7-11, wherein:
the fusion of VAPB with NRG1 comprises SEQ ID NO: 3, or an allelic variant thereof;
the fusion of CADM1 with NRG1 comprises SEQ ID NO: 7, or an allelic variant thereof;
the fusion of CD44 with NRG1 comprises SEQ ID NO: 11, or an allelic variant thereof;
the fusion of SLC3A2 with NRG1 comprises SEQ ID NO: 15, or an allelic variant thereof;
the fusion of VTCN1 with NRG1 comprises SEQ ID NO: 166, or an allelic variant thereof;
the fusion of CDH1 with NRG1 comprises SEQ ID NO: 186, or an allelic variant thereof;
the fusion of CXADR with NRG1 comprises SEQ ID NO: 217, or an allelic variant thereof;
the fusion of GTF2E2 with NRG1 comprises SEQ ID NO: 233, or an allelic variant thereof;
the fusion of CSMD1 with NRG1 comprises SEQ ID NO: 255, or an allelic variant thereof;
the fusion of PTN with NRG1 comprises SEQ ID NO: 313, or an allelic variant thereof;
the fusion of ST14 with NRG1 comprises SEQ ID NO: 330, or an allelic variant thereof;
the fusion of THBS1 with NRG1 comprises SEQ ID NO: 376, or an allelic variant thereof;
the fusion of AGRN with NRG1 comprises SEQ ID NO: 403, or an allelic variant thereof;
the fusion of PVALB with NRG1 comprises SEQ ID NO: 437, or an allelic variant thereof;
the fusion of SLC3A2 with NRG1 comprises SEQ ID NO: 454, or an allelic variant thereof;
the fusion of APP with NRG1 comprises SEQ ID NO: 486, or an allelic variant thereof;
the fusion of WRN with NRG1 comprises SEQ ID NO: 528, or an allelic variant thereof;
the fusion of DAAM1 with NRG1 comprises SEQ ID NO: 605, or an allelic variant thereof;
the fusion of ASPH with NRG1 comprises SEQ ID NO: 635, or an allelic variant thereof;
the fusion of NOTCH2 with NRG1 comprises SEQ ID NO: 693, or an allelic variant thereof;
the fusion of CD74 with NRG1 comprises SEQ ID NO: 717, or an allelic variant thereof;
the fusion of SDC4 with NRG1 comprises SEQ ID NO: 743, or an allelic variant thereof;
the fusion of CD44 with NRG1 comprises SEQ ID NO: 761, or an allelic variant thereof;
the fusion of SLC4A4 with NRG1 comprises SEQ ID NO: 765, or an allelic variant thereof;
the fusion of SDC4 with NRG1 comprises SEQ ID NO: 824, or an allelic variant thereof;
the fusion of ZFAT with NRG1 comprises SEQ ID NO: 828, or an allelic variant thereof; and
the fusion of DSCAML1 with NRG1 comprises SEQ ID NO: 868, or an allelic variant thereof.

13. The polynucleotide according to any one of claims 7-12, wherein:
the portion of exon 1 of VAPB is or comprises SEQ ID NO: 1, or an allelic variant SEQ ID NO: 1;
the portion of exon 7 of CADM1 is or comprises SEQ ID NO: 5, or an allelic variant of SEQ ID NO: 5;
the portion of exon 5 of CD44 is or comprises SEQ ID NO: 9, or an allelic variant SEQ ID NO: 9;
the portion of exon 1 of SLC3A2 is or comprises SEQ ID NO: 13, or an allelic variant SEQ ID NO: 13;
the portion of exon 2 of VTCN1 is or comprises SEQ ID NO: 164, or an allelic variant SEQ ID NO: 164;
the portion of exon 11 of CDH1 is or comprises SEQ ID NO: 184, or an allelic variant SEQ ID NO: 184;
the portion of exon 1 of CXADR is or comprises SEQ ID NO: 215, or an allelic variant SEQ ID NO: 215;
the portion of exon 2 of GTF2E2 is or comprises SEQ ID NO: 231, or an allelic variant SEQ ID NO: 231;
the portion of exon 23 of CSMD1 is or comprises SEQ ID NO: 253, or an allelic variant SEQ ID NO: 253;
the portion of exon 4 of PTN is or comprises SEQ ID NO: 311, or an allelic variant SEQ ID NO: 311;
the portion of exon 11 of ST14 is or comprises SEQ ID NO:328, or an allelic variant SEQ ID NO: 328;
the portion of exon 9 of THBS1 is or comprises SEQ ID NO: 374, or an allelic variant SEQ ID NO: 374;
the portion of exon 12 of AGRN is or comprises SEQ ID NO: 401, or an allelic variant SEQ ID NO: 401;
the portion of exon 4 of PVALB is or comprises SEQ ID NO: 435, or an allelic variant SEQ ID NO: 435;
the portion of exon 2 of SLC3A2 is or comprises SEQ ID NO: 452, or an allelic variant SEQ ID NO: 452;
the portion of exon 2 of NRG1 is or comprises SEQ ID NO: 165, or an allelic variant SEQ ID NO: 165;
the portion of exon 5 of NRG1 is or comprises SEQ ID NO: 14, or an allelic variant SEQ ID NO: 14;
the portion of exon 6 of NRG1 is or comprises SEQ ID NO: 6, or an allelic variant thereof;
the portion of exon 14 of APP is or comprises SEQ ID NO: 484, or an allelic variant thereof;
the portion of exon 33 of WRN is or comprises SEQ ID NO: 526, or an allelic variant thereof;
the portion of exon 1 of DAAM1 is or comprises SEQ ID NO: 603, or an allelic variant thereof;
the portion of exon 22 of ASPH is or comprises SEQ ID NO: 633, or an allelic variant thereof;
the portion of exon 6 of NOTCH2 is or comprises SEQ ID NO: 691, or an allelic variant thereof;
the portion of exon 2 of CD74 is or comprises SEQ ID NO: 715, or an allelic variant thereof;
the portion of exon 2 of SDC4 is or comprises SEQ ID NO: 741, or an allelic variant thereof;
the portion of exon 5 of CD44 is or comprises SEQ ID NO: 759, or an allelic variant thereof;
the portion of exon 14 of SLC4A4 is or comprises SEQ ID NO: 763, or an allelic variant thereof;
a portion of exon 4 of SDC4 is or comprises SEQ ID NO: 822, or an allelic variant thereof;
the portion of exon 12 of ZFAT is or comprises SEQ ID NO: 826, or an allelic variant thereof,
the portion of exon 3 of DSCAML1 is or comprises SEQ ID NO: 866, or an allelic variant thereof; and
the portion of exon 1 of NRG1 is or comprises SEQ ID NO: 604, or an allelic variant thereof.

14. The polynucleotide according to any one of claims 7-12, wherein:
the fusion of VAPB with NRG1 comprises a fusion junction between exon 1 of VAPB and exon 2 of NRG1, preferably the junction between the nucleic acids at position 43 of VAPB and at position 44 of NRG1 of SEQ ID NO: 3;
the fusion of CADM1 with NRG1 comprises a fusion junction between exon 7 of CADM1 and exon 6 of NRG1, preferably the junction between the nucleic acids at position 53 of CADM1 and at position 54 of NRG1 of SEQ ID NO: 7;
the fusion of CD44 with NRG1 comprises a fusion junction between exon 5 of CD44 and exon 2 of NRG1, preferably the junction between the nucleic acids at position 52 of CD44 and at position 53 of NRG1 of SEQ ID NO: 11;
the fusion of SLC3A2 with NRG1 comprises a fusion junction between exon 1 of SLC3A2 and exon 5 of NRG1, preferably the junction between the nucleic acids at position 53 of SLC3A2 and at position 54 of NRG1 of SEQ ID NO: 15;
the fusion of VTCN1 with NRG1 comprises a fusion junction between exon 2 of VTCN1 and exon 2 of NRG1, preferably the junction between the nucleic acids at position 65 of VTCN1 and at position 66 of NRG1 of SEQ ID NO: 166;
the fusion of CDH1 with NRG1 comprises a fusion junction between exon 11 of CDH1 and exon 2 of NRG1, preferably the junction between the nucleic acids at position 119 of CDH1 and at position 120 of NRG1 of SEQ ID NO: 186
the fusion of CXADR with NRG1 comprises a fusion junction between exon 1 of CXADR and exon 2 of NRG1, preferably the junction between the nucleic acids at position 43 of CXADR and at position 44 of NRG1 of SEQ ID NO: 217;
the fusion of GTF2E2 with NRG1 comprises a fusion junction between exon 2 of GTF2E2 and exon 2 of NRG1, preferably the junction between the nucleic acids at position 141 of GTF2E2 and at position 142 of NRG1 of SEQ ID NO: 233;
the fusion of CSMD1 with NRG1 comprises a fusion junction between exon 23 of CSMD1 and exon 6 of NRG1, preferably the junction between the nucleic acids at position 88 of CSMD1 and at position 89 of NRG1 of SEQ ID NO: 255;
the fusion of PTN with NRG1 comprises a fusion junction between exon 4 of PTN and exon 2 of NRG1, preferably the junction between the nucleic acids at position 102 of PTN and at position 103 of NRG1 of SEQ ID NO: 313;
the fusion of ST14 with NRG1 comprises a fusion junction between exon 11 of ST14 and exon 6 of NRG1, preferably the junction between the nucleic acids at position 95 of ST14 and at position 96 of NRG1 of SEQ ID NO: 330;
the fusion of THBS1 with NRG1 comprises a fusion junction between exon 9 of THBS1 and exon 6 of NRG1, preferably the junction between the nucleic acids at position 56 of THBS1 and at position 57 of NRG1 of SEQ ID NO: 376;
the fusion of AGRN with NRG1 comprises a fusion junction between exon 12 of AGRN and exon 6 of NRG1, preferably the junction between the nucleic acids at position 106 of AGRN and at position 107 of NRG1 of SEQ ID NO: 403;
the fusion of PVALB with NRG1 comprises a fusion junction between exon 4 of PVALB and exon 6 of NRG1, preferably the junction between the nucleic acids at position 102 of PVALB and at position 103 of NRG1 of SEQ ID NO: 437;
the fusion of SLC3A2 with NRG1 comprises a fusion junction between exon 2 of SLC3A2 and exon 6 of NRG1, preferably the junction between the nucleic acids at position 93 of SLC3A2 and at position 94 of NRG1 of SEQ ID NO: 454;
the fusion of APP with NRG1 comprises a fusion junction between exon 14 of APP and exon 6 of NRG1, preferably the junction between the nucleic acids at position 54 of APP and at position 55 of NRG1 of SEQ ID NO: 486;
the fusion of WRN with NRG1 comprises a fusion junction between exon 33 of WRN and exon 6 of NRG1, preferably the junction between the nucleic acids at position 96 of WRN and at position 97 of NRG1 of SEQ ID NO: 528;
the fusion of DAAM1 with NRG1 comprises a fusion junction between exon 1 of DAAM1 and exon 1 of NRG1, preferably the junction between the nucleic acids at position 75 of DAAM1 and at position 76 of NRG1 of SEQ ID NO: 605;
the fusion of ASPH with NRG1 comprises a fusion junction between exon 22 of ASPH and exon 2 of NRG1, preferably the junction between the nucleic acids at position 75 of ASPH and at position 76 of NRG1 of SEQ ID NO: 635;
the fusion of NOTCH2 with NRG1 comprises a fusion junction between exon 6 of NOTCH2 and exon 6 of NRG1, preferably the junction between the nucleic acids at position 75 of NOTCH2 and at position 76 of NRG1 of SEQ ID NO: 693;
the fusion of CD74 with NRG1 comprises a fusion junction between exon 2 of CD74 and exon 2 of NRG1, preferably the junction between the nucleic acids at position 75 of CD74 and at position 76 of NRG1 of SEQ ID NO: 717;
the fusion of SDC4 with NRG1 comprises a fusion junction between exon 2 of SDC4 and exon 2 of NRG1, preferably the junction between the nucleic acids at position 75 of SDC4 and at position 76 of NRG1 of SEQ ID NO: 743;
the fusion of CD44 with NRG1 comprises a fusion junction between exon 5 of CD44 and exon 6 of NRG1, preferably the junction between the nucleic acids at position 75 of CD44 and at position 76 of NRG1 of SEQ ID NO: 761;
the fusion of SLC4A4 with NRG1 comprises a fusion junction between exon 14 of SLC4A4 and exon 6 of NRG1, preferably the junction between the nucleic acids at position 75 of SLC4A4 and at position 76 of NRG1 of SEQ ID NO: 765;
the fusion of SDC4 with NRG1 comprises a fusion junction between exon 4 of SDC4 and exon 2 of NRG1, preferably the junction between the nucleic acids at position 75 of SDC4 and at position 76 of NRG1 of SEQ ID NO: 824;
the fusion of ZFAT with NRG1 comprises a fusion junction between exon 12 of ZFAT and exon 6 of NRG1, preferably the junction between the nucleic acids at position 75 of ZFAT and at position 76 of NRG1 of SEQ ID NO: 828; and
the fusion of DSCAML1 with NRG1 comprises a fusion junction between exon 3 of DSCAML1 and exon 2 of NRG1, preferably the junction between the nucleic acids at position 75 of DSCAML1 and at position 76 of NRG1 of SEQ ID NO: 868.

15. The polynucleotide according to any one of the previous claims, wherein the polynucleotide is isolated or purified.

16. The polynucleotide according to any one of the previous claims, wherein any one of the fusions is an in-frame fusion.

17. The polynucleotide according to any one of the previous claims, wherein the polynucleotide is a mammalian polynucleotide, preferably a human polynucleotide.

18. A polypeptide fusion encoded by the polynucleotide according to any one of the previous claims.

19. A vector comprising the polynucleotide according to any one of claims 1-17.

20. A recombinant host cell comprising the polynucleotide of any of claims 1-17 or the vector of claim 19.

21. A method of making the polypeptide fusion of claim 18, comprising maintaining the host cell of claim 20 under conditions suitable for expression of the polynucleotide comprised by the host cell, whereby the polynucleotide is expressed and a polypeptide fusion is produced, followed by isolating or purifying the polypeptide fusion.

22. A method for making a recombinant host cell comprising introducing the vector of claim 19 into a host cell.

23. A detection assay comprising a nucleic acid probe, primer or primer pair for detection of the presence of a polynucleotide fusion according to any one of claims 1-17.

24. A nucleic acid probe, primer or primer pair for detection of a polynucleotide fusion according to any of the claims 1-17.

25. The nucleic acid probe, primer or primer pair of claim 24, having a length of 10-40 nucleotides.

26. The nucleic acid probe, primer or primer pair of claim 24 or 25, wherein the fusion as detected comprises:
the fusion of VAPB with NRG1 comprising or consisting of SEQ ID NO: 3, and preferably includes the nucleic acids at position 43 and 44;
the fusion of CADM1 with NRG1 comprising or consisting of SEQ ID NO: 7, and preferably includes the nucleic acids at position 53 and 54;
the fusion of CD44 with NRG1 comprising or consisting of SEQ ID NO: 11, and preferably includes the nucleic acids at position 52 and 53;
the fusion of SLC3A2 with NRG1 comprising or consisting of SEQ ID NO: 15, and preferably includes the nucleic acids at position 53 and 54;
the fusion of VTCN1 with NRG1 comprising or consisting of SEQ ID NO: 166, and preferably includes the nucleic acids at position 65 and 66;
the fusion of CDH1 with NRG1 comprising or consisting of SEQ ID NO: 186, and preferably includes the nucleic acids at position 119 and 120;
the fusion of CXADR with NRG1 comprising or consisting of SEQ ID NO: 217, and preferably includes the nucleic acids at position 43 and 44;
the fusion of GTF2E2 with NRG1 comprising or consisting of SEQ ID NO: 233, and preferably includes the nucleic acids at position 141 and 142;
the fusion of CSMD1 with NRG1 comprising or consisting of SEQ ID NO: 255, and preferably includes the nucleic acids at position 88 and 89;
the fusion of PTN with NRG1 comprising or consisting of SEQ ID NO: 313, and preferably includes the nucleic acids at position 102 and 103;
the fusion of ST14 with NRG1 comprising or consisting of SEQ ID NO: 330, and preferably includes the nucleic acids at position 95 and 96;
the fusion of THBS1 with NRG1 comprising or consisting of SEQ ID NO: 376, and preferably includes the nucleic acids at position 56 and 57;
the fusion of AGRN with NRG1 comprising or consisting of SEQ ID NO: 403, and preferably includes the nucleic acids at position 106 and 107;
the fusion of PVALB with NRG1 comprising or consisting of SEQ ID NO: 437, and preferably includes the nucleic acids at position 102 and 103;
the fusion of SLC3A2 with NRG1 comprising or consisting of SEQ ID NO: 454, and preferably includes the nucleic acids at position 93 and 94;
the fusion of APP with NRG1 comprising or consisting of SEQ ID NO: 486, and preferably includes the nucleic acids at position 54 and 55;
the fusion of WRN with NRG1 comprising or consisting of SEQ ID NO: 528, and preferably includes the nucleic acids at position 96 and 97;
the fusion of DAAM1 with NRG1 comprising or consisting of SEQ ID NO: 605, and preferably includes the nucleic acids at position 75 and 76;
the fusion of ASPH with NRG1 comprising or consisting of SEQ ID NO: 635, and preferably includes the nucleic acids at position 75 and 76;
the fusion of NOTCH2 with NRG1 comprising or consisting of SEQ ID NO: 693, and preferably includes the nucleic acids at position 75 and 76;
the fusion of CD74 with NRG1 comprising or consisting of SEQ ID NO: 717, and preferably includes the nucleic acids at position 75 and 76;
the fusion of SDC4 with NRG1 comprising or consisting of SEQ ID NO: 743, and preferably includes the nucleic acids at position 75 and 76;
the fusion of CD44 with NRG1 comprising or consisting of SEQ ID NO: 761, and preferably includes the nucleic acids at position 75 and 76;
the fusion of SLC4A4 with NRG1 comprising or consisting of SEQ ID NO: 765, and preferably includes the nucleic acids at position 75 and 76;
the fusion of SDC4 with NRG1 comprising or consisting of SEQ ID NO: 824, and preferably includes the nucleic acids at position 75 and 76;
the fusion of ZFAT with NRG1 comprising or consisting of SEQ ID NO: 828, and preferably includes the nucleic acids at position 75 and 76; and
the fusion of DSCAML1 with NRG1 comprising or consisting of SEQ ID NO: 868, and preferably includes the nucleic acids at position 75 and 76.

27. The nucleic acid probe, primer or primer pair of any one of claims 24-26, wherein:
the probe, primer or primer pair for detection of the fusion of VAPB with NRG1 specifically hybridizes to, or has 95% or more complementary sequence identity with, a sequence comprised by exon 1 from VAPB or a sequence located 5′ of exon 1 and/or to a sequence comprised by exon 2 from NRG1, or a sequence located 3′ of exon 2;
the probe, primer or primer pair for detection of the fusion of CADM1 with NRG1 specifically hybridizes to, or has 95% or more complementary sequence identity with, a sequence comprised by exon 7 from CADM1, or a sequence located 5′ of exon 7 and/or to a sequence comprised by exon 6 from NRG1, or a sequence located 3′ of exon 6;
the probe, primer or primer pair for detection of the fusion of CD44 with NRG1 specifically hybridizes to, or has 95% or more complementary sequence identity with, a sequence comprised by exon 5 from CD44, or a sequence located 5′ of exon 5 and/or to a sequence comprised by exon 2 from NRG1, or a sequence located 3′ of exon 2;
the probe, primer or primer pair for detection of the fusion of transcript version 6 of SLC3A2 with NRG1 specifically hybridizes to, or has 95% or more complementary sequence identity with, a sequence comprised by exon 1 from said SLC3A2 or a sequence located 5′ of exon 1 and/or to a sequence comprised by exon 5 from NRG1, or a sequence located 3′ of exon 5;
the probe, primer or primer pair for detection of the fusion of VTCN1 with NRG1 specifically hybridizes to, or has 95% or more complementary sequence identity with, a sequence comprised by exon 2 from VTCN1, or a sequence located 5′ of exon 2 and/or to a sequence comprised by exon 2 from NRG1, or a sequence located 3′ of exon 2;
the probe, primer or primer pair for detection of the fusion of CDH1 with NRG1 specifically hybridizes to, or has 95% or more complementary sequence identity with, a sequence comprised by exon 11 from CDH1, or a sequence located 5′ of exon 11 and/or to a sequence comprised by exon 2 from NRG1, or a sequence located 3′ of exon 2;
the probe, primer or primer pair for detection of the fusion of CXADR with NRG1 specifically hybridizes to, or has 95% or more complementary sequence identity with, a sequence comprised by exon 1 from CXADR, or a sequence located 5′ of exon 1 and/or to a sequence comprised by exon 2 from NRG1, or a sequence located 3′ of exon 2;
the probe, primer or primer pair for detection of the fusion of GTF2E2 with NRG1 specifically hybridizes to, or has 95% or more complementary sequence identity with, a sequence comprised by exon 2 from GTF2E2, or a sequence located 5′ of exon 2 and/or to a sequence comprised by exon 2 from NRG1, or a sequence located 3′ of exon 2;
the probe, primer or primer pair for detection of the fusion of CSMD1 with NRG1 specifically hybridizes to, or has 95% or more complementary sequence identity with, a sequence comprised by exon 23 from CSMD1, or a sequence located 5′ of exon 23 and/or to a sequence comprised by exon 6 from NRG1, or a sequence located 3′ of exon 6;
the probe, primer or primer pair for detection of the fusion of PTN with NRG1 specifically hybridizes to, or has 95% or more complementary sequence identity with, a sequence comprised by exon 4 from PTN, or a sequence located 5′ of exon 4 and/or to a sequence comprised by exon 2 from NRG1, or a sequence located 3′ of exon 2;
the probe, primer or primer pair for detection of the fusion of ST14 with NRG1 specifically hybridizes to, or has 95% or more complementary sequence identity with, a sequence comprised by exon 11 from ST14, or a sequence located 5′ of exon 11 and/or to a sequence comprised by exon 6 from NRG1, or a sequence located 3′ of exon 6;
the probe, primer or primer pair for detection of the fusion of THBS1 with NRG1 specifically hybridizes to, or has 95% or more complementary sequence identity with, a sequence comprised by exon 9 from THBS1, or a sequence located 5′ of exon 9 and/or to a sequence comprised by exon 6 from NRG1, or a sequence located 3′ of exon 6;
the probe, primer or primer pair for detection of the fusion of AGRN with NRG1 specifically hybridizes to, or has 95% or more complementary sequence identity with, a sequence comprised by exon 12 from AGRN, or a sequence located 5′ of exon 12 and/or to a sequence comprised by exon 6 from NRG1, or a sequence located 3′ of exon 6;
the probe, primer or primer pair for detection of the fusion of PVALB with NRG1 specifically hybridizes to, or has 95% or more complementary sequence identity with, a sequence comprised by exon 4 from PVALB, or a sequence located 5′ of exon 4 and/or to a sequence comprised by exon 6 from NRG1, or a sequence located 3′ of exon 6;
the probe, primer or primer pair for detection of the fusion of transcript version 3 of SLC3A2 with NRG1 specifically hybridizes to, or has 95% or more complementary sequence identity with, a sequence comprised by exon 2 from said SLC3A2, or a sequence located 5′ of exon 2 and/or to a sequence comprised by exon 6 from NRG1, or a sequence located 3′ of exon 6;
the probe, primer or primer pair for detection of the fusion of APP with NRG1 specifically hybridizes to, or has 95% or more complementary sequence identity with, a sequence comprised by exon 14 from APP, or a sequence located 5′ of exon 14 and/or to a sequence comprised by exon 6 from NRG1, or a sequence located 3′ of exon 6;
the probe, primer or primer pair for detection of the fusion of WRN with NRG1 specifically hybridizes to, or has 95% or more complementary sequence identity with, a sequence comprised by exon 33 from WRN, or a sequence located 5′ of exon 33 and/or to a sequence comprised by exon 6 from NRG1, or a sequence located 3′ of exon 6;
the probe, primer or primer pair for detection of the fusion of DAAM1 with NRG1 specifically hybridizes to, or has 95% or more complementary sequence identity with, a sequence comprised by exon 1 from DAAM1, or a sequence located 5′ of exon 1 and/or to a sequence comprised by exon 1 from NRG1, or a sequence located 3′ of exon 1;
the probe, primer or primer pair for detection of the fusion of ASPH with NRG1 specifically hybridizes to, or has 95% or more complementary sequence identity with, a sequence comprised by exon 22 from ASPH, or a sequence located 5′ of exon 22 and/or to a sequence comprised by exon 2 from NRG1, or a sequence located 3′ of exon 2;
the probe, primer or primer pair for detection of the fusion of NOTCH2 with NRG1 specifically hybridizes to, or has 95% or more complementary sequence identity with, a sequence comprised by exon 6 from NOTCH2, or a sequence located 5′ of exon 6 and/or to a sequence comprised by exon 6 from NRG1, or a sequence located 3′ of exon 6;
the probe, primer or primer pair for detection of the fusion of CD74 with NRG1 specifically hybridizes to, or has 95% or more complementary sequence identity with, a sequence comprised by exon 2 from CD74, or a sequence located 5′ of exon 2 and/or to a sequence comprised by exon 2 from NRG1, or a sequence located 3′ of exon 2;
the probe, primer or primer pair for detection of the fusion of SDC4 with NRG1 specifically hybridizes to, or has 95% or more complementary sequence identity with, a sequence comprised by exon 2 from SDC4, or a sequence located 5′ of exon 2 and/or to a sequence comprised by exon 2 from NRG1, or a sequence located 3′ of exon 2;
the probe, primer or primer pair for detection of the fusion of CD44 with NRG1 specifically hybridizes to, or has 95% or more complementary sequence identity with, a sequence comprised by exon 5 from CD44, or a sequence located 5′ of exon 5 and/or to a sequence comprised by exon 6 from NRG1, or a sequence located 3′ of exon 6;
the probe, primer or primer pair for detection of the fusion of SLC4A4 with NRG1 specifically hybridizes to, or has 95% or more complementary sequence identity with, a sequence comprised by exon 14 from SLC4A4, or a sequence located 5′ of exon 14 and/or to a sequence comprised by exon 6 from NRG1, or a sequence located 3′ of exon 6;
the probe, primer or primer pair for detection of the fusion of SDC4 with NRG1 specifically hybridizes to, or has 95% or more complementary sequence identity with, a sequence comprised by exon 4 from SDC4, or a sequence located 5′ of exon 4 and/or to a sequence comprised by exon 2 from NRG1, or a sequence located 3′ of exon 2;
the probe, primer or primer pair for detection of the fusion of ZFAT with NRG1 specifically hybridizes to, or has 95% or more complementary sequence identity with, a sequence comprised by exon 12 from ZFAT, or a sequence located 5′ of exon 12 and/or to a sequence comprised by exon 6 from NRG1, or a sequence located 3′ of exon 6; or
the probe, primer or primer pair for detection of the fusion of DSCAML1 with NRG1 specifically hybridizes to, or has 95% or more complementary sequence identity with, a sequence comprised by exon 3 from DSCAML1, or a sequence located 5′ of exon 3 and/or to a sequence comprised by exon 2 from NRG1, or a sequence located 3′ of exon 2.

28. The nucleic acid probe, primer or primer pair of claim 27, wherein:
exon 1 from VAPB comprises or consists of SEQ ID NO: 17 or an allelic variant thereof;
exon 7 from CADM1 comprises or consists of SEQ ID NO: 39 or an allelic variant thereof;
exon 5 from CD44 comprises or consists of SEQ ID NO: 65 or an allelic variant thereof;
exon 1 from SLC3A2 comprises or consists of SEQ ID NO: 103 or an allelic variant thereof;
exon 2 from VTCN1 comprises or consists of SEQ ID NO: 169 or an allelic variant thereof;
exon 11 from CDH1 comprises or consists of SEQ ID NO: 198 or an allelic variant thereof;
exon 1 from CXADR comprises or consists of SEQ ID NO: 219 or an allelic variant thereof;
exon 2 from GTF2E2 comprises or consists of SEQ ID NO: 236 or an allelic variant thereof;
exon 23 from CSMD1 comprises or consists of SEQ ID NO: 279 or an allelic variant thereof;
exon 4 from PTN comprises or consists of SEQ ID NO: 318 or an allelic variant thereof;
exon 11 from ST14 comprises or consists of SEQ ID NO: 342 or an allelic variant thereof;
exon 9 from THBS1 comprises or consists of SEQ ID NO: 386 or an allelic variant thereof;
exon 12 from AGRN comprises or consists of SEQ ID NO: 416 or an allelic variant thereof;
exon 4 from PVALB comprises or consists of SEQ ID NO: 442 or an allelic variant thereof;
exon 2 from SLC3A2 comprises or consists of SEQ ID NO: 457 or an allelic variant thereof;
exon 14 from APP comprises or consists of SEQ ID NO: 501 or an allelic variant thereof;
exon 33 from WRN comprises or consists of SEQ ID NO: 562 or an allelic variant thereof;
exon 1 from DAAM1 comprises or consists of SEQ ID NO: 606 or an allelic variant thereof;
exon 22 from ASPH comprises or consists of SEQ ID NO: 658 or an allelic variant thereof;
exon 6 from NOTCH2 comprises or consists of SEQ ID NO: 700 or an allelic variant thereof;
exon 2 from CD74 comprises or consists of SEQ ID NO: 720 or an allelic variant thereof;
exon 2 from SDC4 comprises or consists of SEQ ID NO: 746 or an allelic variant thereof;
exon 5 from CD44 comprises or consists of SEQ ID NO: 65 or an allelic variant thereof;
exon 14 from SLC4A4 comprises or consists of SEQ ID NO: 780 or an allelic variant thereof;
exon 4 from SDC4 comprises or consists of SEQ ID NO: 748 or an allelic variant thereof;
exon 12 from ZFAT comprises or consists of SEQ ID NO: 841 or an allelic variant thereof;
exon 3 from DSCAML1 comprises or consists of SEQ ID NO: 872 or an allelic variant thereof; and
exons 1, 2, 5 and 6 from NRG1 comprise or consist of SEQ ID NO: 125, 126, 129 and 130, respectively, or an allelic variant thereof.

29. The nucleic acid probe, primer or primer pair of claim 27, wherein:
the probe, primer or primer pair for detection of the fusion of VAPB with NRG1 specifically hybridizes to, or has 95% or more complementary sequence identity with, a sequence comprised by SEQ ID NO: 17, or to an allelic variant thereof, and/or to a sequence comprised by SEQ ID NO: 153;
the probe, primer or primer pair for detection of the fusion of CADM1 with NRG1 specifically hybridizes to, or has 95% or more complementary sequence identity with, a sequence comprised by SEQ ID NO: 57 and/or to a sequence comprised by SEQ ID NO: 155;
the probe, primer or primer pair for detection of the fusion of CD44 with NRG1 specifically hybridizes to, or has 95% or more complementary sequence identity with, a sequence comprised by SEQ ID NO: 99 and/or to a sequence comprised by SEQ ID NO: 153;
the probe, primer or primer pair for detection of the fusion of SLC3A2 with NRG1 specifically hybridizes to, or has 95% or more complementary sequence identity with, a sequence comprised by SEQ ID NO: 103 and/or to a sequence comprised by SEQ ID NO: 157;
the probe, primer or primer pair for detection of the fusion of VTCN1 with NRG1 specifically hybridizes to, or has 95% or more complementary sequence identity with, a sequence comprised by SEQ ID NO: 181 and/or to a sequence comprised by SEQ ID NO: 153;
the probe, primer or primer pair for detection of the fusion of CDH1 with NRG1 specifically hybridizes to, or has 95% or more complementary sequence identity with, a sequence comprised by SEQ ID NO: 213 and/or to a sequence comprised by SEQ ID NO: 153;
the probe, primer or primer pair for detection of the fusion of CXADR with NRG1 specifically hybridizes to, or has 95% or more complementary sequence identity with, a sequence comprised by SEQ ID NO: 219 and/or to a sequence comprised by SEQ ID NO: 153;
the probe, primer or primer pair for detection of the fusion of GTF2E2 with NRG1 specifically hybridizes to, or has 95% or more complementary sequence identity with, a sequence comprised by SEQ ID NO: 252 and/or to a sequence comprised by SEQ ID NO: 153;
the probe, primer or primer pair for detection of the fusion of CSMD1 with NRG1 specifically hybridizes to, or has 95% or more complementary sequence identity with, a sequence comprised by SEQ ID NO: 309 and/or to a sequence comprised by SEQ ID NO: 155;
the probe, primer or primer pair for detection of the fusion of PTN with NRG1 specifically hybridizes to, or has 95% or more complementary sequence identity with, a sequence comprised by SEQ ID NO: 326 and/or to a sequence comprised by SEQ ID NO: 153;
the probe, primer or primer pair for detection of the fusion of ST14 with NRG1 specifically hybridizes to, or has 95% or more complementary sequence identity with, a sequence comprised by SEQ ID NO: 372 and/or to a sequence comprised by SEQ ID NO: 155;
the probe, primer or primer pair for detection of the fusion of THBS1 with NRG1 specifically hybridizes to, or has 95% or more complementary sequence identity with, a sequence comprised by SEQ ID NO: 399 and/or to a sequence comprised by SEQ ID NO: 155;
the probe, primer or primer pair for detection of the fusion of AGRN with NRG1 specifically hybridizes to, or has 95% or more complementary sequence identity with, a sequence comprised by SEQ ID NO: 433 and/or to a sequence comprised by SEQ ID NO: 155;
the probe, primer or primer pair for detection of the fusion of PVALB with NRG1 specifically hybridizes to, or has 95% or more complementary sequence identity with, a sequence comprised by SEQ ID NO: 450 and/or to a sequence comprised by SEQ ID NO: 155;
the probe, primer or primer pair for detection of the fusion of SLC3A2 with NRG1 specifically hybridizes to, or has 95% or more complementary sequence identity with, a sequence comprised by SEQ ID NO: 482 and/or to a sequence comprised by SEQ ID NO: 155;
the probe, primer or primer pair for detection of the fusion of APP with NRG1 specifically hybridizes to, or has 95% or more complementary sequence identity with, a sequence comprised by SEQ ID NO: 524 and/or to a sequence comprised by SEQ ID NO: 155;
the probe, primer or primer pair for detection of the fusion of WRN with NRG1 specifically hybridizes to, or has 95% or more complementary sequence identity with, a sequence comprised by SEQ ID NO: 601 and/or to a sequence comprised by SEQ ID NO: 155;
the probe, primer or primer pair for detection of the fusion of DAAM1 with NRG1 specifically hybridizes to, or has 95% or more complementary sequence identity with, a sequence comprised by SEQ ID NO: 606 and/or to a sequence comprised by SEQ ID NO: 138;
the probe, primer or primer pair for detection of the fusion of ASPH with NRG1 specifically hybridizes to, or has 95% or more complementary sequence identity with, a sequence comprised by SEQ ID NO: 689 and/or to a sequence comprised by SEQ ID NO: 153;
the probe, primer or primer pair for detection of the fusion of NOTCH2 with NRG1 specifically hybridizes to, or has 95% or more complementary sequence identity with, a sequence comprised by SEQ ID NO: 713 and/or to a sequence comprised by SEQ ID NO: 155;
the probe, primer or primer pair for detection of the fusion of CD74 with NRG1 specifically hybridizes to, or has 95% or more complementary sequence identity with, a sequence comprised by SEQ ID NO: 739 and/or to a sequence comprised by SEQ ID NO: 153;
the probe, primer or primer pair for detection of the fusion of SDC4 with NRG1 specifically hybridizes to, or has 95% or more complementary sequence identity with, a sequence comprised by SEQ ID NO: 757 and/or to a sequence comprised by SEQ ID NO: 153;
the probe, primer or primer pair for detection of the fusion of CD44 with NRG1 specifically hybridizes to, or has 95% or more complementary sequence identity with, a sequence comprised by SEQ ID NO: 99 and/or to a sequence comprised by SEQ ID NO: 155;
the probe, primer or primer pair for detection of the fusion of SLC4A4 with NRG1 specifically hybridizes to, or has 95% or more complementary sequence identity with, a sequence comprised by SEQ ID NO: 820 and/or to a sequence comprised by SEQ ID NO: 155;
the probe, primer or primer pair for detection of the fusion of SDC4 with NRG1 specifically hybridizes to, or has 95% or more complementary sequence identity with, a sequence comprised by SEQ ID NO: 940 and/or to a sequence comprised by SEQ ID NO: 153;
the probe, primer or primer pair for detection of the fusion of ZFAT with NRG1 specifically hybridizes to, or has 95% or more complementary sequence identity with, a sequence comprised by SEQ ID NO: 864 and/or to a sequence comprised by SEQ ID NO: 155; and
the probe, primer or primer pair for detection of the fusion of DSCAML1 with NRG1 specifically hybridizes to, or has 95% or more complementary sequence identity with, a sequence comprised by SEQ ID NO: 938 and/or to a sequence comprised by SEQ ID NO: 153.

30. A first and a second nucleic acid probe for use in an in-situ hybridization assay to detect a polynucleotide fusion of any one of claims 1-17,
wherein the first probe specifically hybridizes to a VAPB sequence which is located 5′ from the nucleic acid of position 43 of SEQ ID NO: 3, and the second probe specifically hybridizes to a NRG1 sequence which is located 3′ from the nucleic acid of position 44 of SEQ ID NO: 3;
wherein the first probe specifically hybridizes to a CADM1 sequence which is located 5′ from the nucleic acid of position 53 of SEQ ID NO: 7, and the second probe specifically hybridizes to a NRG1 sequence which is located 3′ from the nucleic acid of position 54 of SEQ ID NO: 7;
wherein the first probe specifically hybridizes to a CD44 sequence which is located 5′ from the nucleic acid of position 52 of SEQ ID NO: 11, and the second probe specifically hybridizes to a NRG1 sequence which is located 3′ from the nucleic acid of position 53 of SEQ ID NO: 11;
wherein the first probe specifically hybridizes to a SLC3A2 sequence which is located 5′ from the nucleic acid of position 53 of SEQ ID NO: 15, and the second probe specifically hybridizes to a NRG1 sequence which is located 3′ from the nucleic acid of position 54 of SEQ ID NO: 15;
wherein the first probe specifically hybridizes to a VTCN1 sequence which is located 5′ from the nucleic acid of position 65 of SEQ ID NO: 166, and the second probe specifically hybridizes to a NRG1 sequence which is located 3′ from the nucleic acid of position 66 of SEQ ID NO: 166;
wherein the first probe specifically hybridizes to a CDH1 sequence which is located 5′ from the nucleic acid of position 119 of SEQ ID NO: 186, and the second probe specifically hybridizes to a NRG1 sequence which is located 3′ from the nucleic acid of position 120 of SEQ ID NO: 186;
wherein the first probe specifically hybridizes to a CXADR sequence which is located 5′ from the nucleic acid of position 43 of SEQ ID NO: 217, and the second probe specifically hybridizes to a NRG1 sequence which is located 3′ from the nucleic acid of position 44 of SEQ ID NO: 217;
wherein the first probe specifically hybridizes to a GTF2E2 sequence which is located 5′ from the nucleic acid of position 141 of SEQ ID NO: 233, and the second probe specifically hybridizes to a NRG1 sequence which is located 3′ from the nucleic acid of position 142 of SEQ ID NO: 233;
wherein the first probe specifically hybridizes to a CSMD1 sequence which is located 5′ from the nucleic acid of position 88 of SEQ ID NO: 255, and the second probe specifically hybridizes to a NRG1 sequence which is located 3′ from the nucleic acid of position 89 of SEQ ID NO: 255;
wherein the first probe specifically hybridizes to a PTN sequence which is located 5′ from the nucleic acid of position 102 of SEQ ID NO: 313, and the second probe specifically hybridizes to a NRG1 sequence which is located 3′ from the nucleic acid of position 103 of SEQ ID NO: 313;
wherein the first probe specifically hybridizes to a ST14 sequence which is located 5′ from the nucleic acid of position 95 of SEQ ID NO: 330, and the second probe specifically hybridizes to a NRG1 sequence which is located 3′ from the nucleic acid of position 96 of SEQ ID NO: 330;
wherein the first probe specifically hybridizes to a THBS1 sequence which is located 5′ from the nucleic acid of position 56 of SEQ ID NO: 376, and the second probe specifically hybridizes to a NRG1 sequence which is located 3′ from the nucleic acid of position 57 of SEQ ID NO: 376;
wherein the first probe specifically hybridizes to a AGRN sequence which is located 5′ from the nucleic acid of position 106 of SEQ ID NO: 403, and the second probe specifically hybridizes to a NRG1 sequence which is located 3′ from the nucleic acid of position 107 of SEQ ID NO: 403;
wherein the first probe specifically hybridizes to a PVALB sequence which is located 5′ from the nucleic acid of position 102 of SEQ ID NO: 437, and the second probe specifically hybridizes to a NRG1 sequence which is located 3′ from the nucleic acid of position 103 of SEQ ID NO: 437;
wherein the first probe specifically hybridizes to a SLC3A2 sequence which is located 5′ from the nucleic acid of position 93 of SEQ ID NO: 454, and the second probe specifically hybridizes to a NRG1 sequence which is located 3′ from the nucleic acid of position 94 of SEQ ID NO: 454;
wherein the first probe specifically hybridizes to a APP sequence which is located 5′ from the nucleic acid of position 54 of SEQ ID NO: 486, and the second probe specifically hybridizes to a NRG1 sequence which is located 3′ from the nucleic acid of position 55 of SEQ ID NO: 486;
wherein the first probe specifically hybridizes to a WRN sequence which is located 5′ from the nucleic acid of position 96 of SEQ ID NO: 528, and the second probe specifically hybridizes to a NRG1 sequence which is located 3′ from the nucleic acid of position 97 of SEQ ID NO: 528;
wherein the first probe specifically hybridizes to a DAAM1 sequence which is located 5′ from the nucleic acid of position 75 of SEQ ID NO: 605, and the second probe specifically hybridizes to a NRG1 sequence which is located 3′ from the nucleic acid of position 76 of SEQ ID NO: 605;
wherein the first probe specifically hybridizes to a ASPH sequence which is located 5′ from the nucleic acid of position 75 of SEQ ID NO: 635, and the second probe specifically hybridizes to a NRG1 sequence which is located 3′ from the nucleic acid of position 76 of SEQ ID NO: 635;
wherein the first probe specifically hybridizes to a NOTCH2 sequence which is located 5′ from the nucleic acid of position 75 of SEQ ID NO: 693, and the second probe specifically hybridizes to a NRG1 sequence which is located 3′ from the nucleic acid of position 76 of SEQ ID NO: 693;
wherein the first probe specifically hybridizes to a CD74 sequence which is located 5′ from the nucleic acid of position 75 of SEQ ID NO: 717, and the second probe specifically hybridizes to a NRG1 sequence which is located 3′ from the nucleic acid of position 76 of SEQ ID NO: 717;
wherein the first probe specifically hybridizes to a SDC4 sequence which is located 5′ from the nucleic acid of position 75 of SEQ ID NO: 743, and the second probe specifically hybridizes to a NRG1 sequence which is located 3′ from the nucleic acid of position 76 of SEQ ID NO: 743;
wherein the first probe specifically hybridizes to a CD44 sequence which is located 5′ from the nucleic acid of position 75 of SEQ ID NO: 761, and the second probe specifically hybridizes to a NRG1 sequence which is located 3′ from the nucleic acid of position 76 of SEQ ID NO: 761;
wherein the first probe specifically hybridizes to a SLC4A4 sequence which is located 5′ from the nucleic acid of position 75 of SEQ ID NO: 765, and the second probe specifically hybridizes to a NRG1 sequence which is located 3′ from the nucleic acid of position 76 of SEQ ID NO: 765;
wherein the first probe specifically hybridizes to a SDC4 sequence which is located 5′ from the nucleic acid of position 75 of SEQ ID NO: 824, and the second probe specifically hybridizes to a NRG1 sequence which is located 3′ from the nucleic acid of position 76 of SEQ ID NO: 824;
wherein the first probe specifically hybridizes to a ZFAT sequence which is located 5′ from the nucleic acid of position 75 of SEQ ID NO: 828, and the second probe specifically hybridizes to a NRG1 sequence which is located 3′ from the nucleic acid of position 76 of SEQ ID NO: 828; or
wherein the first probe specifically hybridizes to a DSCAML1 sequence which is located 5′ from the nucleic acid of position 75 of SEQ ID NO: 868, and the second probe specifically hybridizes to a NRG1 sequence which is located 3′ from the nucleic acid of position 76 of SEQ ID NO: 868.

31. A first antibody or a set of a first and a second antibody pair for detection of a polypeptide encoded by a polynucleotide fusion according to any of the claims 1-17.

32. A detection assay comprising a first antibody or a set of a first and second antibodies for detection of the presence of a polypeptide encoded by a polynucleotide fusion according to any one of claims 1-17, wherein the first antibody or set of a first and second antibodies preferably is the first antibody or a set of a first and second antibodies of claim 31.

33. The first antibody or set of first and second antibodies of claim 31, or the detection assay according to claim 32, wherein the first antibody binds a polypeptide fusion selected from VAPB-NRG1, CADM1-NRG1, CD44-NRG1, SLC3A2-NRG1, VTCN1-NRG1, CDH1-NRG1, CXADR-NRG1, GTF2E2-NRG1, CSMD1-NRG1, PTN-NRG1, ST14-NRG1, THBS1-NRG1, AGRN-NRG1, PVALB-NRG1, APP-NRG1, WRN-NRG1, ASPH-NRG1, NOTCH2-NRG1, CD74-NRG1, SDC4-NRG1, SLC4A4-NRG1, ZFAT-NRG1 or DSCAML1-NRG1 and the set of first and second antibodies binds VAPB and NRG1 or CADM1 and NRG1, or CD44 and NRG1, SLC3A2 and NRG1, VTCN1 and NRG1, CDH1 and NRG1, CXADR and NRG1, GTF2E2 and NRG1, CSMD1 and NRG1, PTN and NRG1, ST14 and NRG1, THBS1 and NRG1, AGRN and NRG1, PVALB and NRG1, APP and NRG1, WRN and NRG1, ASPH and NRG1, NOTCH2 and NRG1, CD74 and NRG1, SDC4 and NRG1, SLC4A4 and NRG1, ZFAT and NRG1, or DSCAML1 and NRG1 respectively.

34. A method for identifying a polynucleotide fusion, or a polypeptide encoded therefrom, according to any one of the claims 1-17 in a sample, said method comprising testing a sample obtained from a subject to detect the presence of the fusion in the sample.

35. A method for detecting the presence of a polynucleotide fusion, or a polypeptide encoded therefrom, according to any one of the claims 1-17 in a sample, said method comprising testing a sample obtained from a subject to detect the presence of the fusion in the sample.

36. A method for establishing whether an aberrant cell from a subject comprises a polynucleotide fusion, or a polypeptide encoded therefrom, according to any one of claims 1-17, said method comprising testing the polynucleotide or polypeptide contents of the aberrant cell obtained from the subject for the presence of the fusion in the sample.

37. A method for identifying a subject as carrying a polynucleotide fusion, or a polypeptide encoded therefrom, according to any one of claims 1-17, said method comprising testing a sample obtained from a subject to detect the presence of the fusion in the sample.

38. The method of any one of claims 34-37, wherein the testing comprises detecting the fusion by utilizing a binding agent that specifically binds the polynucleotide, such as the nucleic acid probe, primer or primer pair of claim 24-29, or a polypeptide encoded therefrom, or utilizing a binding agent that binds a polynucleotide that comprises the polynucleotide fusion.

39. The method of any one of claims 34-38, wherein the testing comprises amplifying or detecting a sequence that discriminates between the presence and absence of the polynucleotide fusion, or polypeptide encoded therefrom.

40. The method of any one of claims 34-39, wherein the polynucleotide fusion is obtained from an aberrant cell expressing a polynucleotide fusion that comprises an EGF-like domain of NRG1.

41. The method of any one of claims 34-40, wherein the method comprises a step of obtaining the sample from a subject, followed by a step of isolating the polynucleotide or polypeptide encoded therefrom, from the sample.

42. The method of any one of claims 34-41, wherein the method comprises a step of purifying or isolating the polynucleotide or polypeptide from the sample.

43. The method of any one of claims 34-42, wherein the binding agent is or comprises a primer, a primer pair, a probe or an antibody.

44. The method of any one of claims 34-43, wherein the testing is an ex vivo method, preferably an in vitro method.

45. The method of any one of claims 34-44, wherein the binding agent comprises or is associated with a detectable label.

46. The method of any one of claims 34-45, wherein the sample is a liquid biopsy sample or a solid sample, such as a formalin fixed paraffin embedded tissue (FFPE) sample.

47. The method of any one of claims 34-46, wherein the sample comprises blood, serum, plasma, pleural liquid, urine, semen, amniotic fluid or peritoneal fluid.

48. The method of any one of claims 34-47, wherein the sample comprises an aberrant cell, such as a tumor cell or a cancer cell, or the polynucleotide or polypeptide contents thereof.

49. A method of treating a subject having an ErbB-2 and/or ErbB-3 positive cancer or tumor comprising a polynucleotide fusion and/or expressing a fusion polypeptide encoded therefrom, said method comprising administering to the subject an effective amount of an ErbB-2 and/or ErbB-3 targeting agent, wherein the fusion is a fusion according to any one of claims 1-17.

50. A method for inhibiting the progression in a subject of an ErbB-2 and ErbB-3 positive cancer or tumor comprising a polynucleotide fusion and/or expressing a fusion polypeptide encoded therefrom, said method comprising administering to the subject an effective amount of an ErbB-2 and/or ErbB-3 targeting agent, wherein said fusion is a fusion according to any one of claims 1-17.

51. An ErbB-2 and/or ErbB-3 targeting agent for use in the treatment of a subject that has an ErbB-2 and ErbB-3 positive cancer or tumor comprising a polynucleotide fusion and/or expressing a fusion polypeptide encoded therefrom, said treatment comprising administering an effective amount of the ErbB-2 and/or ErbB-3 targeting agent to the subject, wherein said fusion is a fusion according to any one of claims 1-17.

52. A method for diagnosing a subject for an aberrant cell that comprises a polynucleotide fusion according to any one of claims 1-17, or polypeptide encoded therefrom, the method comprising testing a sample obtained from a subject to detect the presence of the fusion in the sample.

53. The method of claim 52, wherein the testing comprises detecting the fusion by utilizing a binding agent that specifically binds the polynucleotide, such as the nucleic acid probe, primer or primer pair of any one of claims 24-29, or a polypeptide encoded therefrom, or utilizing a binding agent that binds a polynucleotide that comprises the polynucleotide fusion.

54. A method for assessing whether a subject suffers from a cancer or tumor or is prone to suffering from a cancer or tumor, the method comprising testing a sample obtained from a subject to detect the presence of a polynucleotide fusion according to any one of claims 1-17, or polypeptide encoded therefrom, in the sample, and assessing that said subject suffers from said cancer or tumor or is prone to suffering from said cancer or tumor, by identifying the presence of said polynucleotide or polypeptide fusion.

55. The method or use according to any one of claims 49-51, wherein the ErbB-2 and/or ErbB-3 targeting agent is selected from the group consisting of a multispecific antibody comprising a first antigen-binding site that binds an extracellular part of ErbB-2 and a second antigen-binding site that binds an extracellular part of ErbB-3, a tyrosine kinase inhibitor of ErbB-2, a monospecific bivalent antibody comprising an antigen-binding site that bind an extracellular part of ErbB-2, a monospecific bivalent antibody comprising an antigen-binding site that bind an extracellular part of ErbB-3, or any combination thereof.

56. The method or use according to any one of claim 49-51, or 55, wherein the ErbB-2 and/or ErbB-3 targeting agent is zenocutuzumab.

57. The method or use according to any one of claims 35-56, wherein the aberrant cell, cancer cell, tumor cell or sample comprises the polynucleotide fusion of claims 1-17, or a polypeptide encoded thereby, and wherein the polynucleotide fusion comprised by the cell or sample further comprises an in frame fusion of a coding sequence that codes for EGF-like domain of NRG1.

58. The method or use according to any one of claims 35-57, wherein the aberrant cell is from a cancer, in particular said cancer is an adenocarcinoma, more in particular a mucinous adenocarcinoma, a pancreatic cancer, in particular a pancreatic adenocarcinoma, more in particular a pancreatic ductal adenocarcinoma, a renal cell carcinoma, a sarcoma, a bladder, a colon, a rectal, colorectal, a gallbladder, a head and neck cancer, a prostrate, a uterus, a breast cancer, an ovarian cancer, a liver cancer, an endometrial cancer, a lung cancer, preferably a non-small cell lung cancer, preferably, more preferably invasive mucinous adenocarcinoma, or a primary or metastatic cancer.

59. An in vivo animal model comprising a polynucleotide fusion according to any one of claims 1-17 and/or expressing a polypeptide fusion encoded therefrom, wherein preferably the polynucleotide fusion or polypeptide fusion comprised by the animal model is comprised by an engrafted aberrant cell present in the animal model or comprised by the genome of the animal model.

60. A method of treatment of the in vivo animal model of claim 59 with an Erb2 and/or Erb3 targeting agent selected from the group consisting of a multispecific antibody comprising a first antigen-binding site that binds an extracellular part of ErbB-2 and a second antigen-binding site that binds an extracellular part of ErbB-3, a tyrosine kinase inhibitor of ErbB-2, a monospecific bivalent antibody comprising an antigen-binding site that bind an extracellular part of ErbB-2, a monospecific bivalent antibody comprising an antigen-binding site that bind an extracellular part of ErbB-3, or any combination thereof, said method comprising administering to the animal said Erb2 and/or Erb3 targeting agent.

61. A first and a second nucleic acid probe for use in an in-situ hybridization assay to detect a genetic rearrangement of VAPB, CADM1, CD44, SLC3A2, VTCN1, CDH1, CXADR, GTF2E2, CSMD1, PTN, ST14, THBS1, AGRN, PVALB, APP, WRN, DAAM1, ASPH, NOTCH2, CD74, SDC4, SLC4A4, ZFAT or DSCAML1 wherein:
the first probe for detection of a genetic rearrangement of VAPB specifically hybridizes to a VAPB sequence which is 5′ from the nucleic acid of position 43 of SEQ ID NO: 1, and the second probe specifically hybridizes to a VAPB sequence which is 3′ from the nucleic acid of position 43 of SEQ ID NO: 1;
the first probe for detection of a genetic rearrangement of CADM1 specifically hybridizes to a CADM1 sequence which is 5′ from the nucleic acid of position 53 of SEQ ID NO: 5, and the second probe specifically hybridizes to a CADM1 sequence which is 3′ from the nucleic acid of position 53 of SEQ ID NO: 5;
the first probe for detection of a genetic rearrangement of CD44 specifically hybridizes to a CD44 sequence which is 5′ from the nucleic acid of position 52 of SEQ ID NO: 9, and the second probe specifically hybridizes to a CD44 sequence which is 3′ from the nucleic acid of position 52 of SEQ ID NO: 9;
the first probe for detection of a genetic rearrangement of SLC3A2 specifically hybridizes to a SLC3A2 sequence which is 5′ from the nucleic acid of position 53 of SEQ ID NO: 13, and the second probe specifically hybridizes to a SLC3A2 sequence which is 3′ from the nucleic acid of position 53 of SEQ ID NO: 13;
the first probe for detection of a genetic rearrangement of VTCN1 specifically hybridizes to a VTCN1 sequence which is 5′ from the nucleic acid of position 65 of SEQ ID NO: 164, and the second probe specifically hybridizes to a VTCN1 sequence which is 3′ from the nucleic acid of position 65 of SEQ ID NO: 164;
the first probe for detection of a genetic rearrangement of CDH1 specifically hybridizes to a CDH1 sequence which is 5′ from the nucleic acid of position 119 of SEQ ID NO: 184, and the second probe specifically hybridizes to a CDH1 sequence which is 3′ from the nucleic acid of position 119 of SEQ ID NO: 184;
the first probe for detection of a genetic rearrangement of CXADR specifically hybridizes to a CXADR sequence which is 5′ from the nucleic acid of position 43 of SEQ ID NO: 215, and the second probe specifically hybridizes to a CXADR sequence which is 3′ from the nucleic acid of position 43 of SEQ ID NO: 215;
the first probe for detection of a genetic rearrangement of GTF2E2 specifically hybridizes to a GTF2E2 sequence which is 5′ from the nucleic acid of position 141 of SEQ ID NO: 231, and the second probe specifically hybridizes to a GTF2E2 sequence which is 3′ from the nucleic acid of position 141 of SEQ ID NO: 231;
the first probe for detection of a genetic rearrangement of CSMD1 specifically hybridizes to a CSMD1 sequence which is 5′ from the nucleic acid of position 88 of SEQ ID NO: 253, and the second probe specifically hybridizes to a CSMD1 sequence which is 3′ from the nucleic acid of position 88 of SEQ ID NO:253;
the first probe for detection of a genetic rearrangement of PTN specifically hybridizes to a PTN sequence which is 5′ from the nucleic acid of position 102 of SEQ ID NO: 311, and the second probe specifically hybridizes to a PTN sequence which is 3′ from the nucleic acid of position 102 of SEQ ID NO: 311;
the first probe for detection of a genetic rearrangement of ST14 specifically hybridizes to a ST14 sequence which is 5′ from the nucleic acid of position 95 of SEQ ID NO: 328, and the second probe specifically hybridizes to a ST14 sequence which is 3′ from the nucleic acid of position 95 of SEQ ID NO: 328;
the first probe for detection of a genetic rearrangement of AGRN specifically hybridizes to a AGRN sequence which is 5′ from the nucleic acid of position 106 of SEQ ID NO: 401, and the second probe specifically hybridizes to a AGRN sequence which is 3′ from the nucleic acid of position 106 of SEQ ID NO: 401;
the first probe for detection of a genetic rearrangement of THBS1 specifically hybridizes to a THBS1 sequence which is 5′ from the nucleic acid of position 56 of SEQ ID NO: 374, and the second probe specifically hybridizes to a THBS1 sequence which is 3′ from the nucleic acid of position 56 of SEQ ID NO: 374;
the first probe for detection of a genetic rearrangement of PVALB specifically hybridizes to a PVALB sequence which is 5′ from the nucleic acid of position 102 of SEQ ID NO: 435, and the second probe specifically hybridizes to a PVALB sequence which is 3′ from the nucleic acid of position 102 of SEQ ID NO: 435;
the first probe for detection of a genetic rearrangement of SLC3A2 specifically hybridizes to a SLC3A2 sequence which is 5′ from the nucleic acid of position 93 of SEQ ID NO: 452, and the second probe specifically hybridizes to a SLC3A2 sequence which is 3′ from the nucleic acid of position 93 of SEQ ID NO: 452;
the first probe for detection of a genetic rearrangement of APP specifically hybridizes to a APP sequence which is 5′ from the nucleic acid of position 54 of SEQ ID NO: 484, and the second probe specifically hybridizes to a APP sequence which is 3′ from the nucleic acid of position 54 of SEQ ID NO: 484;
the first probe for detection of a genetic rearrangement of WRN specifically hybridizes to a WRN sequence which is 5′ from the nucleic acid of position 96 of SEQ ID NO: 526, and the second probe specifically hybridizes to a WRN sequence which is 3′ from the nucleic acid of position 96 of SEQ ID NO: 526;
the first probe for detection of a genetic rearrangement of DAAM1 specifically hybridizes to a DAAM1 sequence which is 5′ from the nucleic acid of position 75 of SEQ ID NO: 603, and the second probe specifically hybridizes to a DAAM1 sequence which is 3′ from the nucleic acid of position 75 of SEQ ID NO: 603;
the first probe for detection of a genetic rearrangement of ASPH specifically hybridizes to a ASPH sequence which is 5′ from the nucleic acid of position 75 of SEQ ID NO: 633, and the second probe specifically hybridizes to a ASPH sequence which is 3′ from the nucleic acid of position 75 of SEQ ID NO: 633;
the first probe for detection of a genetic rearrangement of NOTCH2 specifically hybridizes to a NOTCH2 sequence which is 5′ from the nucleic acid of position 75 of SEQ ID NO: 691, and the second probe specifically hybridizes to a NOTCH2 sequence which is 3′ from the nucleic acid of position 75 of SEQ ID NO: 691;
the first probe for detection of a genetic rearrangement of CD74 specifically hybridizes to a CD74 sequence which is 5′ from the nucleic acid of position 75 of SEQ ID NO: 715, and the second probe specifically hybridizes to a CD74 sequence which is 3′ from the nucleic acid of position 75 of SEQ ID NO: 715;
the first probe for detection of a genetic rearrangement of SDC4 specifically hybridizes to a SDC4 sequence which is 5′ from the nucleic acid of position 75 of SEQ ID NO: 741, and the second probe specifically hybridizes to a SDC4 sequence which is 3′ from the nucleic acid of position 75 of SEQ ID NO: 741;
the first probe for detection of a genetic rearrangement of CD44 specifically hybridizes to a CD44 sequence which is 5′ from the nucleic acid of position 75 of SEQ ID NO: 759, and the second probe specifically hybridizes to a CD44 sequence which is 3′ from the nucleic acid of position 75 of SEQ ID NO: 759;
the first probe for detection of a genetic rearrangement of SLC4A4 specifically hybridizes to a SLC4A4 sequence which is 5′ from the nucleic acid of position 75 of SEQ ID NO: 763, and the second probe specifically hybridizes to a SLC4A4 sequence which is 3′ from the nucleic acid of position 75 of SEQ ID NO: 763;
the first probe for detection of a genetic rearrangement of SDC4 specifically hybridizes to a SDC4 sequence which is 5′ from the nucleic acid of position 75 of SEQ ID NO: 822, and the second probe specifically hybridizes to a SDC4 sequence which is 3′ from the nucleic acid of position 75 of SEQ ID NO: 822;
the first probe for detection of a genetic rearrangement of ZFAT specifically hybridizes to a ZFAT sequence which is 5′ from the nucleic acid of position 75 of SEQ ID NO: 826, and the second probe specifically hybridizes to a ZFAT sequence which is 3′ from the nucleic acid of position 75 of SEQ ID NO: 826; or
the first probe for detection of a genetic rearrangement of DSCAML1 specifically hybridizes to a DSCAML1 sequence which is 5′ from the nucleic acid of position 75 of SEQ ID NO: 866, and the second probe specifically hybridizes to a DSCAML1 sequence which is 3′ from the nucleic acid of position 75 of SEQ ID NO: 866.
